(19) United States
(12) Patent Application Publication
(10) Pub. No.: US 2023/0287138 A1
(43) Pub. Date: Sep. 14, 2023

(54) PROTEIN-DRUG CONJUGATES COMPRISING CAMPTOTHECIN ANALOGS AND METHODS OF USE THEREOF

(71) Applicant: Regneron Pharmaceuticals, Inc., Tarrytown, NY (US)

(72) Inventor: Amy HAN, Hockessin, DE (US)

(73) Assignee: Regneron Pharmaceuticals, Inc., Tarrytown, NY (US)

(21) Appl. No.: 18/095,467

(22) Filed: Jan. 10, 2023

Related U.S. Application Data
(60) Provisional application No. 63/298,786, filed on Jan. 12, 2022.

(51) Int. Cl. C07K 16/30; A61K 31/475; A61P 35/00

(52) U.S. Cl. CPC C07K 16/3015; A61K 31/475; A61P 35/00

(57) ABSTRACT

Described herein are protein-drug conjugates and compositions thereof that are useful, for example, for target-specific delivery of therapeutic moieties, e.g., camptothecin analogs and/or derivatives. In certain embodiments, provided are specific and efficient methods for producing protein-drug constructs (e.g., antibody-drug conjugates) utilizing a combination of transglutaminase and 1,3-cycloaddition techniques. Camptothecin analogs, antibody-drug conjugates, and compositions which comprise glutaminyl-modified antibodies and camptothecin analog payloads and are provided.

### CROSS-REFERENCE TO RELATED APPLICATIONS

This application claims priority to U.S. Provisional Application No. 63/298,786, filed Jan. 12, 2022. The entire contents of this application are incorporated herein by reference in their entirety.

### FIELD OF THE DISCLOSURE

The present disclosure relates to protein-drug conjugates (e.g., antibody-drug conjugates), pharmaceutical compositions, and methods of treating disease therewith. Also provided are methods for producing protein-drug conjugates utilizing a combination of transglutaminase and 1,3-cycloaddition techniques. More specifically, the present disclosure relates to protein-drug conjugates (e.g., antibody-drug conjugates) comprising pro-exatecan and exatecan.

### BACKGROUND OF THE DISCLOSURE

Proliferative diseases are characterized by uncontrolled growth and spread of abnormal cells. If the spread is not controlled, it can result in death. Abnormal proliferation, for example, cancer, is caused by both external factors (e.g., tobacco, chemicals, radiation and infectious organisms) and internal factors (inherited mutations, immune system conditions, the mutations that occur from metabolism). These causal factors may act together or in sequence to initiate or promote abnormal proliferation. Cancer is treated by surgery, radiation, chemotherapy, hormones and immunotherapy. However, there is a need for more effective anti-proliferation drugs.

The ideal anti-proliferation therapy would enable targeted delivery of highly cytotoxic agents to tumor cells and would leave normal cells unaffected. Conventional chemotherapeutic treatment is limited because of the toxic side-effects that arise from effects of the drug on non-cancerous cells. Various approaches to targeted drug delivery have been tried, including the use of conjugates of tumor targeted probes (such as antibodies or growth factors) with toxins such as pseudomonas or diphtheria toxins, which arrest the synthesis of proteins and cells. However, the side effects include reaction of the immune system due to non-human components of the conjugates. Further, the half-life of the drug conjugates was limited due to elimination from the circulation through renal filtration, and schematic degradation, uptake by the reticuloendothelial system (RES), and accumulation in non-targeted organs and tissues.

Another approach uses passive drug carriers such as polymers, liposomes, and polymeric micelles to take advantage of the hyper-permeability of vascular endothelia of tumor tissue. Polymeric drugs and macromolecules accumulate within solid tumors due to an enhanced permeability and retention mechanism. However, barriers of using such targeted deliveries include fast clearance of foreign particles from the blood, and technological hindrances in obtaining highly standardized, pharmaceutically acceptable drug delivery systems with the necessary specificity and selectivity for binding tumor cells.

Protein conjugates, such as antibody conjugates, utilize the selective binding of a binding agent to deliver a payload to targets within tissues of subjects. The payload can be a therapeutic moiety that is capable of taking action at the target.

Several techniques for conjugating linkers and payloads to antibodies are available. Many conjugates are prepared by non-selective covalent linkage to cysteine or lysine residues in the antibody. This non-selective technique can result in a heterogeneous mixture of products with conjugations at different sites and with different numbers of conjugations per antibody. Thus, there is a need in the art for methods and techniques that provide site-selective antibody conjugation.

There is a need in the art for additional safe and effective anti-tumor targeting agents that can bind to various antigens to provide enhanced the treatment of diseases such as cancer for use in monotherapy and combination therapies. In certain embodiments, the present disclosure meets the needs and provides other advantages.

The foregoing discussion is presented solely to provide a better understanding of the nature of the problems confronting the art and should not be construed in any way as an admission as to prior art nor should the citation of any reference herein be construed as an admission that such reference constitutes “prior art” to the instant application.

### SUMMARY OF THE DISCLOSURE

Various non-limiting aspects and embodiments of the disclosure are described below.

In one aspect, the present disclosure provides a compound having a structure according to Formula (A):

BA-(L1-B-L2-P)ₙ (A), wherein:

BA is an antibody or an antigen-binding fragment thereof; L1 is a first linker; B is a moiety comprising a triazole; L2 is a second linker; P is selected from the group consisting of P-I through P-IV:

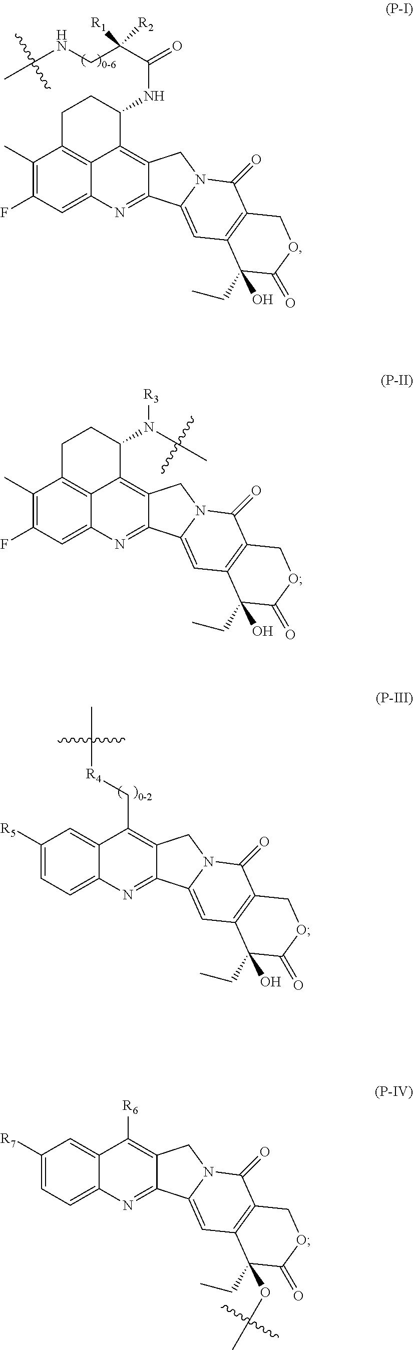

R¹ is hydrogen, C₁₋₆ alkyl, —(CH₂)ᵥ—OH, —(CH₂)ᵥ—NH₂, —(CH₂)ᵥ—C(O)OH, —(CH₂)ᵥ-phenyl, and —(CH₂)ᵥ—SO₂CH₃, wherein v is an integer from 0 to 12;
R² is hydrogen, C₁₋₆ alkyl, —(CH₂)ᵥ—OH, —(CH₂)ᵥ—NH₂, —(CH₂)ᵥ—C(O)OH, —(CH₂)ᵥ-phenyl, and —(CH₂)ᵥ—SO₂CH₃, wherein v is an integer from 0 to 12;
R³ is hydrogen —C₁₋₆ alkyl, —(CH₂)ᵥ—OH, —(CH₂)ᵥ—NH₂, —(CH₂)ᵥ—C(O)OH, —(CH₂)ᵥ-phenyl, —(CH₂)ᵥ—SO₂CH₃, and —CO—(CH₂)ᵥ—O—COCH₃, wherein v is an integer from 0 to 12;
R⁴ is —NH—, —N(—C₁₋₆ alkyl), —N(—C₁₋₆ alkyl)(—SO₂CH₃), —N(—C₁₋₆ alkyl)(—(CH₂)ᵥ—OH), —N(—C₁₋₆ alkyl)(CO—CH₂—NH₂), —N(—C₁₋₆ alkyl)(—(CH₂)ᵥ—O—CH₂—NH—CO—CH₂—NH₂), —N(—C₁₋₆ alkyl)(—CO—CH(NH₂)—(CH₂)ᵥ—COOH), —N(—C₁₋₆ alkyl)(—CO—CH(NH₂)—(CH₂)ᵥ—NH₂), —N(—C₁₋₆ alkyl)(—CO—CH(NH₂)—(CH₂)ᵥ-phenyl), or

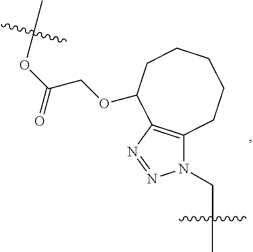

wherein v is an integer from 0 to 12;
R⁵ is H, —OH, —OCH₃, or

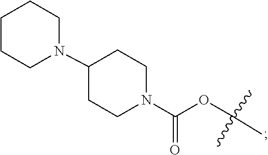 [SMILES: *OC(=O)N1CCC(N2CCCCC2)CC1]

R⁶ is hydrogen —C₁₋₆ alkyl, —(CH₂)ᵥ—OH, —(CH₂)ᵥ—NH₂, —(CH₂)ᵥ-phenyl, —(CH₂)ᵥ—N₃, —(CH₂)ᵥ—NH—CH₂-phenyl, —(CH₂)ᵥ—NMe-CH₂-phenyl-OMe, —(CH₂)ᵥ—NH—(CH₂)ᵥ—N₃, —(CH₂)ᵥ—NH—(CH₂)ᵥ—CCH; —(CH₂)ᵥ—NH—CO—CH(NH₂)—(CH₂)ᵥ-phenyl, —(CH₂)ᵥ—NH—CO—CH(NH₂)—(CH₂)ᵥ—N₃, —(CH₂)ᵥ—NH—CO—CH(NH₂)—(CH₂)ᵥ—NH₂, —(CH₂)ᵥ—NH—CO—CH(NH₂)—(CH₂)ᵥ—COOH,

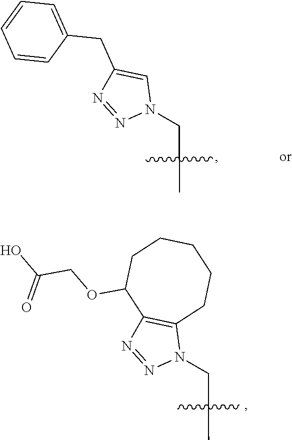

wherein v is an independently an integer from 0 to 12;
R⁷ is H, —OH, —OCH₃, or

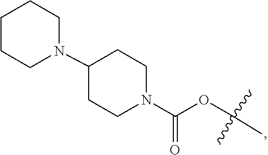 [SMILES: *OC(=O)N1CCC(N2CCCCC2)CC1]

and n is an integer from 1 to 12.

In one embodiment, the first linker L1 is connected to the side chain of a glutamine residue of the BA.

In one embodiment, the BA is an antibody or an antigen-binding fragment thereof.

In one embodiment, the BA is an antibody or an antigen-binding fragment thereof.

In one embodiment, the BA comprises one or more glutamine residues.

In one embodiment, the glutamine residues are naturally present in said BA, or are introduced to the BA by site-specific modification of one or more amino acids.

In one embodiment, the BA is an anti-HER2 antibody, an anti-STEAP2 antibody, an anti-MET antibody, an anti-EGFRVIII antibody, an anti-MUC16 antibody, an anti-PRLR antibody, an anti-PSMA antibody, an anti-FGFR2 antibody, an anti-FOLR1 antibody, an anti-HER2/HER2 bispecific antibody, an anti-MET/MET bispecific antibody, or an antigen-binding fragment thereof.

In one embodiment, the BA is an anti-HER2 antibody.

In one embodiment, the BA targets a cancer selected from the group consisting of breast cancer, ovarian cancer, prostate cancer, lung cancer, liver cancer, and brain cancer.

In one embodiment, the glutamine residue, sometimes denoted as Q, is naturally present in a CH2 or CH3 domain of the BA. In one embodiment, the glutamine residue is introduced to the BA by site-specifically modifying one or more amino acids. In one embodiment, the glutamine residue is Q295 or N297Q.

In one embodiment, L1 comprises C₁₋₆ alkyl, phenyl, aralkyl-NH—, —C(O)—, —(CH₂)ᵤ—NH—C(O)—, —(CH₂)ᵤ—C(O)—NH—, —(CH₂—CH₂—O)ᵥ—, —(CH₂)ᵥ—(O—CH₂—CH₂)ᵥ—C(O)—NH—, a peptide unit comprising from 2 to 4 amino acids, or combinations thereof, each of which may be optionally substituted with one or more of —S—, —S(O₂)—, —C(O)—, —C(O₂)—; and —CO₂H, wherein subscripts u and v are independently an integer from 1 to 8.

In one embodiment, L1 is selected from the group consisting of:

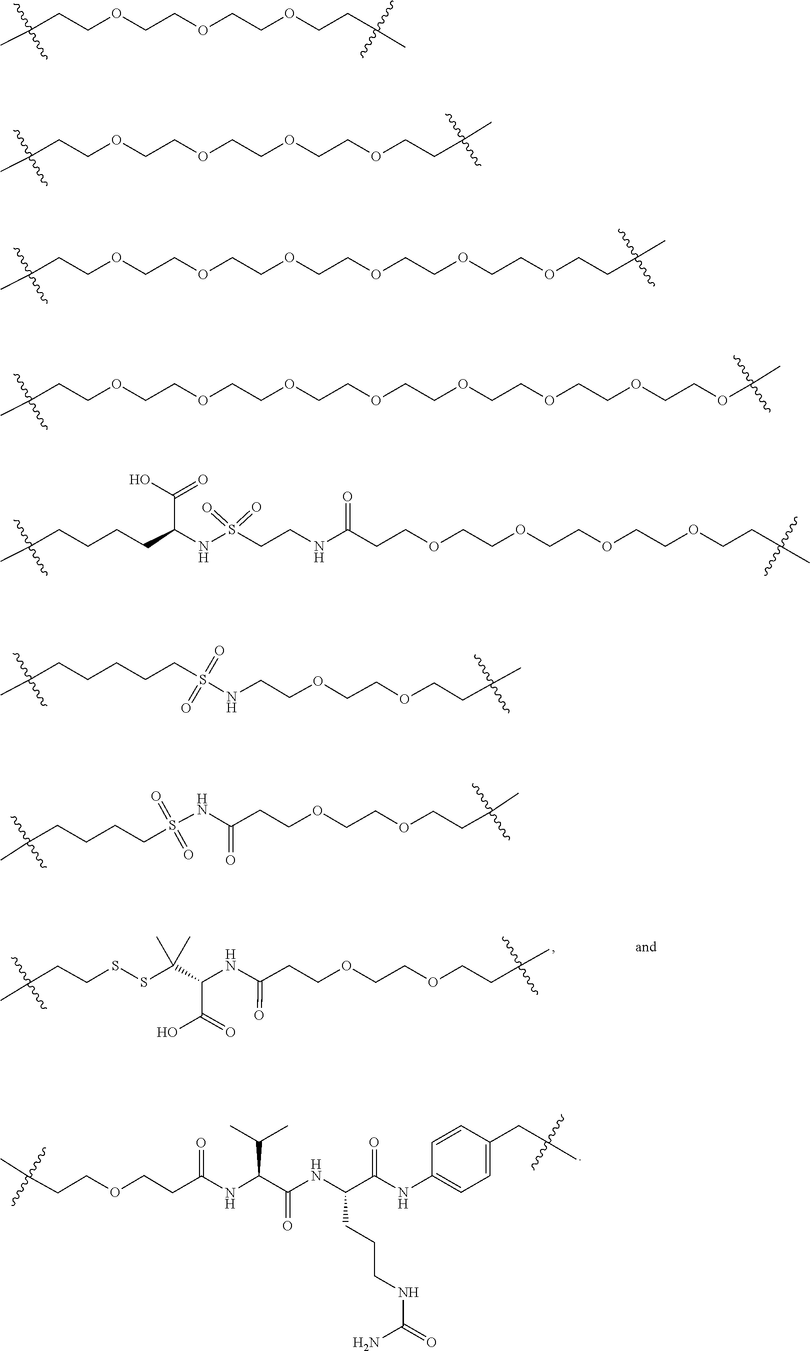

In one embodiment, L1 is

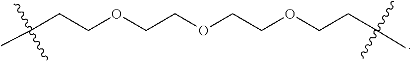 [SMILES: *CCOCCOCCOCC*]

In one embodiment, L1 is selected from the group consisting of:

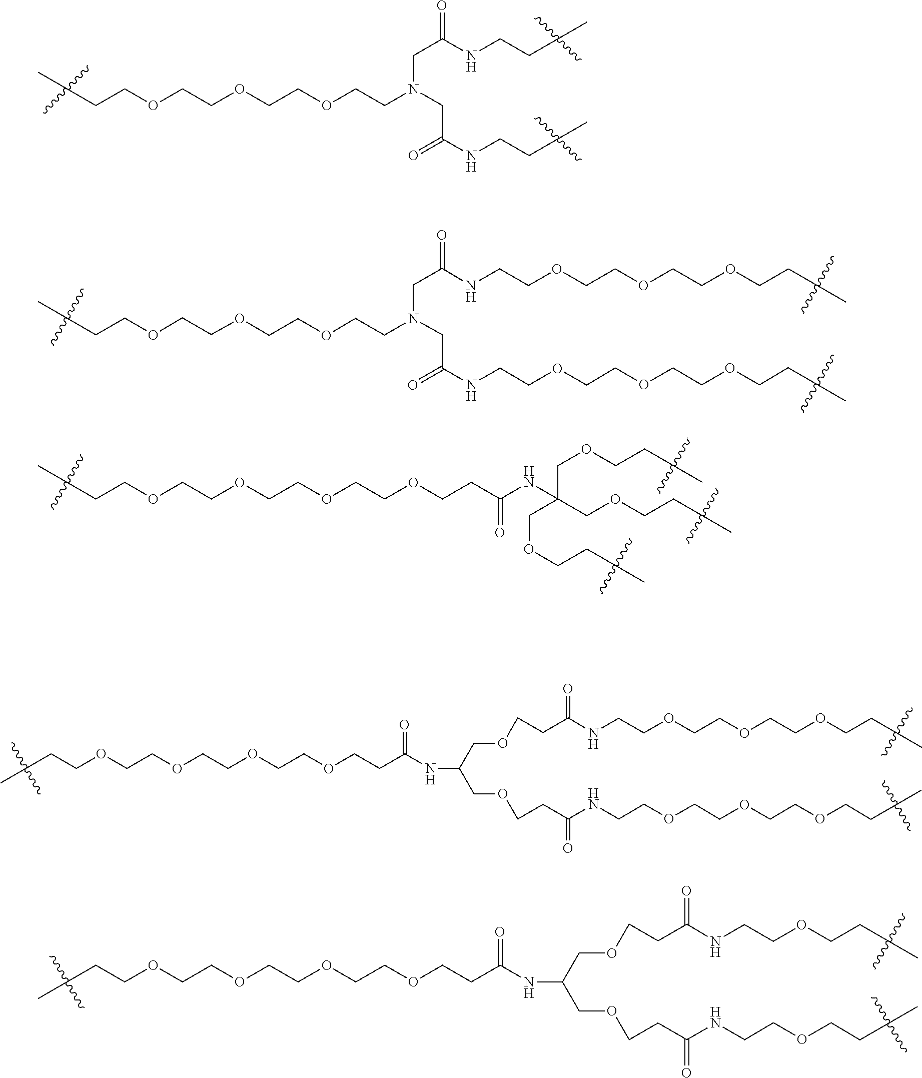 [SMILES: *CCNC(=O)CN(CCOCCOCCOCC*)CC(=O)NCC* ; *CCOCCOCCOCCNC(=O)CN(CCOCCOCCOCC*)CC(=O)NCCOCCOCCOCC* ; *CCOCCOCCOCCOCCC(=O)NC(COCC*)(COCC*)COCC* ; *CCOCCOCCOCCNC(=O)CCOCC(COCCC(=O)NCCOCCOCCOCC*)NC(=O)CCOCCOCCOCCOCC* ; *CCOCCNC(=O)CCOCC(COCCC(=O)NCCOCC*)NC(=O)CCOCCOCCOCCOCC*]

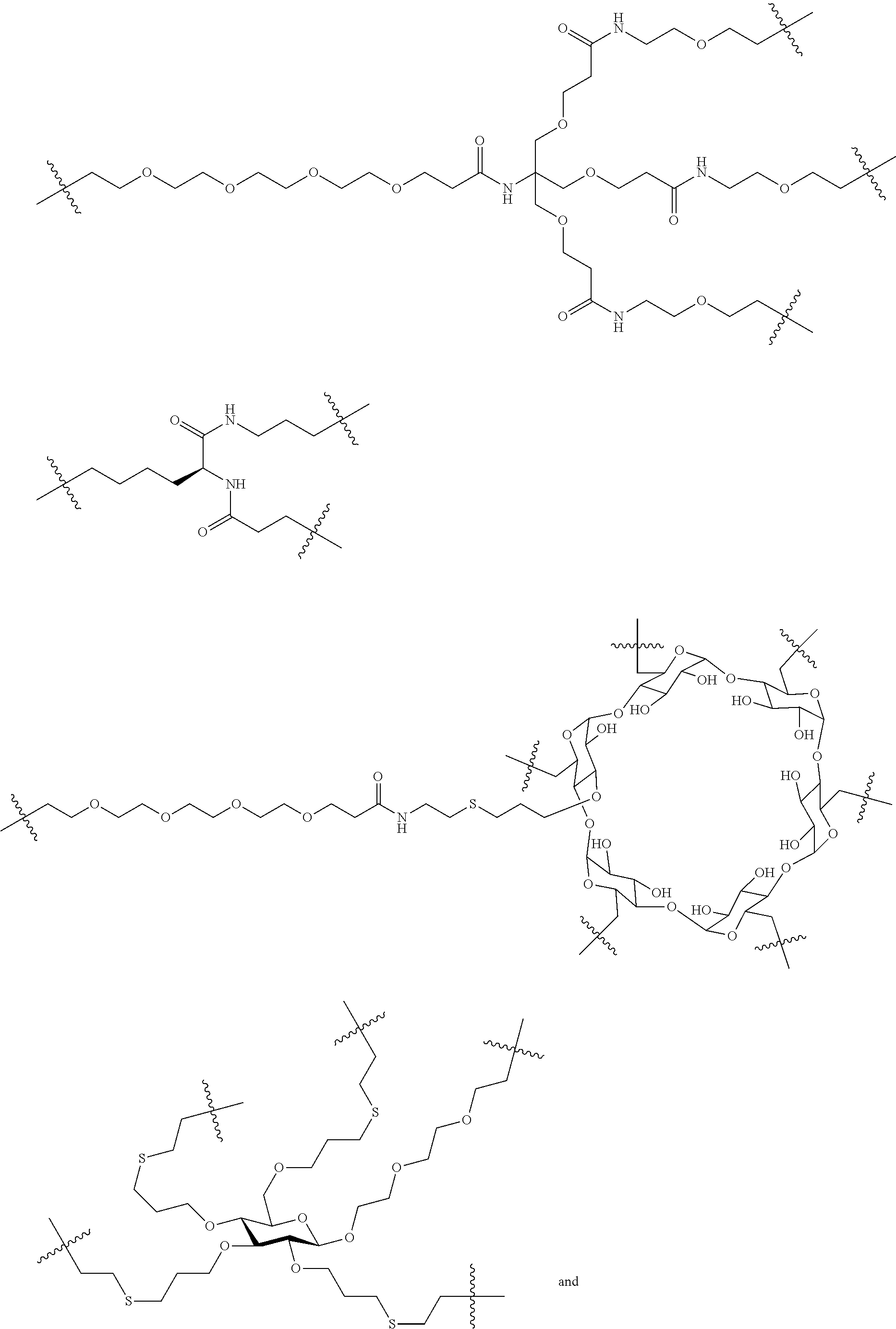

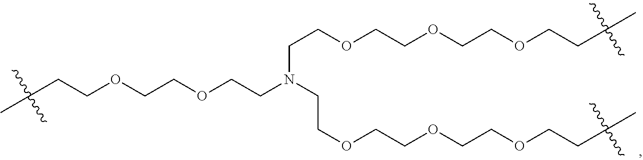 [SMILES: *CCOCCOCCOCCN(CCOCCOCC*)CCOCCOCCOCC*]

or a pharmaceutically acceptable salt thereof.

In one embodiment, B is selected from the group consisting of:

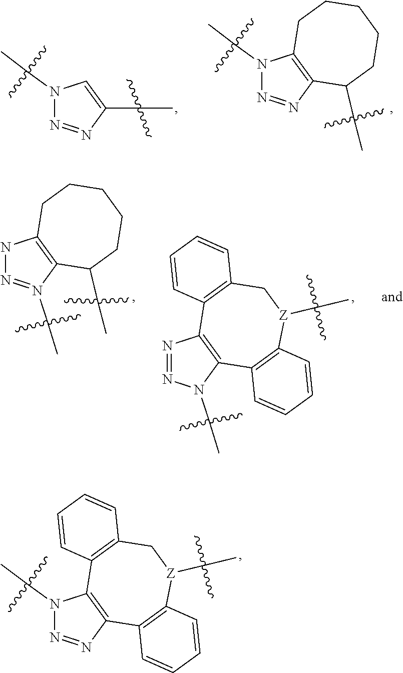

where Z is C or N.

In one embodiment, B is

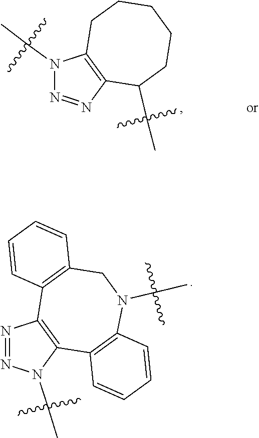 [SMILES: *C1CCCCCc2c1nnn2* ; *N1Cc2ccccc2-c2nnn(*)c2-c2ccccc21]

In one embodiment, L2 has a structure according to Formula (L2):

-SP1-AA-SP2- (L2),

wherein SP1 is absent or a first spacer unit; AA is absent or a peptide unit comprising from 2 to 4 amino acids; SP2 is absent or a second spacer unit covalently attached to the P, provided that at least one of SP1, AA and SP2 is not absent.

In one embodiment, SP1 is absent or selected from the group consisting of

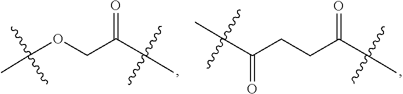 [SMILES: *OCC(*)=O ; *C(=O)CCC(*)=O]

C₁₋₆ alkyl, —(CH₂—CH₂—O)ᵥ—, —NH—, —C(O)—, —NH—C(O)—, —NH—(CH₂)ᵤ—, —NH—(CH₂)ᵤ—C(O)—, —NH—(CH₂—CH₂—O)ᵥ—, —NH—(CH₂—CH₂—O)ᵥ—C(O)—, —NH—(CH₂—CH₂—O)ᵥ—(CH₂)ᵤ—, —NH—(CH₂—CH₂—O)ᵥ—(CH₂)ᵤ—C(O)—, —(CH₂)ᵤ—NH—C(O)—, —NH—(CH₂)ᵤ—NH—C(O)—, —NH—(CH₂)ᵤ—C(O)—NH—, and combinations thereof; wherein subscripts u and v are independently an integer from 1 to 8.

In one embodiment, AA is a peptide unit comprising from 2 to 4 amino acids selected from alanine, glycine, valine, proline, glutamic acid, lysine, phenylalanine, and citrulline, and combinations thereof. In one embodiment, AA is valine-citrulline, glutamic acid-valine-citrulline, glycine-glycine-glycine, or glycine-glycine-glycine-glycine.

In one embodiment, SP2 is absent or selected from the group consisting of

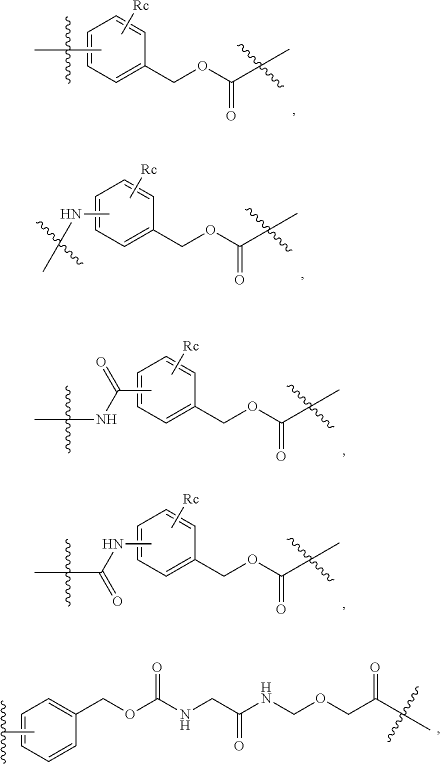

and combinations thereof, wherein R, is independently at each occurrence absent or a group selected from

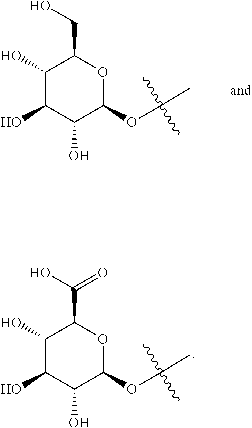 [SMILES: *O[C@@H]1O[C@H](CO)[C@@H](O)[C@H](O)[C@H]1O ; *O[C@@H]1O[C@H](C(=O)O)[C@@H](O)[C@H](O)[C@H]1O]

In one embodiment, L2 is selected from the group consisting of:

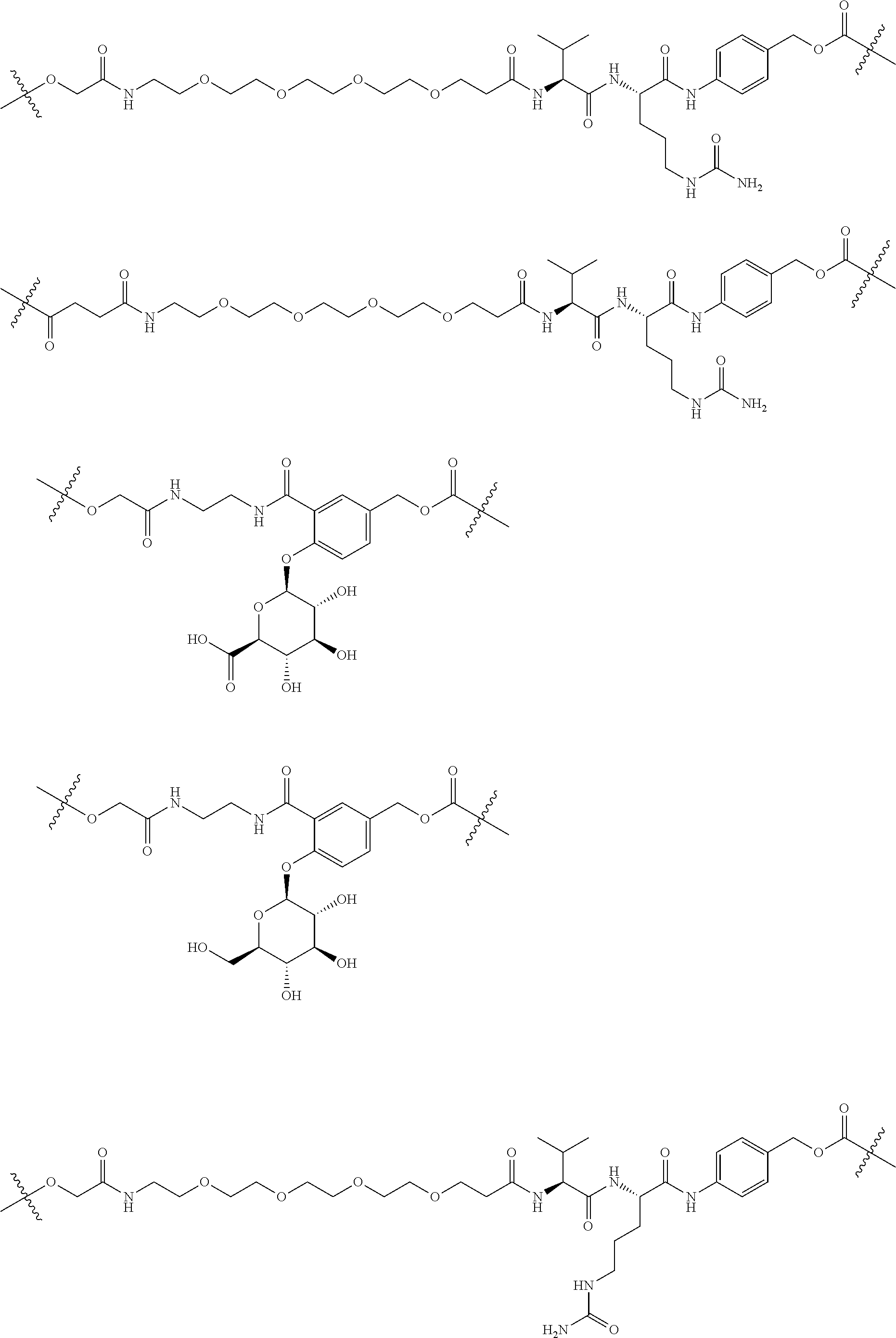 [SMILES: *OCC(=O)NCCOCCOCCOCCOCCC(=O)N[C@H](C(=O)N[C@@H](CCCNC(N)=O)C(=O)Nc1ccc(COC(*)=O)cc1)C(C)C ; *C(=O)CCC(=O)NCCOCCOCCOCCOCCC(=O)N[C@H](C(=O)N[C@@H](CCCNC(N)=O)C(=O)Nc1ccc(COC(*)=O)cc1)C(C)C ; *OCC(=O)NCCNC(=O)c1cc(COC(*)=O)ccc1O[C@@H]1O[C@H](C(=O)O)[C@@H](O)[C@H](O)[C@H]1O ; *OCC(=O)NCCNC(=O)c1cc(COC(*)=O)ccc1O[C@@H]1O[C@H](CO)[C@@H](O)[C@H](O)[C@H]1O ; *OCC(=O)NCCOCCOCCOCCOCCC(=O)N[C@H](C(=O)N[C@@H](CCCNC(N)=O)C(=O)Nc1ccc(COC(*)=O)cc1)C(C)C]

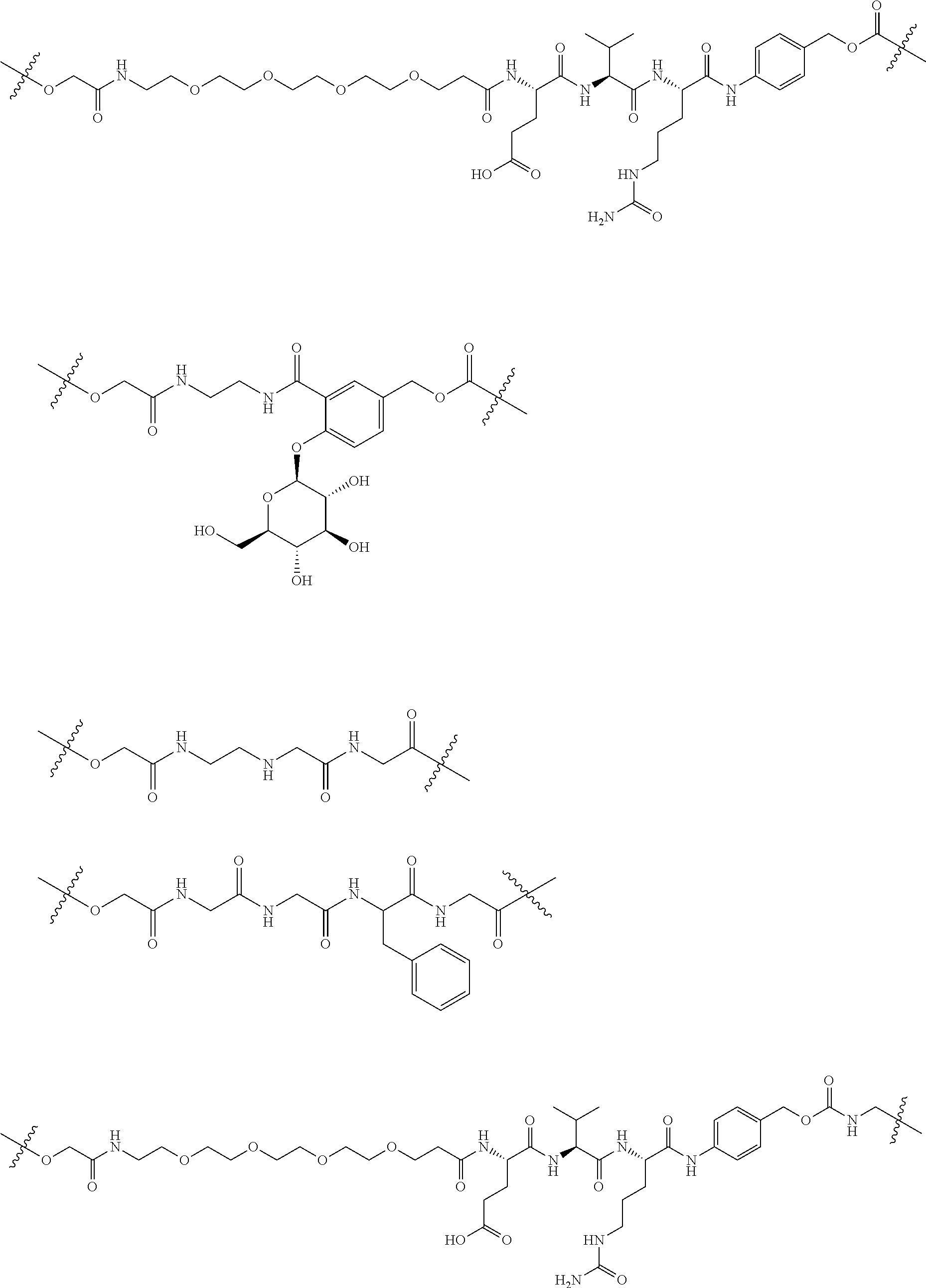 [SMILES: *OCC(=O)NCCOCCOCCOCCOCCC(=O)N[C@@H](CCC(=O)O)C(=O)N[C@H](C(=O)N[C@@H](CCCNC(N)=O)C(=O)Nc1ccc(COC(*)=O)cc1)C(C)C ; *OCC(=O)NCCNC(=O)c1cc(COC(*)=O)ccc1O[C@@H]1O[C@H](CO)[C@@H](O)[C@H](O)[C@H]1O ; *OCC(=O)NCCNCC(=O)NCC(*)=O ; *OCC(=O)NCC(=O)NCC(=O)NC(Cc1ccccc1)C(=O)NCC(*)=O ; *CNC(=O)OCc1ccc(NC(=O)[C@H](CCCNC(N)=O)NC(=O)[C@@H](NC(=O)[C@H](CCC(=O)O)NC(=O)CCOCCOCCOCCOCCNC(=O)CO*)C(C)C)cc1]

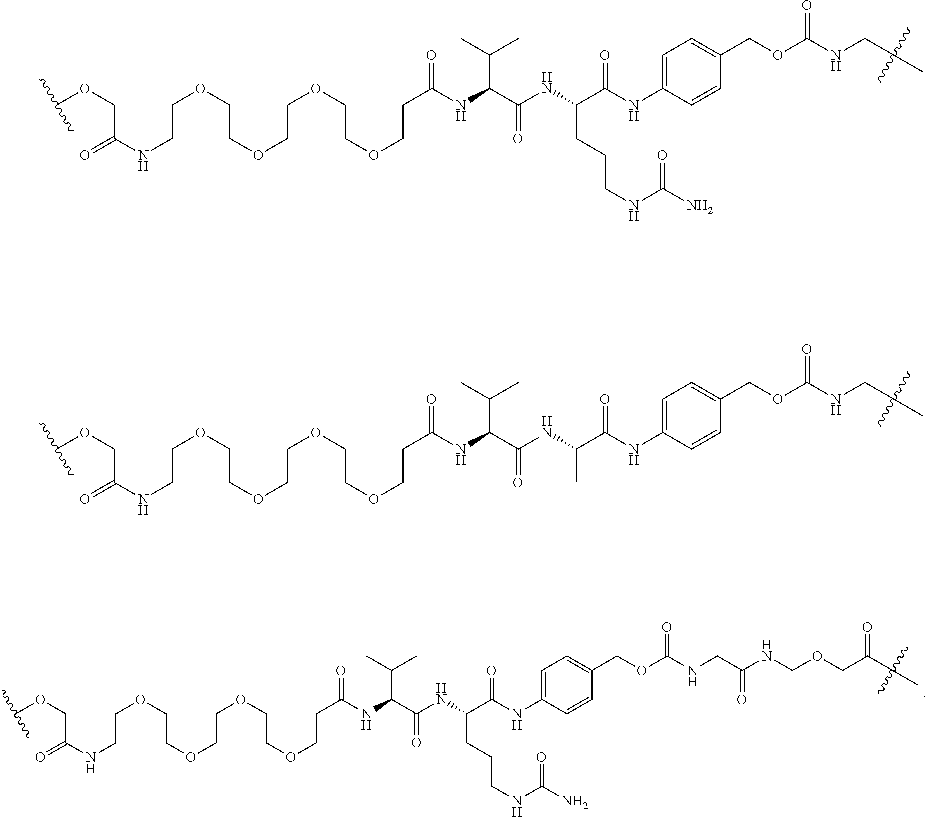 [SMILES: *CNC(=O)OCc1ccc(NC(=O)[C@H](CCCNC(N)=O)NC(=O)[C@@H](NC(=O)CCOCCOCCOCCOCCNC(=O)CO*)C(C)C)cc1 ; *CNC(=O)OCc1ccc(NC(=O)[C@H](C)NC(=O)[C@@H](NC(=O)CCOCCOCCOCCOCCNC(=O)CO*)C(C)C)cc1 ; *OCC(=O)NCCOCCOCCOCCOCCC(=O)N[C@H](C(=O)N[C@@H](CCCNC(N)=O)C(=O)Nc1ccc(COC(=O)NCC(=O)NCOCC(*)=O)cc1)C(C)C]

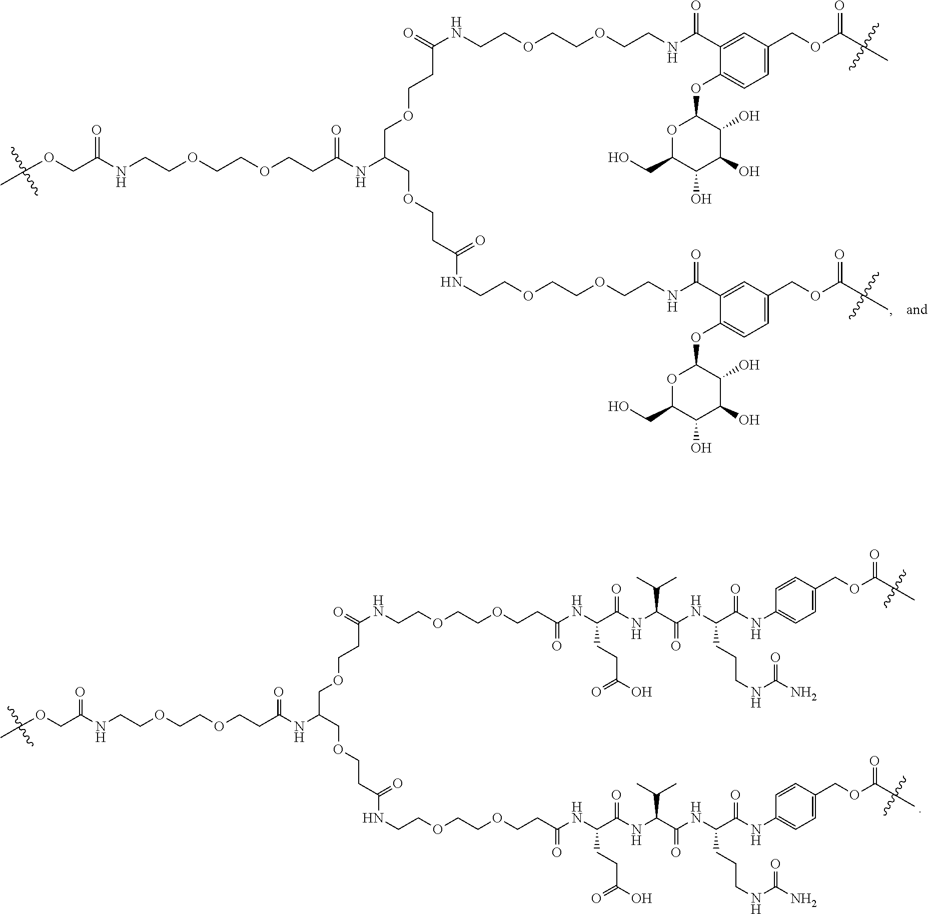 [SMILES: *OCC(=O)NCCOCCOCCC(=O)NC(COCCC(=O)NCCOCCOCCNC(=O)c1cc(COC(*)=O)ccc1O[C@@H]1O[C@H](CO)[C@@H](O)[C@H](O)[C@H]1O)COCCC(=O)NCCOCCOCCNC(=O)c1cc(COC(*)=O)ccc1O[C@@H]1O[C@H](CO)[C@@H](O)[C@H](O)[C@H]1O ; *OCC(=O)NCCOCCOCCC(=O)NC(COCCC(=O)NCCOCCOCCC(=O)N[C@@H](CCC(=O)O)C(=O)N[C@H](C(=O)N[C@@H](CCCNC(N)=O)C(=O)Nc1ccc(COC(*)=O)cc1)C(C)C)COCCC(=O)NCCOCCOCCC(=O)N[C@@H](CCC(=O)O)C(=O)N[C@H](C(=O)N[C@@H](CCCNC(N)=O)C(=O)Nc1ccc(COC(*)=O)cc1)C(C)C]

In one embodiment, n is 4. In one embodiment, n is 2. In one embodiment, n is 8.

In one embodiment, the compound has a structure selected from the group consisting of:

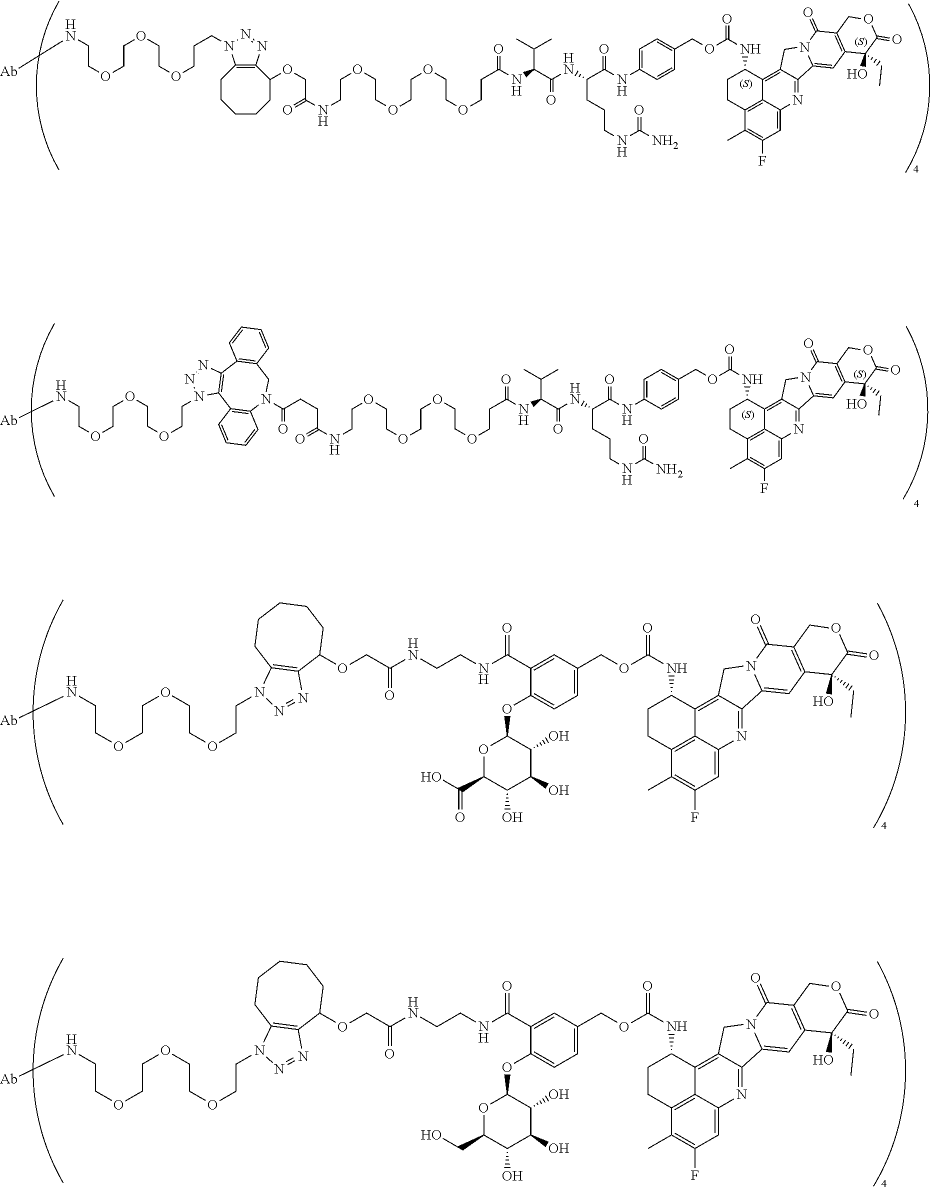

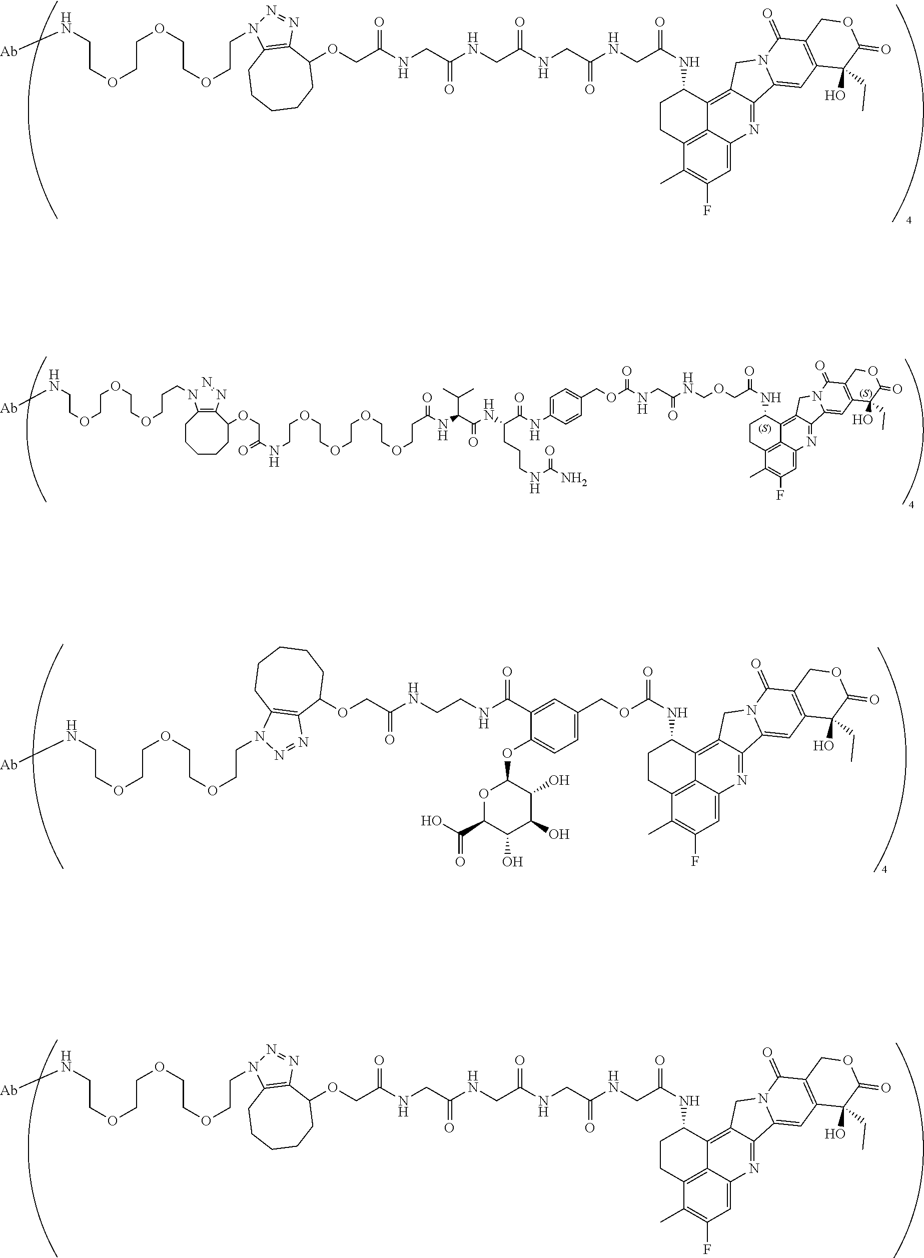

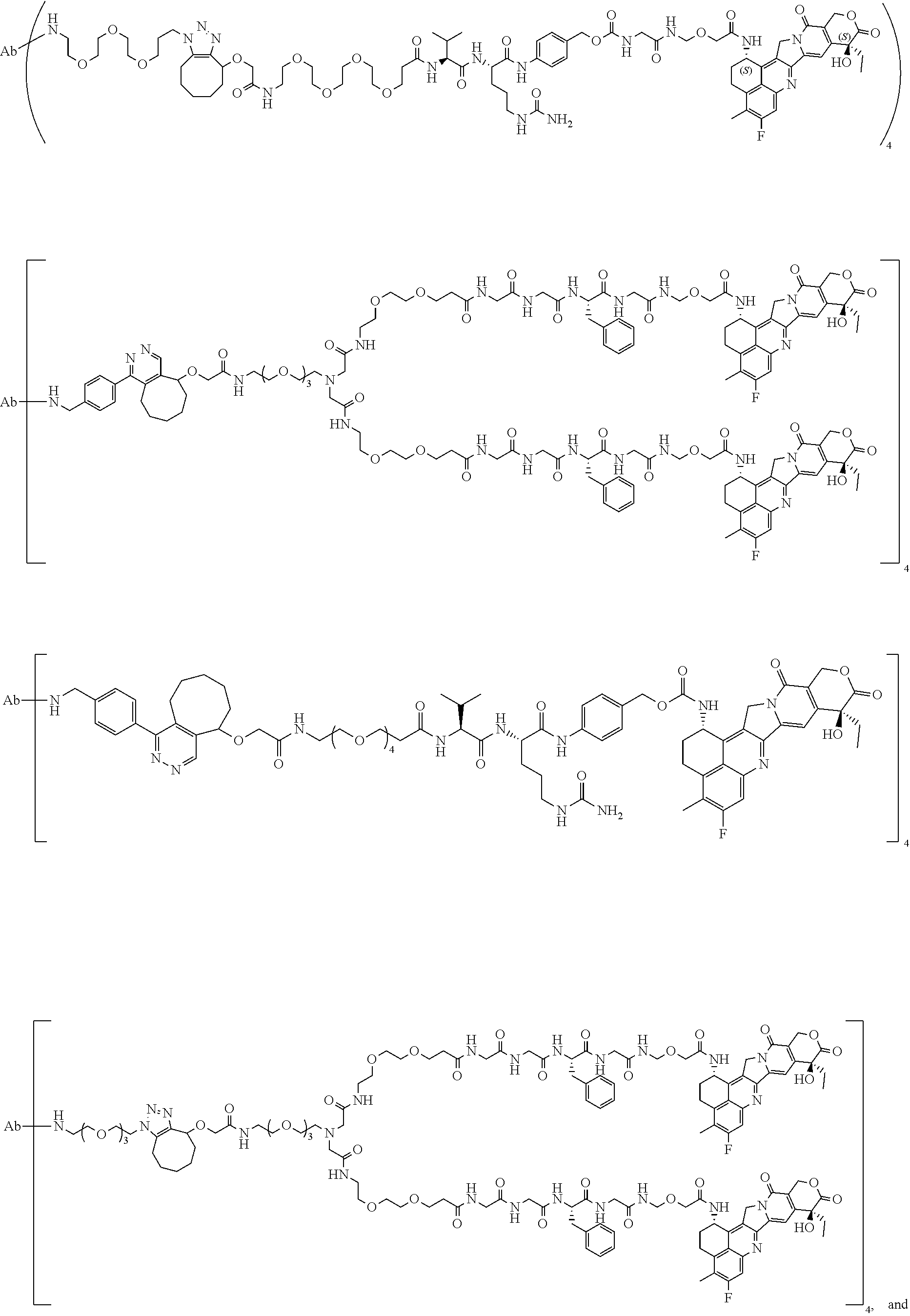

or a pharmaceutically acceptable salt thereof.

In another aspect, the present disclosure presents a compound according to Formula (Alk-L2-P):

Alk-SP1-AA-SP2-P (Alk-L2-P),
wherein Alk is a moiety comprising an alkyne; SP1 is absent or a first spacer unit; AA is absent or a peptide unit comprising from 2 to 4 amino acids; SP2 is absent or a second spacer unit;
P is selected from the group consisting of P-I through P-IV:

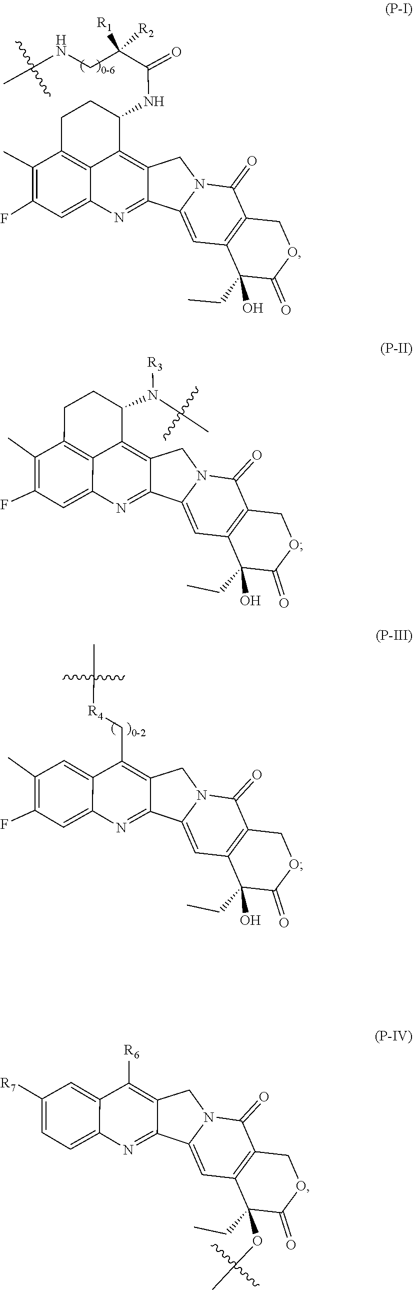

P is selected from the group consisting of P-I through P-IV:

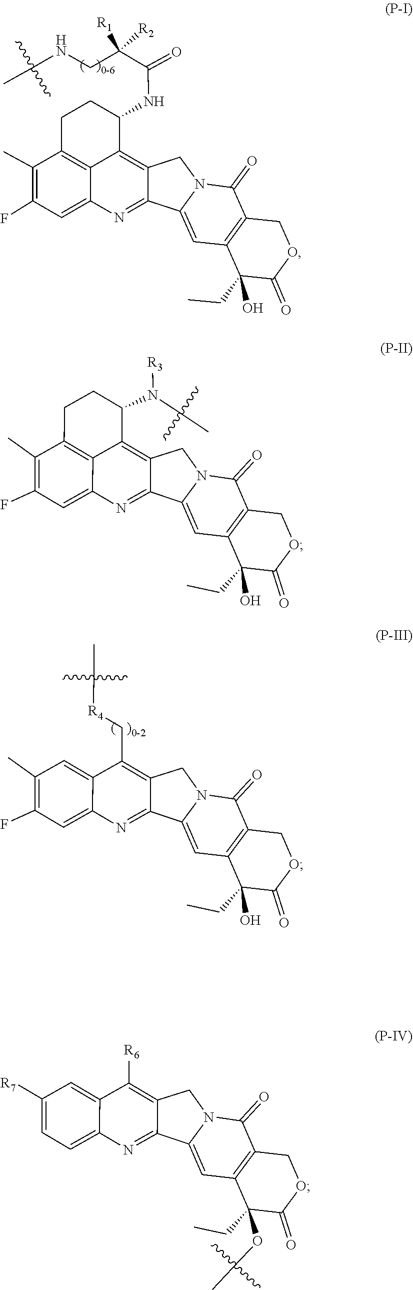

R₁ is hydrogen, C₁₋₆ alkyl, —(CH₂)ᵥ—OH, —(CH₂)ᵥ—NH₂, —(CH₂)ᵥ—C(O)OH, —(CH₂)ᵥ-phenyl, and —(CH₂)ᵥ—SO₂CH₃, wherein v is an integer from 0 to 12;
R² is hydrogen, C₁₋₆ alkyl, —(CH₂)ᵥ—OH, —(CH₂)ᵥ—NH₂, —(CH₂)ᵥ—C(O)OH, —(CH₂)ᵥ-phenyl, and —(CH₂)ᵥ—SO₂CH₃, wherein v is an integer from 0 to 12;
R³ is hydrogen —C₁₋₆ alkyl, —(CH₂)ᵥ—OH, —(CH₂)ᵥ—NH₂, —(CH₂)ᵥ—C(O)OH, —(CH₂)ᵥ-phenyl, —(CH₂)ᵥ—SO₂CH₃, and —CO—(CH₂)ᵥ—O—COCH₃, wherein v is an integer from 0 to 12;
R⁴ is —NH—, —N(—C₁₋₆ alkyl), —N(—C₁₋₆ alkyl)(—SO₂CH₃), —N(—C₁₋₆ alkyl)(—(CH₂)ᵥ—OH), —N(—C₁₋₆ alkyl)(CO—CH₂—NH₂), —N(—C₁₋₆ alkyl)(—(CH₂)ᵥ—O—CH₂—NH—CO—CH₂—NH₂), —N(—C₁₋₆ alkyl)(—CO—CH(NH₂)—(CH₂)ᵥ—COOH), —N(—C₁₋₆ alkyl)(—CO—CH(NH₂)—(CH₂)ᵥ—NH₂), —N(—C₁₋₆ alkyl)(—CO—CH(NH₂)—(CH₂)ᵥ-phenyl), or

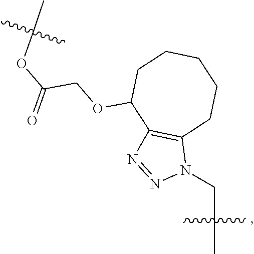

wherein v is an integer from 0 to 12;

R⁵ is H, —OH, —OCH₃, or

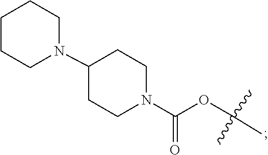 [SMILES: *OC(=O)N1CCC(N2CCCCC2)CC1]

R⁶ is hydrogen —C₁₋₆ alkyl, —(CH₂)ᵥ—OH, —(CH₂)ᵥ—NH₂, —(CH₂)ᵥ-phenyl, —(CH₂)ᵥ—N₃, —(CH₂)ᵥ—NH—CH₂-phenyl, —(CH₂)ᵥ—NMe-CH₂-phenyl-OMe, —(CH₂)ᵥ—NH—(CH₂)ᵥ—N₃, —(CH₂)ᵥ—NH—(CH₂)ᵥ—CCH; —(CH₂)ᵥ—NH—CO—CH(NH₂)—(CH₂)ᵥ-phenyl, —(CH₂)ᵥ—NH—CO—CH(NH₂)—(CH₂)ᵥ—N₃, —(CH₂)ᵥ—NH—CO—CH(NH₂)—(CH₂)ᵥ—NH₂, —(CH₂)ᵥ—NH—CO—CH(NH₂)—(CH₂)ᵥ—COOH,

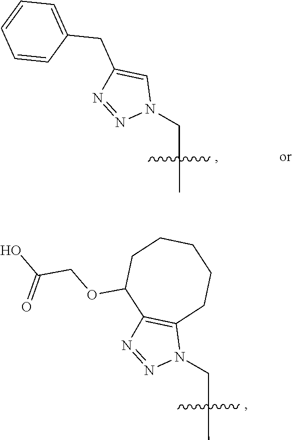

wherein v is an independently an integer from 0 to 12;
R⁷ is H, —OH, —OCH₃, or

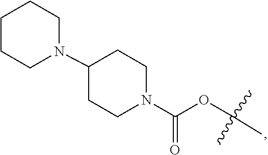 [SMILES: *OC(=O)N1CCC(N2CCCCC2)CC1]

or a pharmaceutically acceptable salt thereof.

In one embodiment, the compound according to Formula (Alk-L2-P) is selected from the group consisting of:

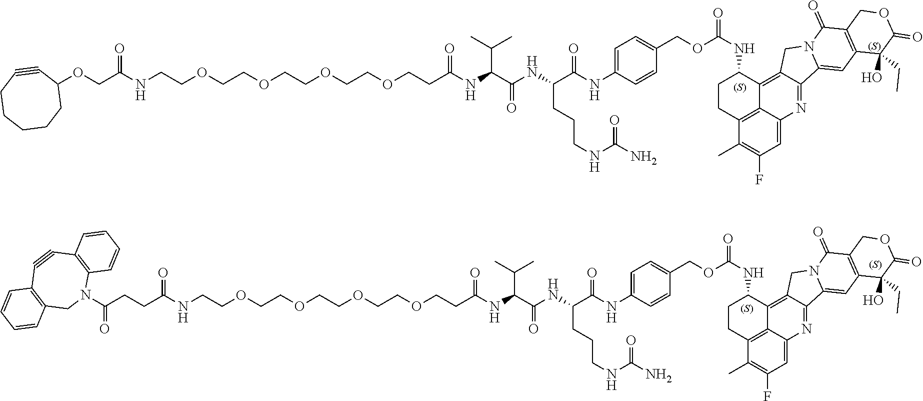

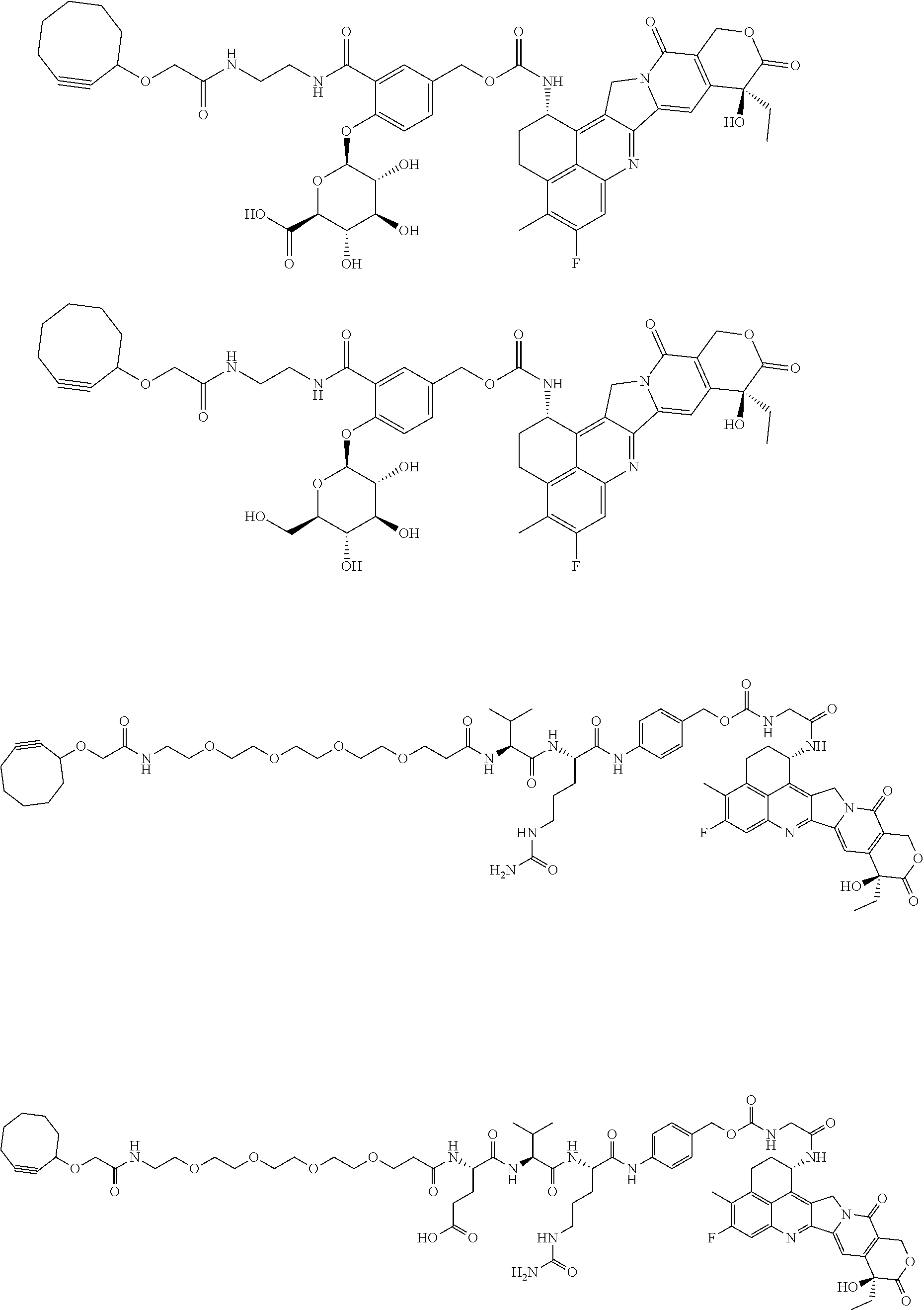

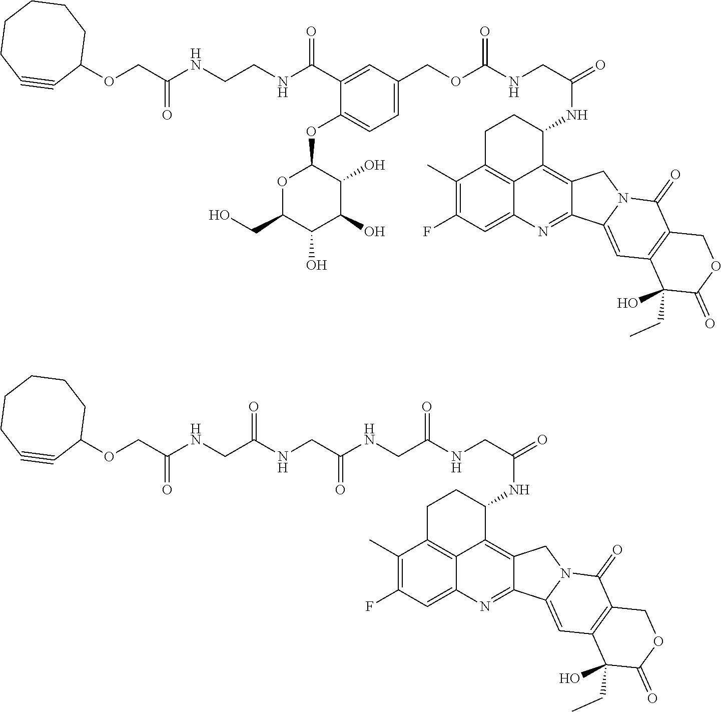 [SMILES: CC[C@@]1(O)C(=O)OCc2c1cc1n(c2=O)Cc2c-1nc1cc(F)c(C)c3c1c2[C@@H](NC(=O)CNC(=O)OCc1ccc(O[C@@H]2O[C@H](CO)[C@@H](O)[C@H](O)[C@H]2O)c(C(=O)NCCNC(=O)COC2C#CCCCCC2)c1)CC3 ; CC[C@@]1(O)C(=O)OCc2c1cc1n(c2=O)Cc2c-1nc1cc(F)c(C)c3c1c2[C@@H](NC(=O)CNC(=O)CNC(=O)CNC(=O)CNC(=O)COC1C#CCCCCC1)CC3]

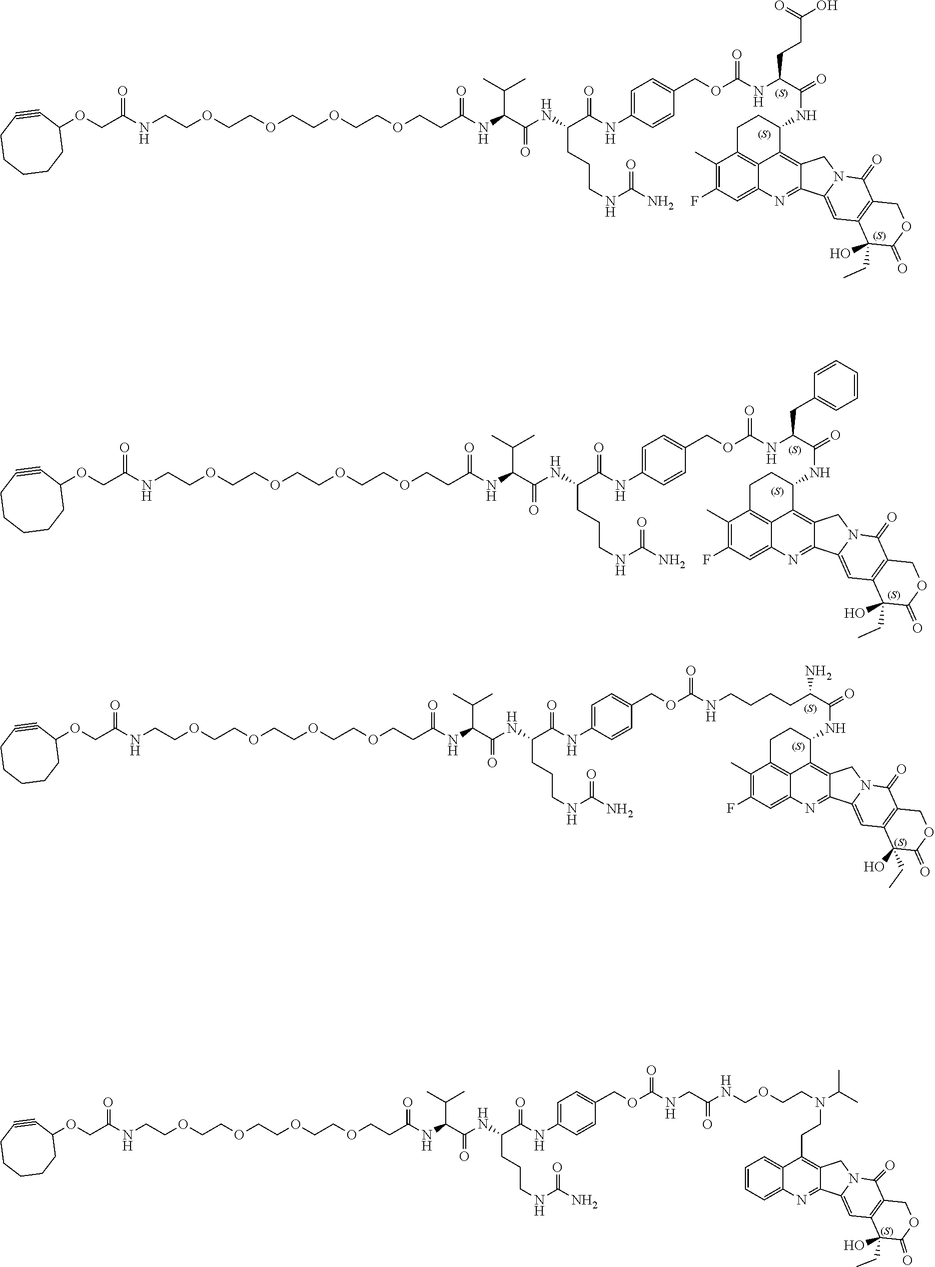

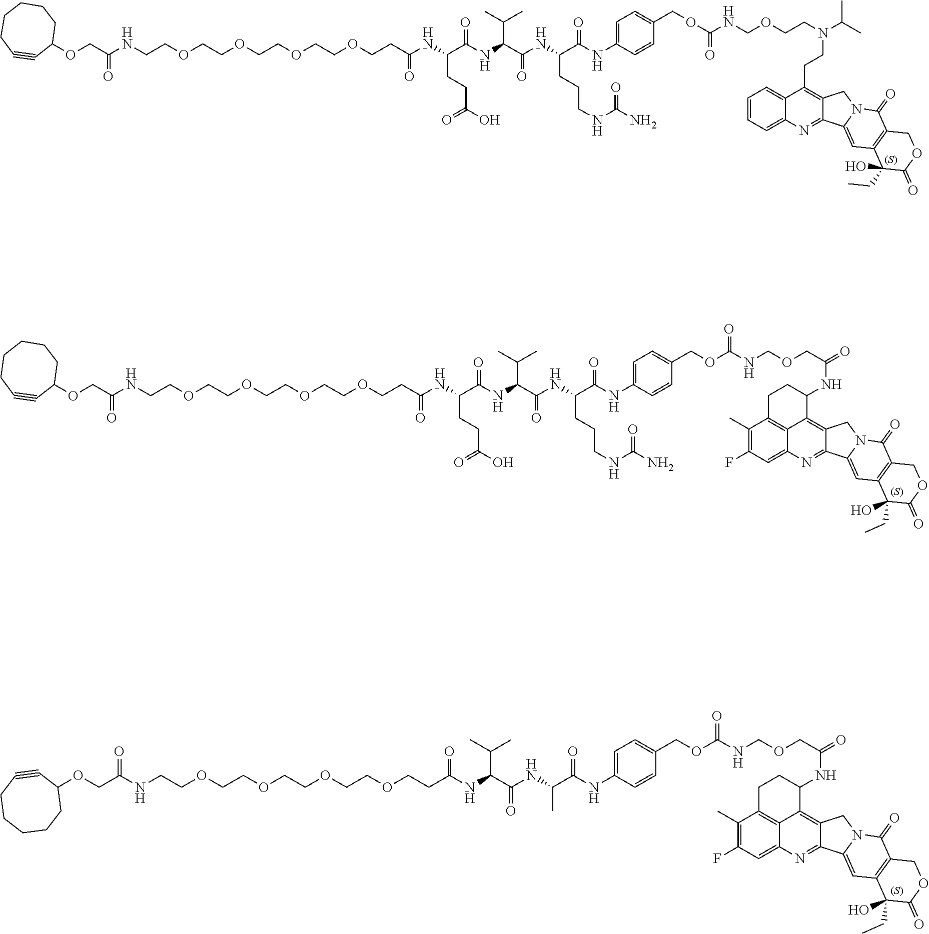

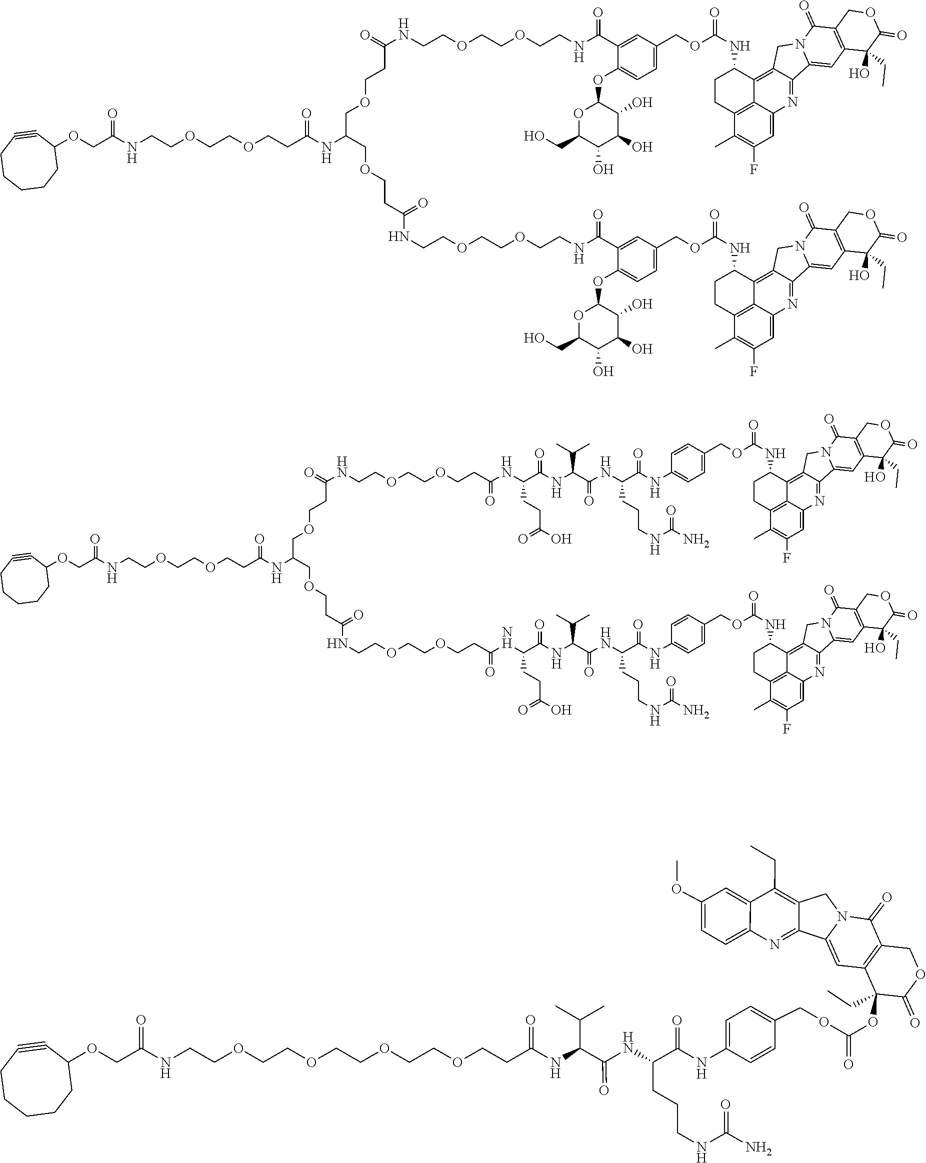

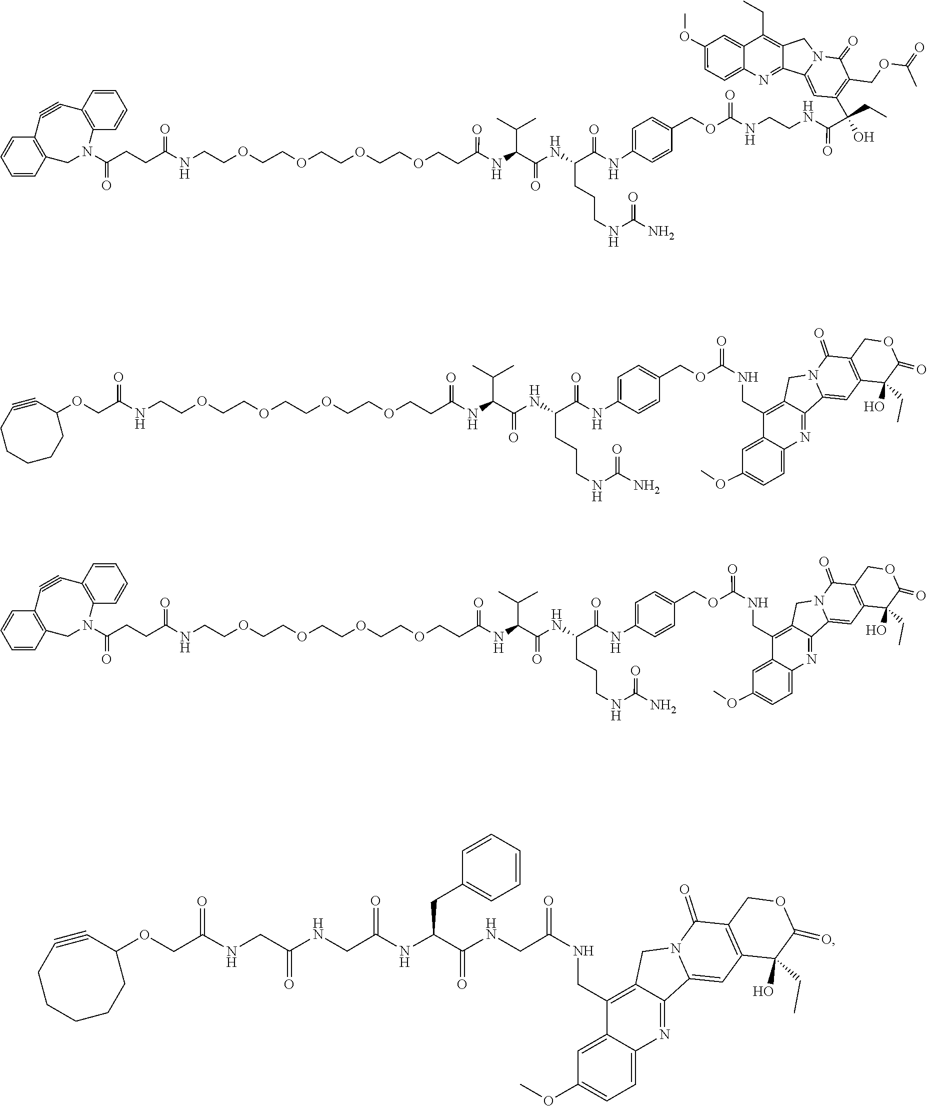

or a pharmaceutically acceptable salt thereof.

In yet another aspect, the present disclosure provides a composition comprising a population of compounds according to any of the above embodiments, having a drug-antibody ratio (DAR) of about 0.5 to about 12.0.

In one embodiment, the population of compounds has a DAR of about 1.0 to about 2.5. In one embodiment, the population of compounds has a DAR of about 2. In one embodiment, the population of compounds has a DAR of about 3.0 to about 4.5. In one embodiment, the population of compounds has a DAR of about 4. In one embodiment, the population of compounds has a DAR of about 6.5 to about 8.5. In one embodiment, the population of compounds has a DAR of about 8.

In yet another aspect, the present disclosure provides a compound having a structure selected from the group consisting of:

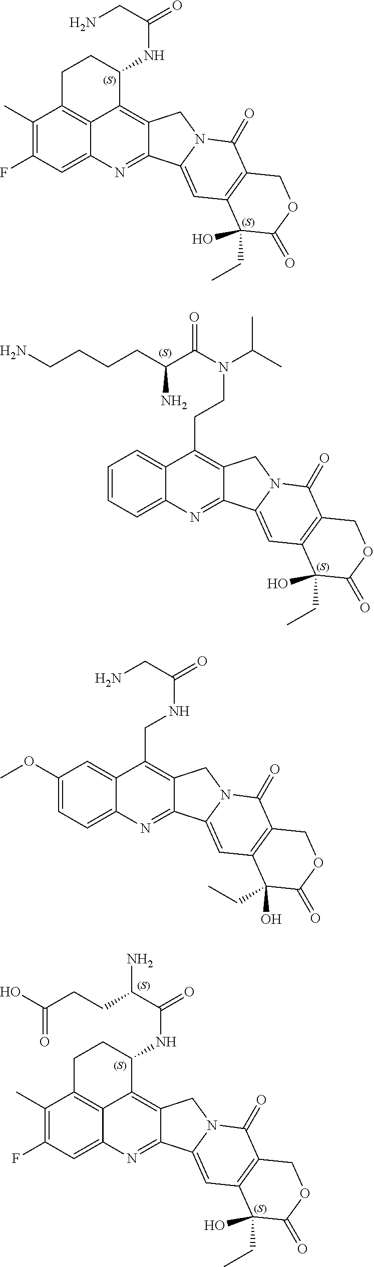

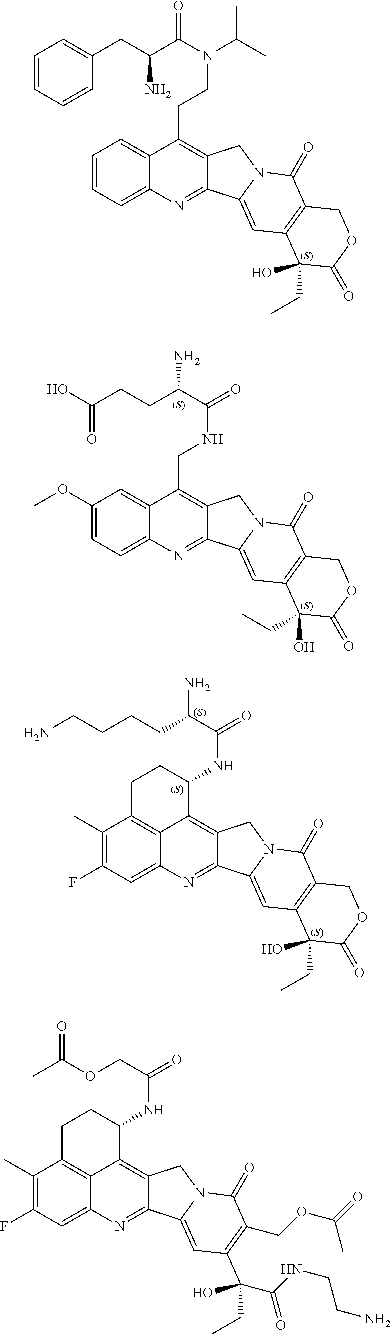

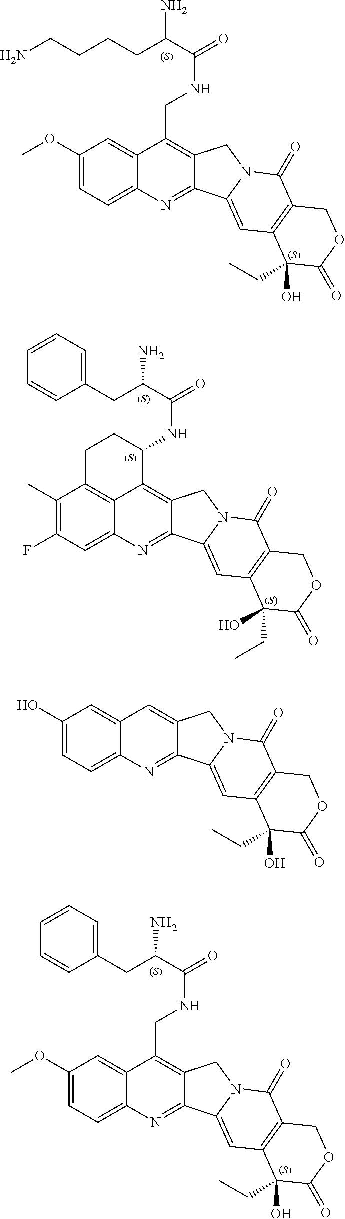

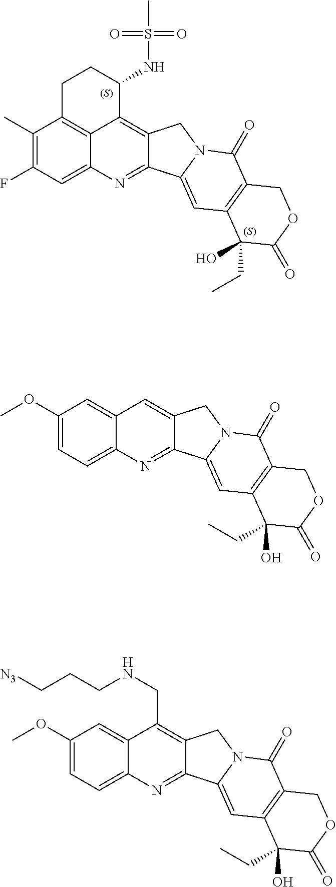

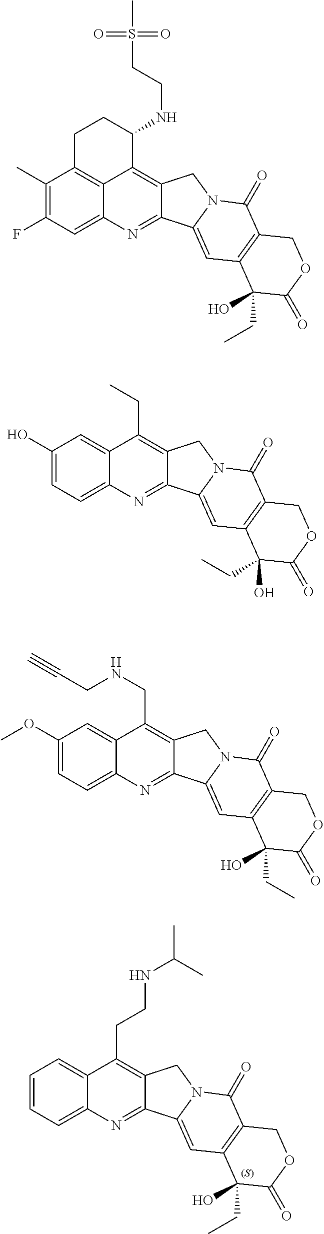

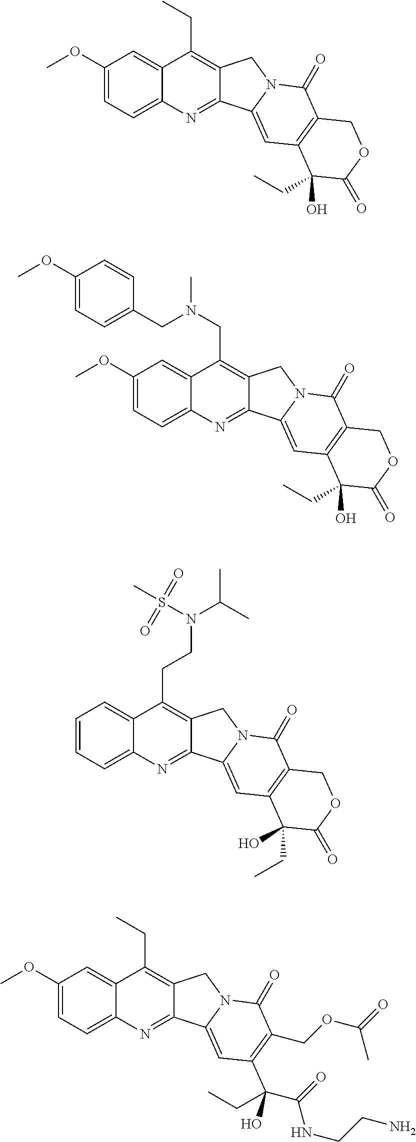 [SMILES: CCc1c2c(nc3ccc(OC)cc13)-c1cc3c(c(=O)n1C2)COC(=O)[C@]3(O)CC ; CC[C@@]1(O)C(=O)OCc2c1cc1n(c2=O)Cc2c-1nc1ccc(OC)cc1c2CN(C)Cc1ccc(OC)cc1 ; CC[C@@]1(O)C(=O)OCc2c1cc1n(c2=O)Cc2c-1nc1ccccc1c2CCN(C(C)C)S(C)(=O)=O ; CCc1c2c(nc3ccc(OC)cc13)-c1cc([C@@](O)(CC)C(=O)NCCN)c(COC(C)=O)c(=O)n1C2]

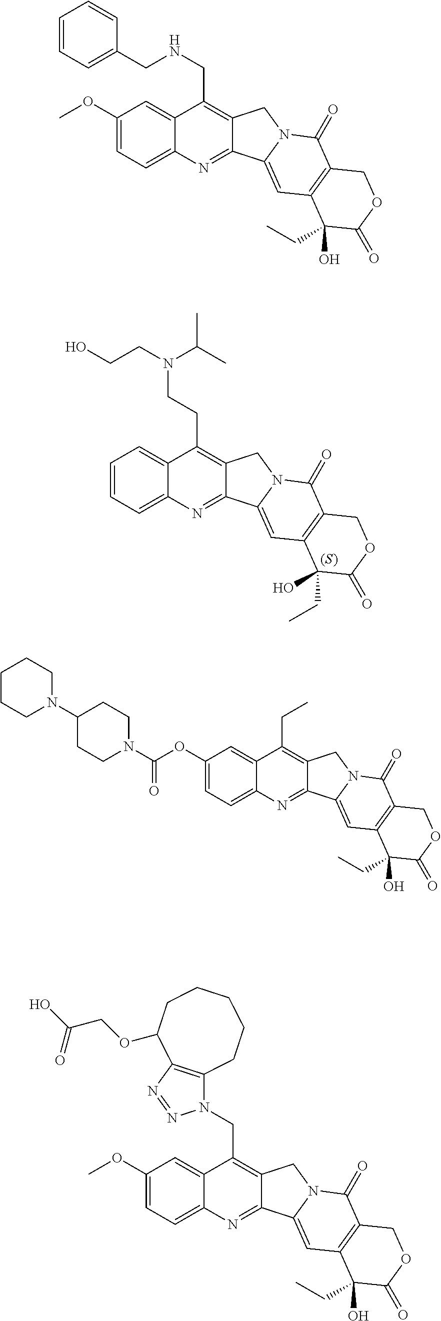

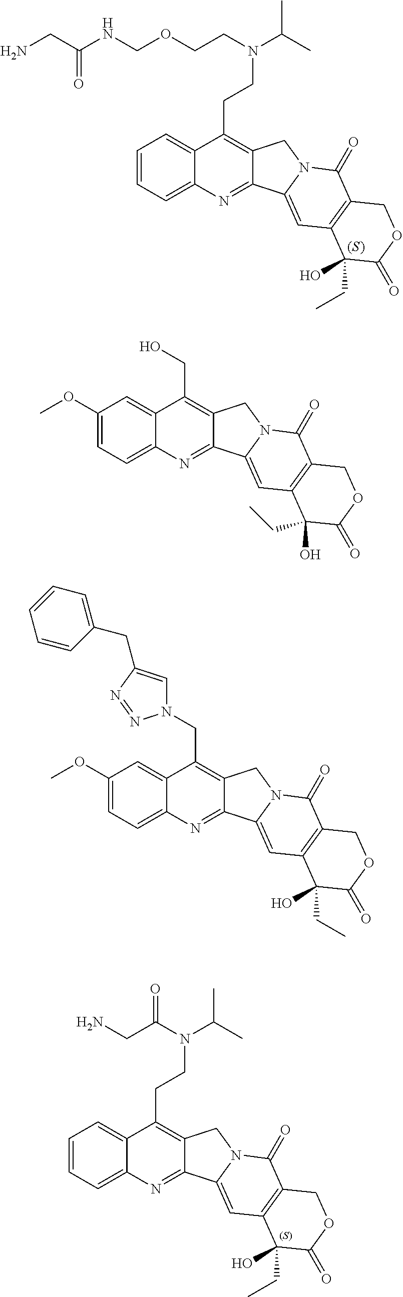

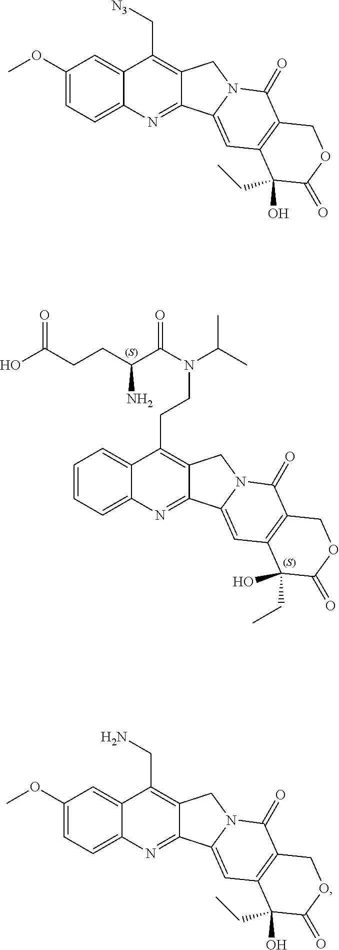

or a pharmaceutically acceptable salt thereof.

In another aspect, the present disclosure provides a pharmaceutical composition comprising the compound according to any one of the above embodiments, and a diluent, a carrier, and/or an excipient.

In another aspect, the present disclosure provides a method of treating a tumor and/or cancer comprising contacting the tumor and/or cancer with the compound according to any of the above embodiments.

In another aspect, the present disclosure provides a method of treating a condition in a subject in need thereof comprising administering to the subject a therapeutically effective amount of the compound according to any one of the above embodiments, or the composition of any one of the above embodiments.

In one embodiment, the condition is cancer. In one embodiment, the cancer is selected from the group consisting of breast cancer, ovarian cancer, prostate cancer, lung cancer, liver cancer, or brain cancer. In one embodiment, the condition is HER2+ breast cancer.

In another aspect, the present disclosure provides a method of selectively delivering a compound into a cell, wherein the compound is according to any one of the above embodiments.

In another aspect, the present disclosure provides a method of selectively targeting an antigen on a surface of a cell with a compound, wherein the compound is according to any one of the above embodiments.

In one embodiment, the cell is a mammalian cell. In one embodiment, the cell is a human cell. In one embodiment, the cell is a cancer cell.

In one embodiment, the cancer cell is selected from the group consisting of a breast cancer cell, an ovarian cancer cell, a prostate cancer cell, a lung cancer cell, a liver cancer cell, or a brain cancer cell.

In another aspect, the present disclosure provides a method of producing a compound having a structure according to Formula (A):

BA-(L1-B-L2-P)ₙ (A),

wherein BA is an antibody or an antigen-binding fragment thereof; L1 is a first linker covalently bound to the side chain of a glutamine residue of the BA; B is a moiety comprising a triazole; L2 is a second linker covalently bound to the P, wherein
P is an antitumor agent selected from the group consisting of P-I through P-IV:

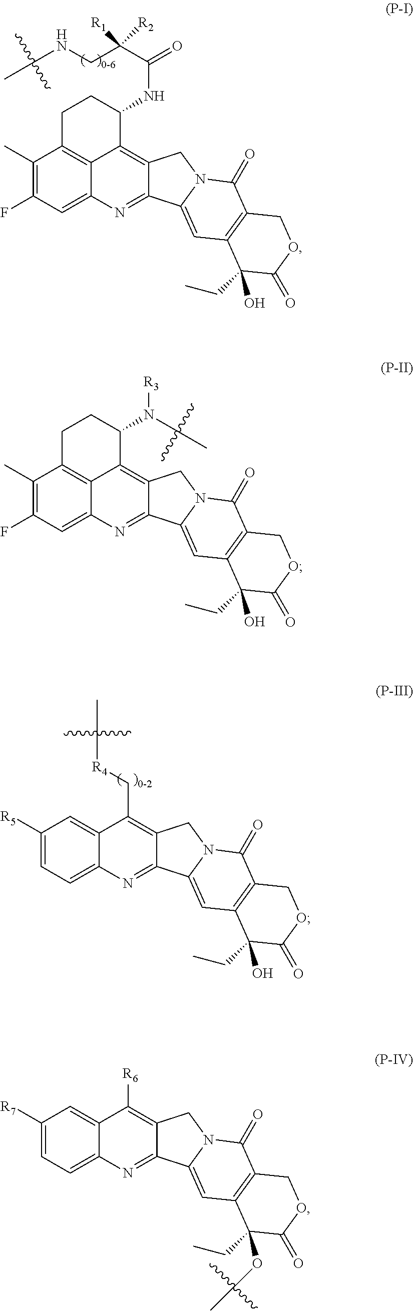

R₁ is hydrogen, C₁₋₆ alkyl, —(CH₂)ᵥ—OH, —(CH₂)ᵥ—NH₂, —(CH₂)ᵥ—C(O)OH, —(CH₂)ᵥ-phenyl, and —(CH₂)ᵥ—SO₂CH₃, wherein v is an integer from 0 to 12;
R² is hydrogen, C₁₋₆ alkyl, —(CH₂)ᵥ—OH, —(CH₂)ᵥ—NH₂, —(CH₂)ᵥ—C(O)OH, —(CH₂)ᵥ-phenyl, and —(CH₂)ᵥ—SO₂CH₃, wherein v is an integer from 0 to 12;
R³ is hydrogen —C₁₋₆ alkyl, —(CH₂)ᵥ—OH, —(CH₂)ᵥ—NH₂, —(CH₂)ᵥ—C(O)OH, —(CH₂)ᵥ-phenyl, —(CH₂)ᵥ—SO₂CH₃, and —CO—(CH₂)ᵥ—O—COCH₃, wherein v is an integer from 0 to 12;
R⁴ is —NH—, —N(—C₁₋₆ alkyl), —N(—C₁₋₆ alkyl)(—SO₂CH₃), —N(—C₁₋₆ alkyl)(—(CH₂)ᵥ—OH), —N(—C₁₋₆ alkyl)(CO—CH₂—NH₂), —N(—C₁₋₆ alkyl)(—(CH₂)ᵥ—O—CH₂—NH—CO—CH₂—NH₂), —N(—C₁₋₆ alkyl)(—CO—CH(NH₂)—(CH₂)ᵥ—COOH), —N(—C₁₋₆ alkyl)(—CO—CH(NH₂)—(CH₂)ᵥ—NH₂), —N(—C₁₋₆ alkyl)(—CO—CH(NH₂)—(CH₂)ᵥ-phenyl), or

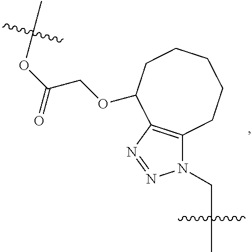

wherein v is an integer from 0 to 12;
R⁵ is H, —OH, —OCH₃, or

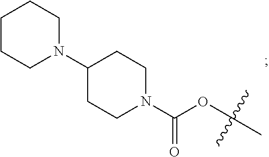 [SMILES: *OC(=O)N1CCC(N2CCCCC2)CC1]

R⁶ is hydrogen —C₁₋₆ alkyl, —(CH₂)ᵥ—OH, —(CH₂)ᵥ—NH₂, —(CH₂)ᵥ-phenyl, —(CH₂)ᵥ—N₃, —(CH₂)ᵥ—NH—CH₂-phenyl, —(CH₂)ᵥ—NMe-CH₂-phenyl-OMe, —(CH₂)ᵥ—NH—(CH₂)ᵥ—N₃, —(CH₂)ᵥ—NH—(CH₂)ᵥ—CCH; —(CH₂)ᵥ—NH—CO—CH(NH₂)—(CH₂)ᵥ-phenyl, —(CH₂)ᵥ—NH—CO—CH(NH₂)—(CH₂)ᵥ—N₃, —(CH₂)ᵥ—NH—CO—CH(NH₂)—(CH₂)ᵥ—NH₂, —(CH₂)ᵥ—NH—CO—CH(NH₂)—(CH₂)ᵥ—COOH,

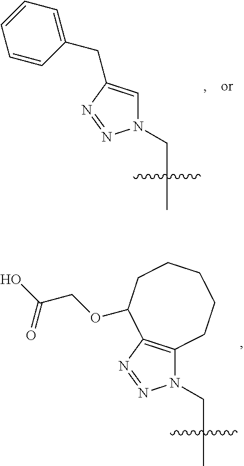

wherein v is an independently an integer from 0 to 12;
R⁷ is H, —OH, —OCH₃, or

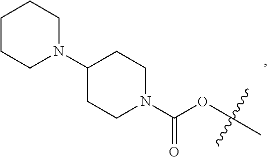 [SMILES: *OC(=O)N1CCC(N2CCCCC2)CC1]

wherein the method comprises the steps of:
a) contacting, in the presence of a transglutaminase, the BA comprising at least one glutamine residue with a compound L1-B′,
b) contacting the product of step a) with one or more equivalents of a compound B″-L2-P, wherein the group B″ is capable of covalently attaching to the group B′,
wherein one of the groups B′ and B″ is selected from —N₃ and

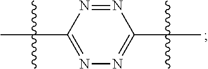 [SMILES: *c1nnc(*)nn1]

and the other of the groups B′ and B″ is selected from

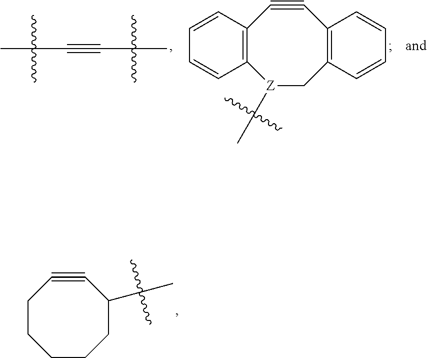

where Z is C or N; and
c) isolating the produced compound of Formula (A).

In one embodiment, the compound of Formula (A) has a structure selected from the group consisting of:

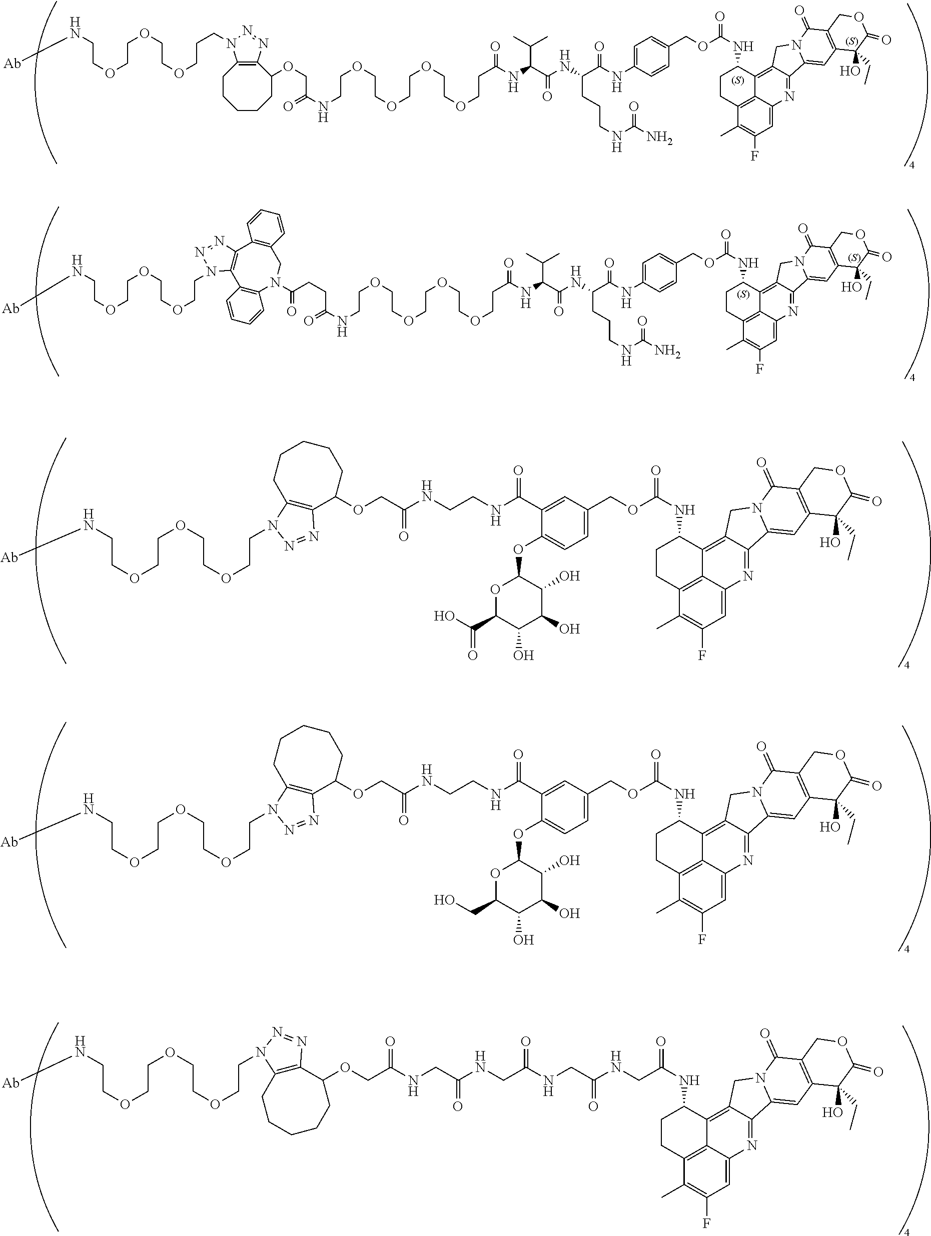

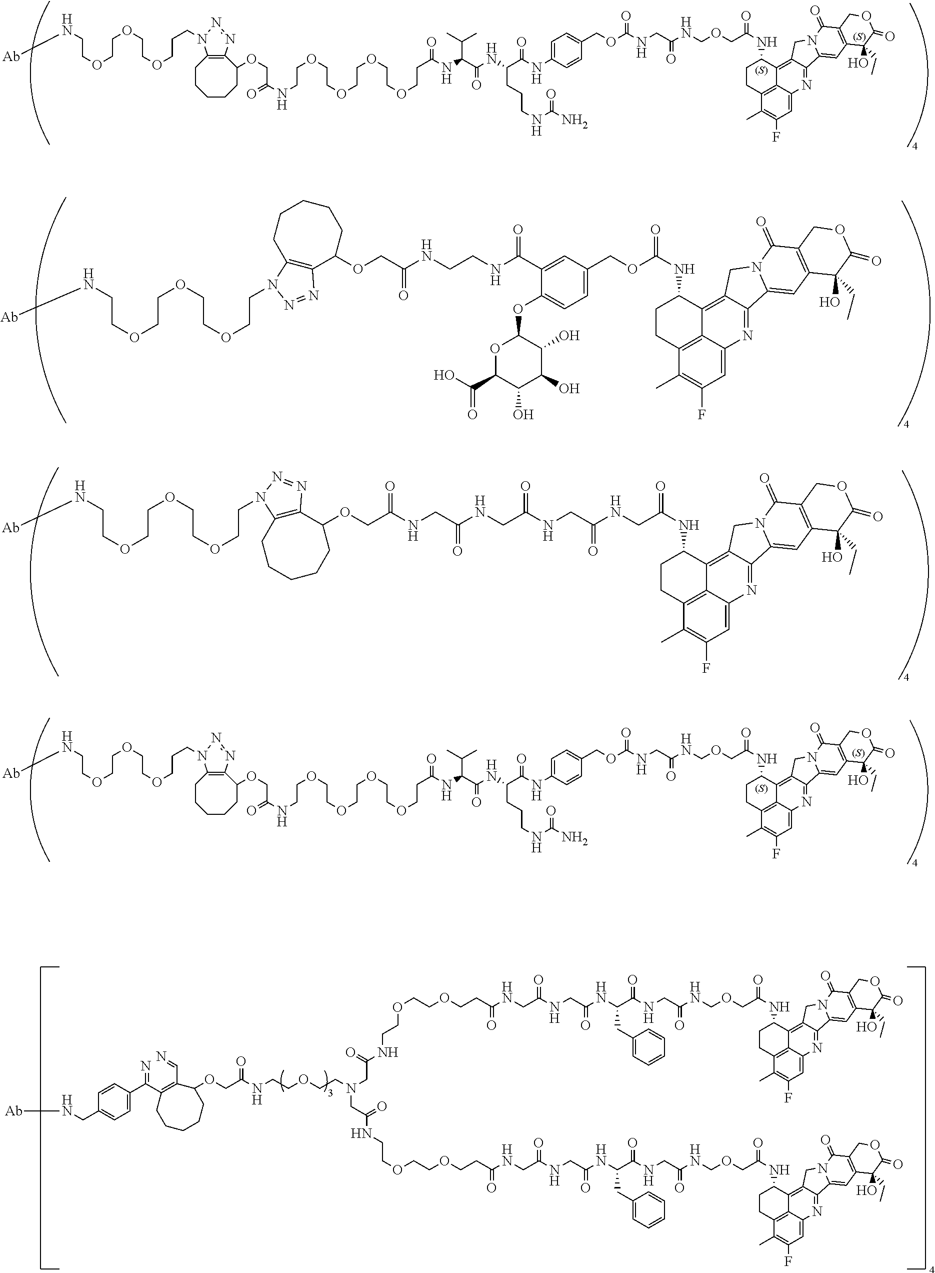

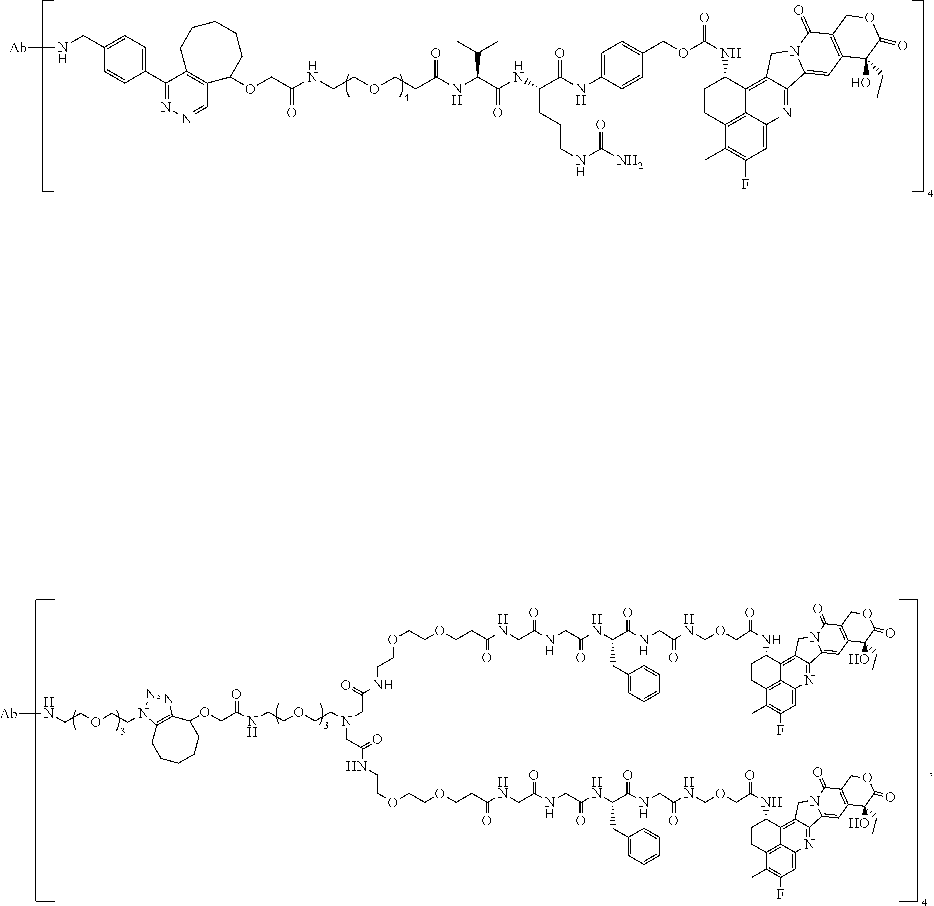

or a pharmaceutically acceptable salt thereof.

These and other aspects of the present disclosure will become apparent to those skilled in the art after a reading of the following detailed description of the disclosure, including the appended claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a schematic of a two-step site-specific generation of ADCs according to an embodiment of the present disclosure.

FIG. 2 is a plot of EC₅₀ values and maximum % kill in SK-BR-3 cells of free exatecan, control ADC Trastuzumab deruxtecan (DS8201a), and two ADCs according to the present disclosure.

FIG. 3 is a plot of EC₅₀ values and maximum % kill in SK-BR-3 cells of free exatecan and five ADCs according to the present disclosure.

### DETAILED DESCRIPTION

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive. Therefore, specific structural and functional details disclosed herein are not to be interpreted as limiting, but merely as a representative basis for teaching one skilled in the art to variously employ the present disclosure.

### Definitions

Unless defined otherwise, all technical and scientific terms used herein have the same meaning as commonly understood by one of ordinary skill in the art to which this disclosure belongs.

As used in this specification and the appended claims, the singular forms “a”, “an”, and “the” include plural references unless the context clearly dictates otherwise. Thus, for example, a reference to “a method” includes one or more methods, and/or steps of the type described herein and/or which will become apparent to those persons skilled in the art upon reading this disclosure.

The terms “treat” or “treatment” of a state, disorder or condition include: (1) preventing, delaying, or reducing the incidence and/or likelihood of the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof or at least one clinical or sub-clinical symptom thereof; or (3) relieving the disease, i.e., causing regression of the state, disorder or condition or at least one of its clinical or sub-clinical symptoms. The benefit to a subject to be treated is either statistically significant or at least perceptible to the patient or to the physician. In some embodiments, treatment comprises methods wherein cells are ablated in such manner where disease is indirectly impacted. In certain embodiments, treatment comprises depleting immune cells as a hematopoietic conditioning regimen prior to therapy.

A “subject” or “patient” or “individual” or “animal”, as used herein, refers to humans, veterinary animals (e.g., cats, dogs, cows, horses, sheep, pigs, etc.) and experimental animal models of diseases (e.g., mice, rats). In a preferred embodiment, the subject is a human.

As used herein the term “effective” applied to dose or amount refers to that quantity of a compound or pharmaceutical composition that is sufficient to result in a desired activity upon administration to a subject in need thereof. Note that when a combination of active ingredients is administered, the effective amount of the combination may or may not include amounts of each ingredient that would have been effective if administered individually. The exact amount required will vary from subject to subject, depending on the species, age, and general condition of the subject, the severity of the condition being treated, the particular drug or drugs employed, the mode of administration, and the like.

The phrase “pharmaceutically acceptable salt”, as used in connection with compositions of the disclosure, refers to any salt suitable for administration to a patient. Suitable salts include, but are not limited to, those disclosed in Berge et al., “Pharmaceutical Salts”, J. Pharm. Sci., 1977, 66:1, incorporated herein by reference. Examples of salts include, but are not limited to, acid derived, base derived, organic, inorganic, amine, and alkali or alkaline earth metal salts, including but not limited to calcium salts, magnesium salts, potassium salts, sodium salts, salts of hydrochloric acid, hydrobromic acid, sulfuric acid, nitric acid, phosphoric acid, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methane sulfonic acid, ethane sulfonic acid, p toluene sulfonic acid, salicylic acid, and the like. In some examples, a payload described herein (e.g., a rifamycin analog described herein) comprises a tertiary amine, where the nitrogen atom in the tertiary amine is the atom through which the payload is bonded to a linker or a linker-spacer. In such instances, bonding to the tertiary amine of the payload yields a quaternary amine in the linker-payload molecule. The positive charge on the quaternary amine can be balanced by a counter ion (e.g., chloro, bromo, iodo, or any other suitably charged moiety such as those described herein).

Ranges can be expressed herein as from “about” or “approximately” one particular value and/or to “about” or “approximately” another particular value. When such a range is expressed, another embodiment includes from the one particular value and/or to the other particular value.

By “comprising” or “containing” or “including” is meant that at least the named compound, element, particle, or method step is present in the composition or article or method, but does not exclude the presence of other compounds, materials, particles, or method steps, even if the other such compounds, material, particles, or method steps have the same function as what is named.

Compounds of the present disclosure include those described generally herein, and are further illustrated by the classes, subclasses, and species disclosed herein. As used herein, the following definitions shall apply unless otherwise indicated. For purposes of this disclosure, the chemical elements are identified in accordance with the Periodic Table of the Elements, CAS version, Handbook of Chemistry and Physics, 75th Ed. Additionally, general principles of organic chemistry are described in “Organic Chemistry”, Thomas Sorrell, University Science Books, Sausalito: 1999, and “March's Advanced Organic Chemistry”, 5th Ed., Ed.: Smith, M. B. and March, J., John Wiley & Sons, New York: 2001, the entire contents of which are hereby incorporated by reference.

As used herein, the term “alkyl” is given its ordinary meaning in the art and may include saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 1-20 carbon atoms in its backbone (e.g., C₁-C₂₀ for straight chain, C₂-C₂₀ for branched chain), and alternatively, about 1-10 carbon atoms, or about 1 to 6 carbon atoms. In some embodiments, a cycloalkyl ring has from about 3-10 carbon atoms in their ring structure where such rings are monocyclic or bicyclic, and alternatively about 5, 6 or 7 carbons in the ring structure. In some embodiments, an alkyl group may be a lower alkyl group, wherein a lower alkyl group comprises 1-4 carbon atoms (e.g., C₁-C₄ for straight chain lower alkyls).

As used herein, the term “alkenyl” refers to an alkyl group, as defined herein, having one or more double bonds.

As used herein, the term “alkynyl” refers to an alkyl group, as defined herein, having one or more triple bonds.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring.

The term “halogen” means F, Cl, Br, or I; the term “halide” refers to a halogen radical or substituent, namely —F, —Cl, —Br, or —I.

The term “adduct”, e.g., “an adduct of group B′” of the present disclosure encompasses any moiety comprising the product of an addition reaction, e.g., an addition reaction of group B′, independent of the synthetic steps taken to produce the moiety.

The term “covalent attachment” means formation of a covalent bond, i.e., a chemical bond that involves sharing of one or more electron pairs between two atoms. Covalent bonding may include different interactions, including but not limited to σ-bonding, τr-bonding, metal-to-metal bonding, agostic interactions, bent bonds, and three-center two-electron bonds. When a first group is said to be “capable of covalently attaching” to a second group, this means that the first group is capable of forming a covalent bond with the second group, directly or indirectly, e.g., through the use of a catalyst or under specific reaction conditions. Non-limiting examples of groups capable of covalently attaching to each other may include, e.g., an amine and a carboxylic acid (forming an amide bond), a diene and a dienophile (via a Diels-Alder reaction), and an azide and an alkyne (forming a triazole via a 1,3-cycloaddition reaction).

As described herein, compounds of the disclosure may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this disclosure are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Unless otherwise stated, structures depicted herein are also meant to include all isomeric (e.g., enantiomeric, diastereomeric, and geometric (or conformational)) forms of the structure; for example, the R and S configurations for each asymmetric center, (Z) and (E) double bond isomers, and (Z) and (E) conformational isomers. Therefore, single stereochemical isomers as well as enantiomeric, diastereomeric, and geometric (or conformational) mixtures of the present compounds are within the scope of the disclosure.

Unless otherwise stated, all tautomeric forms of the compounds of the disclosure are within the scope of the disclosure.

Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ¹¹C- or ¹³C- or ¹⁴C-enriched carbon are within the scope of this disclosure.

It is also to be understood that the mention of one or more method steps does not preclude the presence of additional method steps or intervening method steps between those steps expressly identified. Similarly, it is also to be understood that the mention of one or more components in a device or system does not preclude the presence of additional components or intervening components between those components expressly identified.

Unless otherwise stated, all crystalline forms of the compounds of the disclosure and salts thereof are also within the scope of the disclosure. The compounds of the disclosure may be isolated in various amorphous and crystalline forms, including without limitation forms which are anhydrous, hydrated, non-solvated, or solvated. Example hydrates include hemihydrates, monohydrates, dihydrates, and the like. In some embodiments, the compounds of the disclosure are anhydrous and non-solvated. By “anhydrous” is meant that the crystalline form of the compound contains essentially no bound water in the crystal lattice structure, i.e., the compound does not form a crystalline hydrate.

As used herein, “crystalline form” is meant to refer to a certain lattice configuration of a crystalline substance. Different crystalline forms of the same substance typically have different crystalline lattices (e.g., unit cells) which are attributed to different physical properties that are characteristic of each of the crystalline forms. In some instances, different lattice configurations have different water or solvent content. The different crystalline lattices can be identified by solid state characterization methods such as by X-ray powder diffraction (PXRD). Other characterization methods such as differential scanning calorimetry (DSC), thermogravimetric analysis (TGA), dynamic vapor sorption (DVS), solid state NMR, and the like further help identify the crystalline form as well as help determine stability and solvent/water content.

Crystalline forms of a substance include both solvated (e.g., hydrated) and non-solvated (e.g., anhydrous) forms. A hydrated form is a crystalline form that includes water in the crystalline lattice. Hydrated forms can be stoichiometric hydrates, where the water is present in the lattice in a certain water/molecule ratio such as for hemihydrates, monohydrates, dihydrates, etc. Hydrated forms can also be non-stoichiometric, where the water content is variable and dependent on external conditions such as humidity.

In some embodiments, the compounds of the disclosure are substantially isolated. By “substantially isolated” is meant that a particular compound is at least partially isolated from impurities. For example, in some embodiments a compound of the disclosure comprises less than about 50%, less than about 40%, less than about 30%, less than about 20%, less than about 15%, less than about 10%, less than about 5%, less than about 2.5%, less than about 1%, or less than about 0.5% of impurities. Impurities generally include anything that is not the substantially isolated compound including, for example, other crystalline forms and other substances.

Certain groups, moieties, substituents, and atoms are depicted with a wavy line. The wavy line can intersect or cap a bond or bonds. The wavy line indicates the atom through which the groups, moieties, substituents, or atoms are bonded. For example, a phenyl group that is substituted with a propyl group depicted as:

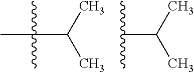

has the following structure:

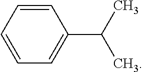

The expression “HER2” or “human epidermal growth factor receptor 2” refers to a member of the human epidermal growth factor receptor family. The protein is also known as NEU; NGL; HER2; TKR1; CD340; HER-2; MLN 19; HER-2/neu. HER2 can refer to the amino acid sequence as set forth in NCBI accession No. NP_004439.2. Amplification or over-expression of this oncogene has been shown to play an important role in the development and progression of certain aggressive types of breast cancer. In recent years the protein has become an important biomarker and target of therapy for approximately 30% of breast cancer patient. All references to proteins, polypeptides and protein fragments herein are intended to refer to the human version of the respective protein, polypeptide or protein fragment unless explicitly specified as being from a non-human species. Thus, the expression “HER2” means human HER2 unless specified as being from a non-human species, e.g., “mouse HER2,” “monkey HER2,” etc.

The phrase “an antibody that binds HER2” or an “anti-HER2 antibody” includes antibodies and antigen-binding fragments thereof that specifically recognize HER2.

The phrase an “anti-HER2/HER2” antibody, e.g., an “anti-HER2/HER2 bispecific antibody” includes antibodies and antigen-binding fragments thereof that specifically recognize two different HER2 epitopes. In some embodiments, bispecific antibodies and antigen-binding fragments thereof comprise a first antigen-binding domain (D1) which specifically binds a first epitope of human HER2 and a second antigen-binding domain (D2) which specifically binds a second epitope of human HER2.

The expression “STEAP2,” as used herein, refers to six-transmembrane epithelial antigen of prostate 2. STEAP2 is an integral, six-transmembrane-spanning protein that is highly expressed in prostate epithelial cells and is a cell-surface marker for prostate cancer, for example STEAP2 was found to be expressed in significant levels on an LNCaP prostate cell line (Porkka, et al. Lab Invest 2002, 82:1573-1582). STEAP2 (UniProtKB/Swiss-Prot: Q8NFT2.3) is a 490-amino acid protein encoded by STEAP2 gene located at the chromosomal region 7q21 in humans.

As used herein, “an antibody that binds STEAP2” or an “anti-STEAP2 antibody” includes antibodies and antigen-binding fragments thereof that specifically recognize STEAP2.

The phrase “an antibody that binds MET” or an “anti-MET antibody” includes antibodies and antigen-binding fragments thereof that specifically recognize MET. The expressions “MET,” “c-Met,” and the like, as used herein, refer to the human membrane spanning receptor tyrosine kinase.

The phrase an “anti-MET/MET” antibody, e.g., an “anti-MET/MET bispecific antibody” includes antibodies and antigen-binding fragments thereof that specifically recognize two different MET epitopes. In some embodiments, bispecific antibodies and antigen-binding fragments thereof comprise a first antigen-binding domain (D1) which specifically binds a first epitope of human MET and a second antigen-binding domain (D2) which specifically binds a second epitope of human MET.

All amino acid abbreviations used in this disclosure are those accepted by the United States Patent and Trademark Office as set forth in 37 C.F.R. § 1.822(B)(J).

The term “protein” means any amino acid polymer having more than about 20 amino acids covalently linked via amide bonds. As used herein, “protein” includes biotherapeutic proteins, recombinant proteins used in research or therapy, trap proteins and other Fc-fusion proteins, chimeric proteins, antibodies, monoclonal antibodies, human antibodies, bispecific antibodies, antibody fragments, nanobodies, recombinant antibody chimeras, scFv fusion proteins, cytokines, chemokines, peptide hormones, and the like. Proteins can be produced using recombinant cell-based production systems, such as the insect bacculovirus system, yeast systems (e.g., Pichia sp.), mammalian systems (e.g., CHO cells and CHO derivatives like CHO-K1 cells).

All references to proteins, polypeptides and protein fragments herein are intended to refer to the human version of the respective protein, polypeptide or protein fragment unless explicitly specified as being from a non-human species. Thus, the expression “STEAP2” means human STEAP2 unless specified as being from a non-human species, e.g., “mouse STEAP2,” “monkey STEAP2,” etc.

The amino acid sequence of an antibody can be numbered using any known numbering schemes, including those described by Kabat et al., (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Pluckthun, J. Mol. Biol., 2001, 309:657-70 (“AHo” numbering scheme). Unless otherwise specified, the numbering scheme used herein is the Kabat numbering scheme. However, selection of a numbering scheme is not intended to imply differences in sequences where they do not exist, and one of skill in the art can readily confirm a sequence position by examining the amino acid sequence of one or more antibodies. Unless stated otherwise, the “EU numbering scheme” is generally used when referring to a residue in an antibody heavy chain constant region (e.g., as reported in Kabat et al., supra).

The term “glutaminyl-modified antibody” refers to an antibody with at least one covalent linkage from a glutamine side chain to a primary amine compound of the present disclosure. In particular embodiments, the primary amine compound is linked through an amide linkage on the glutamine side chain. In certain embodiments, the glutamine is an endogenous glutamine. In other embodiments, the glutamine is an endogenous glutamine made reactive by polypeptide engineering (e.g., via amino acid deletion, insertion, substitution, or mutation on the polypeptide). In additional embodiments, the glutamine is polypeptide engineered with an acyl donor glutamine-containing tag (e.g., glutamine-containing peptide tags, Q-tags or TGase recognition tag).

The term “TGase recognition tag” refers to a sequence of amino acids comprising an acceptor glutamine residue and that when incorporated into (e.g., appended to) a polypeptide sequence, under suitable conditions, is recognized by a TGase and leads to cross-linking by the TGase through a reaction between an amino acid side chain within the sequence of amino acids and a reaction partner. The recognition tag may be a peptide sequence that is not naturally present in the polypeptide comprising the TGase recognition tag. In some embodiments, the TGase recognition tag comprises at least one Gln. In some embodiments, the TGase recognition tag comprises an amino acid sequence XXQX (SEQ ID NO: 1935), wherein X is any amino acid (e.g., conventional amino acid Leu, Ala, Gly, Ser, Val, Phe, Tyr, His, Arg, Asn, Glu, Asp, Cys, Gln, lie, Met, Pro, Thr, Lys, or Trp or nonconventional amino acid). In some embodiments, the acyl donor glutamine-containing tag comprises an amino acid sequence selected from the group consisting of LLQGG (SEQ ID NO:1936), LLQG (SEQ ID NO:1937), LSLSQG (SEQ ID NO:1938), gGGLLQGG (SEQ ID NO:1939), gLLQG (SEQ ID NO:1940), LLQ, gSPLAQSHGG (SEQ ID NO:1941), gLLQGGG (SEQ ID NO:1942), gLLQGG (SEQ ID NO:1943), gLLQ (SEQ ID NO:1944), LLQLLQGA (SEQ ID NO:1945), LLQGA (SEQ ID NO:1946), LLQYQGA (SEQ ID NO:1947), LLQGSG (SEQ ID NO:1948), LLQYQG (SEQ ID NO:1949), LLQLLQG (SEQ ID NO:1950), SLLQG (SEQ ID NO:1951), LLQLQ (SEQ ID NO:1952), LLQLLQ (SEQ ID NO:1953), and LLQGR (SEQ ID NO:1954). See for example, WO2012059882, the entire contents of which are incorporated herein.

The term “antibody,” as used herein, means any antigen-binding molecule or molecular complex comprising at least one complementarity determining region (CDR) that specifically binds to or interacts with a particular antigen. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or VH) and a heavy chain constant region. The heavy chain constant region comprises three domains, CH1, CH2, and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different embodiments, the FRs of the antibody (or antigen-binding portion thereof) can be identical to the human germline sequences, or can be naturally or artificially modified. An amino acid consensus sequence can be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody can be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA can be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain can be of any size or amino acid composition and will generally comprise at least one CDR which is adjacent to or in frame with one or more framework sequences. In antigen-binding fragments having a VH domain associated with a VL domain, the VH and VL domains can be situated relative to one another in any suitable arrangement. For example, the variable region can be dimeric and contain VH-VH, VH-VL or VL-VL dimers.

Alternatively, the antigen-binding fragment of an antibody can contain a monomeric VH or VL domain.

In certain embodiments, an antigen-binding fragment of an antibody can contain at least one variable domain covalently linked to at least one constant domain. Non-limiting, exemplary configurations of variable and constant domains that can be found within an antigen-binding fragment of an antibody of the present description include: (i) VH—CH1; (ii) VH—CH2; (iii) VH—CH3; (iv) VH—CH1-CH2; (V) VH—CH1-CH2-CH3; (vi) VH—CH2-CH3; (vii) VH—CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed herein, the variable and constant domains can be either directly linked to one another or can be linked by a full or partial hinge or linker region. A hinge region can consist of at least 2 (e.g., 5, 10, 15, 20, 40, 60, or more) amino acids which result in a flexible or semi-flexible linkage between adjacent variable and/or constant domains in a single polypeptide molecule.

Moreover, an antigen-binding fragment of an antibody of the present description can comprise a homo-dimer or hetero-dimer (or other multimer) of any of the variable and constant domain configurations listed herein in non-covalent association with one another and/or with one or more monomeric VH or VL domain (e.g., by disulfide bond(s)).

As with full antibody molecules, antigen-binding fragments can be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antibody format, including the exemplary bispecific antibody formats disclosed herein, can be adapted for use in the context of an antigen-binding fragment of an antibody of the present description using routine techniques available in the art.

The antibodies of the present description can function through complement-dependent cytotoxicity (CDC) or antibody-dependent cell-mediated cytotoxicity (ADCC). “Complement-dependent cytotoxicity” (CDC) refers to lysis of antigen-expressing cells by an antibody of the description in the presence of complement. “Antibody-dependent cell-mediated cytotoxicity” (ADCC) refers to a cell-mediated reaction in which nonspecific cytotoxic cells that express Fc receptors (FcRs) (e.g., Natural Killer (NK) cells, neutrophils, and macrophages) recognize bound antibody on a target cell and thereby lead to lysis of the target cell. CDC and ADCC can be measured using assays that are well known and available in the art. (See, e.g., U.S. Pat. Nos. 5,500,362 and 5,821,337, and Clynes et al. (1998) Proc. Natl. Acad. Sci. (USA) 95:652-656). The constant region of an antibody is important in the ability of an antibody to fix complement and mediate cell-dependent cytotoxicity. Thus, the isotype of an antibody can be selected on the basis of whether it is desirable for the antibody to mediate cytotoxicity.

In certain embodiments, the antibodies of the description, e.g., anti-HER2 antibodies, or anti-HER2/HER2 bispecific antibodies, or anti-MET antibodies, or anti-MET/MET bispecific antibodies, or anti-STEAP2 antibodies, are human antibodies. The term “human antibody,” as used herein, is intended to include antibodies having variable and constant regions derived from human germline immunoglobulin sequences. The human antibodies of the description can include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies can, in some embodiments, be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (See, e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

Human antibodies can exist in two forms that are associated with hinge heterogeneity. In one form, an immunoglobulin molecule comprises a stable four chain construct of approximately 150-160 kDa in which the dimers are held together by an interchain heavy chain disulfide bond. In a second form, the dimers are not linked via inter-chain disulfide bonds and a molecule of about 75-80 kDa is formed composed of a covalently coupled light and heavy chain (half-antibody). These forms have been extremely difficult to separate, even after affinity purification. The frequency of appearance of the second form in various intact IgG isotypes is due to, but not limited to, structural differences associated with the hinge region isotype of the antibody. A single amino acid substitution in the hinge region of the human IgG4 hinge can significantly reduce the appearance of the second form (Angal et al. (1993) Molecular Immunology 30: 105) to levels typically observed using a human IgG1 hinge. The instant description encompasses antibodies having one or more mutations in the hinge, CH2 or CH3 region which can be desirable, for example, in production, to improve the yield of the desired antibody form.

The antibodies of the description can be isolated or purified antibodies. An “isolated antibody” or “purified antibody,” as used herein, means an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody” for purposes of the present description. For example, an antibody that has been purified from at least one component of a reaction or reaction sequence, is a “purified antibody” or results from purifying the antibody. An isolated antibody also includes an antibody in situ within a recombinant cell. Isolated antibodies are antibodies that have been subjected to at least one purification or isolation step. According to certain embodiments, an isolated antibody or purified antibody can be substantially free of other cellular material and/or chemicals.

The antibodies disclosed herein can comprise one or more amino acid substitutions, insertions and/or deletions in the framework and/or CDR regions of the heavy and light chain variable domains as compared to the corresponding germline sequences from which the antibodies were derived. Such mutations can be readily ascertained by comparing the amino acid sequences disclosed herein to germline sequences available from, for example, public antibody sequence databases. The present description includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived).

Furthermore, the antibodies of the present description can contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, improved drug-to-antibody ratio (DAR) for antibody-drug conjugates, etc. Antibodies and antigen-binding fragments obtained in this general manner are encompassed within the present description.

The term “aglycosylated antibody” refers to an antibody that does not comprise a glycosylation sequence that might interfere with a transglutamination reaction, for instance an antibody that does not have saccharide group at N297 on one or more heavy chains. In particular embodiments, an antibody heavy chain has an N297 mutation. In other words, the antibody is mutated to no longer have an asparagine residue at position 297 according to the EU numbering system as disclosed by Kabat et al. In particular embodiments, an antibody heavy chain has an N297Q or an N297D mutation. Such an antibody can be prepared by site-directed mutagenesis to remove or disable a glycosylation sequence or by site-directed mutagenesis to insert a glutamine residue at site apart from any interfering glycosylation site or any other interfering structure. Such an antibody also can be isolated from natural or artificial sources. Aglycosylated antibodies also include antibodies comprising a T299 or S298P or other mutations, or combinations of mutations that result in a lack of glycosylation.

The term “deglycosylated antibody” refers to an antibody in which a saccharide group at is removed to facilitate transglutaminase-mediated conjugation. Saccharides include, but are not limited to, N-linked oligosaccharides. In some embodiments, deglycosylation is performed at residue N297. In some embodiments, removal of saccharide groups is accomplished enzymatically, included but not limited to via PNGase.

The term “epitope” refers to an antigenic determinant that interacts with a specific antigen binding site in the variable region of an antibody molecule known as a paratope. A single antigen can have more than one epitope. Thus, different antibodies can bind to different areas on an antigen and can have different biological effects. Epitopes can be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope can include moieties of saccharides, phosphoryl groups, or sulfonyl groups on the antigen.

The terms “conjugated protein” or “conjugated antibody” as used herein refers to a protein or an antibody covalently linked to one or more chemical moieties. The chemical moiety can include an amine compound of the present disclosure. Linkers (L) and payloads (D) suitable for use with the present disclosure are described in detail herein. In particular embodiments, a conjugated antibody comprising a therapeutic moiety is an antibody-drug conjugate (ADC), also referred to as an antibody-payload conjugate, or an antibody-linker-payload conjugate.

The term “Drug-to-Antibody Ratio” or (DAR) is the average number of therapeutic moieties, e.g., drugs, conjugated to a binding agent of the present disclosure.

The term “Linker Antibody Ratio” or (LAR), also denoted as the lower case I in some embodiments, is the average number of reactive primary amine compounds conjugated to a binding agent of the present disclosure. Such binding agents, e.g., antibodies, can be conjugated with primary amine compounds comprising, e.g., a suitable azide or alkyne. The resulting binding agent, which is functionalized with an azide or an alkyne can subsequently react with a therapeutic moiety comprising the corresponding azide or alkyne via the 1,3-cycloaddition reaction.

The phrase “pharmaceutically acceptable amount” refers to an amount effective or sufficient in treating, reducing, alleviating, or modulating the effects or symptoms of at least one health problem in a subject in need thereof. For example, a pharmaceutically acceptable amount of an antibody or antibody-drug conjugate is an amount effective for modulating a biological target using the antibody or antibody-drug-conjugates provided herein. Suitable pharmaceutically acceptable amounts include, but are not limited to, from about 0.001% up to about 10%, and any amount in between, such as about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, or about 10% of an antibody or antibody-drug-conjugate provided herein.

The phrase “reaction pH” refers to the pH of a reaction after all reaction components or reactants have been added.

The term “substantial identity” or “substantially identical,” when referring to a nucleic acid or fragment thereof, indicates that, when optimally aligned with appropriate nucleotide insertions or deletions with another nucleic acid (or its complementary strand), there is nucleotide sequence identity in at least about 95%, and more preferably at least about 96%, 97%, 98% or 99% of the nucleotide bases, as measured by any well-known algorithm of sequence identity, such as FASTA, BLAST or gap, as discussed below. A nucleic acid molecule having substantial identity to a reference nucleic acid molecule can, in certain instances, encode a polypeptide having the same or substantially similar amino acid sequence as the polypeptide encoded by the reference nucleic acid molecule.

As applied to polypeptides, the term “substantial similarity” or “substantially similar” means that two peptide sequences, when optimally aligned, such as by the programs gAP or BESTFIT using default gap weights, share at least 95% sequence identity, even more preferably at least 98% or 99% sequence identity. Preferably, residue positions which are not identical differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of similarity can be adjusted upwards to correct for the conservative nature of the substitution. Means for making this adjustment are well-known to those of skill in the art. See, e.g., Pearson (1994) Methods Mol. Biol. 24: 307-331. Examples of groups of amino acids that have side chains with similar chemical properties include (1) aliphatic side chains: glycine, alanine, valine, leucine and isoleucine; (2) aliphatic-hydroxyl side chains: serine and threonine; (3) amide-containing side chains: asparagine and glutamine; (4) aromatic side chains: phenylalanine, tyrosine, and tryptophan; (5) basic side chains: lysine, arginine, and histidine; (6) acidic side chains: aspartate and glutamate, and (7) sulfur-containing side chains are cysteine and methionine. In some embodiments, conservative amino acids substitution groups are: valine-leucine-isoleucine, phenylalanine-tyrosine, lysine-arginine, alanine-valine, glutamate-aspartate, and asparagine-glutamine.

Alternatively, a conservative replacement is any change having a positive value in the PAM250 log-likelihood matrix disclosed in Gonnet et al. (1992) Science 256: 1443-1445. A “moderately conservative” replacement is any change having a nonnegative value in the PAM250 log-likelihood matrix.

Sequence similarity for polypeptides, which is also referred to as sequence identity, is typically measured using sequence analysis software. Protein analysis software matches similar sequences using measures of similarity assigned to various substitutions, deletions and other modifications, including conservative amino acid substitutions. For instance, gCG software contains programs such as gap and Bestfit which can be used with default parameters to determine sequence homology or sequence identity between closely related polypeptides, such as homologous polypeptides from different species of organisms or between a wild type protein and a mutein thereof. See, e.g., gCG Version 6.1. Polypeptide sequences also can be compared using FASTA using default or recommended parameters, a program in gCG Version 6.1. FASTA (e.g., FASTA2 and FASTA3) provides alignments and percent sequence identity of the regions of the best overlap between the query and search sequences (Pearson (2000) supra). Another particular algorithm when comparing a sequence of the description to a database containing a large number of sequences from different organisms is the computer program BLAST, especially BLASTP or TBLASTN, using default parameters. See, e.g., Altschul et al. (1990) J. Mol. Biol. 215:403-410 and Altschul et al. (1997) Nucleic Acids Res. 25:3389-402.

### Protein-Drug Conjugate Compounds

According to the foregoing objective and others, the present disclosure provides protein-drug conjugate compounds, e.g., antibody-drug conjugate compounds, and precursors and intermediates thereof, pharmaceutical compositions, and methods for treating certain diseases in a subject in need of such treatment. According to the disclosure, the protein-drug conjugate compounds provided herein comprise a glutaminyl-modified binding agent conjugated with a primary amine compound linked to a therapeutic moiety, e.g., exatecan or pro-exatecan moiety, as described herein.

In one aspect, the present disclosure provides compounds comprising a binding agent according to the present disclosure, (e.g., an antibody or a fragment thereof), having one or more glutamine residues conjugated to one or more compounds (e.g., exatecan or pro-exatecan), via a first linker, a unit comprising a triazole, and a second linker. Illustrative non-limiting examples include Formula (A) described herein. In specific embodiments of a protein-drug conjugate according to the disclosure, wherein the binding agent is an antibody, (e.g., a monoclonal antibody), the term “antibody drug conjugate” or ADC is optionally used.

In one aspect, the present disclosure provides a compound having a structure according to Formula (A):

BA-(L1-B-L2-P)ₙ (A), wherein:
BA is an antibody or an antigen-binding fragment thereof;
L1 is a first linker;
B is a moiety comprising a triazole;
L2 is a second linker;
P is selected from the group consisting of P-I through P-IV:

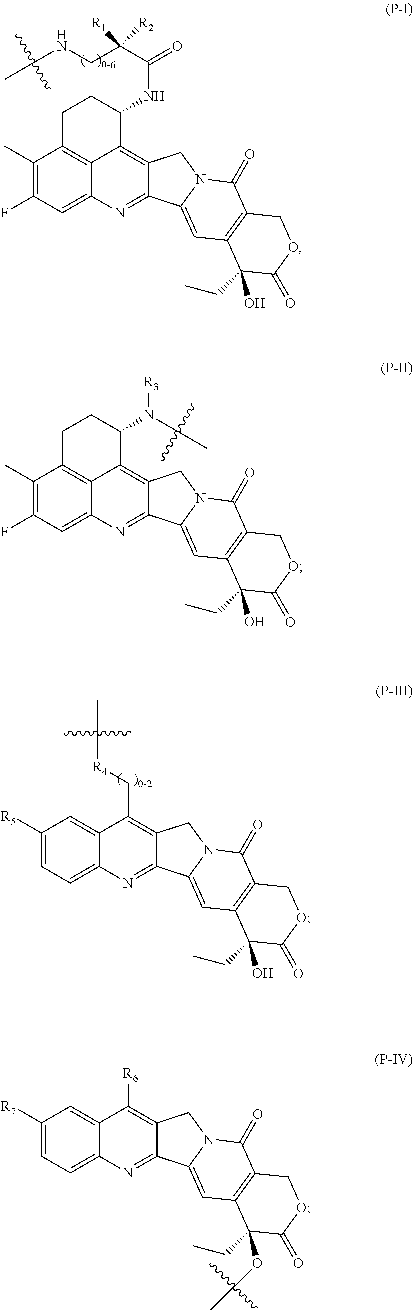

R₁ is hydrogen, C₁₋₆ alkyl, —(CH₂)ᵥ—OH, —(CH₂)ᵥ—NH₂, —(CH₂)ᵥ—C(O)OH, —(CH₂)ᵥ-phenyl, and —(CH₂)ᵥ—SO₂CH₃, wherein v is an integer from 0 to 12;
R² is hydrogen, C₁₋₆ alkyl, —(CH₂)ᵥ—OH, —(CH₂)ᵥ—NH₂, —(CH₂)ᵥ—C(O)OH, —(CH₂)ᵥ-phenyl, and —(CH₂)ᵥ—SO₂CH₃, wherein v is an integer from 0 to 12;
R³ is hydrogen —C₁₋₆ alkyl, —(CH₂)ᵥ—OH, —(CH₂)ᵥ—NH₂, —(CH₂)ᵥ—C(O)OH, —(CH₂)ᵥ-phenyl, —(CH₂)ᵥ—SO₂CH₃, and —CO—(CH₂)ᵥ—O—COCH₃, wherein v is an integer from 0 to 12;
R⁴ is —NH—, —N(—C₁₋₆ alkyl), —N(—C₁₋₆ alkyl)(—SO₂CH₃), —N(—C₁₋₆ alkyl)(—(CH₂)ᵥ—OH), —N(—C₁₋₆ alkyl)(CO—CH₂—NH₂), —N(—C₁₋₆ alkyl)(—(CH₂)ᵥ—O—CH₂—NH—CO—CH₂—NH₂), —N(—C₁₋₆ alkyl)(—CO—CH(NH₂)—(CH₂)ᵥ—COOH), —N(—C₁₋₆ alkyl)(—CO—CH(NH₂)—(CH₂)ᵥ—NH₂), —N(—C₁₋₆ alkyl)(—CO—CH(NH₂)—(CH₂)ᵥ-phenyl), or

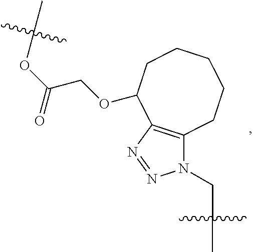

wherein v is an integer from 0 to 12;
R₅ is H, —OH, —OCH₃, or

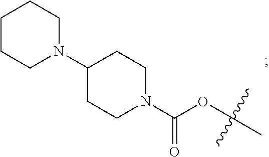 [SMILES: *OC(=O)N1CCC(N2CCCCC2)CC1]

R⁶ is hydrogen —C₁₋₆ alkyl, —(CH₂)ᵥ—OH, —(CH₂)ᵥ—NH₂, —(CH₂)ᵥ-phenyl, —(CH₂)ᵥ—N₃, —(CH₂)ᵥ—NH—CH₂-phenyl, —(CH₂)ᵥ—NMe-CH₂-phenyl-OMe, —(CH₂)ᵥ—NH—(CH₂)ᵥ—N₃, —(CH₂)ᵥ—NH—(CH₂)ᵥ—CCH; —(CH₂)ᵥ—NH—CO—CH(NH₂)—(CH₂)ᵥ-phenyl, —(CH₂)ᵥ—NH—CO—CH(NH₂)—(CH₂)ᵥ—N₃, —(CH₂)ᵥ—NH—CO—CH(NH₂)—(CH₂)ᵥ—NH₂, —(CH₂)ᵥ—NH—CO—CH(NH₂)—(CH₂)ᵥ—COOH,

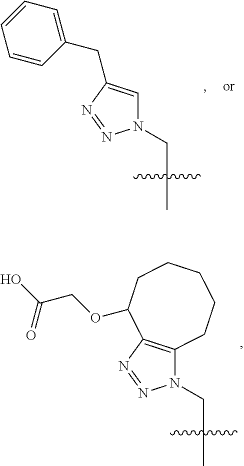

wherein v is an independently an integer from 0 to 12;
R⁷ is H, —OH, —OCH₃, or

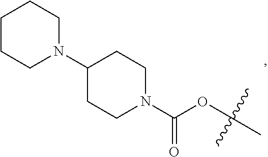 [SMILES: *OC(=O)N1CCC(N2CCCCC2)CC1]

n is an integer from 1 to 12.

In one embodiment, the -L1-B-L2-P of the compound of Formula (A) has the following structure:

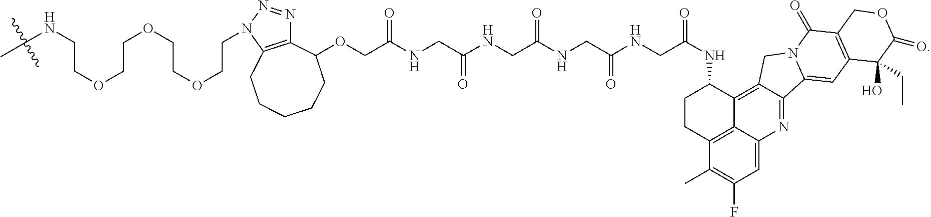 [SMILES: *NCCOCCOCCOCCn1nnc2c1CCCCCC2OCC(=O)NCC(=O)NCC(=O)NCC(=O)NCC(=O)N[C@H]1CCc2c(C)c(F)cc3nc4c(c1c23)Cn1c-4cc2c(c1=O)COC(=O)[C@]2(O)CC]

In one embodiment, the -L1-B-L2-P of the compound of Formula (A) has the following structure:

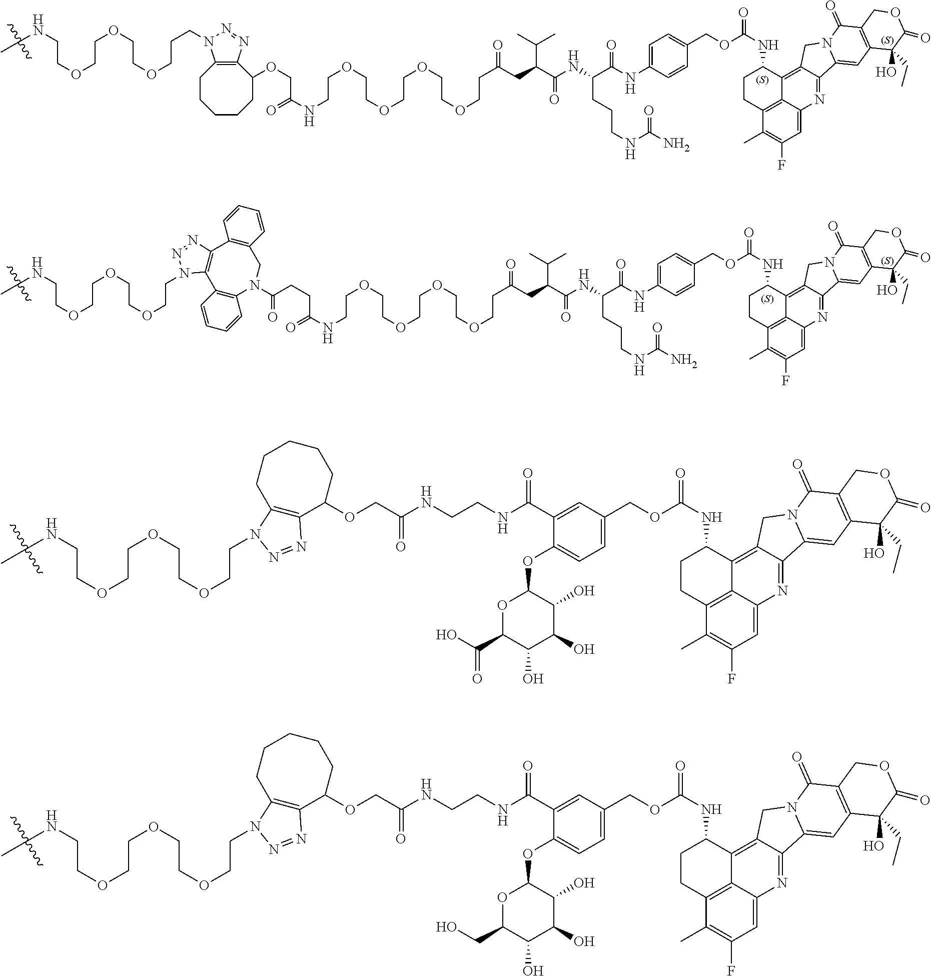

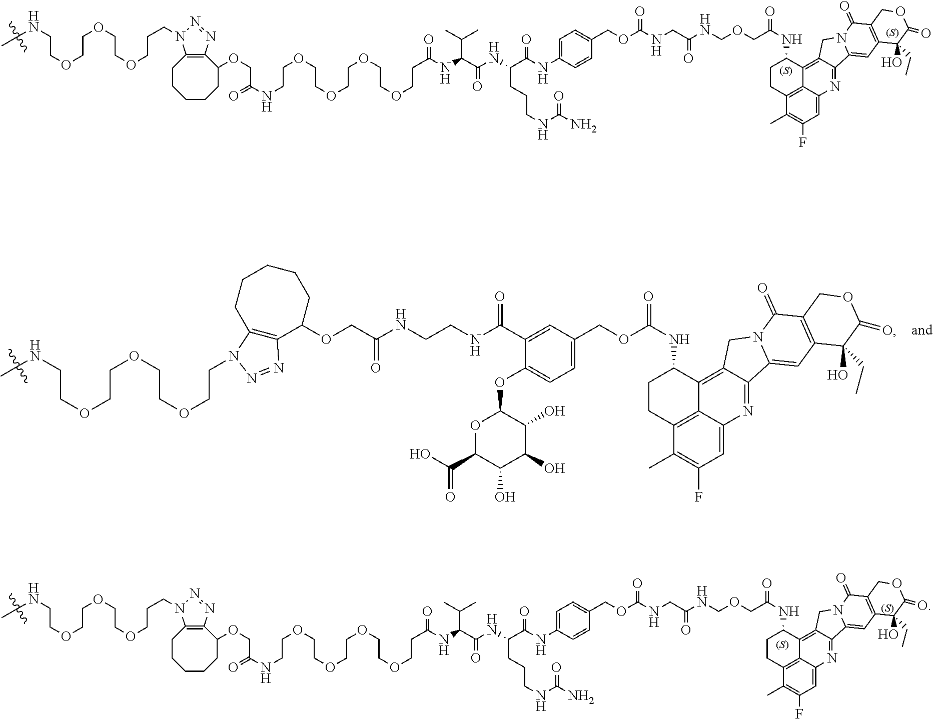

In one embodiment, the compound of the present disclosure has a structure selected from the group consisting of:

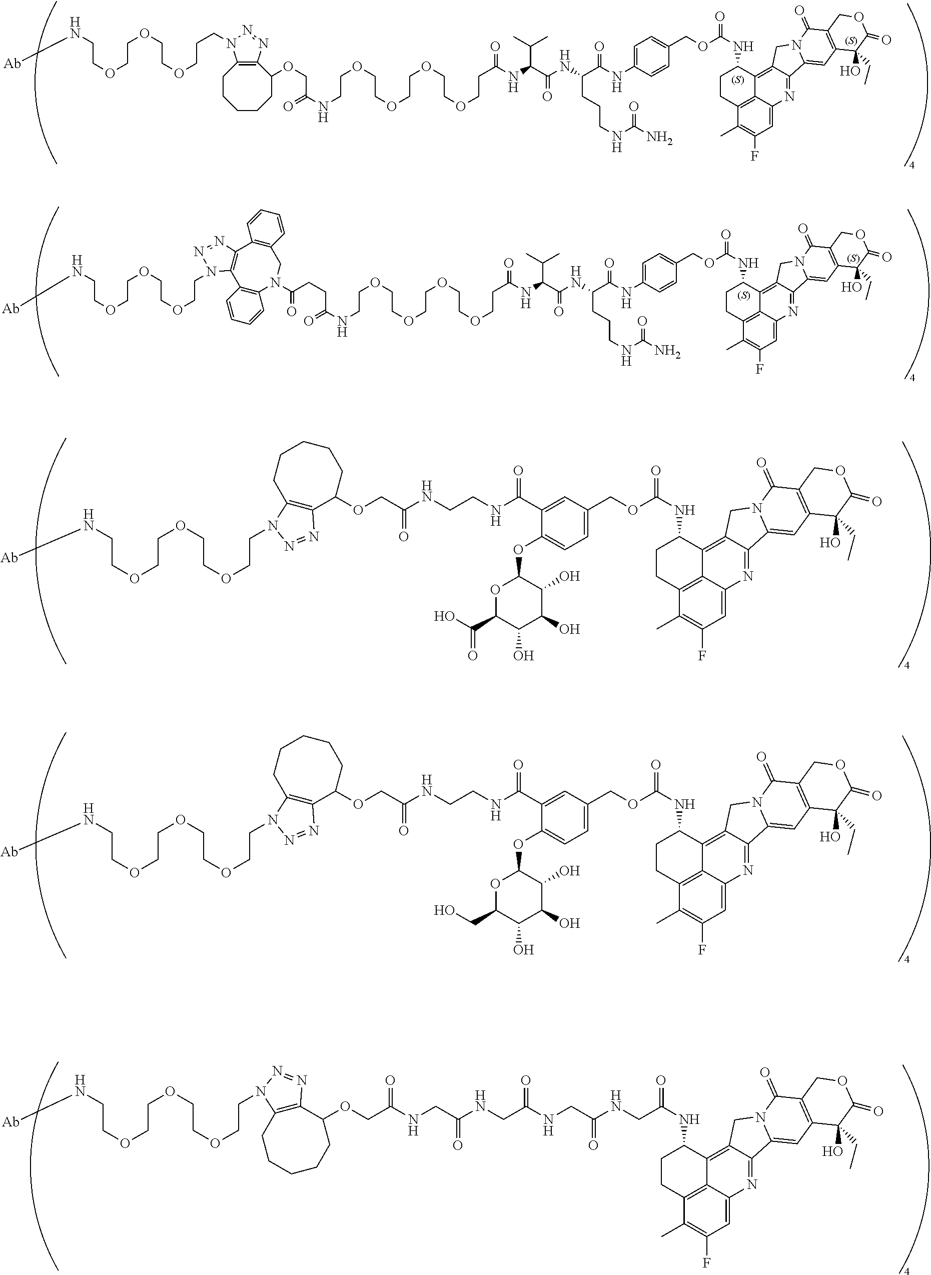

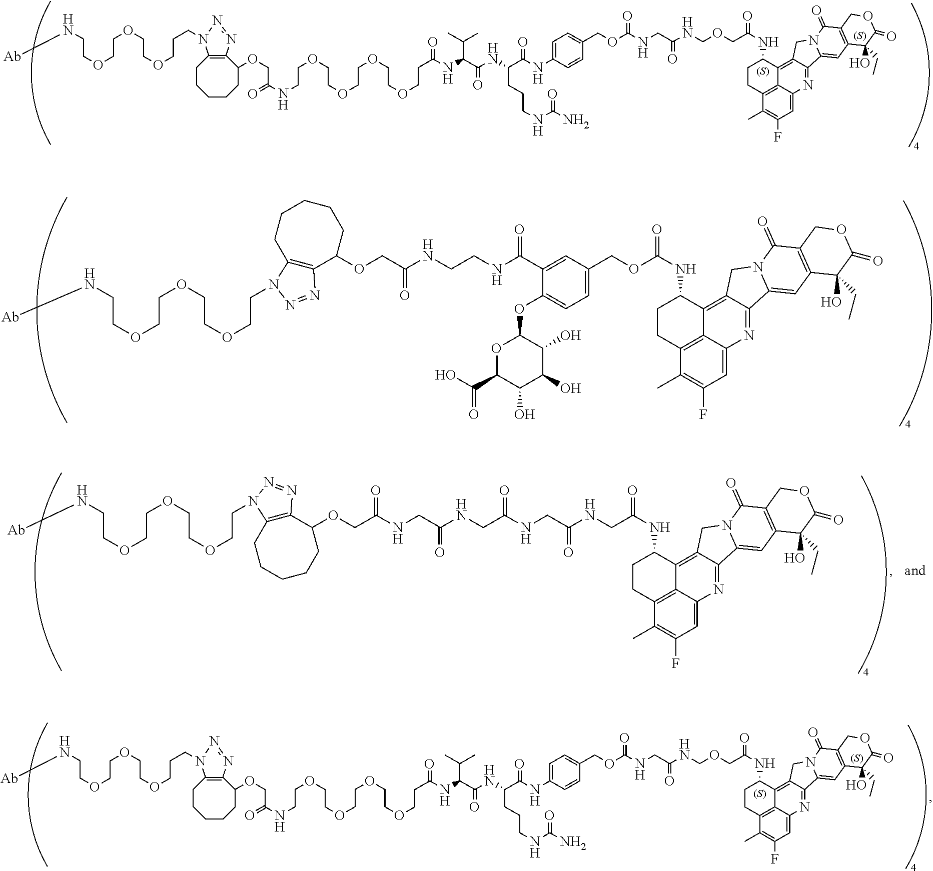

or a pharmaceutically acceptable salt thereof.

### Linker L1

In certain embodiments, linker L1 is covalently attached to the amine of a glutamine residue of the binding agent BA.

In certain embodiments, linker L1 comprises an alkyl (e.g., a C₁₋₂₀ alkyl, or a C₁₋₁₂ alkyl, or a C₁₋₆ alkyl), a phenyl, aralkyl-NH—, —C(O)—, —(CH₂)ᵤ—NH—C(O)—, —(CH₂)ᵤ—C(O)—NH—, —(CH₂—CH₂—O)ᵥ—, —(CH₂)ᵤ—(O—CH₂—CH₂)ᵥ—C(O)—NH—, a peptide unit comprising from 2 to 4 amino acids, or combinations thereof, each of which may be optionally substituted with one or more of —S—, —S(O₂)—, —C(O)—, —C(O₂)—; or —CO₂H, wherein subscripts u and v are independently an integer from 1 to 8.

In certain embodiments, the free (unconjugated) linker L1 comprises a primary amine for attachment to the glutamine residue via a transglutamination reaction.

In one embodiment, linker L1 comprises one or more polyethylene glycol (PEG) units. In one embodiment, L1 comprises 2, or 3, or 4, or 5, or 6, or 7, or 8, or 9, or 10 PEG units.

In one embodiment, linker L1 comprises a disulfide (—S—S—) bond.

In one embodiment, linker L1 comprises a —S(O₂)— moiety.

In one embodiment, one or more carbons on linker L1 is substituted with —CO₂H.

In one embodiment, linker L1 comprises a peptide unit comprising from 2 to 4 amino acids, or a peptide unit comprising 2 amino acids, a peptide unit comprising 3 amino acids, or a peptide unit comprising 4 amino acids.

In one embodiment, linker L1 comprises a peptide unit comprising 2 amino acids selected from alanine, glycine, valine, phenylalanine, proline, glutamic acid, and citrulline, and combinations thereof. In one particular embodiment, linker L1 comprises a valine-citrulline unit.

In one embodiment, linker L1 is selected from the group consisting of:

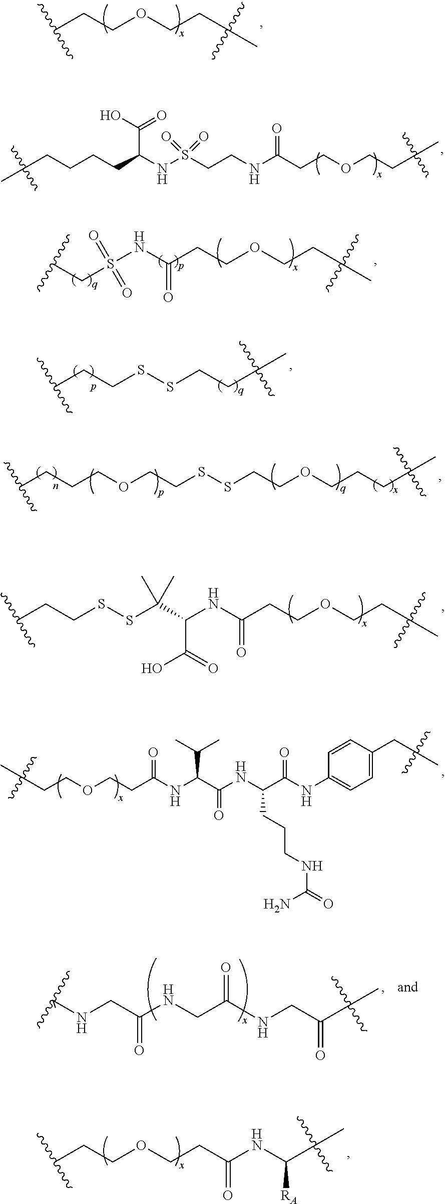

wherein R_{A} is a group comprising an alkyne, an azide, a tetrazine, a trans-cyclooctene, a maleimide, an amine, a ketone, an aldehyde, a carboxylic acid, an ester, a thiol, a sulfonic acid, a tosylate, a halide, a silane, a cyano group, a carbohydrate group, a biotin group, a lipid residue, and wherein subscripts x, n, p and q are independently an integer from 0 to 12, and combinations thereof.

In one embodiment, linker L1 is selected from the group consisting of:

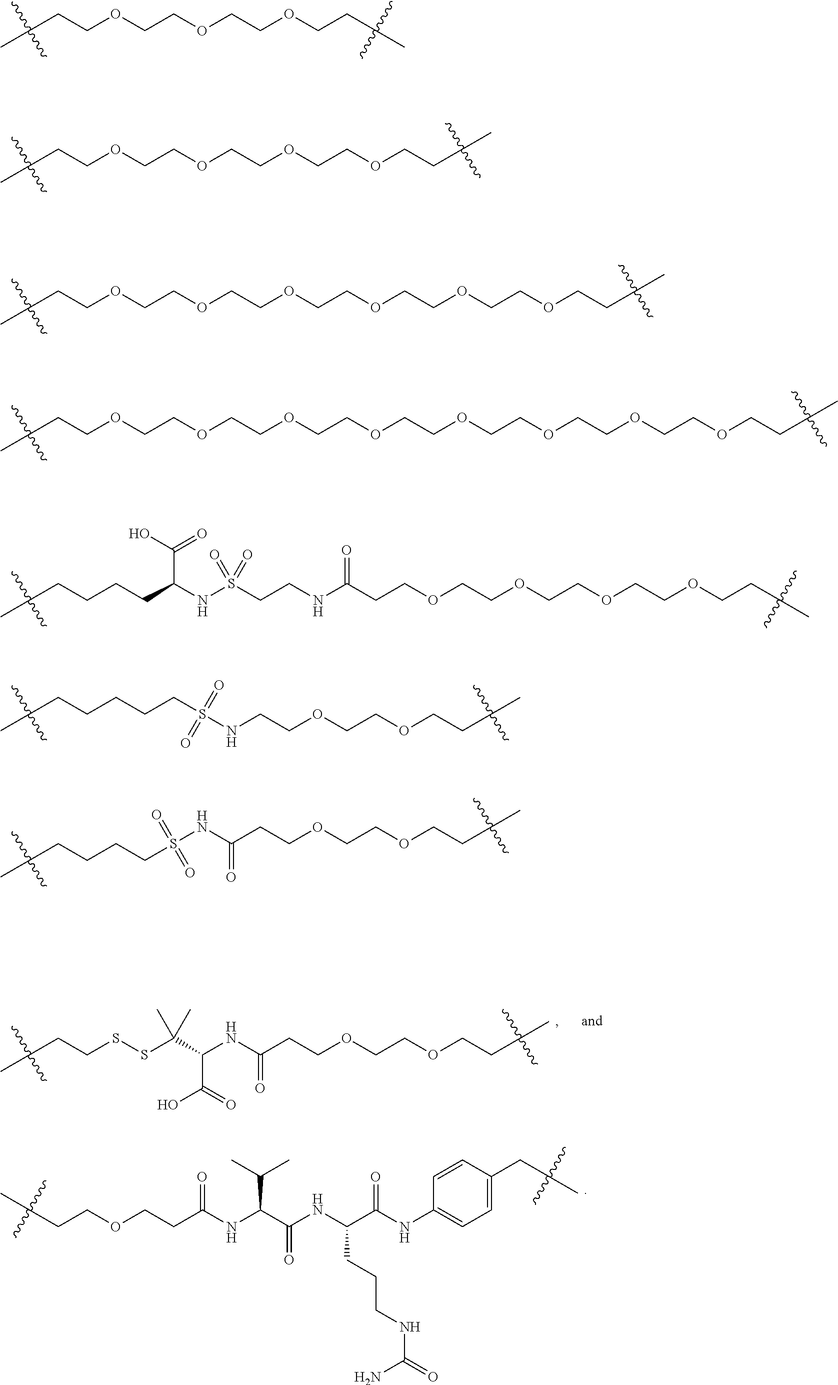

In one embodiment, L1 is

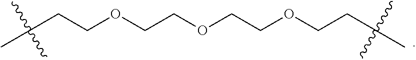 [SMILES: *CCOCCOCCOCC*]

In another embodiment, L1 is selected from the group consisting of:

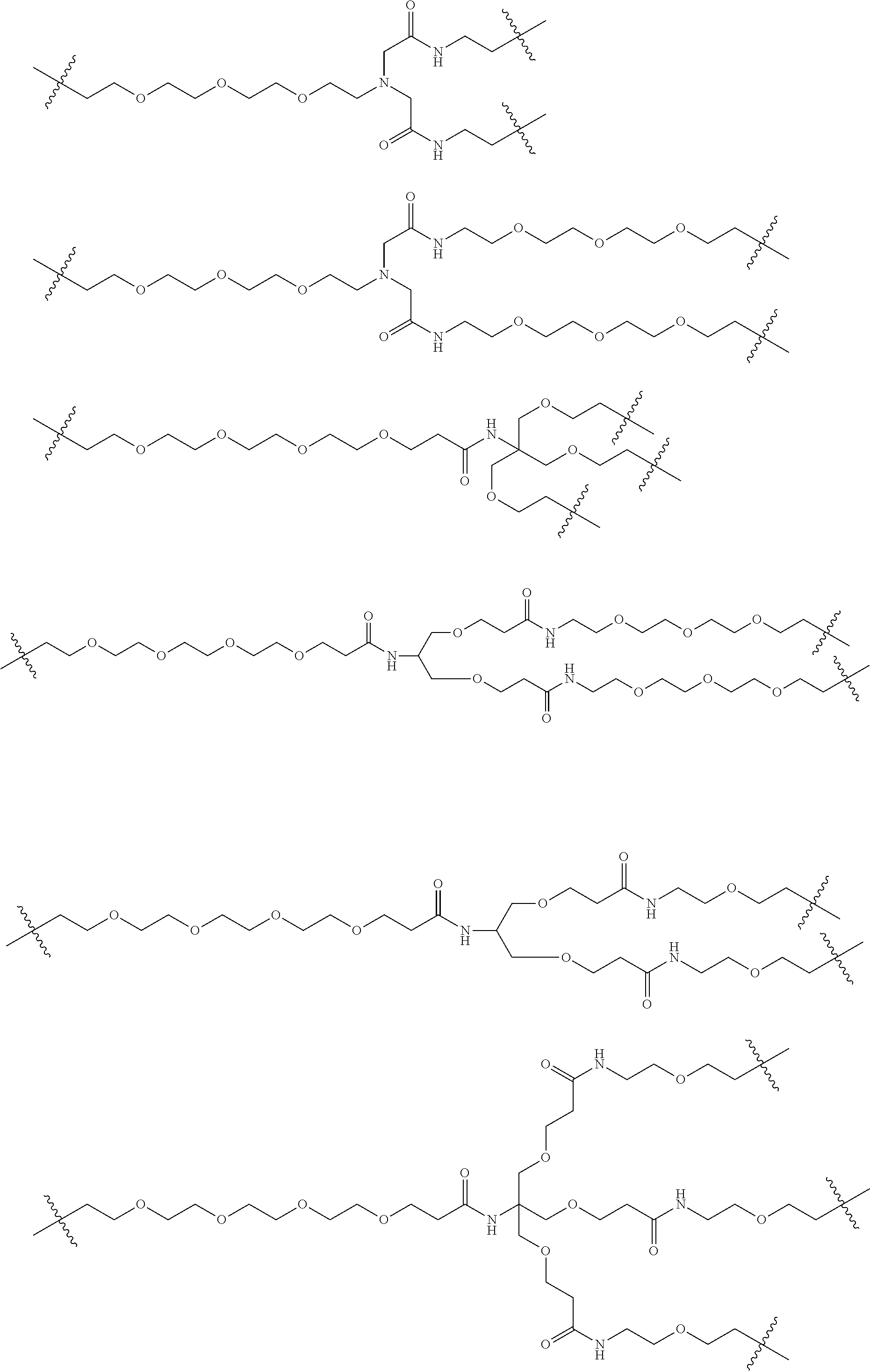 [SMILES: *CCNC(=O)CN(CCOCCOCCOCC*)CC(=O)NCC* ; *CCOCCOCCOCCNC(=O)CN(CCOCCOCCOCC*)CC(=O)NCCOCCOCCOCC* ; *CCOCCOCCOCCOCCC(=O)NC(COCC*)(COCC*)COCC* ; *CCOCCOCCOCCNC(=O)CCOCC(COCCC(=O)NCCOCCOCCOCC*)NC(=O)CCOCCOCCOCCOCC* ; *CCOCCNC(=O)CCOCC(COCCC(=O)NCCOCC*)NC(=O)CCOCCOCCOCCOCC* ; *CCOCCNC(=O)CCOCC(COCCC(=O)NCCOCC*)(COCCC(=O)NCCOCC*)NC(=O)CCOCCOCCOCCOCC*]

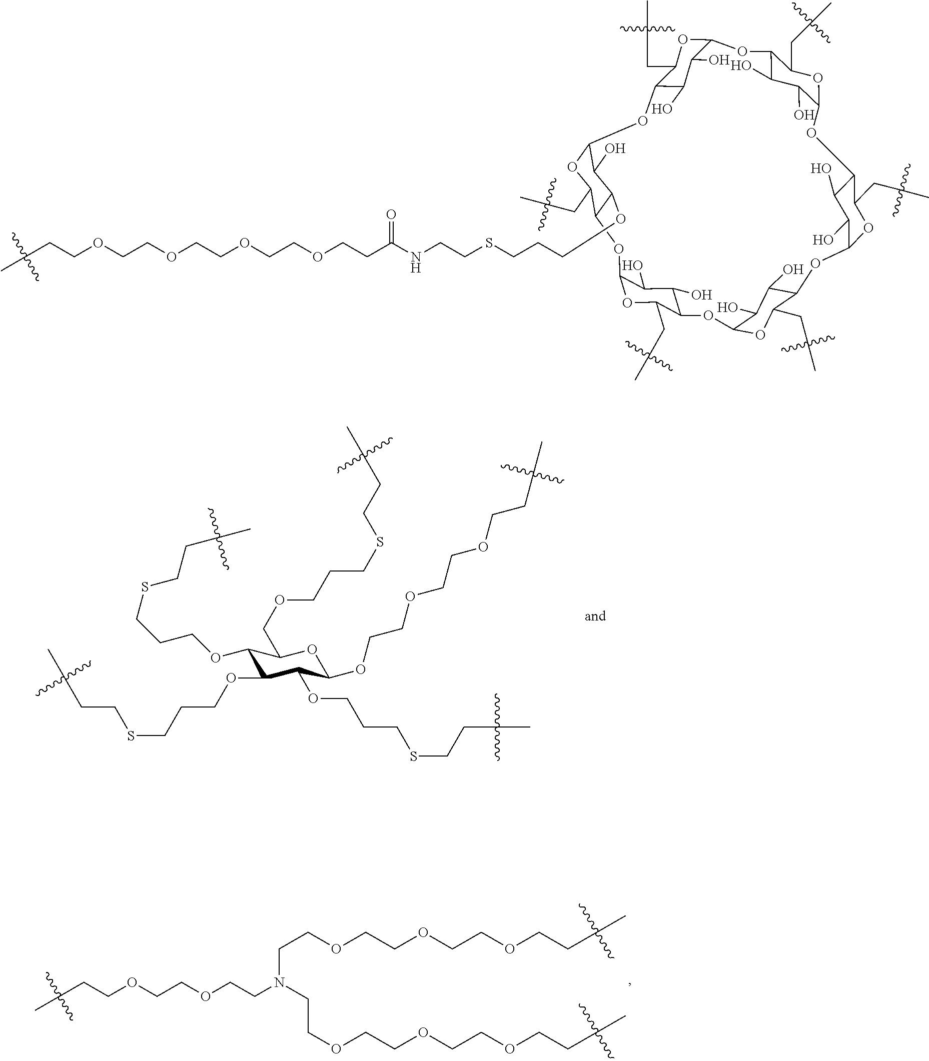

or a pharmaceutically acceptable salt thereof.

### Triazole-Comprising Moiety B

In one aspect, B is a compound comprising a triazole

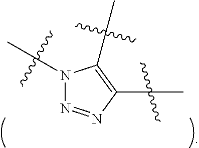

In one embodiment, B is selected from the group consisting of:

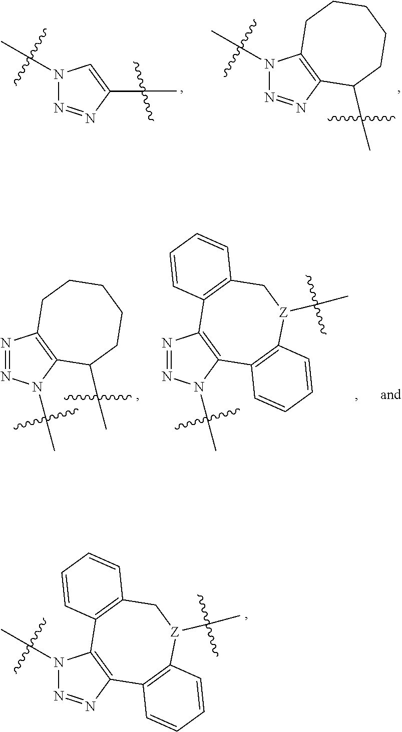

where Z is C or N

In one embodiment, B is

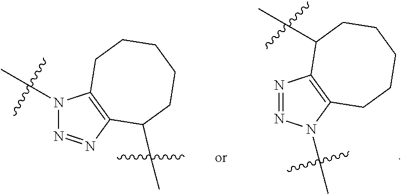 [SMILES: *C1CCCCCc2c1nnn2* ; *C1CCCCCc2c1nnn2*]

In another embodiment,
B is

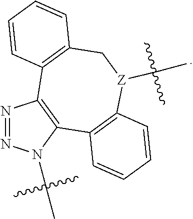

### Linker L2

In certain aspects of the present disclosure, linker L2 has a structure according to Formula (L2):

-SP1-AA-SP2- (L2),

wherein:
SP1 is absent or a first spacer unit;
AA is absent or a peptide unit comprising from 2 to 4 amino acids;
SP2 is absent or a second spacer unit covalently attached to the P, provided that at least one of
SP1, AA and SP2 is not absent.

In one embodiment, SP1 is absent.

In one embodiment, SP1 is selected from the group consisting of

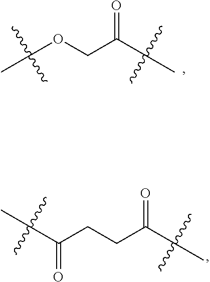 [SMILES: *OCC(*)=O ; *C(=O)CCC(*)=O]

C₁₋₆ alkyl, —(CH₂—CH₂—O)ᵥ—, —NH—, —C(O)—, —NH—C(O)—, —NH—(CH₂)ᵤ—, —NH—(CH₂)ᵤ—C(O)—, —NH—(CH₂—CH₂—O)ᵥ—, —NH—(CH₂—CH₂—O)ᵥ—C(O)—, —NH—(CH₂—CH₂—O)ᵥ—(CH₂)ᵤ—, —NH—(CH₂—CH₂—O)ᵥ—(CH₂)ᵤ—C(O)—, —(CH₂)ᵤ—NH—C(O)—, —NH—(CH₂)ᵤ—NH—C(O)—, —NH—(CH₂)ᵤ—C(O)—NH—, and combinations thereof; wherein subscripts u and v are independently an integer from 1 to 8.

In one embodiment, AA is absent.

In one embodiment, AA is a peptide unit comprising from 2 to 4 amino acids selected from alanine, glycine, valine, proline, glutamic acid, lysine, phenylalanine, and citrulline, and combinations thereof.

In one embodiment, AA is valine-citrulline, glutamic acid-valine-citrulline, glycine-glycine-glycine, or glycine-glycine-glycine-glycine.

In one embodiment, AA is valine-citrulline.

In one embodiment, AA is glutamic acid-valine-citrulline.

In one embodiment, AA is glycine-glycine-glycine.

In one embodiment, AA is glycine-glycine-glycine-glycine

In one embodiment, SP2 is absent.

In one embodiment, SP2 is selected from the group consisting of

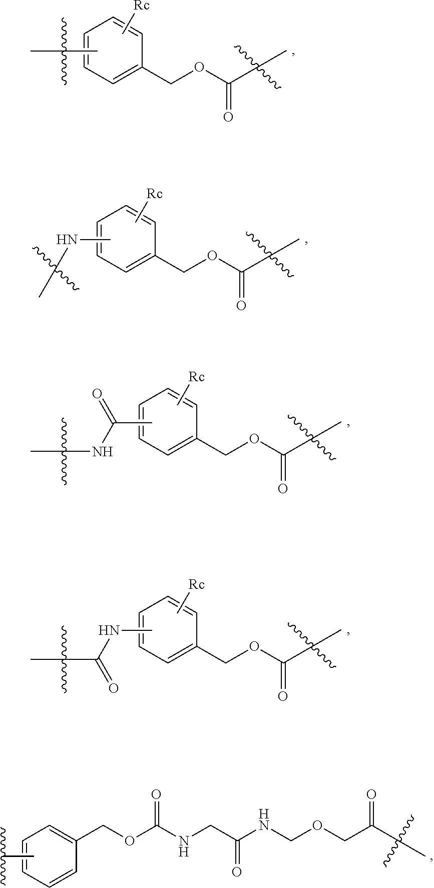

and combinations thereof, wherein R, is independently at
each occurrence absent or a group selected from

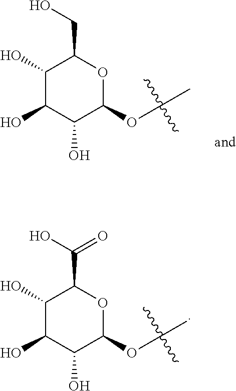

In certain embodiments, L2 is selected from the group consisting of:

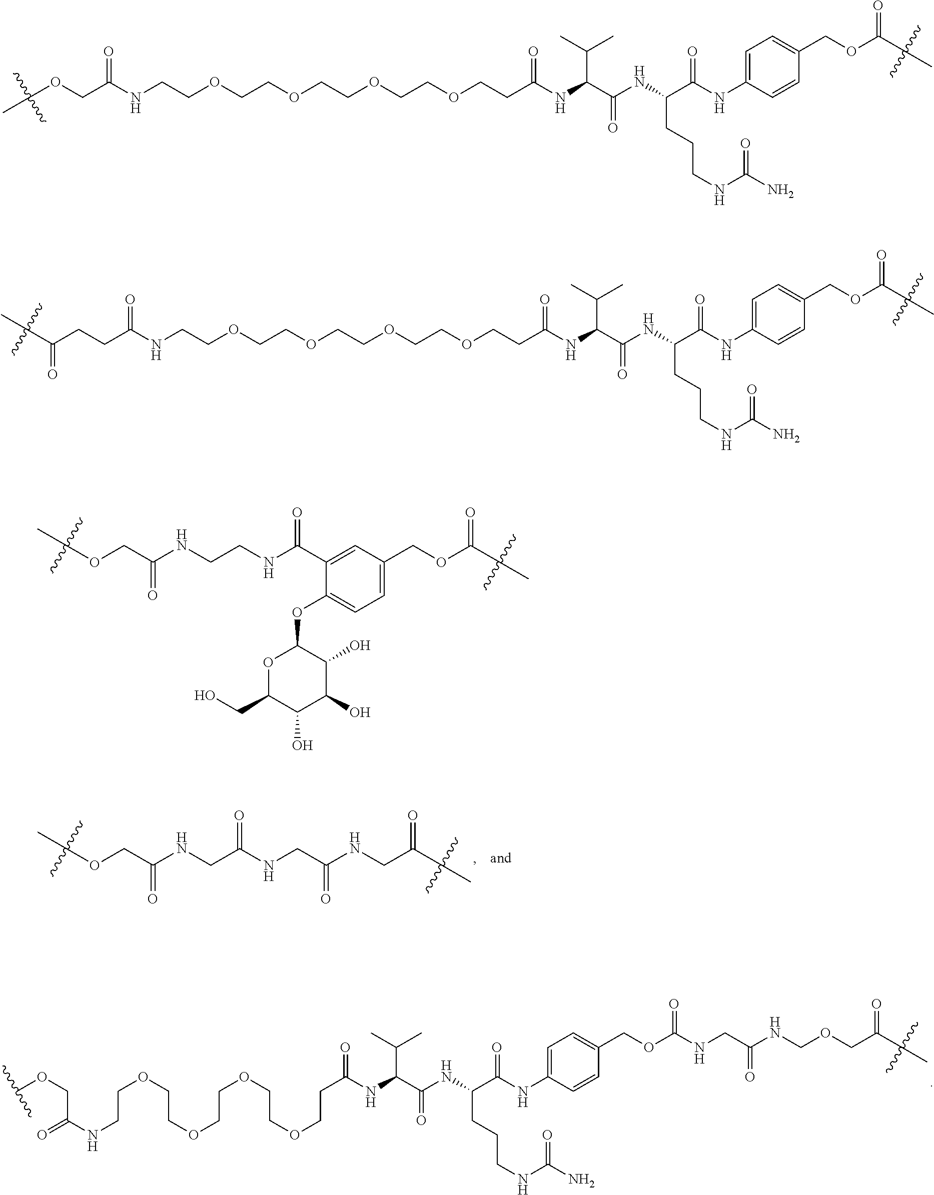 [SMILES: *OCC(=O)NCCOCCOCCOCCOCCC(=O)N[C@H](C(=O)N[C@@H](CCCNC(N)=O)C(=O)Nc1ccc(COC(*)=O)cc1)C(C)C ; *C(=O)CCC(=O)NCCOCCOCCOCCOCCC(=O)N[C@H](C(=O)N[C@@H](CCCNC(N)=O)C(=O)Nc1ccc(COC(*)=O)cc1)C(C)C ; *OCC(=O)NCCNC(=O)c1cc(COC(*)=O)ccc1O[C@@H]1O[C@H](CO)[C@@H](O)[C@H](O)[C@H]1O ; *OCC(=O)NCC(=O)NCC(=O)NCC(*)=O ; *OCC(=O)NCCOCCOCCOCCOCCC(=O)N[C@H](C(=O)N[C@@H](CCCNC(N)=O)C(=O)Nc1ccc(COC(=O)NCC(=O)NCOCC(*)=O)cc1)C(C)C]

### Payloads

In certain embodiments, the payloads of the present disclosure are camptothecin analogs and/or derivatives.

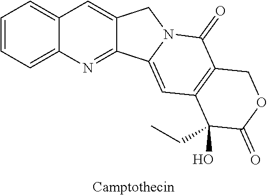 [SMILES: CC[C@@]1(O)C(=O)OCc2c1cc1n(c2=O)Cc2cc3ccccc3nc2-1]

Camptothecin (CPT), shown above, is a topoisomerase poison. It was discovered in 1966 by M. E. Wall and M. C. Wani in systematic screening of natural products for anticancer drugs. It was isolated from the bark and stem of Camptotheca acuminata (Camptotheca, Happy tree), a tree native to China used as a cancer treatment in Traditional Chinese Medicine. Camptothecin showed remarkable anticancer activity in preliminary clinical trials. However, it has low solubility, so synthetic and medicinal chemists have developed numerous syntheses of camptothecin and various derivatives to increase the benefits of the chemical, with good results. Four camptothecin analogs have been approved and are used in cancer chemotherapy today: topotecan, irinotecan, belotecan, and deruxtecan (Dxd).

Trastuzumab deruxtecan (T-Dxd, aka DS8201a) is an antibody-drug conjugate that includes a human epidermal growth factor receptor 2 (HER2)-directed antibody trastuzumab and a topoisomerase I inhibitor conjugate deruxtecan (Dxd, a derivative of exatecan). It was approved for use in the United States in December 2019.

Exatecan, shown below, is a camptothecin analog.

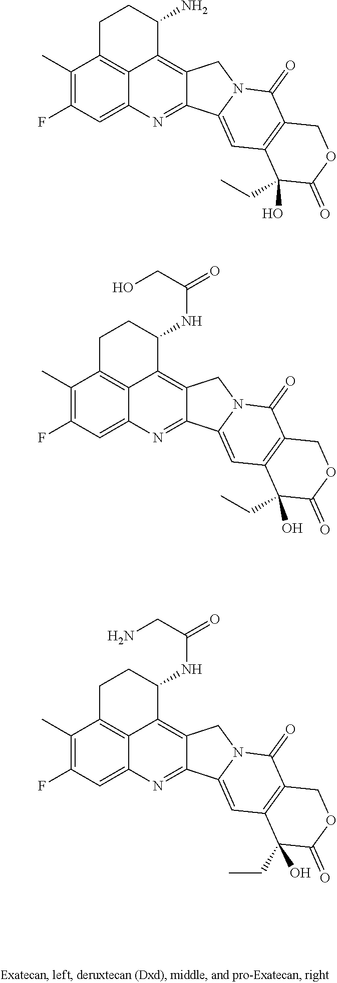 [SMILES: CC[C@@]1(O)C(=O)OCc2c1cc1n(c2=O)Cc2c-1nc1cc(F)c(C)c3c1c2[C@@H](N)CC3 ; CC[C@@]1(O)C(=O)OCc2c1cc1n(c2=O)Cc2c-1nc1cc(F)c(C)c3c1c2[C@@H](NC(=O)CO)CC3 ; CC[C@@]1(O)C(=O)OCc2c1cc1n(c2=O)Cc2c-1nc1cc(F)c(C)c3c1c2[C@@H](NC(=O)CN)CC3]

In one embodiment, the payload of the present disclosure is Exatecan.

In certain embodiments, the payload of the present disclosure is a compound having the structure P2 (pro-Exatecan):

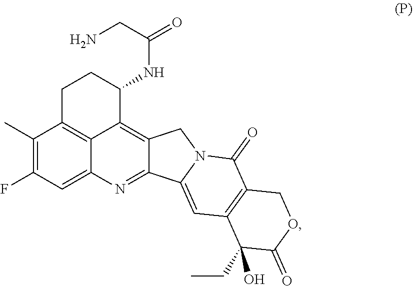 [SMILES: CC[C@@]1(O)C(=O)OCc2c1cc1n(c2=O)Cc2c-1nc1cc(F)c(C)c3c1c2[C@@H](NC(=O)CN)CC3]

or a pharmaceutically acceptable salt thereof.

In certain embodiments, the present disclosure provides a payload having the structure selected from the group consisting of P′-A to P′-IV:

P′-I through P′-IV:

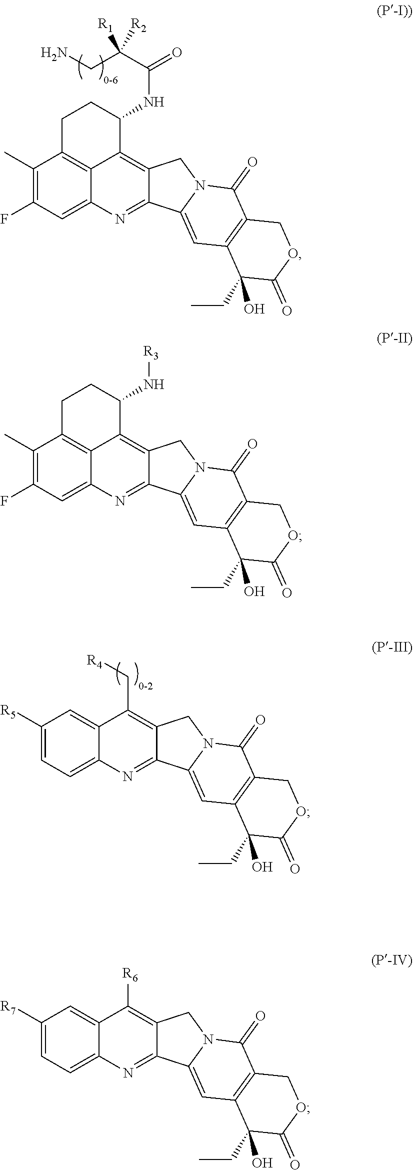

or a pharmaceutically acceptable salt thereof, wherein:
R₁ is hydrogen, C₁₋₆ alkyl, —(CH₂)ᵥ—OH, —(CH₂)ᵥ—NH₂, —(CH₂)ᵥ—C(O)OH, —(CH₂)ᵥ-phenyl, and —(CH₂)ᵥ—SO₂CH₃, wherein v is an integer from 0 to 12;
R² is hydrogen, C₁₋₆ alkyl, —(CH₂)ᵥ—OH, —(CH₂)ᵥ—NH₂, —(CH₂)ᵥ—C(O)OH, —(CH₂)ᵥ-phenyl, and —(CH₂)ᵥ—SO₂CH₃, wherein v is an integer from 0 to 12;
R³ is hydrogen —C₁₋₆ alkyl, —(CH₂)ᵥ—OH, —(CH₂)ᵥ—NH₂, —(CH₂)ᵥ—C(O)OH, —(CH₂)ᵥ-phenyl, —(CH₂)ᵥ—SO₂CH₃, and —CO—(CH₂)ᵥ—O—COCH₃, wherein v is an integer from 0 to 12;
R⁴ is —NH—, —N(—C₁₋₆ alkyl), —N(—C₁₋₆ alkyl)(—SO₂CH₃), —N(—C₁₋₆ alkyl)(—(CH₂)ᵥ—OH), —N(—C₁₋₆ alkyl)(CO—CH₂—NH₂), —N(—C₁₋₆ alkyl)(—(CH₂)ᵥ—O—CH₂—NH—CO—CH₂—NH₂), —N(—C₁₋₆ alkyl)(—CO—CH(NH₂)—(CH₂)ᵥ—COOH), —N(—C₁₋₆ alkyl)(—CO—CH(NH₂)—(CH₂)ᵥ—NH₂), —N(—C₁₋₆ alkyl)(—CO—CH(NH₂)—(CH₂)ᵥ-phenyl), or

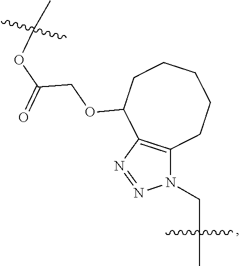

wherein v is an integer from 0 to 12;
R⁵ is H, —OH, —OCH₃, or

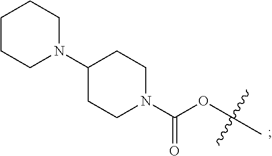 [SMILES: *OC(=O)N1CCC(N2CCCCC2)CC1]

R⁶ is hydrogen —C₁₋₆ alkyl, —(CH₂)ᵥ—OH, —(CH₂)ᵥ—NH₂, —(CH₂)ᵥ-phenyl, —(CH₂)ᵥ—N₃, —(CH₂)ᵥ—NH—CH₂-phenyl, —(CH₂)ᵥ—NMe-CH₂-phenyl-OMe, —(CH₂)ᵥ—NH—(CH₂)ᵥ—N₃, —(CH₂)ᵥ—NH—(CH₂)ᵥ—CCH; —(CH₂)ᵥ—NH—CO—CH(NH₂)—(CH₂)ᵥ-phenyl, —(CH₂)ᵥ—NH—CO—CH(NH₂)—(CH₂)ᵥ—N₃, —(CH₂)ᵥ—NH—CO—CH(NH₂)—(CH₂)ᵥ—NH₂, —(CH₂)ᵥ—NH—CO—CH(NH₂)—(CH₂)ᵥ—COOH,

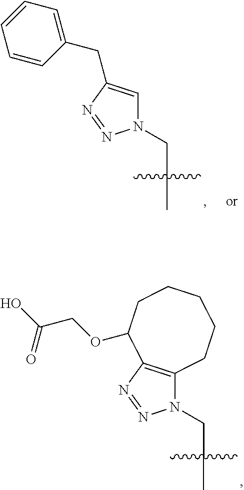

wherein v is an independently an integer from 0 to 12, and
R⁷ is H, —OH, —OCH₃, or

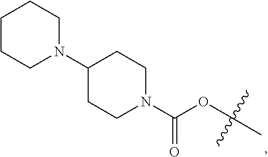 [SMILES: *OC(=O)N1CCC(N2CCCCC2)CC1]

or a pharmaceutically acceptable salt thereof.

In certain embodiments, the payload of the present disclosure is a compound having the structure selected from the group consisting of:

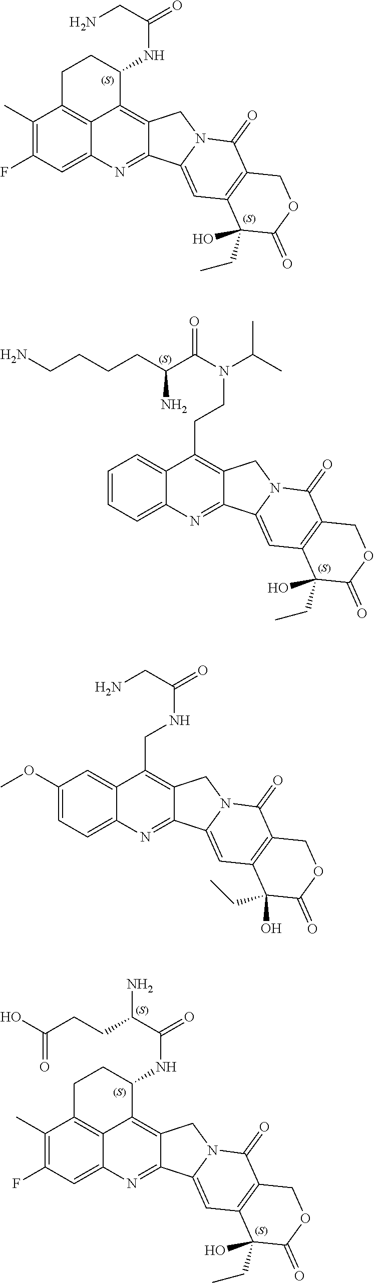

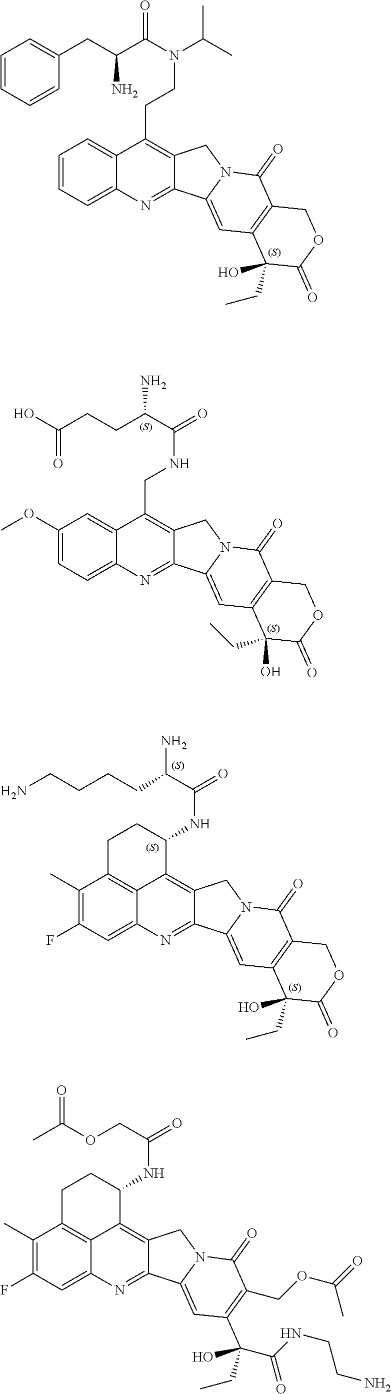

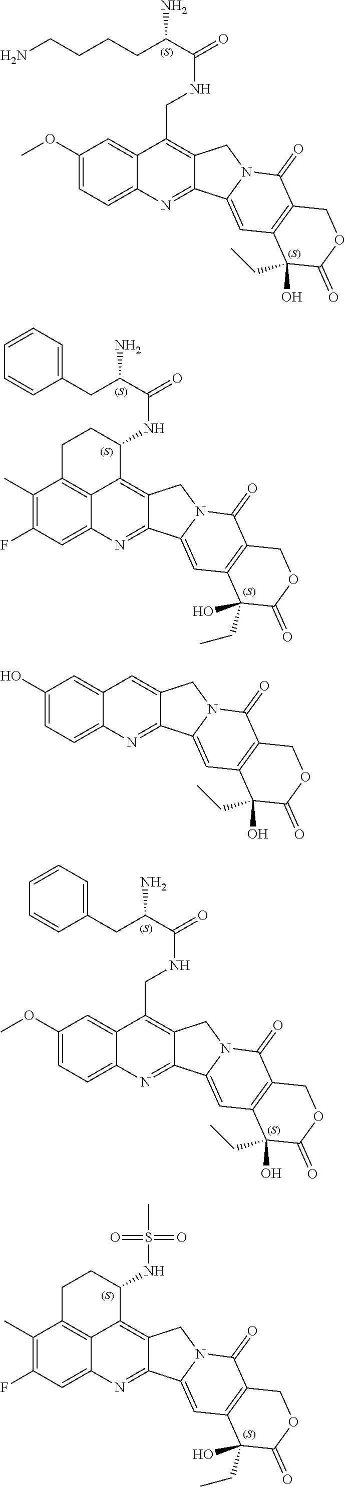

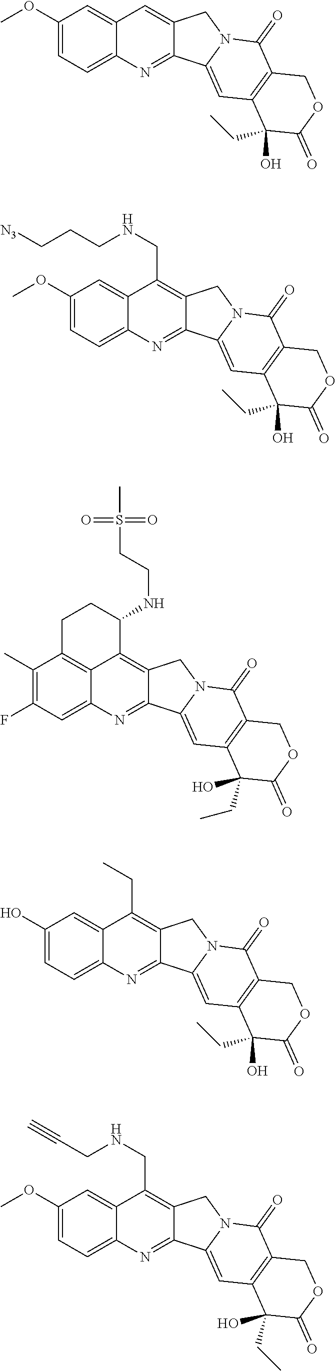 [SMILES: CC[C@@]1(O)C(=O)OCc2c1cc1n(c2=O)Cc2cc3cc(OC)ccc3nc2-1 ; CC[C@@]1(O)C(=O)OCc2c1cc1n(c2=O)Cc2c-1nc1ccc(OC)cc1c2CNCCCN=[N+]=[N-] ; CC[C@@]1(O)C(=O)OCc2c1cc1n(c2=O)Cc2c-1nc1cc(F)c(C)c3c1c2[C@@H](NCCS(C)(=O)=O)CC3 ; CCc1c2c(nc3ccc(O)cc13)-c1cc3c(c(=O)n1C2)COC(=O)[C@]3(O)CC ; C#CCNCc1c2c(nc3ccc(OC)cc13)-c1cc3c(c(=O)n1C2)COC(=O)[C@]3(O)CC]

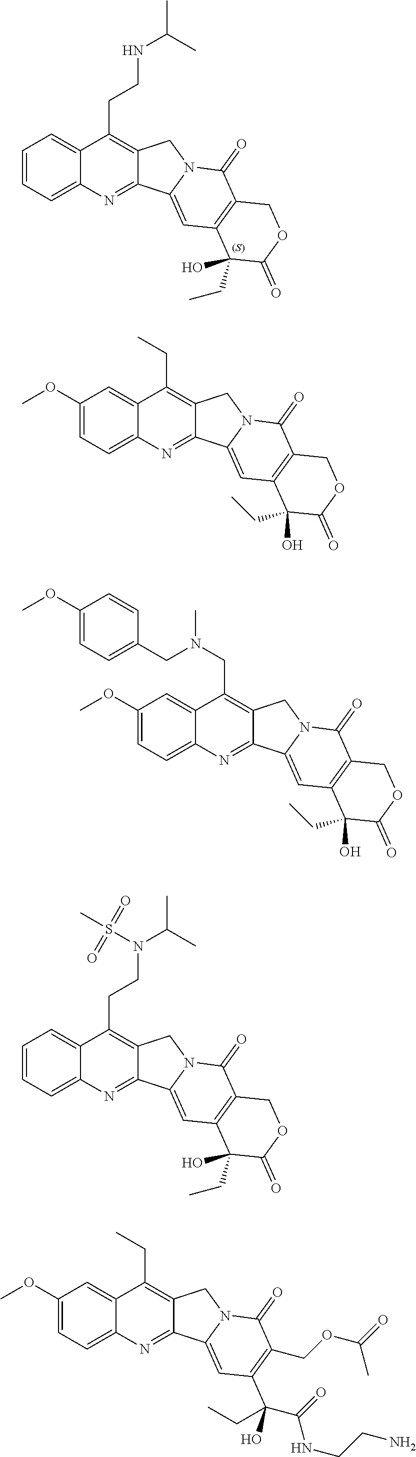

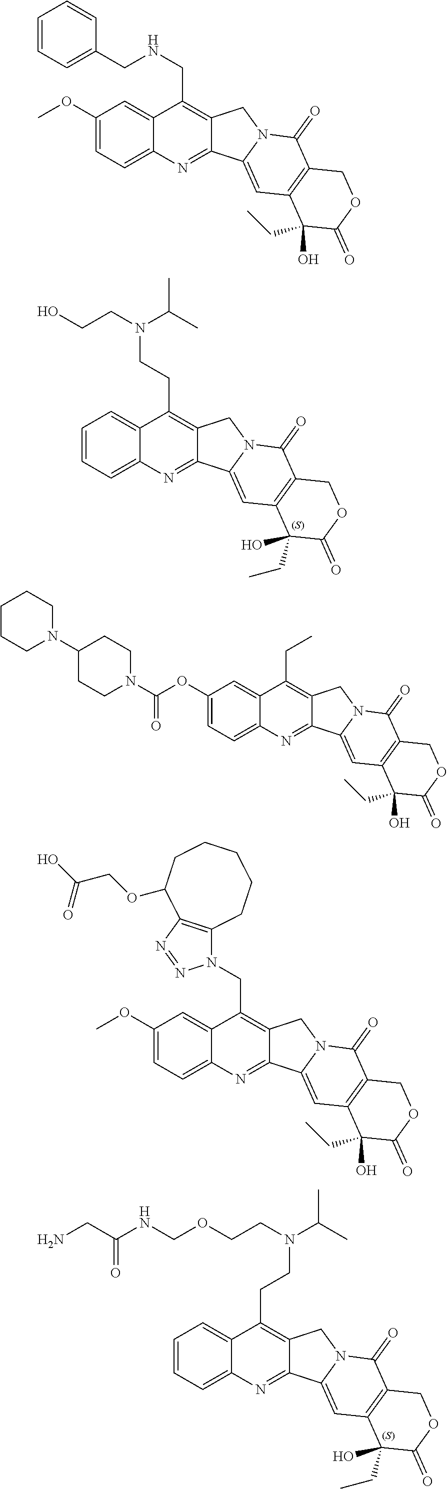

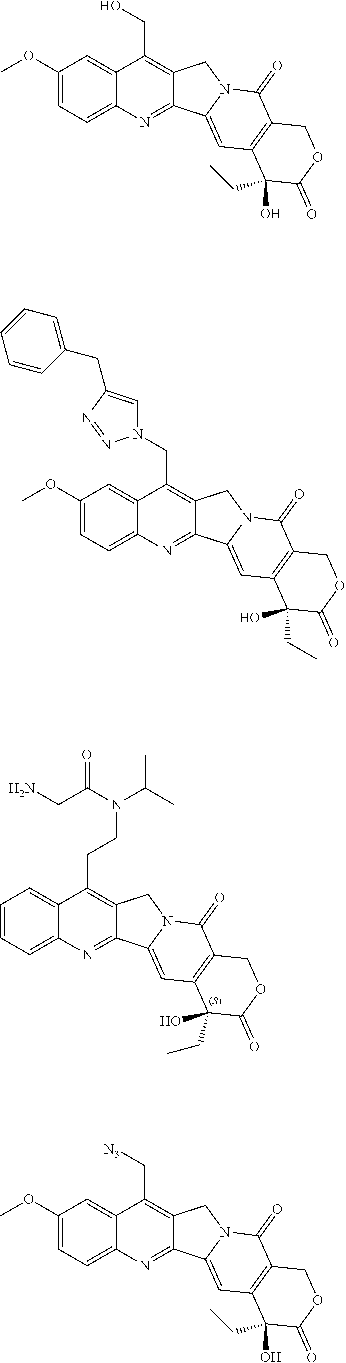

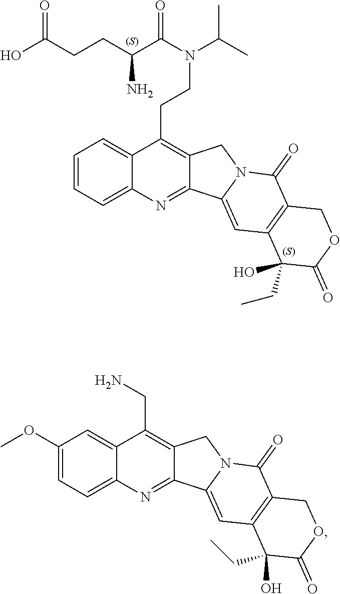

or a pharmaceutically acceptable salt thereof.

The present disclosure also relates to a pharmaceutical composition comprising a therapeutically effective amount of the compound as described above or a pharmaceutically acceptable salt thereof and one or more pharmaceutically acceptable carriers, diluents, or excipients.

### Linker-Payloads (L2-P)

In another aspect, the present disclosure provides a compound according to Formula (Alk-L2-P):

Alk-SP1-AA-SP2-P (Alk-L2-P)

wherein:
Alk is a moiety comprising an alkyne;
SP1 is absent or a first spacer unit;
AA is absent or a peptide unit comprising from 2 to 4 amino acids;
SP2 is absent or a second spacer unit;
P is selected from the group consisting of P-I through P-IV:

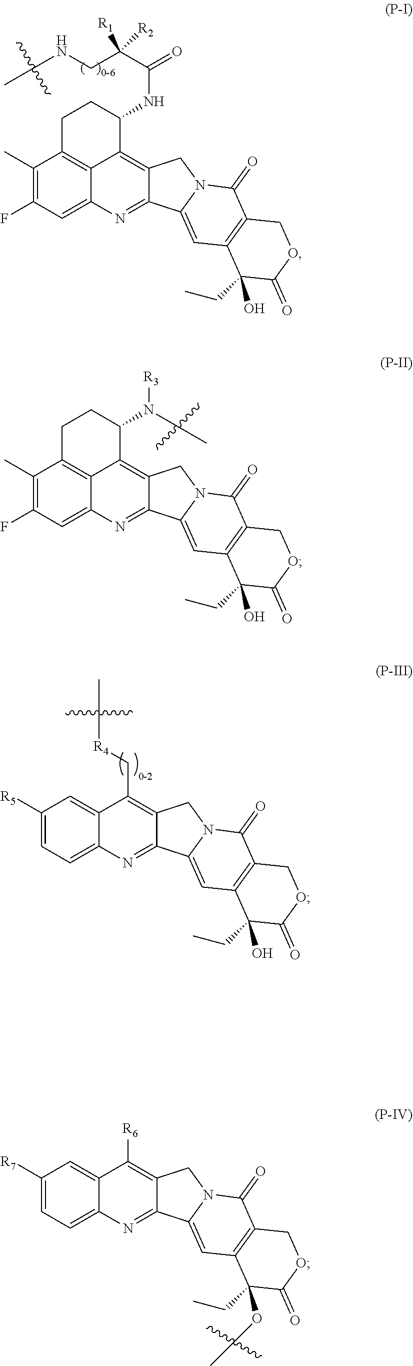

R¹ is hydrogen, C₁₋₆ alkyl, —(CH₂)ᵥ—OH, —(CH₂)ᵥ—NH₂, —(CH₂)ᵥ—C(O)OH, —(CH₂)ᵥ-phenyl, and —(CH₂)ᵥ—SO₂CH₃, wherein v is an integer from 0 to 12;
R² is hydrogen, C₁₋₆ alkyl, —(CH₂)ᵥ—OH, —(CH₂)ᵥ—NH₂, —(CH₂)ᵥ—C(O)OH, —(CH₂)ᵥ-phenyl, and —(CH₂)ᵥ—SO₂CH₃, wherein v is an integer from 0 to 12;
R³ is hydrogen —C₁₋₆ alkyl, —(CH₂)ᵥ—OH, —(CH₂)ᵥ—NH₂, —(CH₂)ᵥ—C(O)OH, —(CH₂)ᵥ-phenyl, —(CH₂)ᵥ—SO₂CH₃, and —CO—(CH₂)ᵥ—O—COCH₃, wherein v is an integer from 0 to 12;
R⁴ is —NH—, —N(—C₁₋₆ alkyl), —N(—C₁₋₆ alkyl)(—SO₂CH₃), —N(—C₁₋₆ alkyl)(—(CH₂)ᵥ—OH), —N(—C₁₋₆ alkyl)(CO—CH₂—NH₂), —N(—C₁₋₆ alkyl)(—(CH₂)ᵥ—O—CH₂—NH—CO—CH₂—NH₂), —N(—C₁₋₆ alkyl)(—CO—CH(NH₂)—(CH₂)ᵥ—COOH), —N(—C₁₋₆ alkyl)(—CO—CH(NH₂)—(CH₂)ᵥ—NH₂), —N(—C₁₋₆ alkyl)(—CO—CH(NH₂)—(CH₂)ᵥ-phenyl), or

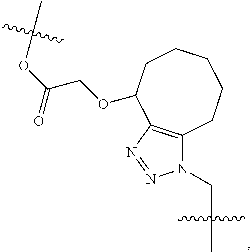

wherein v is an integer from 0 to 12;
R⁵ is H, —OH, —OCH₃, or

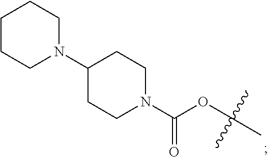 [SMILES: *OC(=O)N1CCC(N2CCCCC2)CC1]

R⁶ is hydrogen —C₁₋₆ alkyl, —(CH₂)ᵥ—OH, —(CH₂)ᵥ—NH₂, —(CH₂)ᵥ-phenyl, —(CH₂)ᵥ—N₃, —(CH₂)ᵥ—NH—CH₂-phenyl, —(CH₂)ᵥ—NMe-CH₂-phenyl-OMe, —(CH₂)ᵥ—NH—(CH₂)ᵥ—N₃, —(CH₂)ᵥ—NH—(CH₂)ᵥ—CCH; —(CH₂)ᵥ—NH—CO—CH(NH₂)—(CH₂)ᵥ-phenyl, —(CH₂)ᵥ—NH—CO—CH(NH₂)—(CH₂)ᵥ—N₃, —(CH₂)ᵥ—NH—CO—CH(NH₂)—(CH₂)ᵥ—NH₂, —(CH₂)ᵥ—NH—CO—CH(NH₂)—(CH₂)ᵥ—COOH,

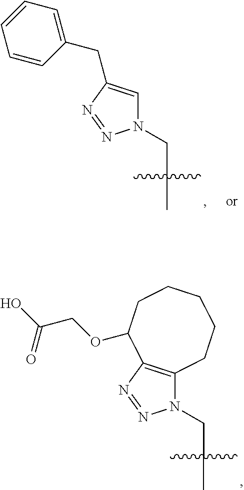

wherein v is an independently an integer from 0 to 12; and
R⁷ is H, —OH, —OCH₃, or

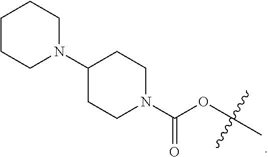 [SMILES: *OC(=O)N1CCC(N2CCCCC2)CC1]

In certain embodiments, Alk is selected from the group consisting of:

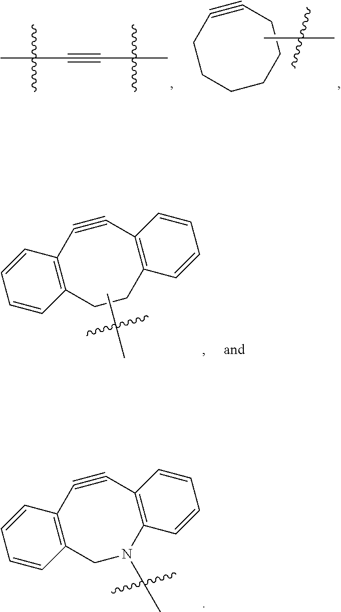

In some embodiments, SP1 is absent.

In some embodiments, AA is absent.

In some embodiments, SP2 is absent.

In some embodiments, SP1 is present and is a moiety as described above.

In some embodiments, AA is present and is a moiety as described above.

In some embodiments, SP2 is present and is a moiety as described above.

In certain embodiments, the compound according to Formula (Alk-L2-P) is selected from the group consisting of:

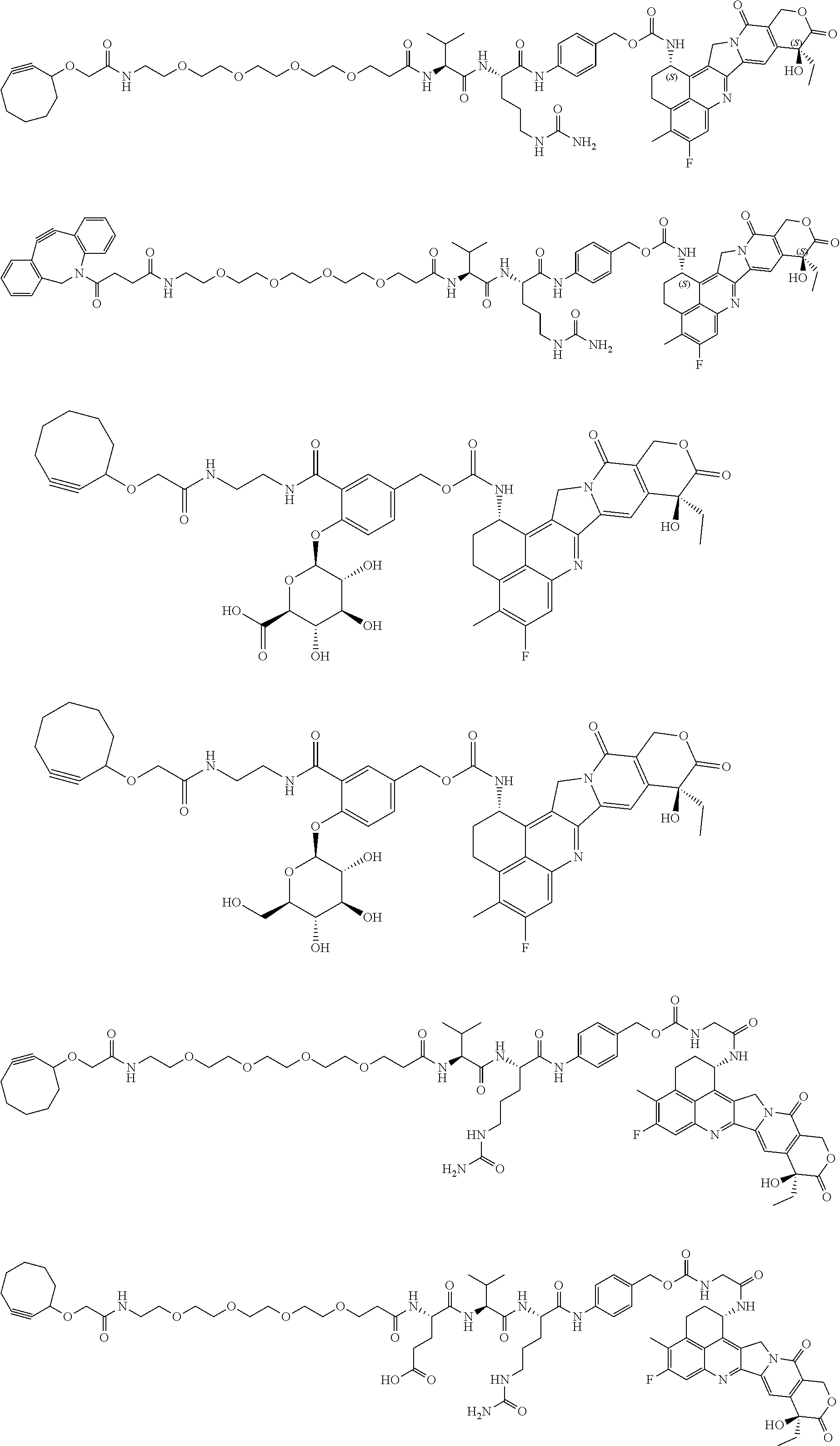

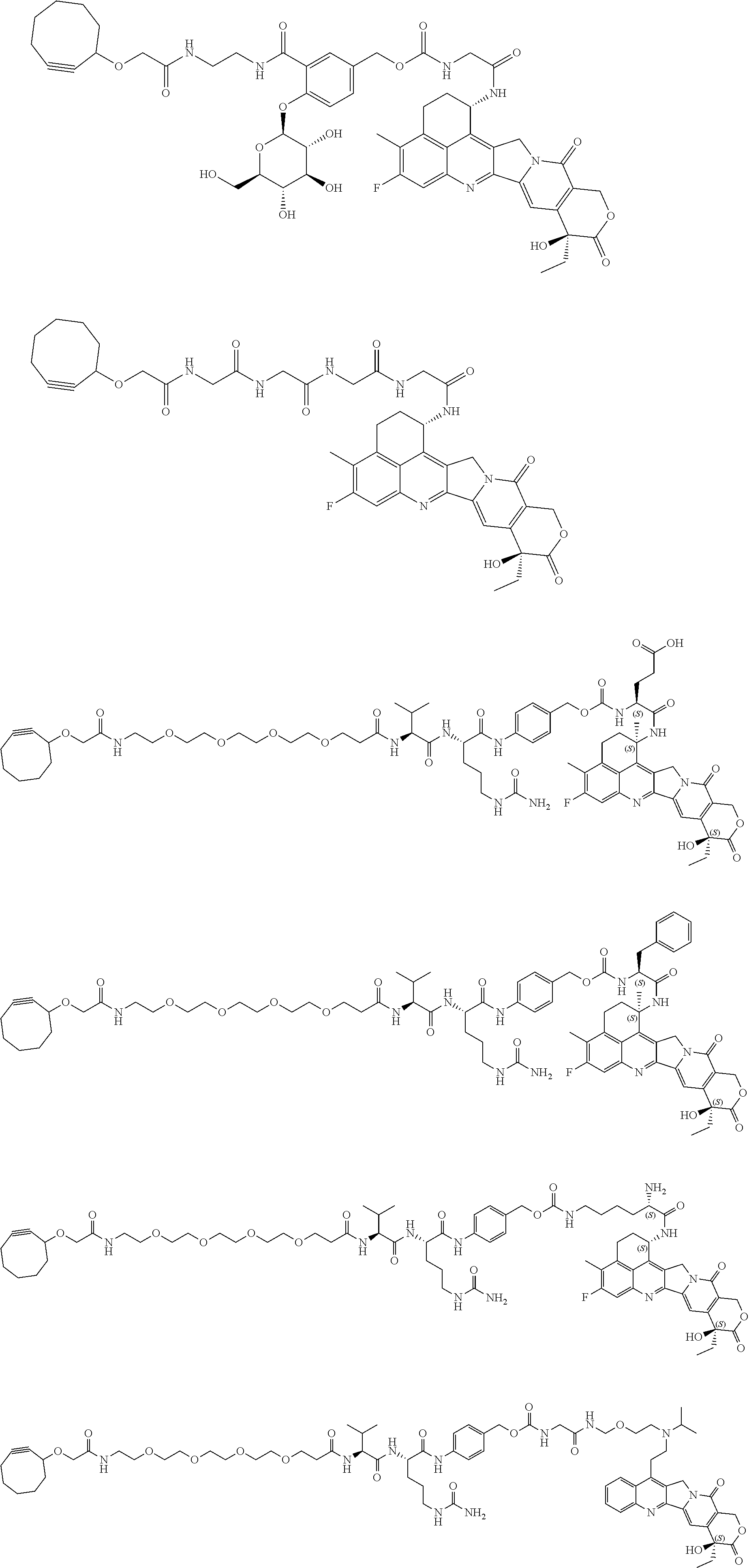

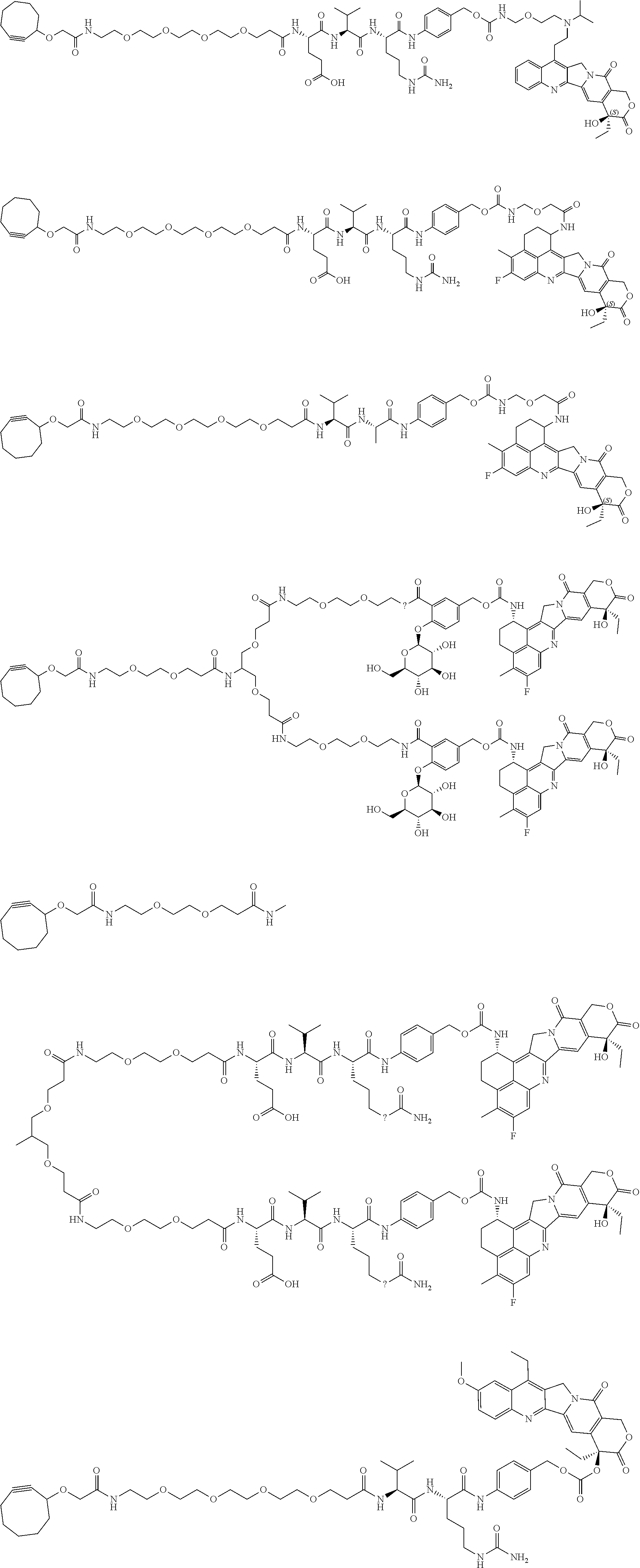

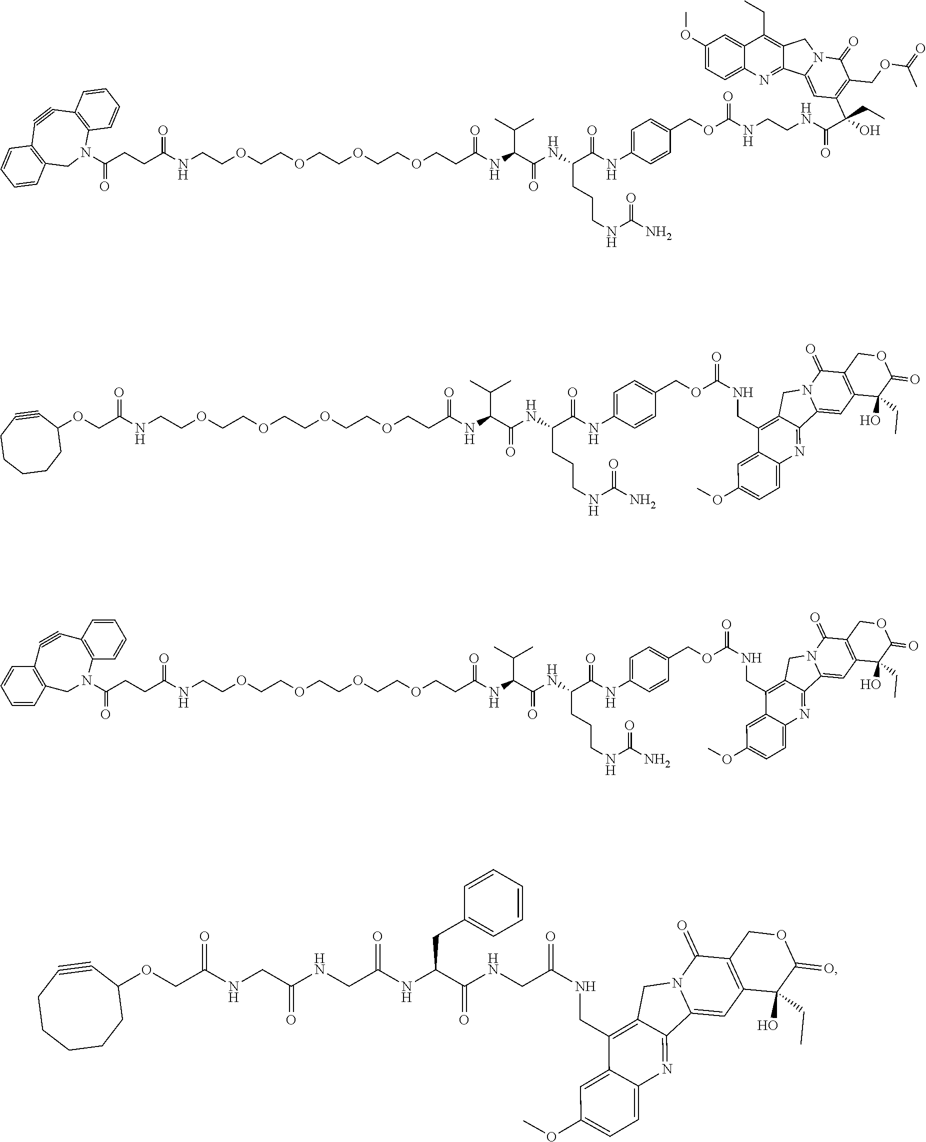

or a pharmaceutically acceptable salt thereof.

### Binding Agents

In one embodiment, the effectiveness of the protein-drug conjugate embodiments described herein depend on the selectivity of the binding agent to bind its binding partner. In one embodiment of the present disclosure, the binding agent is any molecule capable of binding with some specificity to a given binding partner. In one embodiment, the binding agent is within a mammal where the interaction can result in a therapeutic use. In an alternative embodiment, the binding agent is in vitro where the interaction can result in a diagnostic use. In some aspects, the binding agent is capable of binding to a cell or cell population.

Suitable binding agents of the present disclosure include proteins that bind to a binding partner, wherein the binding agent comprises one or more glutamine residues. Suitable binding agents include, but are not limited to, antibodies, lymphokines, hormones, growth factors, viral receptors, interleukins, or any other cell binding or peptide binding molecules or substances.

In one embodiment the binding agent is an antibody. In certain embodiments, the antibody is selected from monoclonal antibodies, polyclonal antibodies, antibody fragments (Fab, Fab′, and F(ab)2, minibodies, diabodies, triabodies, and the like). Antibodies herein can be humanized using methods described in U.S. Pat. No. 6,596,541 and US Publication No. 2012/0096572, each incorporated by reference in their entirety. In certain embodiments of the protein-drug conjugate compounds of the present disclosure, BA is a humanized monoclonal antibody. For example, BA can be a monoclonal antibody that binds HER2, MET, or STEAP2. In certain embodiments of the protein-drug conjugate compounds of the present disclosure, BA is a bispecific antibody, e.g., an anti-HER2/HER2 bispecific antibody, or an anti-MET/MET bispecific antibody.

In the present disclosure, the antibody can be any antibody deemed suitable to the practitioner of skill. In some embodiments, the antibody comprises at least one glutamine residue in at least one polypeptide chain sequence. In certain embodiments, the antibody comprises one or more gln295 residues. In certain embodiments, the antibody comprises two heavy chain polypeptides, each with one gln295 residue. In further embodiments, the antibody comprises one or more glutamine residues at a site other than a heavy chain 295. Such antibodies can be isolated from natural sources or engineered to comprise one or more glutamine residues. Techniques for engineering glutamine residues into an antibody polypeptide chain are within the skill of the practitioners in the art. In certain embodiments, the antibody is aglycosylated.

The antibody can be in any form known to those of skill in the art. In certain embodiments, the antibody comprises a light chain. In certain embodiments, the light chain is a kappa light chain. In certain embodiments, the light chain is a lambda light chain.

In certain embodiments, the antibody comprises a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the antibody is an antibody fragment. In some aspects, the antibody fragment is an Fv fragment. In some aspects, the antibody fragment is a Fab fragment. In some aspects, the antibody fragment is a F(ab′)2 fragment. In some aspects, the antibody fragment is a Fab′ fragment. In some aspects, the antibody fragment is an scFv (sFv) fragment. In some aspects, the antibody fragment is an scFv-Fc fragment.

In some embodiments, the antibody is a monoclonal antibody. In some embodiments, the antibody is a polyclonal antibody.

In some embodiments, the antibody is a chimeric antibody. In some embodiments, the antibody is a humanized antibody. In some embodiments, the antibody is a human antibody.

The antibody can have binding specificity for any antigen deemed suitable to those of skill in the art. In certain embodiments, the antigen is a transmembrane molecule (e.g., receptor) or a growth factor. Exemplary antigens include, but are not limited to, molecules such as renin; a growth hormone, including human growth hormone and bovine growth hormone; growth hormone releasing factor; parathyroid hormone; thyroid stimulating hormone; lipoproteins; alpha1-antitrypsin; insulin A-chain; insulin B-chain; proinsulin; follicle stimulating hormone; calcitonin; luteinizing hormone; glucagon; clotting factors such as factor vmc, factor IX, tissue factor (TF), and von Willebrands factor; anti-clotting factors such as Protein C; atrial natriuretic factor; lung surfactant; a plasminogen activator, such as urokinase or human urine or tissue-type plasminogen activator (t-PA); bombesin; thrombin; hemopoietic growth factor; tumor necrosis factor-alpha and -beta; enkephalinase; RANTES (regulated on activation normally T-cell expressed and secreted); human macrophage inflammatory protein (MIP-1-alpha); a serum albumin, such as human serum albumin; Muellerian-inhibiting substance; relaxin A-chain; relaxin B-chain; prorelaxin; mouse gonadotropin-associated peptide; a microbial protein, such as betalactamase; DNase; 19E; a cytotoxic T-lymphocyte associated antigen (CTLA), such as CTLA-4; inhibin; activin; vascular endothelial growth factor (VEGF); receptors for hormones or growth factors; protein A or D; rheumatoid factors; a neurotrophic factor such as bone-derived neurotrophic factor (BDNF), neurotrophin-3, -4, -5, or -6 (NT-3, NT4, NT-5, or NT-6), or a nerve growth factor such as NGF-β; platelet-derived growth factor (PDGF); fibroblast growth factor such as aFGF and bFGF; fibroblast growth factor receptor 2 (FGFR2), epidermal growth factor (EGF); transforming growth factor (TGF) such as TGF-alpha and TGF-beta, including TGF-β1, TGF-β2, TGF-β, TGF-β4, or TGF-β5; insulin-like growth factor-I and -2 (IGF-I and IGF-2); des(I-3)-IGF-I (brain IGF-I), insulin-like growth factor binding proteins, EpCAM, gD3, FLT3, PSMA, PSCA, MUC1, MUC16, STEAP, STEAP2, CEA, TENB2, EphA receptors, EphB receptors, folate receptor, FOLRI, mesothelin, cripto, alphavbeta6, integrins, VEGF, VEGFR, EGFR, transferrin receptor, IRTAI, IRTA2, IRTA3, IRTA4, IRTA5; CD proteins such as CD2, CD3, CD4, CD5, CD6, CD8, CDII, CD14, CD19, CD20, CD21, CD22, CD25, CD26, CD28, CD30, CD33, CD36, CD37, CD38, CD40, CD44, CD52, CD55, CD56, CD59, CD70, CD79, CD80, CD81, CD103, CD105, CD134, CD137, CD138, CD152, or an antibody which binds to one or more tumor-associated antigens or cell-surface receptors disclosed in US Publication No. 2008/0171040 or US Publication No. 2008/0305044 and incorporated in their entirety by reference; erythropoietin; osteoinductive factors; immunotoxins; a bone morphogenetic protein (BMP); an interferon, such as interferon-alpha, -beta, and -gamma; colony stimulating factors (CSFs), e.g., M-CSF, gM-CSF, and g-CSF; interleukins (ILs), e.g., IL-1 to IL-10; superoxide dismutase; T-cell receptors; surface membrane proteins; decay accelerating factor; viral antigen such as, for example, a portion of the HIV envelope; transport proteins; homing receptors; addressins; regulatory proteins; integrins, such as CDIIa, CDIIb, CDIIc, CD18, an ICAM, VLA-4 and VCAM; a tumor associated antigen such as AFP, ALK, B7H4, BAGE proteins, β-catenin, brc-abl, BRCA1, BORIS, CA9 (carbonic anhydrase IX), caspase-8, CD20, CD40, CD123, CDK4, CEA, CLEC12A, c-kit, cMET, CTLA4, cyclin-B1, CYP1B1, EGFR, EGFRVIII, endoglin, Epcam, EphA2, ErbB2/HER2, ErbB3/HER3, ErbB4/HER4, ETV6-AML, Fra-1, FOLR1, gAGE proteins (e.g., gAGE-1, -2), gD2, gD3, globoH, glypican-3, gM3, gp100, HER2, HLA/B-raf, HLA/EBNA1, HLA/k-ras, HLA/MAGE-A3, hTERT, IGF1R, LGR5, LMP2, MAGE proteins (e.g., MAGE-1, -2, -3, -4, -6, and -12), MART-1, mesothelin, mL-IAP, Muc1, Muc16 (CA-125), MET, MUM1, NA17, NGEP, NY-BR1, NY-BR62, NY-BR85, NY-ESO1, OX40, p15, p53, PAP, PAX3, PAX5, PCTA-1, PDGFR-α, PDGFR-β, PDGF-A, PDGF-B, PDGF-C, PDGF-D, PLAC1, PRLR, PRAME, PSCA, PSGR, PSMA (FOLH1), RAGE proteins, Ras, RGS5, Rho, SART-1, SART-3, STEAP1, STEAP2, STn, survivin, TAG-72, TGF-β, TMPRSS2, Tn, TNFRSF17, TRP-1, TRP-2, tyrosinase, and uroplakin-3, and fragments of any of the herein-listed polypeptides.

Exemplary antigens also include, but are not limited to, BCMA, SLAMF7, B7H4, gPNMB, UPK3A, and LGR5. Exemplary antigens also include, but are not limited to, MUC16, PSMA, STEAP2, and HER2.

In some embodiments, antigens also include, but are not limited to, hematologic targets, e.g., CD22, CD30, CD33, CD79a, and CD79b.

Some embodiments herein are target specific for therapeutic or diagnostic use. In one embodiment, binding agents are prepared to interact with and bind to antigens defined as tumor antigens, which include antigens specific for a type of tumor or antigens that are shared, overexpressed or modified on a particular type of tumor. Examples include: alpha-actinin-4 with lung cancer, ARTC1 with melanoma, BCR-ABL fusion protein with chronic myeloid leukemia, B-RAF, CLPP or Cdc27 with melanoma, CASP-8 with squamous cell carcinoma, and hsp70-2 with renal cell carcinoma as well as the following shared tumor-specific antigens, for example: BAGE-1, gAGE, gnTV, KK-LC-1, MAGE-A2, NA88-A, TRP2-INT2. In some embodiments, the antigen is PRLR or HER2. In some embodiments, the antibody binds STEAP2, MUC16, EGFR, EGFRVIII, FGR2, or PRLR.

In some embodiments, the antigens include HER2. In some embodiments, the antigens include STEAP2. In some embodiments, the antigens include MET. In some embodiments, the antigens include EGFRVIII. In some embodiments, the antigens include MUC16. In some embodiments, the antigens include PRLR. In some embodiments, the antigens include PSMA. In some embodiments, the antigens include FGFR2.

In some embodiments, the BA is an anti-HER2 antibody, an anti-STEAP2 antibody, an anti-MET antibody, an anti-EGFRVIII antibody, an anti-MUC16 antibody, an anti-PRLR antibody, an anti-PSMA antibody, or an anti-FGFR2 antibody, an anti-HER2/HER2 bispecific antibody, an anti-MET/MET bispecific antibody, or an anti-FOLR1 antibody, or an antigen-binding fragment thereof.

In some embodiments, the BA targets a cancer selected from the group consisting of breast cancer, ovarian cancer, prostate cancer, lung cancer, liver cancer, or brain cancer.

### Anti-HER2 Antibodies Suitable for Protein-Drug Conjugates

In some embodiments, the antibody is an anti HER2 antibody. In some embodiments, the antibody is trastuzumab, pertuzumab (2C4) or margetuximab (MGAH22). In some embodiment, the antibody is trastuzumab. According to certain embodiments, protein-drug conjugates, e.g., ADCs, according to the disclosure comprise anti-HER2 antibody. In some embodiment, the anti-HER2 antibody may include those described in WO 2019/212965 A1.

In some embodiments, the antibody is an anti-HER2/HER2 bispecific antibody, which comprises a first antigen-binding domain (D1) which specifically binds a first epitope of human HER2 and a second antigen-binding domain (D2) which specifically binds a second epitope of human HER2.

In certain embodiments, D1 and D2 domains of an anti-HER2/HER2 bispecific antibody are non-competitive with one another. Non-competition between D1 and D2 for binding to HER2 means that, the respective monospecific antigen binding proteins from which D1 and D2 were derived do not compete with one another for binding to human HER2. Exemplary antigen-binding protein competition assays are known in the art.

In certain embodiments, D1 and D2 bind to different (e.g., non-overlapping, or partially overlapping) epitopes on HER2.

In one non-limiting embodiment, the present disclosure provides protein-drug conjugates comprising a bispecific antigen-binding molecule comprising:
a first antigen-binding domain (D1); and
a second antigen-binding domain (D2);
wherein D1 specifically binds a first epitope of human HER2; and
wherein D2 specifically binds a second epitope of human HER2.

Anti-HER2/HER2 bispecific antibodies may be constructed using the antigen-binding domains of two separate monospecific anti-HER2 antibodies. For example, a collection of monoclonal monospecific anti-HER2 antibodies may be produced using standard methods known in the art. The individual antibodies thus produced may be tested pairwise against one another for cross-competition to a HER2 protein. If two different anti-HER2 antibodies are able to bind to HER2 at the same time (i.e., do not compete with one another), then the antigen-binding domain from the first anti-HER2 antibody and the antigen-binding domain from the second, non-competitive anti-HER2 antibody can be engineered into a single anti-HER2/HER2 bispecific antibody in accordance with the present disclosure.

According to the present disclosure, a bispecific antigen-binding molecule can be a single multifunctional polypeptide, or it can be a multimeric complex of two or more polypeptides that are covalently or non-covalently associated with one another. As will be made evident by the present disclosure, any antigen binding construct which has the ability to simultaneously bind two separate, non-identical epitopes of the HER2 molecule is regarded as a bispecific antigen-binding molecule. Any of the bispecific antigen-binding molecules described herein, or variants thereof, may be constructed using standard molecular biological techniques (e.g., recombinant DNA and protein expression technology) as will be known to a person of ordinary skill in the art.

In another aspect, the disclosure provides a pharmaceutical composition comprising a recombinant human antibody or fragment thereof which specifically binds HER2 and a pharmaceutically acceptable carrier. In one non-limiting embodiment, the antibody may bind two separate epitopes on the HER2 protein, i.e., the antibody is a HER2/HER2 bispecific antibody. In a related aspect, the disclosure features a composition which is a combination of an anti-HER2/HER2 antibody and a second therapeutic agent. In one embodiment, the second therapeutic agent is any agent that is advantageously combined with an anti-HER2/HER2 antibody. Additional combination therapies and co-formulations involving the anti-HER2/HER2 bispecific antibodies of the present disclosure are disclosed elsewhere herein.

In another aspect, the disclosure provides therapeutic methods for targeting/killing tumor cells expressing HER2 using an anti-HER2/HER2 bispecific antibody of the disclosure, wherein the therapeutic methods comprise administering a therapeutically effective amount of a pharmaceutical composition comprising an anti-HER2/HER2 antibody of the disclosure to a subject in need thereof. In some cases, the anti-HER2/HER2 antibodies (or antigen-binding fragments thereof) can be used for treating breast cancer, or may be modified to be more cytotoxic by methods, including but not limited to, modified Fc domains to increase ADCC (see e.g., Shield et al. (2002) JBC 277:26733), radioimmunotherapy, antibody-drug conjugates, or other methods for increasing the efficiency of tumor ablation.

The present disclosure also includes the use of an anti-HER2 antibody of the disclosure in the manufacture of a medicament for the treatment of a disease or disorder (e.g., cancer) related to or caused by HER2-expressing cells. In one aspect, the disclosure relates to a compound comprising an anti-HER2 antibody or antigen-binding fragment, or a HER2/HER2 bispecific antibody, as disclosed herein, for use in medicine. In one aspect, the disclosure relates to a compound comprising an antibody-drug conjugate (ADC) as disclosed herein, for use in medicine.

In yet another aspect, the disclosure provides bispecific anti-HER2/HER2 antibodies for diagnostic applications, such as, e.g., imaging reagents.

### Antibody Conjugation

Techniques and linkers for conjugating to residues of an antibody or antigen binding fragment are known in the art. Exemplary amino acid attachments that can be used in the context of this aspect, e.g., lysine (see, e.g., U.S. Pat. No. 5,208,020; US 2010/0129314; Hollander et al., Bioconjugate Chem., 2008, 19:358-361; WO 2005/089808; U.S. Pat. No. 5,714,586; US 2013/0101546; and US 2012/0585592), cysteine (see, e.g., US 2007/0258987; WO 2013/055993; WO 2013/055990; WO 2013/053873; WO 2013/053872; WO 2011/130598; US 2013/0101546; and U.S. Pat. No. 7,750,116), selenoysteine (see, e.g., WO 2008/122039; and Hofer et al., Proc. Natl. Acad. Sci., USA, 2008, 105:12451-12456), formyl glycine (see, e.g., Carrico et al., Nat. Chem. Biol., 2007, 3:321-322; Agarwal et al., Proc. Natl. Acad. Sci., USA, 2013, 110:46-51, and Rabuka et al., Nat. Protocols, 2012, 10:1052-1067), non-natural amino acids (see, e.g., WO 2013/068874, and WO 2012/166559), and acidic amino acids (see, e.g., WO 2012/05982). Lysine conjugation can also proceed through NHS (N-hydroxy succinimide). Linkers can also be conjugated to cysteine residues, including cysteine residues of a cleaved interchain disulfide bond, by forming a carbon bridge between thiols (see, e.g., U.S. Pat. Nos. 9,951,141, and 9,950,076). Linkers can also be conjugated to an antigen-binding protein via attachment to carbohydrates (see, e.g., US 2008/0305497, WO 2014/065661, and Ryan et al., Food & Agriculture Immunol., 2001, 13:127-130) and disulfide linkers (see, e.g., WO 2013/085925, WO 2010/010324, WO 2011/018611, and Shaunak et al., Nat. Chem. Biol., 2006, 2:312-313). Site specific conjugation techniques can also be employed to direct conjugation to particular residues of the antibody or antigen binding protein (see, e.g., Schumacher et al. J Clin Immunol (2016) 36 (Suppl 1): 100). In specific embodiments discussed in more detail below, Site specific conjugation techniques, include glutamine conjugation via transglutaminase (see e.g., Schibli, Angew Chemie Inter Ed. 2010, 49, 9995).

Payloads according to the disclosure linked through lysine and/or cysteine, e.g., via a maleimide or amide conjugation, are included within the scope of the present disclosure.

In some embodiments, the protein-drug conjugates of the present disclosure are produced according to a two-step process, where Step 1 is lysine-based linker conjugation, e.g., with an NHS-ester linker, and Step 2 is a payload conjugation reaction (e.g., a 1,3-cycloaddition reaction).

In some embodiments, the protein-drug conjugates of the present disclosure are produced according to a two-step process, where Step 1 is cysteine-based linker conjugation, e.g., with a maleimide linker, and Step 2 is a payload conjugation reaction (e.g., a 1,3-cycloaddition reaction).

In some embodiments, the protein-drug conjugates of the present disclosure are produced according to a two-step process, where Step 1 is transglutaminase-mediated site specific conjugation and Step 2 is a payload conjugation reaction (e.g., a 1,3-cycloaddition reaction).

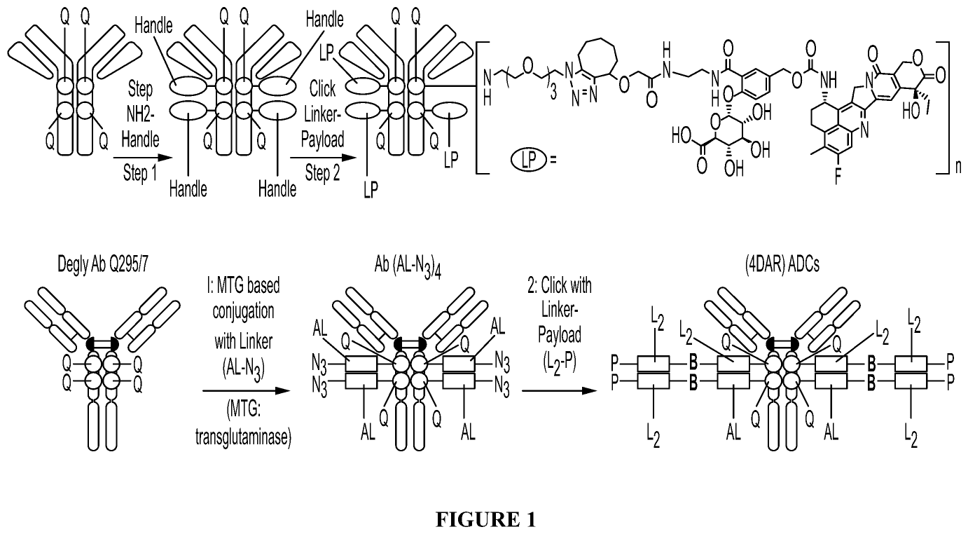

FIG. 1 depicts the structures of the site-specific ADCs according to the disclosure that were generated using two-step conjugation methods: Step 1: conjugating a deglycosylated or N297Q mutated antibody with amine-PEG3-azido linker (AL-N₃) site-specifically to Ab-Q295/7 sites via MTG mediated conjugation to generate azido functionalized antibody (Ab(AL)₄); and Step 2: attaching the Linker-payload (L₂P#) to the antibody via a [3+2]cycloaddition of alkyne with Ab(AL)₄. All ADCs with the linker-payloads conjugated on Q295/297 sites of the antibodies.

### Step 1: Transglutaminase Mediated Site Specific Conjugation

In some embodiments, proteins (e.g., antibodies) may be modified in accordance with known methods to provide glutaminyl modified proteins. Techniques for conjugating antibodies and primary amine compounds are known in the art. Site specific conjugation techniques are employed herein to direct conjugation to glutamine using glutamine conjugation via transglutaminase (see e.g., Schibli, Angew Chemie Inter Ed. 2010, 49, 9995).

Primary amine-comprising compounds (e.g., linkers L1) of the present disclosure can be conjugated to one or more glutamine residues of a binding agent (e.g., a protein, e.g., an antibody) via transglutaminase-based chemo-enzymatic conjugation (see, e.g., Dennler et al., Protein Conjugate Chem. 2014, 25, 569-578, and WO 2017/147542). For example, in the presence of transglutaminase, one or more glutamine residues of an antibody can be coupled to a primary amine linker compound. Briefly, in some embodiments, a binding agent having a glutamine residue (e.g., a gln295, i.e. Q295 residue) is treated with a primary amine-containing linker L1, described above, in the presence of the enzyme transglutaminase. In certain embodiments, the binding agent is aglycosylated. In certain embodiments, the binding agent is deglycosylated.

In certain embodiments, the binding agent (e.g., a protein, e.g., an antibody) comprises at least one glutamine residue in at least one polypeptide chain sequence. In certain embodiments, the binding agent comprises two heavy chain polypeptides, each with one gln295 residue. In further embodiments, the binding agent comprises one or more glutamine residues at a site other than a heavy chain 295.

In some embodiments, a binding agent, such as an antibody, can be prepared by site-directed mutagenesis to insert a glutamine residue at a site without resulting in disabled antibody function or binding. For example, included herein are antibodies bearing Asn297Gln (N297Q) mutation(s) as described herein. In certain embodiments, the binding agent comprises two heavy chain polypeptides, each with one gln295 and one gln297 residue.

In some embodiments, an antibody having a gln295 residue and/or an N297Q mutation contains one or more additional naturally occurring glutamine residues in their variable regions, which can be accessible to transglutaminase and therefore capable of conjugation to a linker or a linker-payload. An exemplary naturally occurring glutamine residue can be found, e.g., at Q55 of the light chain. In such instances, the binding agent, e.g., antibody, conjugated via transglutaminase can have a higher than expected LAR value (e.g., a LAR higher than 4). Any such antibodies can be isolated from natural or artificial sources.

In certain embodiments of the disclosure, the linker-antibody ratio or LAR is from 1, 2, 3, 4, 5, 6, 7, or 8 linker L1 molecules per antibody. In some embodiments, the LAR is from 1 to 8. In some embodiments, the LAR is from 1 to 6. In certain embodiments, the LAR is from 2 to 4. In some cases, the LAR is from 2 to 3. In certain cases, the LAR is from 0.5 to 3.5. In some embodiments, the LAR is about 1, or about 1.5, or about 2, or about 2.5, or about 3, or about 3.5. In some embodiments, the LAR is 2. In some embodiments, the LAR is 4.

### Step 2: Payload Conjugation Reaction

In certain embodiments, linkers L1 according to the present disclosure comprise at least one reactive group B′ capable of further reaction after transglutamination. In these embodiments, the glutaminyl-modified protein (e.g., antibody) is capable of further reaction with a reactive payload compound or a reactive linker-payload compound (e.g., L2-P as disclosed herein), to form a protein-payload conjugate. More specifically, the reactive linker-payload compound L2-P may comprise a reactive group B″ that is capable of reacting with the reactive group B′ of the linker L1. In certain embodiments, a reactive group B′ according to the present disclosure comprises a moiety that is capable of undergoing a 1,3-cycloaddition reaction. In certain embodiments, the reactive group B′ is an azide. In certain embodiments, the reactive group B″ comprises an alkyne (e.g., a terminal alkyne, or an internal strained alkyne). In certain embodiments of the present disclosure the reactive group B′ is compatible with the binding agent and transglutamination reaction conditions.

In certain embodiments of the disclosure, linker L1 molecules comprise a one reactive group B′. In certain embodiments of the disclosure, linker L1 molecules comprise more than one reactive group B′.

In certain embodiments, the reactive linker-payload L2-P comprises one payload molecule (n=1). In certain other embodiments, the reactive linker-payload L2-P comprises two or more payload molecules (n 2). In certain embodiments, the reactive linker-payload L2-P comprises from 1 to 12 payload molecules, or from 1 to 10 payload molecules, or from 1 to 8 payload molecules, or from 1 to 6 payload molecules, or from 1 to 4 payload molecules, or from 1 to 2 payload molecules.

In certain embodiments, the reactive linker-payload L2-P comprises one payload molecule. When such L2-P is reacted with a BA-L1-B′ (e.g., wherein B′ is an azide, BA-L1-N₃), the DAR will be about equal to the LAR of the BA-L1-B′. For example, if L2-P comprising one payload molecule is reacted with a BA-L1-B′ having a LAR of 4 (e.g., via Q295 and N297Q transglutamination), the resulting protein-drug conjugate will have a DAR of 4.

In certain embodiments, the reactive linker-payload L2-P comprises 2 payload molecules. When such L2-P is reacted with a BA-L1-B′, the DAR will be about 2 times the LAR of the BA-L1-B′. For example, if L2-P comprising 2 payload molecules is reacted with a BA-L1-B′ having a LAR of 4 (e.g., via Q295 and N297Q transglutamination), the resulting protein-drug conjugate will have a DAR of 8.

In certain embodiments of the disclosure, the drug-antibody ratio or DAR (e.g., abbreviated as the lower case letter n) is from about 1 to about 30, or from about 1 to about 24, or from about 1 to about 20, or from about 1 to about 16, or from about 1 to about 12, or from about 1 to about 10, or from about 1 to about 8, or about 1, 2, 3, 4, 5, 6, 7, or 8 payload molecules per antibody. In some embodiments, the DAR is from 1 to 30. In some embodiments, the DAR is from 1 to 24. In some embodiments, the DAR is from 1 to 16. In some embodiments, the DAR is from 1 to 8. In some embodiments, the DAR is from 1 to 6. In certain embodiments, the DAR is from 2 to 4. In some cases, the DAR is from 2 to 3. In certain cases, the DAR is from 0.5 to 3.5. In some embodiments, the DAR is 4.

In one aspect, the present disclosure provides a method of producing a compound having a structure a compound having a structure according to Formula (A):

BA-(L1-B-L2-P)ₙ (A)

wherein:
BA is an antibody or an antigen-binding fragment thereof;
L1 is a first linker covalently bound to the side chain of a glutamine residue of the BA;
B is a moiety comprising a triazole;
L2 is a second linker covalently bound to the P;
P is an antitumor agent selected from the group consisting of P-I through P-IV:

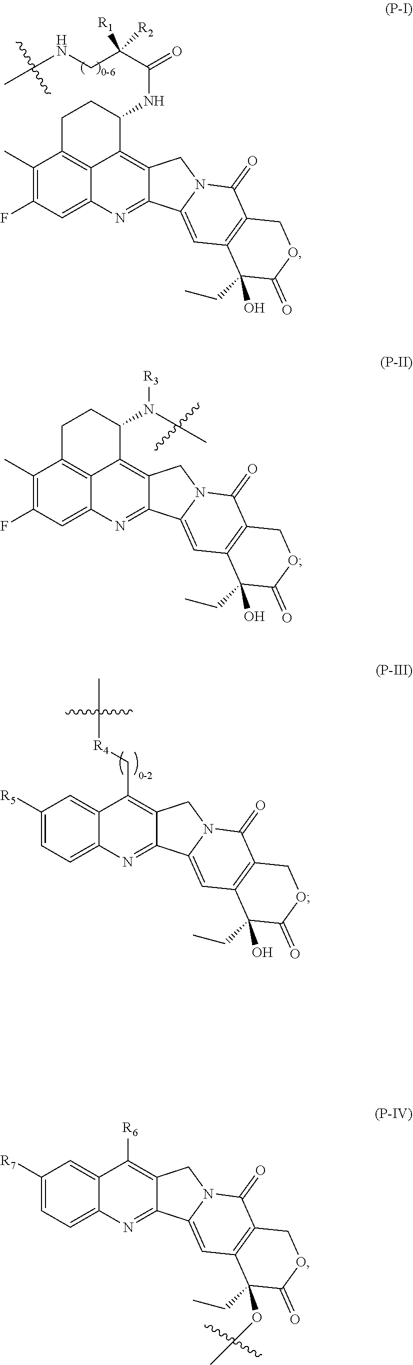

R¹ is hydrogen, C₁₋₆ alkyl, —(CH₂)ᵥ—OH, —(CH₂)ᵥ—NH₂, —(CH₂)ᵥ—C(O)OH, —(CH₂)ᵥ-phenyl, and —(CH₂)ᵥ—SO₂CH₃, wherein v is an integer from 0 to 12;
R² is hydrogen, C₁₋₆ alkyl, —(CH₂)ᵥ—OH, —(CH₂)ᵥ—NH₂, —(CH₂)ᵥ—C(O)OH, —(CH₂)ᵥ-phenyl, and —(CH₂)ᵥ—SO₂CH₃, wherein v is an integer from 0 to 12;
R³ is hydrogen —C₁₋₆ alkyl, —(CH₂)ᵥ—OH, —(CH₂)ᵥ—NH₂, —(CH₂)ᵥ—C(O)OH, —(CH₂)ᵥ-phenyl, —(CH₂)ᵥ—SO₂CH₃, and —CO—(CH₂)ᵥ—O—COCH₃, wherein v is an integer from 0 to 12;
R⁴ is —NH—, —N(—C₁₋₆ alkyl), —N(—C₁₋₆ alkyl)(—SO₂CH₃), —N(—C₁₋₆ alkyl)(—(CH₂)ᵥ—OH), —N(—C₁₋₆ alkyl)(CO—CH₂—NH₂), —N(—C₁₋₆ alkyl)(—(CH₂)ᵥ—O—CH₂—NH—CO—CH₂—NH₂), —N(—C₁₋₆ alkyl)(—CO—CH(NH₂)—(CH₂)ᵥ—COOH), —N(—C₁₋₆ alkyl)(—CO—CH(NH₂)—(CH₂)ᵥ—NH₂), —N(—C₁₋₆ alkyl)(—CO—CH(NH₂)—(CH₂)ᵥ-phenyl), or

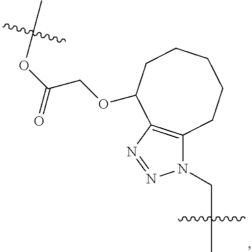

wherein v is an integer from 0 to 12;
R⁵ is H, —OH, —OCH₃, or

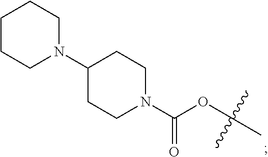 [SMILES: *OC(=O)N1CCC(N2CCCCC2)CC1]

R⁶ is hydrogen —C₁₋₆ alkyl, —(CH₂)ᵥ—OH, —(CH₂)ᵥ—NH₂, —(CH₂)ᵥ-phenyl, —(CH₂)ᵥ—N₃, —(CH₂)ᵥ—NH—CH₂-phenyl, —(CH₂)ᵥ—NMe-CH₂-phenyl-OMe, —(CH₂)ᵥ—NH—(CH₂)ᵥ—N₃, —(CH₂)ᵥ—NH—(CH₂)ᵥ—CCH; —(CH₂)ᵥ—NH—CO—CH(NH₂)—(CH₂)ᵥ-phenyl, —(CH₂)ᵥ—NH—CO—CH(NH₂)—(CH₂)ᵥ—N₃, —(CH₂)ᵥ—NH—CO—CH(NH₂)—(CH₂)ᵥ—NH₂, —(CH₂)ᵥ—NH—CO—CH(NH₂)—(CH₂)ᵥ—COOH,

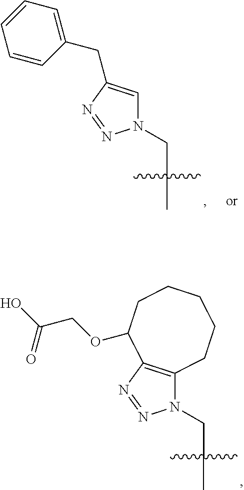

wherein v is an independently an integer from 0 to 12;
R⁷ is H, —OH, —OCH₃, or

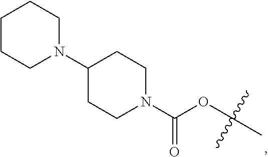 [SMILES: *OC(=O)N1CCC(N2CCCCC2)CC1]

wherein the method comprises the steps of:
wherein the method comprises the steps of:
a) contacting, in the presence of a transglutaminase, the BA comprising at least one glutamine residue with a compound L1-B′,
b) contacting the product of step a) with one or more equivalents of a compound B″-L2-P, wherein the group B″ is capable of covalently attaching to the group B′, wherein one of the groups B′ and B″ is selected from —N₃ and

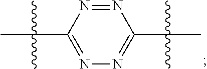 [SMILES: *c1nnc(*)nn1]

and the other of the groups B′ and B″ is selected from

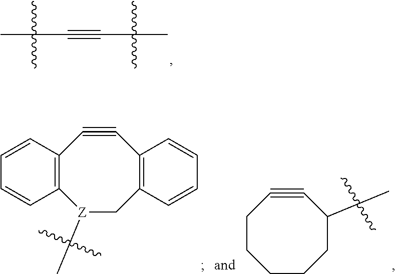

where Z is C or N; and
c) isolating the produced compound of Formula (A).

In one embodiment, the group B′ is an azide (—N₃).

In one embodiment, the group B″ comprises an alkyne. In one embodiment, the group B″ is selected from

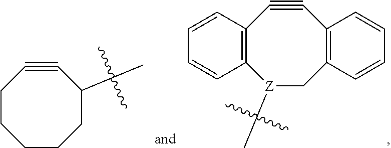

where Z is C or N.

In one embodiment, the glutamine residue Gln is naturally present in a CH2 or CH3 domain of the BA. In another embodiment, the glutamine residue Gln is introduced to the BA by modifying one or more amino acids. In one embodiment, the Gln is Q295 or N297Q.

In one embodiment, the transglutaminase is microbial transglutaminase (MTG). In one embodiment, the transglutaminase is bacterial transglutaminase (BTG).

### Therapeutic Formulation and Administration

The present disclosure provides pharmaceutical compositions comprising the protein-drug conjugates of the present disclosure.

In one aspect, the present disclosure provides compositions comprising a population of protein-drug conjugates according to the present disclosure having a drug-antibody ratio (DAR) of about 0.5 to about 30.0.

In one embodiment, the composition has a DAR of about 1.0 to about 2.5.

In one embodiment, the composition has a DAR of about 2.

In one embodiment, the composition has a DAR of about 3.0 to about 4.5.

In one embodiment, the composition has a DAR of about 4.

In one embodiment, the composition has a DAR of about 6.5 to about 8.5.

In one embodiment, the composition has a DAR of about 8.

In one embodiment, the composition has a DAR of about 10 to about 14.

In one embodiment, the composition has a DAR of about 12.

In one embodiment, the composition has a DAR of about 14 to about 18.

In one embodiment, the composition has a DAR of about 16.

In one embodiment, the composition has a DAR of about 20 to about 24.5.

In one embodiment, the composition has a DAR of about 24.

The compositions of the disclosure are formulated with suitable carriers, excipients, and other agents that provide improved transfer, delivery, tolerance, and the like. A multitude of appropriate formulations can be found in the formulary known to all pharmaceutical chemists: Remington's Pharmaceutical Sciences, Mack Publishing Company, Easton, Pa. These formulations include, for example, powders, pastes, ointments, jellies, waxes, oils, lipids, lipid (cationic or anionic) containing vesicles (such as LIPOFECTIN™, Life Technologies, Carlsbad, Calif.), DNA conjugates, anhydrous absorption pastes, oil-in-water and water-in-oil emulsions, emulsions carbowax (polyethylene glycols of various molecular weights), semi-solid gels, and semi-solid mixtures containing carbowax. See also Powell et al. “Compendium of excipients for parenteral formulations” PDA (1998) J Pharm Sci Technol 52:238-311.

The dose of a protein-drug conjugate administered to a patient may vary depending upon the age and the size of the patient, target disease, conditions, route of administration, and the like. The suitable dose is typically calculated according to body weight or body surface area. When a protein-drug conjugate of the present disclosure is used for therapeutic purposes in an adult patient, it may be advantageous to intravenously administer the protein-drug conjugate of the present disclosure normally at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. Effective dosages and schedules for administering a protein-drug conjugate may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351).

Various delivery systems are known and can be used to administer the pharmaceutical composition of the disclosure, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

A pharmaceutical composition of the present disclosure can be delivered subcutaneously or intravenously with a standard needle and syringe. In addition, with respect to subcutaneous delivery, a pen delivery device readily has applications in delivering a pharmaceutical composition of the present disclosure. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present disclosure. Examples include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPEN™, OPTIPEN PRO™, OPTIPEN STARLET™, and OPTICLIK™ (Sanofi-Aventis, Frankfurt, Germany), to name only a few. Examples of disposable pen delivery devices having applications in subcutaneous delivery of a pharmaceutical composition of the present disclosure include, but are not limited to the SOLOSTAR™ pen (Sanofi-Aventis), the FLEXPEN™ (Novo Nordisk), and the KWIKPEN™ (Eli Lilly), the SURECLICK™ Autoinjector (Amgen, Thousand Oaks, Calif.), the PENLET™ (Haselmeier, Stuttgart, Germany), the EPIPEN (Dey, L. P.), and the HUMIRA™ Pen (Abbott Labs, Abbott Park IL), to name only a few.

In certain situations, the pharmaceutical composition can be delivered in a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Fla. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule.

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 5 to about 500 mg per dosage form in a unit dose; especially in the form of injection, it is preferred that the aforesaid antibody is contained in about 5 to about 100 mg and in about 10 to about 250 mg for the other dosage forms.

### Therapeutic Uses of the Protein-Drug Conjugates, Linker-Payloads and Payloads

In another aspect, the protein-drug conjugates, e.g., ADCs, disclosed herein are useful, inter alia, for the treatment, prevention and/or amelioration of a disease, disorder or condition in need of such treatment.

In one embodiment, the present invention provides a method of treating a condition in a subject in need thereof comprising administering to the subject a therapeutically effective amount of a compound (e.g., an antibody-drug conjugate, a linker-payload and/or a payload) according to the disclosure, or the composition comprising any compound according to the present disclosure.

In one embodiment, the protein-drug conjugates, e.g., ADCs, disclosed herein are useful for treating cancer. In one embodiment, the protein-drug conjugates, e.g., ADCs, disclosed herein are useful for treating a cancer selected from the group consisting of breast cancer, ovarian cancer, prostate cancer, lung cancer, liver cancer, or brain cancer. In one embodiment, the protein-drug conjugates, e.g., ADCs, disclosed herein are useful for treating HER2+ breast cancer. In one embodiment, the protein-drug conjugates, e.g., ADCs, disclosed herein are useful for treating prostate cancer.

In one aspect, the present disclosure provides a method of selectively delivering a compound into a cell. In one embodiment, the method of selectively delivering a compound into a cell comprises linking the compound to a targeted antibody. In one embodiment, the compound is a payload as described above. In one embodiment, the cell is a mammalian cell. In one embodiment, the cell is a human cell. In one embodiment, the cell is a cancer cell. In one embodiment, the cancer cell is selected from the group consisting of a breast cancer cell, an ovarian cancer cell, a prostate cancer cell, a lung cancer cell, a liver cancer cell, or a brain cancer cell.

In certain embodiments, the present disclosure provides a method of selectively delivering into a cell a compound having the structure selected from the group consisting of:

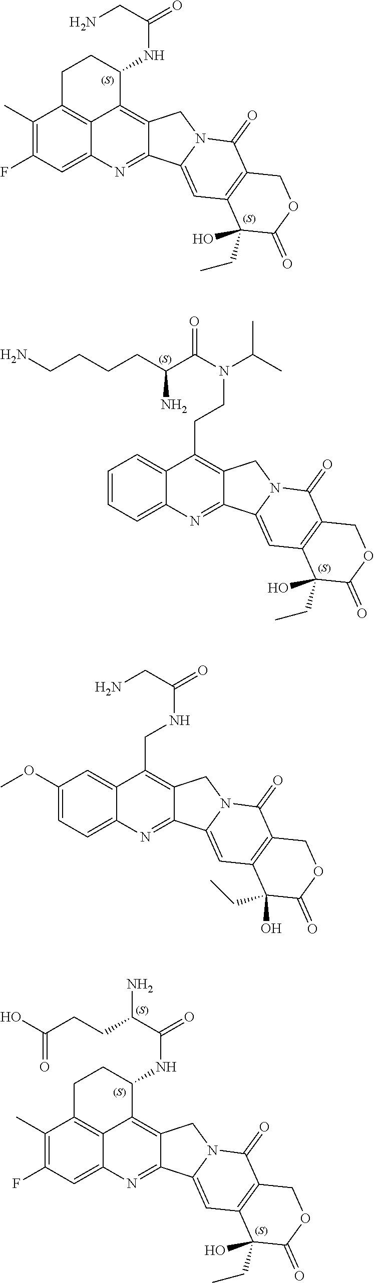

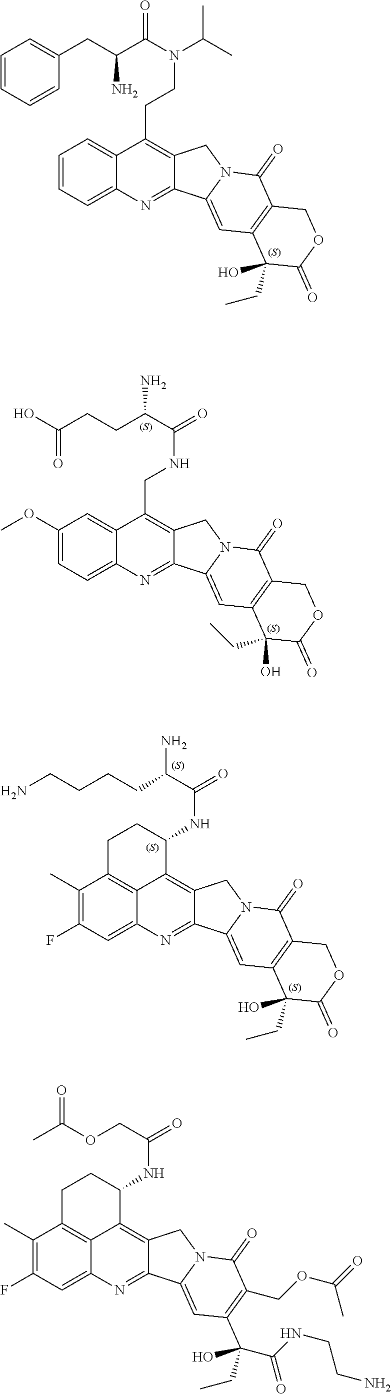

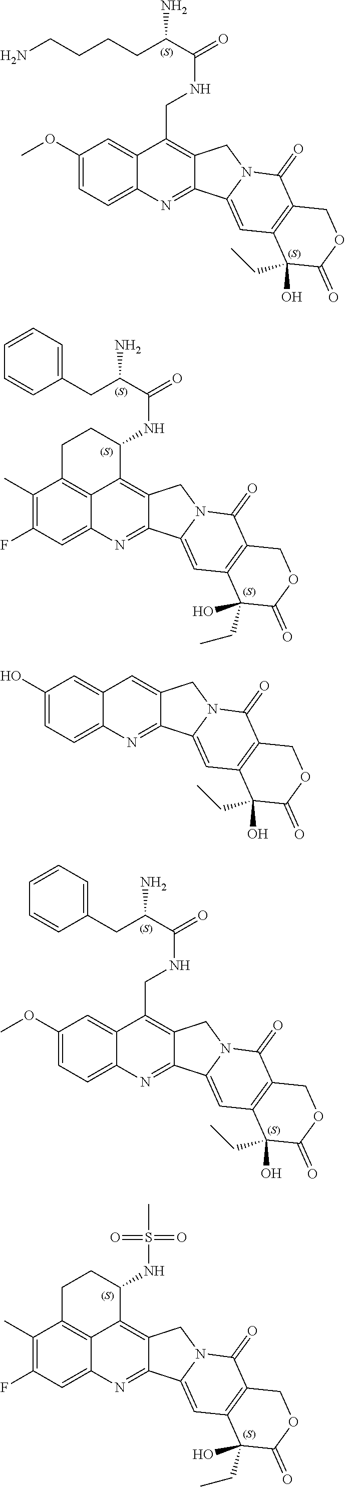

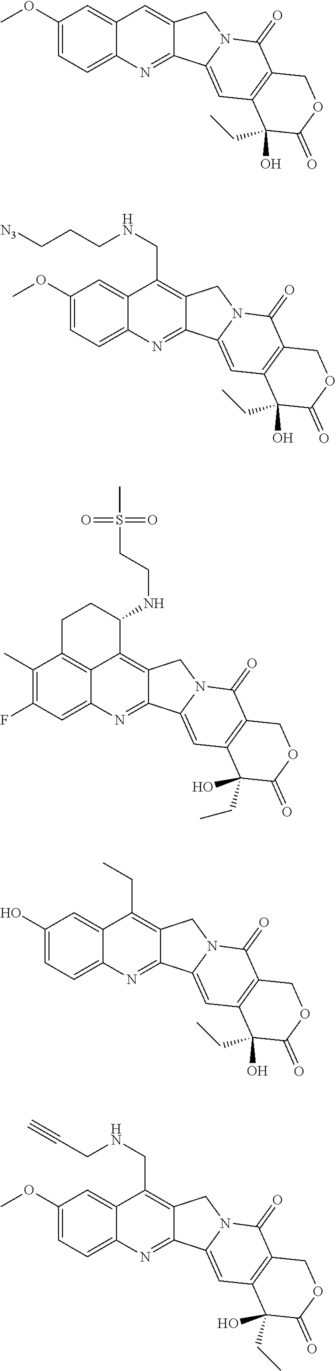 [SMILES: CC[C@@]1(O)C(=O)OCc2c1cc1n(c2=O)Cc2cc3cc(OC)ccc3nc2-1 ; CC[C@@]1(O)C(=O)OCc2c1cc1n(c2=O)Cc2c-1nc1ccc(OC)cc1c2CNCCCN=[N+]=[N-] ; CC[C@@]1(O)C(=O)OCc2c1cc1n(c2=O)Cc2c-1nc1cc(F)c(C)c3c1c2[C@@H](NCCS(C)(=O)=O)CC3 ; CCc1c2c(nc3ccc(O)cc13)-c1cc3c(c(=O)n1C2)COC(=O)[C@]3(O)CC ; C#CCNCc1c2c(nc3ccc(OC)cc13)-c1cc3c(c(=O)n1C2)COC(=O)[C@]3(O)CC]

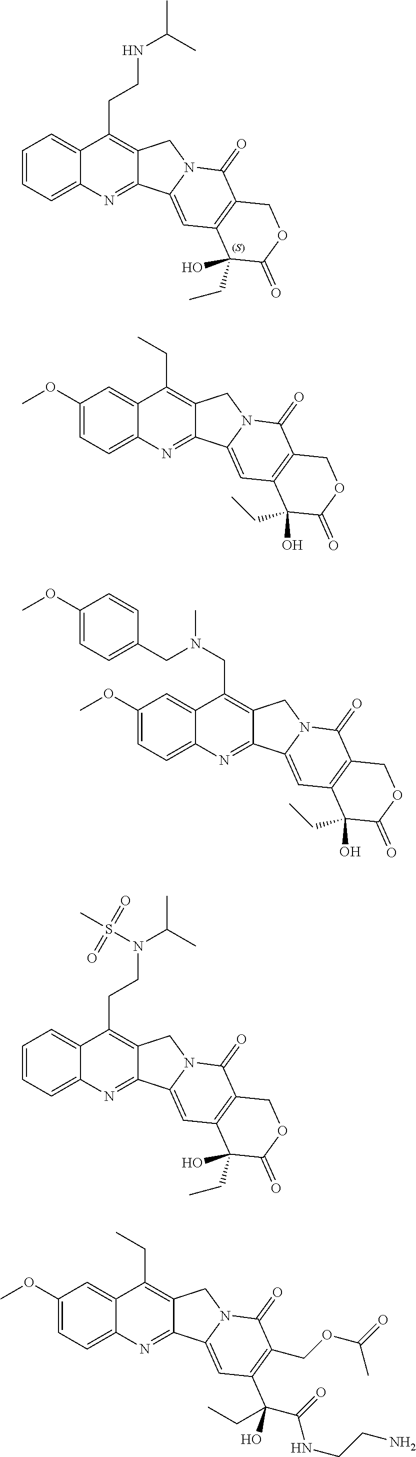

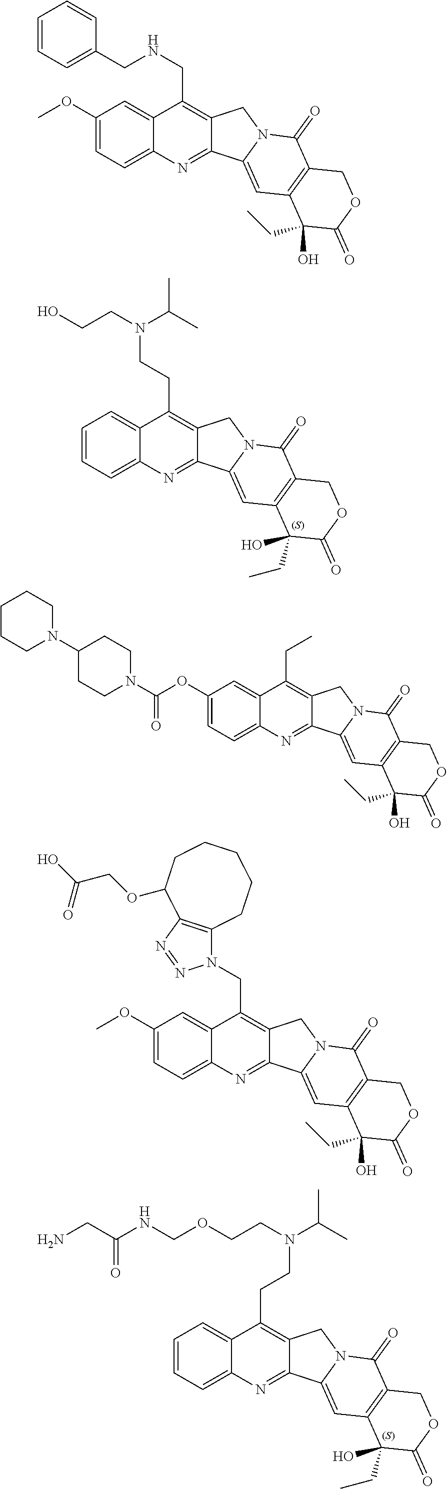

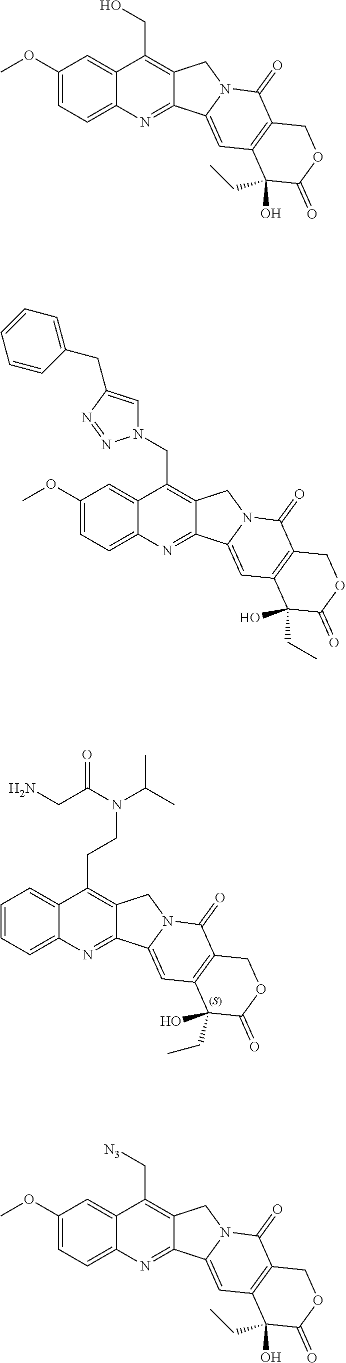

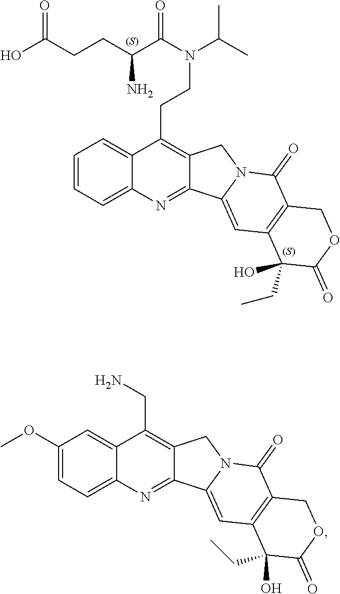

or a pharmaceutically acceptable salt thereof.

In one aspect, the present disclosure provides a method of selectively targeting an antigen on a surface of a cell with a compound. In one embodiment, the method of selectively targeting an antigen on a surface of a cell with a compound comprises linking the compound to a targeted antibody. In one embodiment, the compound is a payload as described above. In one embodiment, the cell is a mammalian cell. In one embodiment, the cell is a human cell. In one embodiment, the cell is a cancer cell. In one embodiment, the cancer cell is selected from the group consisting of a breast cancer cell, an ovarian cancer cell, a prostate cancer cell, a lung cancer cell, a liver cancer cell, or a brain cancer cell.

In certain embodiments, the present disclosure provides a method of selectively targeting an antigen on a surface of a cell with a compound having the selected from the group consisting of:

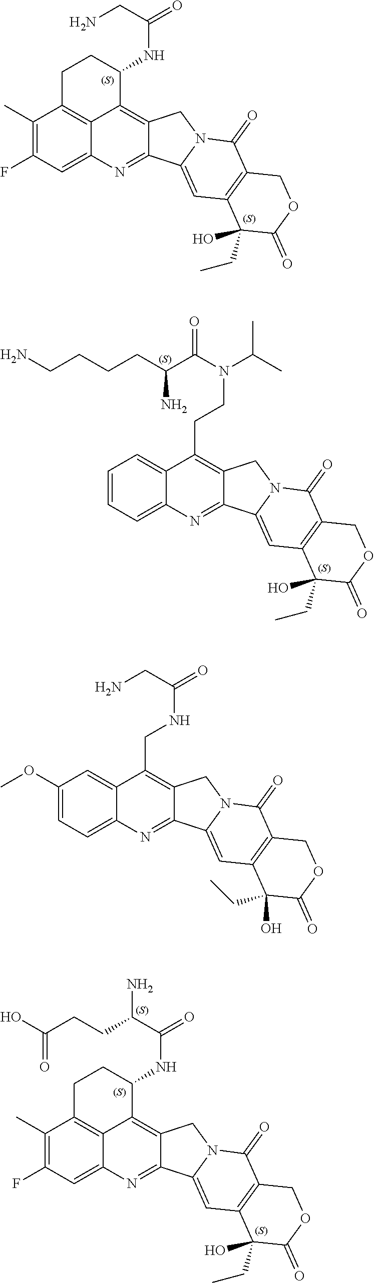

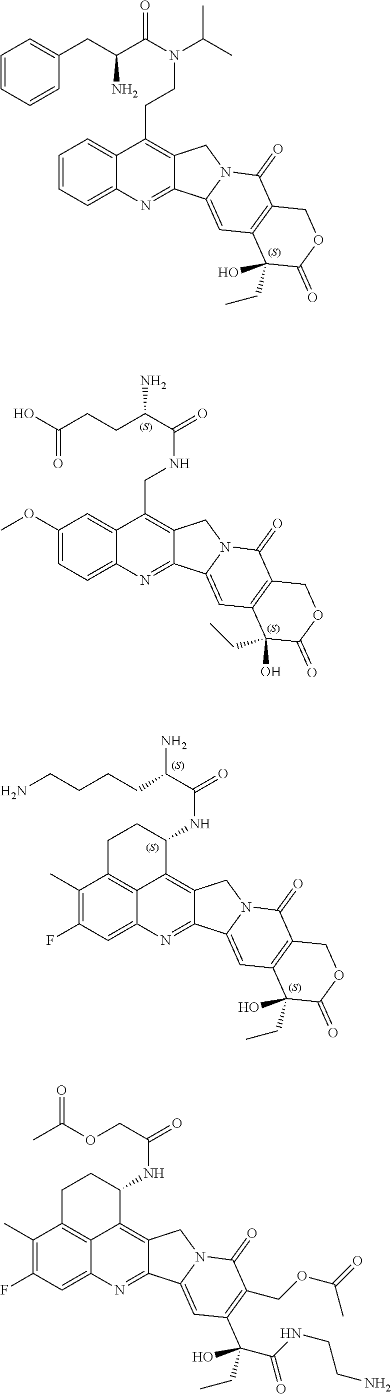

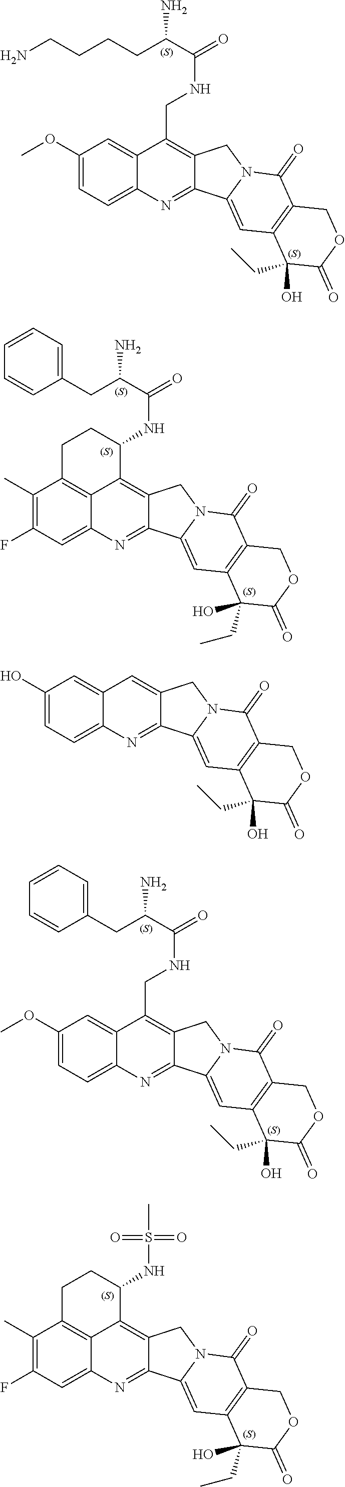

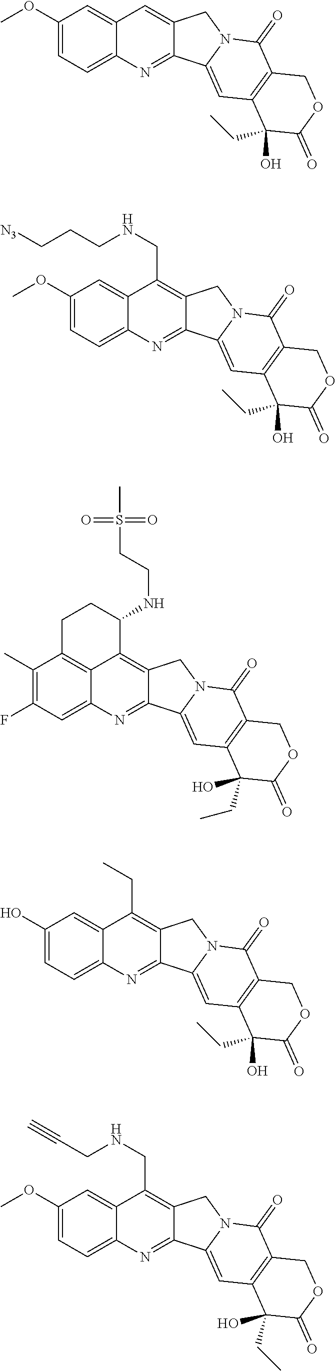 [SMILES: CC[C@@]1(O)C(=O)OCc2c1cc1n(c2=O)Cc2cc3cc(OC)ccc3nc2-1 ; CC[C@@]1(O)C(=O)OCc2c1cc1n(c2=O)Cc2c-1nc1ccc(OC)cc1c2CNCCCN=[N+]=[N-] ; CC[C@@]1(O)C(=O)OCc2c1cc1n(c2=O)Cc2c-1nc1cc(F)c(C)c3c1c2[C@@H](NCCS(C)(=O)=O)CC3 ; CCc1c2c(nc3ccc(O)cc13)-c1cc3c(c(=O)n1C2)COC(=O)[C@]3(O)CC ; C#CCNCc1c2c(nc3ccc(OC)cc13)-c1cc3c(c(=O)n1C2)COC(=O)[C@]3(O)CC]

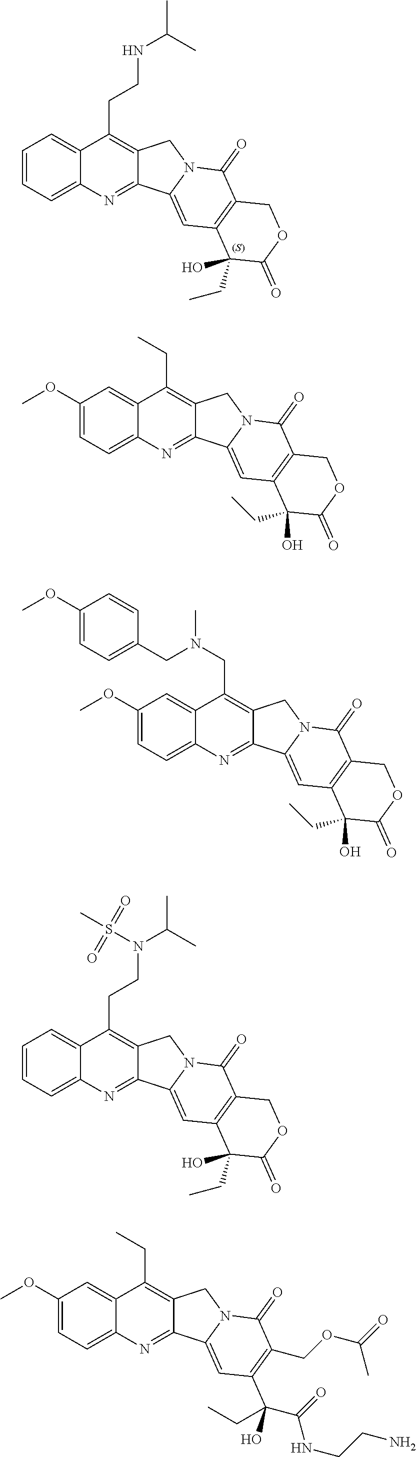

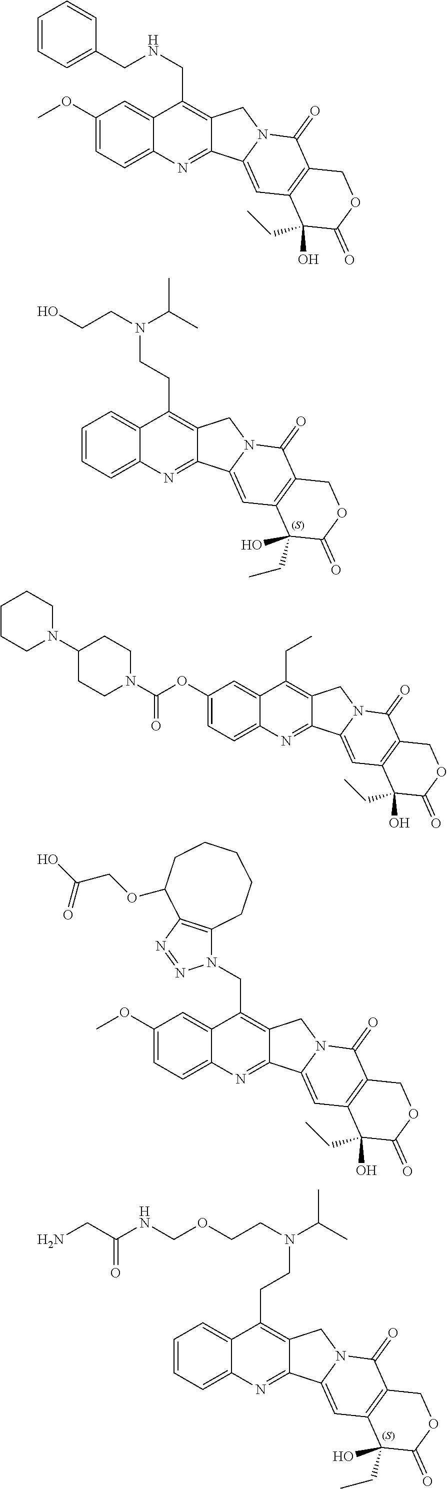

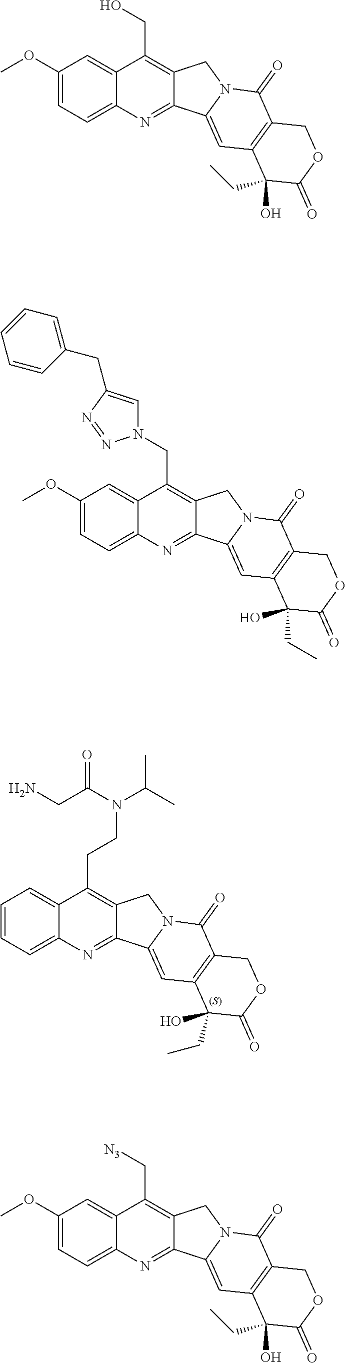

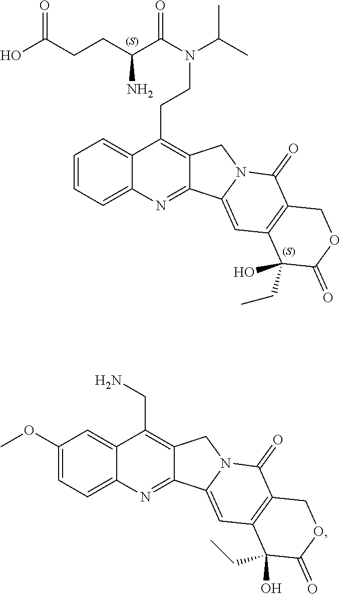

or a pharmaceutically acceptable salt thereof.

In certain embodiments, the present disclosure provides a method of treating a tumor and/or cancer comprising contacting the tumor and/or cancer with a compound having the structure selected from the group consisting of:

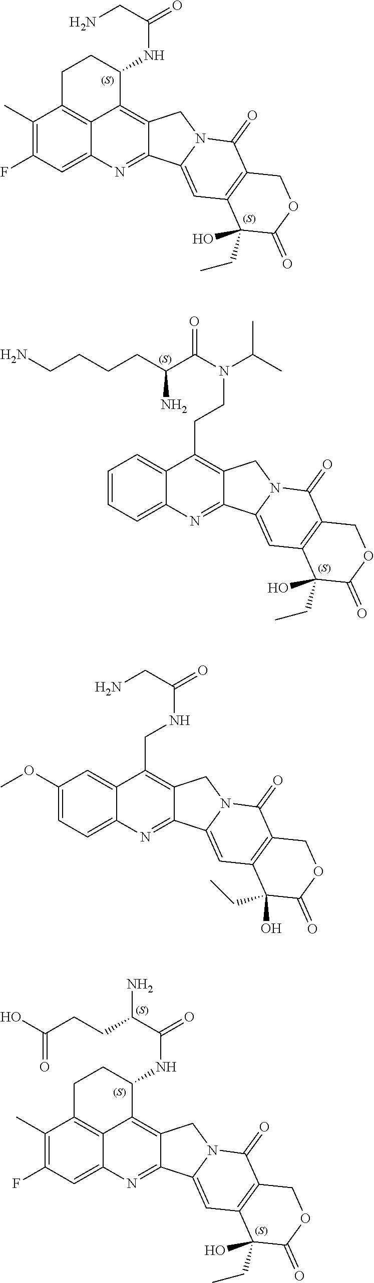

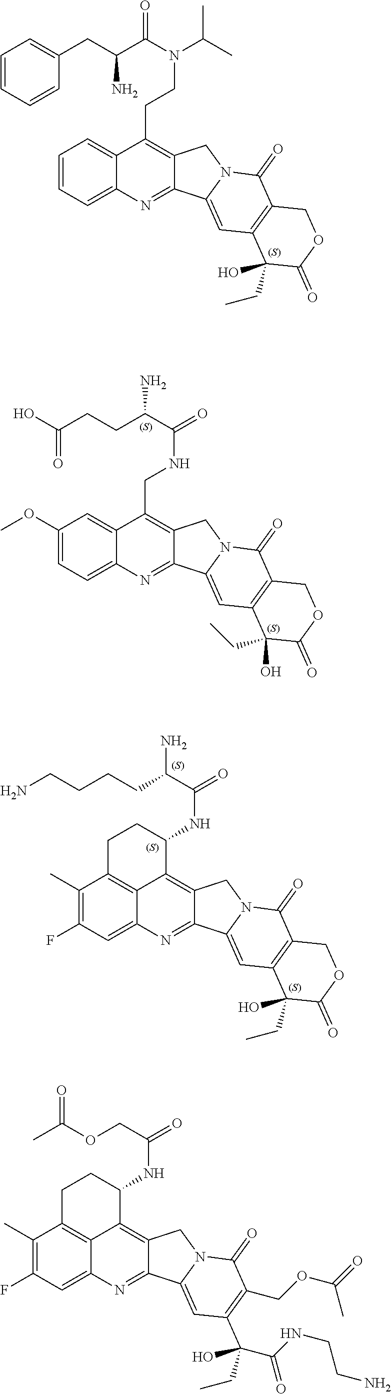

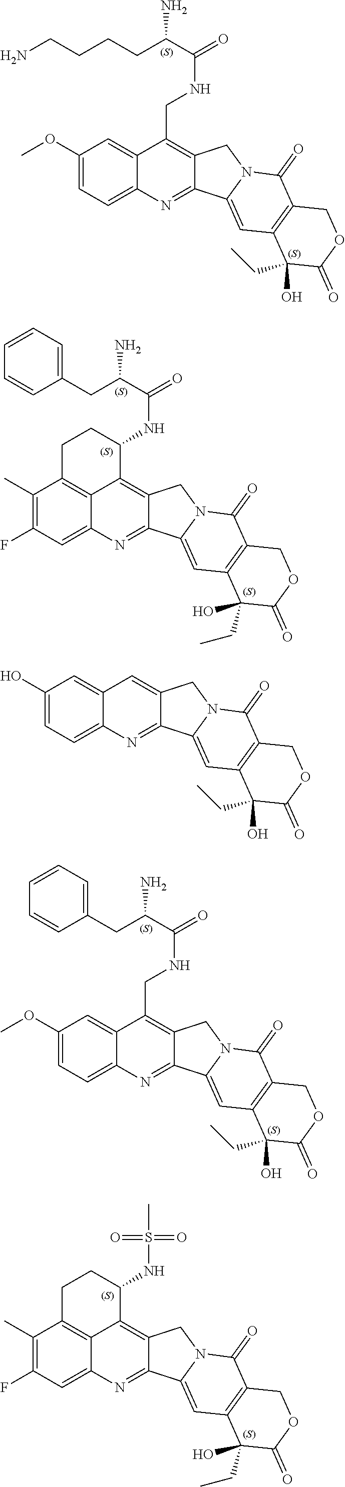

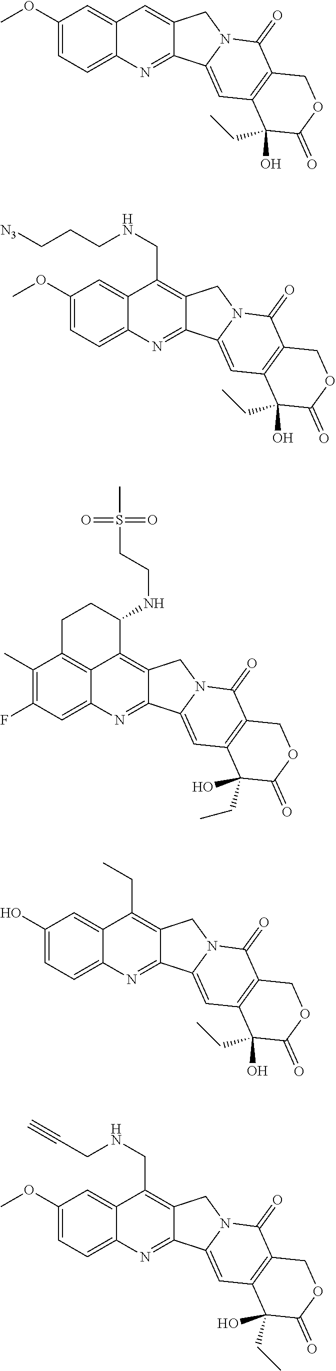 [SMILES: CC[C@@]1(O)C(=O)OCc2c1cc1n(c2=O)Cc2cc3cc(OC)ccc3nc2-1 ; CC[C@@]1(O)C(=O)OCc2c1cc1n(c2=O)Cc2c-1nc1ccc(OC)cc1c2CNCCCN=[N+]=[N-] ; CC[C@@]1(O)C(=O)OCc2c1cc1n(c2=O)Cc2c-1nc1cc(F)c(C)c3c1c2[C@@H](NCCS(C)(=O)=O)CC3 ; CCc1c2c(nc3ccc(O)cc13)-c1cc3c(c(=O)n1C2)COC(=O)[C@]3(O)CC ; C#CCNCc1c2c(nc3ccc(OC)cc13)-c1cc3c(c(=O)n1C2)COC(=O)[C@]3(O)CC]

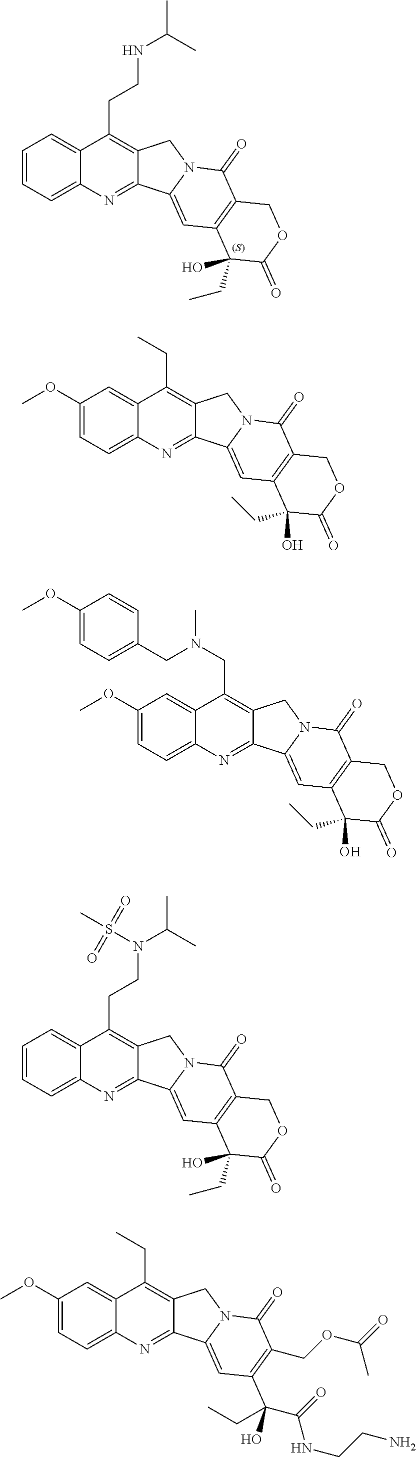

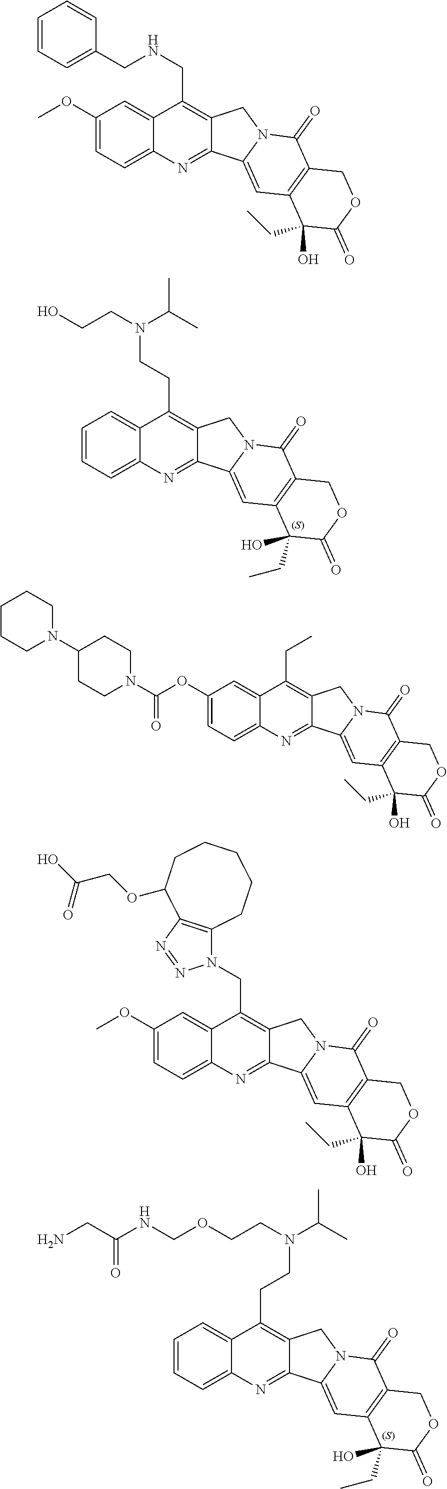

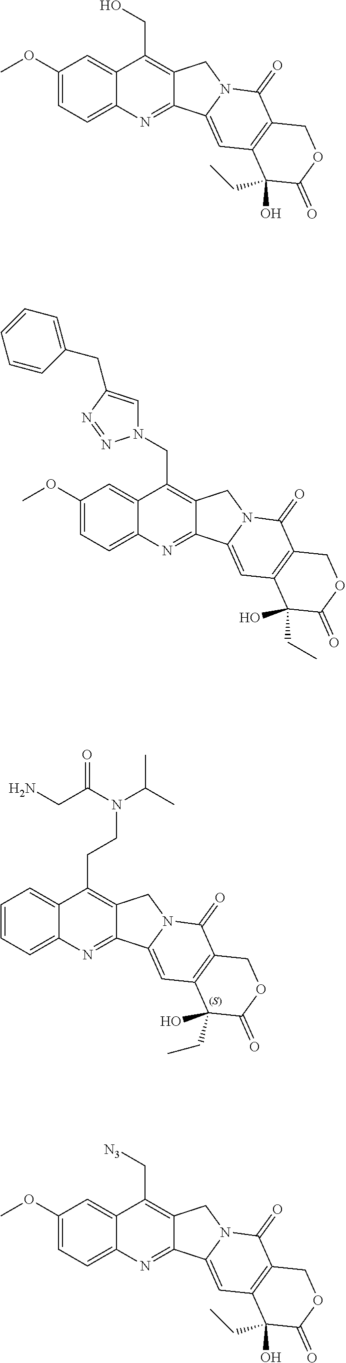

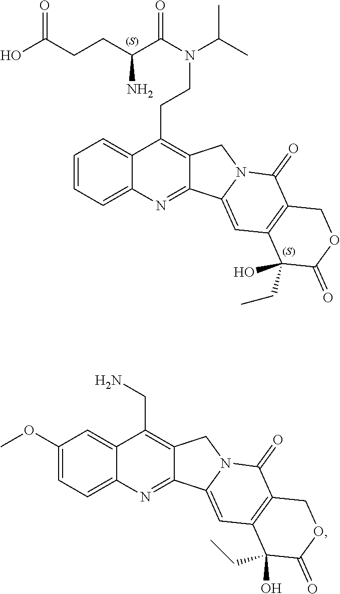

or a pharmaceutically acceptable salt thereof.

### Anti-HER2 Antibody-Drug Conjugates

In certain embodiments, the protein-drug conjugates, e.g., ADCs, disclosed herein are useful, inter alia, for the treatment, prevention and/or amelioration of any disease or disorder associated with or mediated by HER2 expression or activity, or treatable by binding HER2 without competing against modified LDL, or and/or promoting HER2 receptor internalization and/or decreasing cell surface receptor number.

The protein-drug conjugates of the present disclosure (and therapeutic compositions comprising the same) are useful, inter alia, for treating any disease or disorder in which stimulation, activation and/or targeting of an immune response would be beneficial. In particular, the anti-HER2 protein-drug conjugates, including both monospecific anti-HER2 antibodies and bispecific anti-HER2/HER2 antibodies of the present disclosure can be used for the treatment, prevention and/or amelioration of any disease or disorder associated with or mediated by HER2 expression or activity or the proliferation of HER2+ cells. The mechanism of action by which the therapeutic methods of the present disclosure are achieved include killing of the cells expressing HER2 in the presence of effector cells, for example, by CDC, apoptosis, ADCC, phagocytosis, or by a combination of two or more of these mechanisms. Cells expressing HER2 which can be inhibited or killed using the protein-drug conjugates of the present disclosure include, for example, breast tumor cells.

In one embodiment, the protein-drug conjugates of the present disclosure (and therapeutic compositions and dosage forms comprising same) comprise a bispecific antigen-binding molecule comprising:
a first antigen-binding domain (D1); and
a second antigen-binding domain (D2);
wherein D1 specifically binds a first epitope of human HER2; and
wherein D2 specifically binds a second epitope of human HER2.

In one embodiment of the above, D1 and D2 do not compete with one another for binding to human HER2.

The protein-drug conjugates of the present disclosure can be used to treat, e.g., primary and/or metastatic tumors arising in the prostate, bladder, cervix, lung, colon, kidney, breast, pancreas, stomach, uterus, and/or ovary. In certain embodiments, the protein-drug conjugates of the present disclosure are used to treat one or more of the following cancers: prostate cancer, bladder cancer, cervical cancer, lung cancer, colon cancer, kidney cancer, breast cancer, pancreatic cancer, stomach cancer, uterine cancer, and ovarian cancer. According to certain embodiments of the present disclosure, the anti-HER2 antibodies or anti-HER2/HER2 bispecific antibodies are useful for treating a patient afflicted with a breast cancer cell that is IHC2+ or more. According to other related embodiments of the present disclosure, methods are provided comprising administering an anti-HER2 antibody or an anti-HER2/HER2 antibody as disclosed herein to a patient who is afflicted with a breast cancer cell that is IHC2+ or more. Analytic/diagnostic methods known in the art, such as tumor scanning, etc., can be used to ascertain whether a patient harbors a tumor that is castrate-resistant.

In certain embodiments, the present disclosure also includes methods for treating residual cancer in a subject. The term “residual cancer” means the existence or persistence of one or more cancerous cells in a subject following treatment with an anti-cancer therapy.

The protein-drug conjugates of the present disclosure (and therapeutic compositions comprising the same) are useful, inter alia, for treating any disease or disorder in which stimulation, activation and/or targeting of an immune response would be beneficial. In particular, protein-drug conjugates comprising the anti-HER2 antibodies or anti HER2/HER2 antibodies of the present disclosure can be used for the treatment, prevention and/or amelioration of any disease or disorder associated with or mediated by HER2 expression or activity or the proliferation of HER2+ cells. The mechanism of action by which the therapeutic methods of the present disclosure are achieved include killing of the cells expressing HER2 in the presence of effector cells, for example, by CDC, apoptosis, ADCC, phagocytosis, or by a combination of two or more of these mechanisms. Cells expressing HER2 which can be inhibited or killed using the protein-drug conjugates of the present disclosure include, for example, breast tumor cells.

According to certain aspects, the present disclosure provides methods for treating a disease or disorder associated with HER2 expression (e.g., breast cancer) comprising administering one or more of the anti-HER2 protein-drug conjugates or anti-HER2/HER2 bispecific protein-drug conjugates described elsewhere herein to a subject after the subject has been determined to have breast cancer (e.g., and IHC2+ breast cancer). For example, the present disclosure includes methods for treating breast cancer comprising administering protein-drug conjugate comprising an anti-HER2 antibody or antigen-binding molecule or an anti-HER2/HER2 bispecific antibody or antigen-binding molecule to a patient 1 day, 2 days, 3 days, 4 days, 5 days, 6 days, 1 week, 2 weeks, 3 weeks or 4 weeks, 2 months, 4 months, 6 months, 8 months, 1 year, or more after the subject has received hormone therapy (e.g., anti-androgen therapy).

In certain embodiments, the present disclosure also includes the use of an anti-HER2 antibody of the present disclosure in the manufacture of a medicament for the treatment of a disease or disorder (e.g., cancer) related to or caused by HER2-expressing cells. In one aspect, the present disclosure relates to a protein-drug conjugate comprising an anti-HER2 antibody or antigen-binding fragment or an anti-HER2/HER2 bispecific antibody or antigen-binding fragment, as disclosed herein, for use in medicine. In one aspect, the present disclosure relates to a compound comprising an antibody-drug conjugate (ADC) as disclosed herein, for use in medicine. Combination Therapies and Formulations

The present disclosure provides methods which comprise administering a pharmaceutical composition comprising any of the exemplary protein-drug conjugates (e.g., antibody-drug conjugates), linker-payloads and payloads described herein in combination with one or more additional therapeutic agents. Exemplary additional therapeutic agents that may be combined with or administered in combination with protein-drug conjugates (e.g., antibody-drug conjugates), linker-payloads and payloads of the present disclosure include, e.g., a HER2 antagonist (e.g., an anti-HER2 antibody [e.g., trastuzumab] or a small molecule inhibitor of HER2 or an anti-HER2 antibody-drug conjugate, or an anti-HER2/HER2 bispecific antibody or an anti-HER2/HER2 bispecific antibody-drug conjugate), an EGFR antagonist (e.g., an anti-EGFR antibody [e.g., cetuximab or panitumumab] or small molecule inhibitor of EGFR [e.g., gefitinib or erlotinib]), an antagonist of another EGFR family member such as HER2/ErbB2, ErbB3 or ErbB4 (e.g., anti-ErbB2, anti-ErbB3 or anti-ErbB4 antibody or small molecule inhibitor of ErbB2, ErbB3 or ErbB4 activity), an antagonist of EGFRvIII (e.g., an antibody that specifically binds EGFRvIII), a cMET antagonist (e.g., an anti-cMET antibody), an IGF1R antagonist (e.g., an anti-IGF1R antibody), a B-raf inhibitor (e.g., vemurafenib, sorafenib, gDC-0879, PLX-4720), a PDGFR-α inhibitor (e.g., an anti-PDGFR-α antibody), a PDGFR-β inhibitor (e.g., an anti-PDGFR-β antibody), a VEGF antagonist (e.g., a VEGF-Trap, see, e.g., U.S. Pat. No. 7,087,411 (also referred to herein as a “VEGF-inhibiting fusion protein”), anti-VEGF antibody (e.g., bevacizumab), a small molecule kinase inhibitor of VEGF receptor (e.g., sunitinib, sorafenib or pazopanib)), a DLL4 antagonist (e.g., an anti-DLL4 antibody disclosed in US 2009/0142354), an Ang2 antagonist (e.g., an anti-Ang2 antibody disclosed in US 2011/0027286 such as H1H685P), a FOLH1 (PSMA) antagonist, a PRLR antagonist (e.g., an anti-PRLR antibody), a STEAP1 or STEAP2 antagonist (e.g., an anti-STEAP1 antibody or an anti-STEAP2 antibody), a TMPRSS2 antagonist (e.g., an anti-TMPRSS2 antibody), a MSLN antagonist (e.g., an anti-MSLN antibody), a CA9 antagonist (e.g., an anti-CA9 antibody), a uroplakin antagonist (e.g., an anti-uroplakin antibody), etc.

Other agents that may be beneficially administered in combination with the protein-drug conjugates (e.g., antibody-drug conjugates), linker-payloads and payloads of the disclosure include cytokine inhibitors, including small-molecule cytokine inhibitors and antibodies that bind to cytokines such as IL-1, IL-2, IL-3, IL-4, IL-5, IL-6, IL-8, IL-9, IL-11, IL-12, IL-13, IL-17, IL-18, or to their respective receptors. The pharmaceutical compositions of the present disclosure (e.g., pharmaceutical compositions comprising an anti-HER2, an anti-HER2/HER2 bispecific, an anti-MET, an anti-MET/MET bispecific, or an anti-STEAP2 protein-drug conjugate (e.g., antibody-drug conjugate as disclosed herein) may also be administered as part of a therapeutic regimen comprising one or more therapeutic combinations selected from “ICE”: ifosfamide (e.g., Ifex®), carboplatin (e.g., Paraplatin®), etoposide (e.g., Etopophos®, Toposar®, VePesid®, VP-16); “DHAP”: dexamethasone (e.g., Decadron®), cytarabine (e.g., Cytosar-U®, cytosine arabinoside, ara-C), cisplatin (e.g., Platinol®-AQ); and “ESHAP”: etoposide (e.g., Etopophos®, Toposar®, VePesid®, VP-16), methylprednisolone (e.g., Medrol®), high-dose cytarabine, cisplatin (e.g., Platinol®-AQ).

The present disclosure also includes therapeutic combinations comprising any of the protein-drug conjugates (e.g., antibody-drug conjugates), linker-payloads and payloads mentioned herein and an inhibitor of one or more of HER2, VEGF, Ang2, DLL4, EGFR, ErbB2, ErbB3, ErbB4, EGFRvIII, cMet, IGF1R, B-raf, PDGFR-α, PDGFR-β, FOLH1 (PSMA), PRLR, STEAP1, STEAP2, TMPRSS2, MSLN, CA9, uroplakin, or any of the aforementioned cytokines, wherein the inhibitor is an aptamer, an antisense molecule, a ribozyme, an siRNA, a peptibody, a nanobody or an antibody fragment (e.g., Fab fragment; F(ab′)2 fragment; Fd fragment; Fv fragment; scFv; dAb fragment; or other engineered molecules, such as diabodies, triabodies, tetrabodies, minibodies and minimal recognition units). The antigen-binding molecules of the disclosure may also be administered and/or co-formulated in combination with antivirals, antibiotics, analgesics, corticosteroids and/or NSAIDs. The antigen-binding molecules of the disclosure may also be administered as part of a treatment regimen that also includes radiation treatment and/or conventional chemotherapy.

The additional therapeutically active component(s) may be administered just prior to, concurrent with, or shortly after the administration of an antigen-binding molecule of the present disclosure; (for purposes of the present disclosure, such administration regimens are considered the administration of an antigen-binding molecule “in combination with” an additional therapeutically active component).

The present disclosure includes pharmaceutical compositions in which protein-drug conjugates (e.g., antibody-drug conjugates), linker-payloads and/or payloads of the present disclosure are co-formulated with one or more of the additional therapeutically active component(s) as described elsewhere herein.

### Administration Regimens

According to certain embodiments of the present disclosure, multiple doses of a protein-drug conjugate (e.g., an anti-HER2, an anti-HER2/HER2 bispecific, an anti-MET, an anti-MET/MET bispecific, or an anti-STEAP2 antibody-drug conjugate), linker-payload and/or a payload may be administered to a subject over a defined time course. The methods according to this aspect of the disclosure comprise sequentially administering to a subject multiple doses of a protein-drug conjugate (e.g., an anti-HER2, an anti-HER2/HER2 bispecific, an anti-MET, an anti-MET/MET bispecific, or an anti-STEAP2 antibody-drug conjugate), linker-payload and/or a payload of the disclosure. As used herein, “sequentially administering” means that each dose of a protein-drug conjugate (e.g., an anti-HER2, an anti-HER2/HER2 bispecific, an anti-MET, an anti-MET/MET bispecific, or an anti-STEAP2 antibody-drug conjugate), linker-payload and/or a payload is administered to the subject at a different point in time, e.g., on different days separated by a predetermined interval (e.g., hours, days, weeks or months). The present disclosure includes methods which comprise sequentially administering to the patient a single initial dose of a protein-drug conjugate (e.g., an anti-HER2, an anti-HER2/HER2 bispecific, an anti-MET, an anti-MET/MET bispecific, or an anti-STEAP2 antibody-drug conjugate), linker-payload and/or a payload, followed by one or more secondary doses of the protein-drug conjugate (e.g., an anti-HER2, an anti-HER2/HER2 bispecific, an anti-MET, an anti-MET/MET bispecific, or an anti-STEAP2 antibody-drug conjugate), linker-payload and/or payload, and optionally followed by one or more tertiary doses of the a protein-drug conjugate (e.g., an anti-HER2, an anti-HER2/HER2 bispecific, an anti-MET, an anti-MET/MET bispecific, or an anti-STEAP2 antibody-drug conjugate), linker-payload and/or payload.

The terms “initial dose,” “secondary doses,” and “tertiary doses,” refer to the temporal sequence of administration of the protein-drug conjugate (e.g., an anti-HER2, an anti-HER2/HER2 bispecific, an anti-MET, an anti-MET/MET bispecific, or an anti-STEAP2 antibody-drug conjugate), linker-payload and/or payload of the disclosure. Thus, the “initial dose” is the dose which is administered at the beginning of the treatment regimen (also referred to as the “baseline dose”); the “secondary doses” are the doses which are administered after the initial dose; and the “tertiary doses” are the doses which are administered after the secondary doses. The initial, secondary, and tertiary doses may all contain the same amount of the protein-drug conjugate (e.g., an anti-HER2, or an anti-HER2/HER2 bispecific, an anti-MET, an anti-MET/MET bispecific, or an anti-STEAP2 antibody-drug conjugate), linker-payload and/or payload, but generally may differ from one another in terms of frequency of administration. In certain embodiments, however, the amount of the protein-drug conjugate (e.g., an anti-HER2, an anti-HER2/HER2 bispecific, an anti-MET, an anti-MET/MET bispecific, or an anti-STEAP2 antibody-drug conjugate), linker-payload and/or payload contained in the initial, secondary and/or tertiary doses varies from one another (e.g., adjusted up or down as appropriate) during the course of treatment. In certain embodiments, two or more (e.g., 2, 3, 4, or 5) doses are administered at the beginning of the treatment regimen as “loading doses” followed by subsequent doses that are administered on a less frequent basis (e.g., “maintenance doses”).

In one exemplary embodiment of the present disclosure, each secondary and/or tertiary dose is administered 1 to 26 (e.g., 1, 1½, 2, 2½, 3, 3½, 4, 4½, 5, 5½, 6, 6½, 7, 7½, 8, 8½, 9, 9½, 10, 10½, 11, 11½, 12, 12½, 13, 13½, 14, 14½, 15, 15½, 16, 16½, 17, 17½, 18, 18½, 19, 19½, 20, 20½, 21, 21½, 22, 22½, 23, 23½, 24, 24½, 25, 25½, 26, 26½, or more) weeks after the immediately preceding dose. The phrase “the immediately preceding dose,” as used herein, means, in a sequence of multiple administrations, the dose of a protein-drug conjugate (e.g., an anti-HER2, an anti-HER2/HER2 bispecific, an anti-MET, an anti-MET/MET bispecific, or an anti-STEAP2 antibody-drug conjugate), linker-payload and/or payload which is administered to a patient prior to the administration of the very next dose in the sequence with no intervening doses.

The methods according to this aspect of the disclosure may comprise administering to a patient any number of secondary and/or tertiary doses of a protein-drug conjugate (e.g., an anti-HER2, an anti-HER2/HER2 bispecific, an anti-MET, an anti-MET/MET bispecific, or an anti-STEAP2 antibody-drug conjugate), linker-payload and/or payload. For example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In embodiments involving multiple secondary doses, each secondary dose may be administered at the same frequency as the other secondary doses. For example, each secondary dose may be administered to the patient 1 to 2 weeks after the immediately preceding dose. Similarly, in embodiments involving multiple tertiary doses, each tertiary dose may be administered at the same frequency as the other tertiary doses. For example, each tertiary dose may be administered to the patient 2 to 4 weeks after the immediately preceding dose. Alternatively, the frequency at which the secondary and/or tertiary doses are administered to a patient can vary over the course of the treatment regimen. The frequency of administration may also be adjusted during the course of treatment by a physician depending on the needs of the individual patient following clinical examination.

### EXAMPLES

The following examples illustrate specific aspects of the instant description. The examples should not be construed as limiting, as the examples merely provide specific understanding and practice of the embodiments and their various aspects.

Abbreviations

| | |
|---|---|
| ADC | Antibody-drug conjugate |
| Aglycosylated | Antibody does not have any glycan |
| antibody | |
| aq. | Aqueous |
| Boc | N-tert-butoxycarbonyl |
| BupH | Thermo Scientific Prod# 28372, containing 100 mM |
| | sodium phosphate and 150 mM sodium chloride, |
| | potassium free, pH was adjusted from 7.2 to 7.6-7.8 |
| | MQ, unless otherwise noted. |
| COT | Cyclooctynol |
| Da | Dalton |
| DAR | Drug to antibody ratio. |
| DCM | Dichloromethane |
| DIBAC | Dibenz[b,f]azocine, 11,12-didehydro-5,6-dihydro- |
| DIBAC-Suc | Dibenz[b,f]azocine-5(6H)-butanoic acid, 11,12-didehydro |
| DIBACT | 3H-Benzo[c]-1,2,3-triazolo[4,5-e][1]benzazocine, |
| | 8,9-dihydro- |
| DIPEA | Diisopropylethylamine |
| DMF | N,N-dimethylformamide |
| DMSO | Dimethylsulfoxide |
| EC | Enzyme commission |
| ELSD | Evaporating light scattering detector |
| Equiv. | Equivalent |
| ESI | Electrospray ionization |
| g | Gram |
| GUL-azide | (((S)-5-(4-azidobutanamido)-1-carboxypentyl)carbamoyl)- |
| | L-glutamic acid |
| HATU | 2-(7-Aza-1H-benzotriazole-1-yl)-1,1,3,3- |
| | tetramethyluronium hexafluorophosphate |
| HC | Heavy chain of immunoglobulin |
| HEK | Human embryonic kidney (cells) |
| HOBt | 1-Hydroxybenzotriazole |
| HPLC | High performance liquid chromatography |
| hr or hrs | Hours |
| LC | Light chain of immunoglobulin |
| LC | Liquid chromatography |
| MALDI | Matrix-assisted laser desorption/ionization |
| mg | milligrams |
| min | minutes |
| mL | milliliters |
| mmh | myc-myc-hexahistidine tag |
| μL | microliters |
| mM | millimolar |
| μM | micromolar |
| MS | Mass spectrometry |
| MSD | Mass-selective detector |
| MW | Molecular weight |
| NHS | N-hydroxy succinimide |
| nM | nanomolar |
| NMR | Nuclear magnetic resonance |
| PAB | Para-aminobezyloxy(carbonyl) |
| PBS | 10 mM sodium phosphate buffer and 150 mM sodium |
| | chloride |
| PBSg | 10 mM phosphate, 150 mM sodium chloride, 5% glycerol |
| PEG | Polyethyleneglycol |
| ppm | Parts per million (chemical shift) |
| PPTS | pyridinium p-toluenesulfonate |
| RP | Reversed phase |
| RT | Room temperature |
| Sat. | Saturated |
| SDS-PAGE | Sodium dodecylsulfate polyacrylamide gel electrophoresis |
| SEC | Size exclusion chromatography |
| Suc | Succinic acid |
| TCEP | Tris(2-carboxyethyl)phosphine hydrochloride |
| TEA | Triethylamine |
| TFA | Trifluoroacetic acid |
| TG | Transglutaminase |
| THF | Tetrahydrofuran |
| TOF | Time-of-flight |
| UPLC | Ultra Performance Liquid Chromatography |
| UV | Ultraviolet |
| VC | Valine-citrulline |

Exatecan mesylate (P1), Belotecan HCl salt (P8), 10-hydroxy camptothecin (P14), SN38 (P16), and Irinotecan (P19) were commercially available from MOE or Bide Pharm; compound 7A-1 was commercially available from TCI; compound 7-2 was commercially available from Accela. Linkers 5-1 were synthesized as described in WO2018089373 and WO2020146541; linkers 6-1 were synthesized as described in WO2020146541.

### Example 1: Synthesis of Pro-Exatecan (ProEXT) (Scheme 1)

The payloads according to the present disclosure and their properties are listed in Table-1, below.

TABLE 1
List of Payloads

| | | | | | | | Exact |
|---|---|---|---|---|---|---|---|
| | | | | | | Purity | mass |
| P# | Code | Structures | cLogP | MF | MW | (%) | (M + H)⁺ |
| P1 | Exatecan (mesylate) | 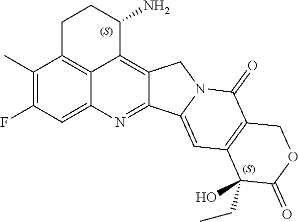 | 1.45 | C₂₄H₂₂FN₃O₄ · CH₄O₃S | 435.5 + 96.1 | 97 | 435.16 |
| P2 | Pro-Exatecan (GlyEXT) | 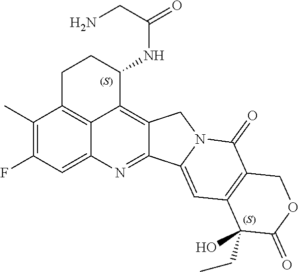 | 0.44 | C₂₆H₂₅FN₄O₅ | 492.5 | 99 | 492.18 |
| P3 | (GluEXT) | 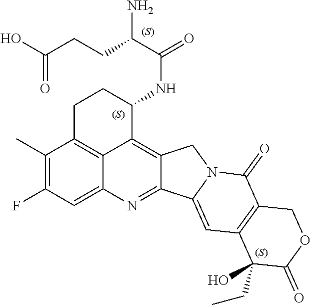 | −1.61 | C₂₉H₂₉FN₄O₇ | 564.6 | 98 | 565.2 |
| P4 | (LysEXT) | 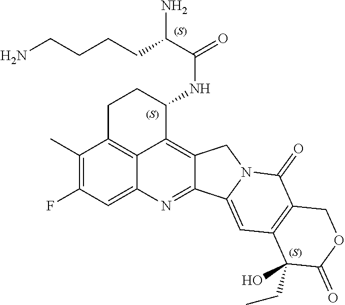 | 0.73 | C₃₀H₃₄FN₅O₅ | 563.6 | 99 | 564.2 |
| P5 | (PheEXT) | 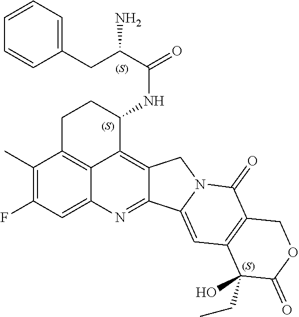 | 2.67 | C₃₃H₃₁FN₄O₅ | 582.6 | 99 | 583.3 |
| P6 | (MsEXT) | 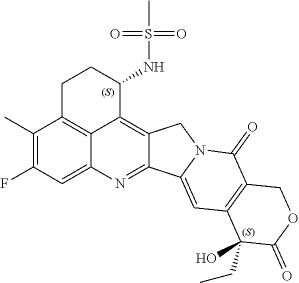 | 0.86 | C₂₅H₂₄FN₃O₆S | 513.5 | 99 | 514.1 |
| P7 | (MsEthEXT) | 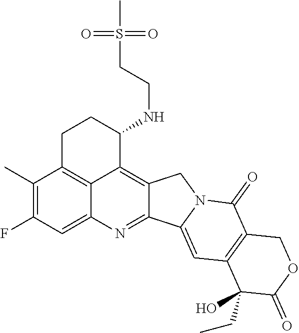 [SMILES: CC[C@@]1(O)C(=O)OCc2c1cc1n(c2=O)Cc2c-1nc1cc(F)c(C)c3c1c2[C@@H](NCCS(C)(=O)=O)CC3] | 0.66 | C₂₇H₂₈FN₃O₆S | 541.6 | 99 | 541.9 |
| P8 | (Belotecan, HCl salt) | 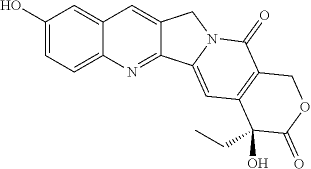 | 1.69 | C₂₅H₂₇N₃O4 · HCl | 433.5 + 36.5 | 99 | 434.2 |
| P9 | (MSBLT) | 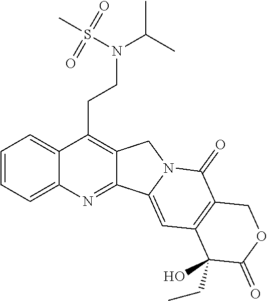 [SMILES: CC[C@@]1(O)C(=O)OCc2c1cc1n(c2=O)Cc2c-1nc1ccccc1c2CCN(C(C)C)S(C)(=O)=O] | 0.95 | C₂₆H₂₉N₃O₆S | 511.6 | 97 | 512.3 |
| P10 | (HOEthBLT) | 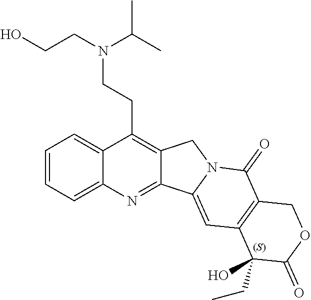 | 1.53 | C₂₇H₃₁N₃O₅ | 477.6 | 99 | 478.2 |
| P11 | (ProBLT) | 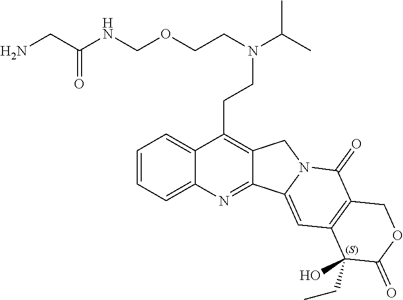 | 0.38 | C₃₀H₃₇N₅O₆ | 563.7 | 99 | 564.3 |
| P31 | | 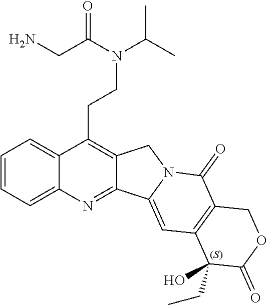 | 0.53 | C₂₇H30N₄O5 | 490.6 | >90 | 491.1 |
| P32 | | 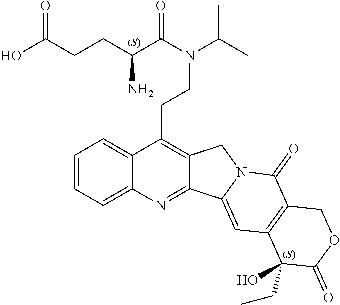 | −1.52 | C30H34N407 | 562.6 | >90 | 563.2 |
| P33 | | 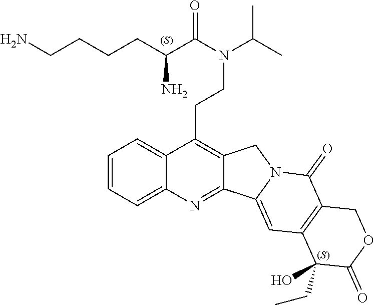 | 0.80 | C₃₁H₃₉N₅O₅ | 561.7 | >90 | 562.3 |
| P34 | | 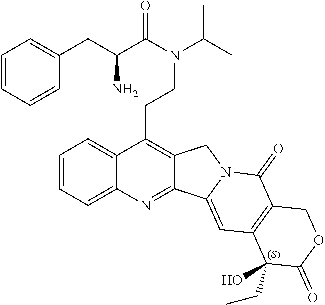 | 2.75 | C₃₄H₃₆N₄O₅ | 580.7 | >90 | 581.3 |
| P12 | Dxd | 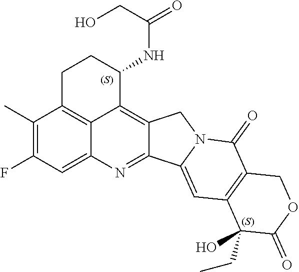 | 0.55 | C₂₆H₂₄FN₃O₆ | 493.5 | >95 | 494.3 |
| P13 | | 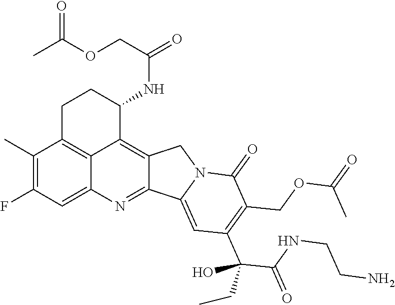 [SMILES: CC[C@@](O)(C(=O)NCCN)c1cc2n(c(=O)c1COC(C)=O)Cc1c-2nc2cc(F)c(C)c3c2c1[C@@H](NC(=O)COC(C)=O)CC3] | 0.61 | C₃₂H₃₆FN₅O₈ | 637.7 | >99 | 638.3 [M + H] |
| P14 | | 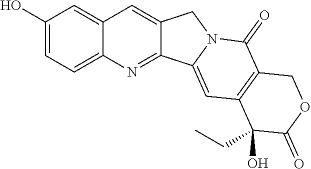 [SMILES: CC[C@@]1(O)C(=O)OCc2c1cc1n(c2=O)Cc2cc3cc(O)ccc3nc2-1] | 0.92 | C₂₀H₁₆N₂O₅ | 364.4 | 99 | 365.1 |
| P15 | | 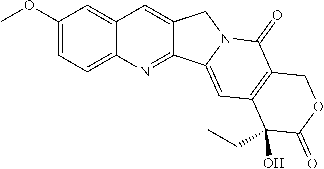 [SMILES: CC[C@@]1(O)C(=O)OCc2c1cc1n(c2=O)Cc2cc3cc(OC)ccc3nc2-1] | 1.06 | C₂₁H₁₈N₂O₅ | 378.4 | 95 | 379.1 |
| P16 | SN38 | 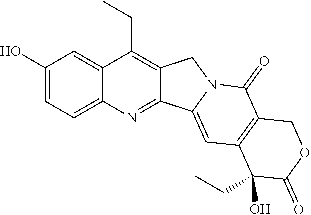 [SMILES: CCc1c2c(nc3ccc(O)cc13)-c1cc3c(c(=O)n1C2)COC(=O)[C@]3(O)CC] | 1.87 | C₂₂H₂₀N₂O₅ | 392.4 | 98 | 393.1 |
| P17 | | 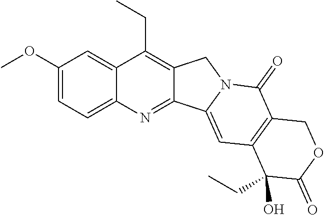 [SMILES: CCc1c2c(nc3ccc(OC)cc13)-c1cc3c(c(=O)n1C2)COC(=O)[C@]3(O)CC] | 2.02 | C₂₃H₂₂N₂O₅ | 406.4 | 94 | 407.1 |
| P18 | | 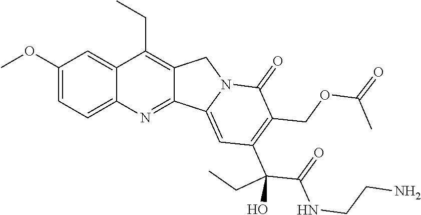 [SMILES: CCc1c2c(nc3ccc(OC)cc13)-c1cc([C@@](O)(CC)C(=O)NCCN)c(COC(C)=O)c(=O)n1C2] | 0.42 | C₂₇H₃₂N₄O₆ | 508.6 | 99 | 449.2 (M − OAc) |
| P19 | Irinotecan | 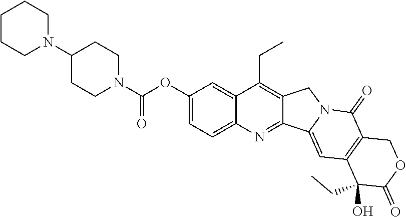 [SMILES: CCc1c2c(nc3ccc(OC(=O)N4CCC(N5CCCCC5)CC4)cc13)-c1cc3c(c(=O)n1C2)COC(=O)[C@]3(O)CC] | 2.78 | C₃₃H₃₈N₄O₆ | 586.7 | 97 | 587.2 |
| P20 | | 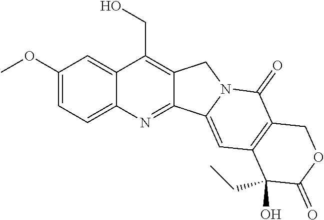 [SMILES: CC[C@@]1(O)C(=O)OCc2c1cc1n(c2=O)Cc2c-1nc1ccc(OC)cc1c2CO] | 0.30 | C₂₂H₂₀N₂O₆ | 408.4 | 90 | 409.1 |
| P21 | | 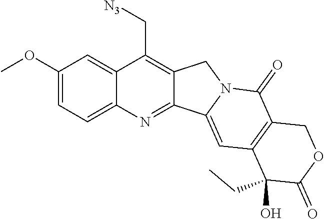 [SMILES: CC[C@@]1(O)C(=O)OCc2c1cc1n(c2=O)Cc2c-1nc1ccc(OC)cc1c2CN=[N+]=[N-]] | 1.12 | C₂₂H₁₉N₅O₅ | 433.4 | 95 | 434.1 |
| P22 | | 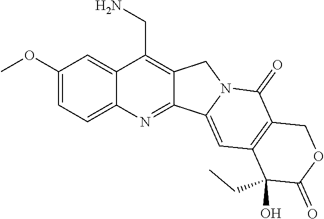 [SMILES: CC[C@@]1(O)C(=O)OCc2c1cc1n(c2=O)Cc2c-1nc1ccc(OC)cc1c2CN] | 0.19 | C₂₂H₂₁N₃O₅ | 407.4 | 99 | 408.3 |
| P41 | | 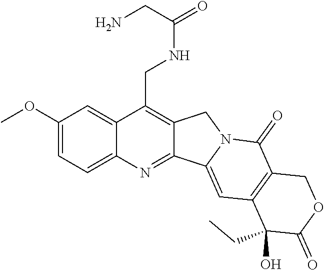 [SMILES: CC[C@@]1(O)C(=O)OCc2c1cc1n(c2=O)Cc2c-1nc1ccc(OC)cc1c2CNC(=O)CN] | −0.92 | C24H24N4O6 | 464.5 | 96 | 465.2 |
| P42 | | 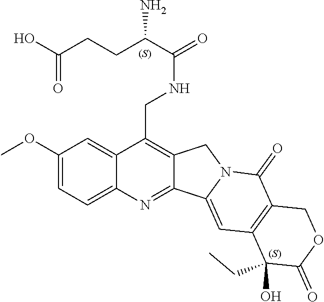 | −2.97 | C27H28N4O8 | 536.5 | 95 | 537.2 |
| P43 | | 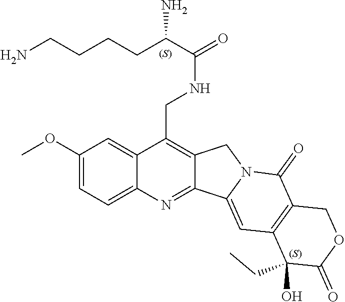 | −0.62 | C28H33N5O6 | 535.6 | 95 | 536.3 |
| P44 | | 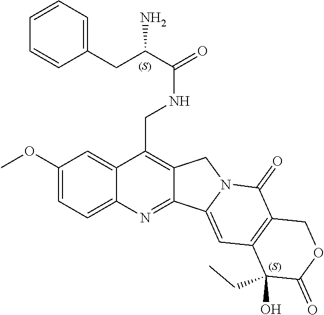 | 1.31 | C31H30N4O6 | 554.6 | 98 | 555.2 |
| P23 | | 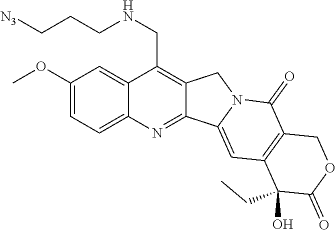 [SMILES: CC[C@@]1(O)C(=O)OCc2c1cc1n(c2=O)Cc2c-1nc1ccc(OC)cc1c2CNCCCN=[N+]=[N-]] | 0.81 | C₂₅H₂₆N₆O₅ | 490.5 | 99 | 491.2 |
| P24 | | 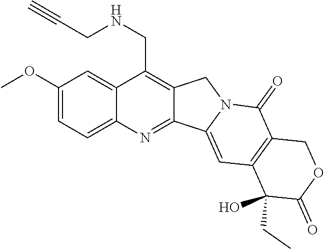 [SMILES: C#CCNCc1c2c(nc3ccc(OC)cc13)-c1cc3c(c(=O)n1C2)COC(=O)[C@]3(O)CC] | 0.85 | C₂₅H₂₃N₃O₅ | 445.5 | 99 | 446.3 |
| P25 | | 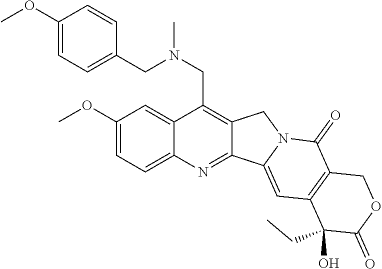 [SMILES: CC[C@@]1(O)C(=O)OCc2c1cc1n(c2=O)Cc2c-1nc1ccc(OC)cc1c2CN(C)Cc1ccc(OC)cc1] | 2.57 | C₃₁H₃₁N₃O₆ | 541.6 | 98 | 542.2 |
| P26 | | 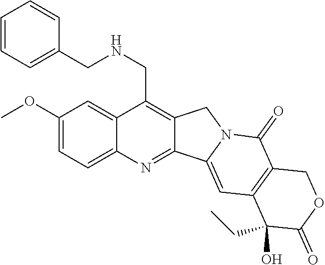 [SMILES: CC[C@@]1(O)C(=O)OCc2c1cc1n(c2=O)Cc2c-1nc1ccc(OC)cc1c2CNCc1ccccc1] | 2.35 | C₂₉H₂₇N₃O₅ | 497.6 | 99 | 498.2 |
| P27 | | 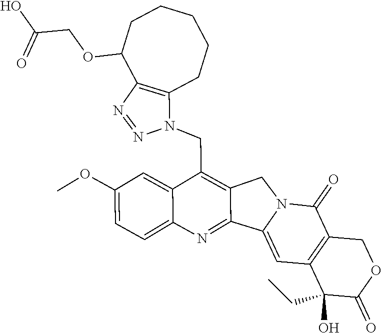 [SMILES: CC[C@@]1(O)C(=O)OCc2c1cc1n(c2=O)Cc2c-1nc1ccc(OC)cc1c2Cn1nnc2c1CCCCCC2OCC(=O)O] | 1.44 | C₃₂H₃₃N₅O₈ | 615.6 | 99 | 616.3 |
| P28 | | 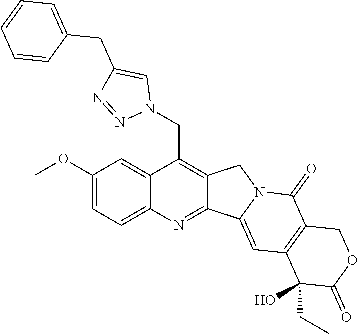 [SMILES: CC[C@@]1(O)C(=O)OCc2c1cc1n(c2=O)Cc2c-1nc1ccc(OC)cc1c2Cn1cc(Cc2ccccc2)nn1] | 2.80 | C₃₁H₂₇N₅O₅ | 549.6 | 96 | 550.1 |

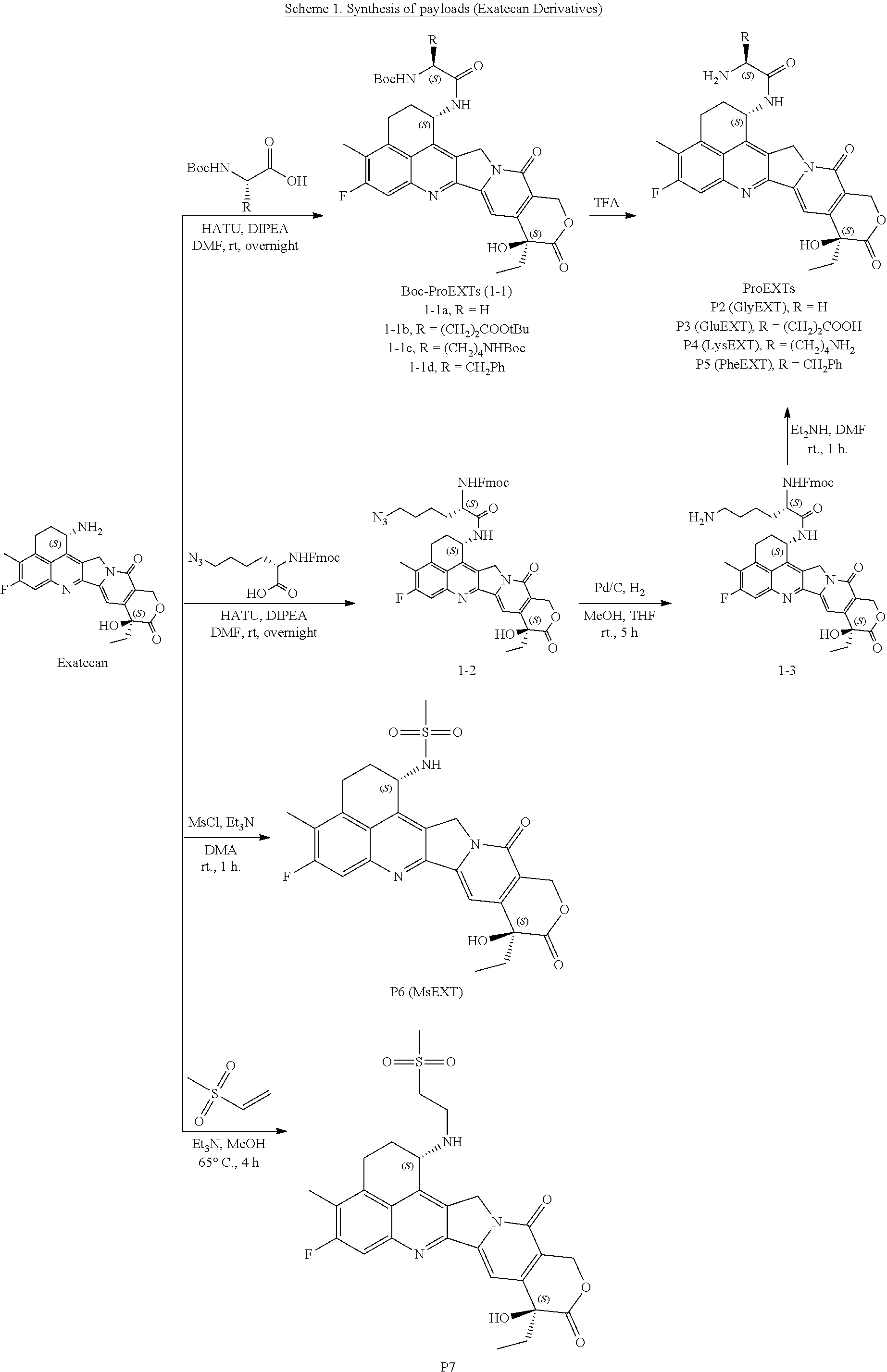

### Example 1A: General Procedure A for Synthesis of Protected ProEXTs (1-1)

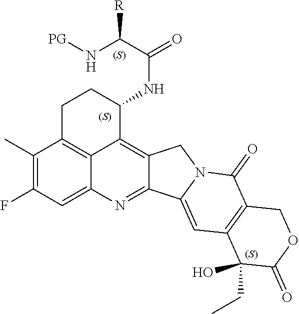

To a solution of protected amino acid (1.0 equiv.) in DMF (50 mM) were added exatecan mesylate (1.0 equiv.), HATU (1.5 equiv.) and DIPEA (2.0 equiv.) successively, and the reaction mixture was stirred at room temperature overnight, which was monitored by LCMS. The resulting mixture was directly purified by reversed phase flash chromatography (0-100% acetonitrile in aq. TFA (0.01%)) to give compound 1-1 as a light yellow solid.

### Example 1A-1: tert-Butyl N-({[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}methyl)carbamate (Boc-GlyEXT, 1-1a)

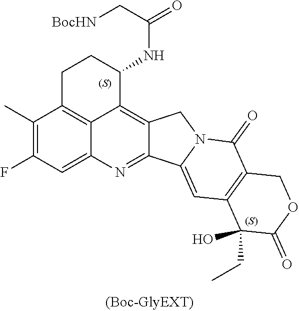

Following the general procedure A starting from Boc-Gly-OH (18 mg, 0.10 mmol), Boc-GlyEXT (1-1a) (45 mg, 75% yield) was obtained as a light yellow solid. ESI m/z: 593.3 (M+H)⁺.

### Example 1A-2: tert-butyl (4S)-4-{[(tert-butoxy)carbonyl]amino}-4-{[(10S,23S)-10-ethyl-18-fluoro-10-hydroxy-19-methyl-5,9-dioxo-8-oxa-4,15-diazahexacyclo[[14.7.1.0²,¹⁴.0⁴,¹³.0⁶,¹¹.0²⁰,²⁴]]tetracosa-1,6(11),12,14,16,18,20(24)-heptaen-23-yl]carbamoyl}butanoate Boc-Glu(OtBu)EXT, 1-1b)

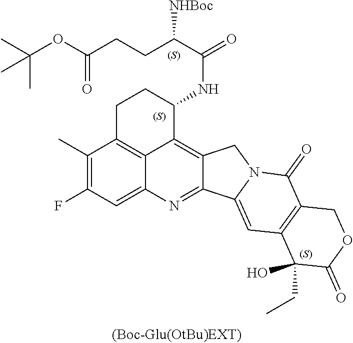

Following the general procedure A starting from N-Boc-Glu(OtBu)-OH (CAS: 13726-84-6, 50 mg, 0.16 mmol), Boc-Glu(OtBu)EXT (1-1b) (91 mg, 77% yield) was obtained as a yellow solid. ESI m/z: 721.3 (M+H)⁺.

### Example 1A-3: tert-butyl N-[(1 S)-5-{[(tert-butoxy)carbonyl]amino}-1-{[(10S,23S)-10-ethyl-18-fluoro-10-hydroxy-19-methyl-5,9-dioxo-8-oxa-4,15-diazahexacyclo[[14.7.1.0²,¹⁴.0⁴,¹³.0⁶,¹¹.0²⁰,²⁴]]tetracosa-1,6(11),12,14,16,18,20(24)-heptaen-23-yl]carbamoyl}pentyl]carbamate (Boc-Lys(Boc)EXT, 1-1c)

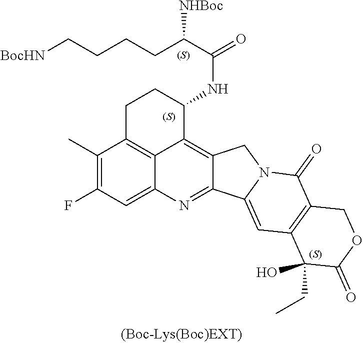

Following the general procedure A starting from Boc-Lys(Boc)-OH (CAS: 2483-46-7, 0.12 g, 0.35 mmol), Boc-Lys(Boc)EXT (1-1c) (0.17 g, 64% yield) was obtained as a grey solid. ESI m/z: 764.2 (M+H)⁺; 664.3 (M-Boc+H)⁺.

### Example 1A-4: (9H-fluoren-9-yl)methyl N-[(1S)-1-{[(10S,23S)-10-ethyl-18-fluoro-10-hydroxy-19-methyl-5,9-dioxo-8-oxa-4,15-diazahexacyclo[[14.7.1.0²,¹⁴.0⁴,¹³.0⁶,¹¹.0²⁰,²⁴]]tetracosa-1,6(11),12,14,16,18,20(24)-heptaen-23-yl]carbamoyl}-2-phenylethyl]carbamate (Fmoc-PheEXT, 1-1d)

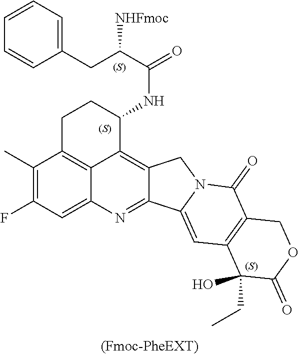

Following the general procedure A starting from N-Fmoc-Phe-OH (CAS: 35661-40-6, 50 mg, 0.13 mmol), N-Fmoc-PheEXT (1-1d) (73 mg, 70% yield) was obtained as a yellow solid. ESI m/z: 805.2 (M+H)⁺.

### Example 1A-5: 2-Amino-N-[(10S,23S)-10-ethyl-18-fluoro-10-hydroxy-19-methyl-5,9-dioxo-8-oxa-4,15-diazahexacyclo[[14.7.1.0²,¹⁴.0⁴,¹³.0⁶,¹¹.0²⁰,²⁴]]tetracosa-1,6(11),12,14,16,18,20(24)-heptaen-23-yl]acetamide (GlyEXT, P2)

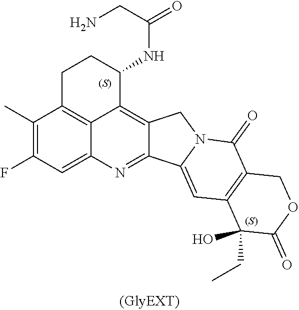

To a solution of Boc-GlyEXT (1-1a) (45 mg, 75 μmol) in DCM (3 mL) was added TFA (1 mL), and the mixture was stirred at room temperature for 2 hours, which was monitored by LCMS. The resulting mixture was concentrated in vacuo and the residue was purified by prep-HPLC (5-95% acetonitrile in aq. TFA (0.3%)) to give GlyEXT (P2) (35 mg, 90% yield) as a white solid. ESI m/z: 493.2 (M+H)⁺.

### Example 1A-6: (4S)-4-amino-4-{[(10S,23S)-10-ethyl-18-fluoro-10-hydroxy-19-methyl-5,9-dioxo-8-oxa-4,15-diazahexacyclo[[14.7.1.0²,¹⁴.0⁴,¹³.0⁶,¹¹.0²⁰,²⁴]]tetracosa-1,6(11),12,14,16,18,20(24)-heptaen-23-yl]carbamoyl}butanoic acid (GluEXT, P3)

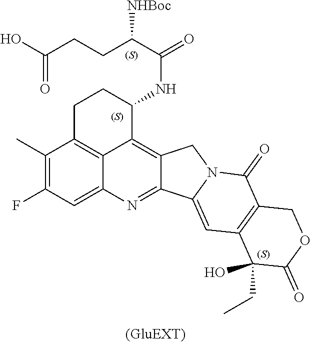

Following the similar procedure as P2 except starting from 1-1b (60 mg, 83 μmol) instead of 1-1a, P3 (34 mg, 60% yield, TFA salt) was obtained as a yellow solid. ESI m/z: 565.2 (M+H)⁺. ¹H NMR (400 MHz, DMSO_{d6}) δ 9.00 (d, J=8.0 Hz, 1H), 8.34-8.13 (m, 2H), 7.86-7.83 (m, 1H), 7.33 (s, 1H), 6.55 (s, 1H), 5.62-5.57 (m, 1H), 5.43-5.38 (m, 3H), 5.07-5.02 (m, 1H), 3.78 (t, J=6.0 Hz, 1H), 3.22-3.12 (m, 2H), 2.43 (s, 3H), 2.33-2.15 (m, 4H), 1.98-1.79 (m, 4H), 0.88 (t, J=7.2 Hz, 3H) ppm. ¹⁹F NMR (376 MHz, DMSO_{d6}) 6-73.5, -110.9 ppm.

### Example 1A-7: (2S)-2,6-diamino-N-[(10S,23S)-10-ethyl-18-fluoro-10-hydroxy-19-methyl-5,9-dioxo-8-oxa-4,15-diazahexacyclo[[14.7.1.0²,¹⁴.0⁴,¹³.0⁶,¹¹.0²⁰,²⁴]]tetracosa-1,6(11),12,14,16,18,20(24)-heptaen-23-yl]hexanamide (LysEXT, P4)

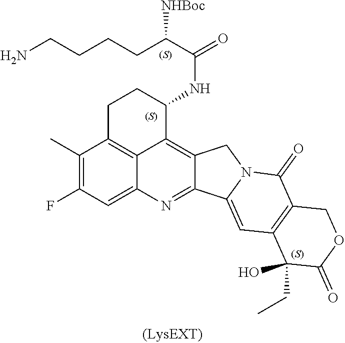

Following the similar procedure as P2 except starting from 1-1c (50 mg, 65 μmol) instead of 1-1a, P4 (34 mg, 66% yield, TFA salt) was obtained as a yellow solid. ESI m/z: 564.2 (M+H)⁺. ¹H NMR (400 MHz, DMSO_{d6}) δ 9.09-9.06 (m, 1H), 8.27 (s, 3H), 7.85-7.83 (m, 1H), 7.67 (s, 2H), 7.35 (s, 1H), 6.57 (s, 1H), 5.58-5.52 (m, 1H), 5.48-5.43 (m, 2H), 5.38-5.33 (m, 1H), 5.13-5.08 (m, 1H), 3.78 (s, 1H), 3.24-3.15 (m, 2H), 2.76 (s, 2H), 2.42 (s, 3H), 2.30-2.14 (m, 2H), 1.90-1.84 (m, 2H), 1.80-1.70 (m, 2H), 1.55-1.46 (m, 2H), 1.39-1.28 (m, 2H), 0.89 (t, J=7.2 Hz, 3H) ppm. ¹⁹F NMR (376 MHz, DMSO_{d6}) δ −73.6, -110.9 ppm.

### Example 1A-8: (2S)-2-amino-N-[(10S,23S)-10-ethyl-18-fluoro-10-hydroxy-19-methyl-5,9-dioxo-8-oxa-4,15-diazahexacyclo[[14.7.1.0²,¹⁴.0⁴,¹³.0⁶,¹¹.0²⁰,²⁴]]tetracosa-1,6(11),12,14,16,18,20(24)-heptaen-23-yl]-3-phenylpropanamide (PheEXT, P5)

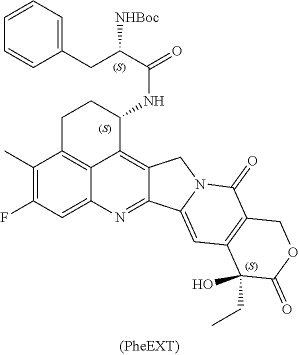

To a solution of compound 1-1d (50 mg, 62 μmol) in DMF (1 mL) was added diethylamine (0.1 mL), and the reaction mixture was stirred at room temperature for 30 minutes, which was monitored by LCMS. The resulting mixture was purified by prep-HPLC twice (5-95% acetonitrile in aq. TFA (0.01%)) to give P5 (14 mg, 39% yield) as a light yellow solid. ESI m/z: 583.3 (M+H)⁺. ¹H NMR (400 MHz, DMSO_{d6}) δ 8.85 (d, J=8.4 Hz, 1H), 8.31 (s, 3H), 7.86-7.83 (m, 1H), 7.32 (s, 1H), 7.02 (s, 1H), 7.00 (s, 1H), 6.91 (t, J=7.6 Hz, 2H), 6.85 (t, J=7.2 Hz, 1H), 6.56 (s, 1H), 5.59-5.52 (m, 1H), 5.47-5.41 (m, 2H), 5.25-5.20 (m, 1H), 4.62-4.58 (m, 1H), 4.05 (s, 1H), 3.27-3.10 (m, 2H), 3.05-2.95 (m, 2H), 2.43 (s, 3H), 2.33-2.10 (m, 2H), 1.98-1.85 (m, 2H), 0.93-0.89 (m, 3H) ppm. ¹⁹F NMR (376 MHz, DMSO_{d6}) δ −73.5, -111.0 ppm.

### Example 1B: Alternative Synthesis of LysEXT (P4)

### Example 11B-1: (9H-fluoren-9-yl)methyl N-[(1 S)-5-azido-1-{[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}pentyl]carbamate (Fmoc-Lys(Azido)EXT, 1-2)

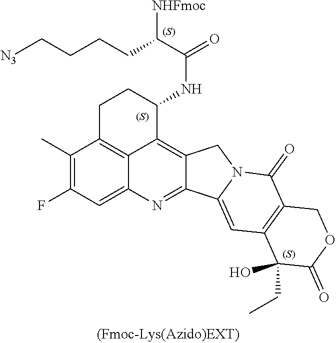

Following the general procedure A starting from Fmoc-L-azidolysine (CAS: 159610-89-6, 41 mg, 0.10 mmol), Fmoc-Lys(Azido)EXT (1-2) (40 mg, 92% yield) was obtained as a light yellow solid. ¹H NMR (400 MHz, DMSO_{d6}) 68.56 (d, J=8.4 Hz, 1H), 7.89-7.86 (m, 2H), 7.80 (d, J=11.0 Hz, 1H), 7.75-7.54 (m, 3H), 7.44-7.35 (m, 2H), 7.35-7.20 (m, 3H), 6.52 (br s, 1H), 5.55-5.51 (m, 1H), 5.39 (d, J=16.2 Hz, 1H), 5.33-5.22 (m, 2H), 5.09 (d, J=19.0 Hz, 1H), 4.32-4.13 (m, 3H), 4.10-3.95 (m, 1H), 3.34-3.24 (m, 2H), 3.16-3.12 (m, 2H), 2.40 (s, 3H), 2.23-2.09 (m, 2H), 1.89-1.77 (m, 2H), 1.68-1.62 (m, 2H), 1.49-1.42 (m, 2H), 1.40-1.22 (m, 2H), 0.87 (t, J=7.3 Hz, 3H) ppm. ¹⁹F NMR (376 MHz, DMSO_{d6}) δ −73.9, −111.2 ppm.

### Example 11B-2: (9H-fluoren-9-yl)methyl N-[(1S)-5-amino-1-{[(10S,23S)-10-ethyl-18-fluoro-10-hydroxy-19-methyl-5,9-dioxo-8-oxa-4,15-diazahexacyclo[[14.7.1.0²,¹⁴.0⁴,¹³.0⁶,¹¹.0²⁰,²⁴]]tetracosa-1,6(11),12,14,16,18,20(24)-heptaen-23-yl]carbamoyl}pentyl]carbamate (1-3)

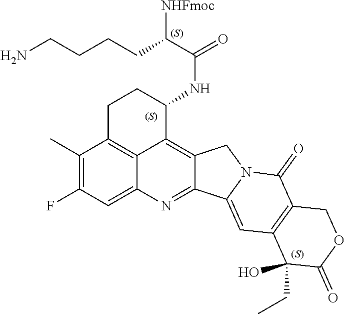

To a yellow solution of compound 1-2 (80 mg, 86 μmol) in methanol (15 mL) and THF (5 mL) was added palladium on carbon (50 mg, 10% Pd) under nitrogen protection. The mixture was stirred at room temperature under hydrogen atmosphere for 5 hours, which was monitored by LCMS. The resulting mixture was filtered through Celite and the filtrate was concentrated. The residue was purified by prep-HPLC (5-95% acetonitrile in aq. TFA (0.01%)) to give compound 1-3 (70 mg, crude) as a light yellow solid. ESI m/z 393.5 (M/2+H)⁺. ¹⁹F NMR (376 MHz, DMSO_{d6}) δ −73.5, -111.1 ppm.

### Example 11B-3: (2S)-2,6-diamino-N-[(10S,23S)-10-ethyl-18-fluoro-10-hydroxy-19-methyl-5,9-dioxo-8-oxa-4,15-diazahexacyclo[[14.7.1.0²,¹⁴.0⁴,¹³.0⁶,¹¹.0²⁰,²⁴]]tetracosa-1,6(11),12,14,16,18,20(24)-heptaen-23-yl]hexanamide (LysEXT, P4)

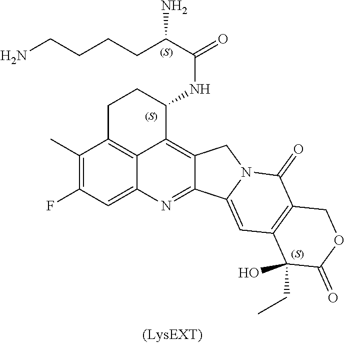

To a solution of compound 1-3 (10 mg, crude, obtained above) in DMF (1 mL) was added diethylamine (0.2 mL), and the reaction mixture was stirred at room temperature for an hour, which was monitored by LCMS. The resulting solution was directly separated by prep-HPLC (5-95% acetonitrile in aq. TFA (0.01%)) to give P4 (5 mg, 41% yield from 1-2, TFA salt) as a light yellow solid. ESI m/z: 564.2 (M+H)⁺.

### Example 1C: Synthesis of N-[(10S,23S)-10-ethyl-18-fluoro-10-hydroxy-19-methyl-5,9-dioxo-8-oxa-4,15-diazahexacyclo[[14.7.1.0²,¹⁴.0⁴,¹³.0⁶,¹¹.0²⁰,²⁴]]tetracosa-1,6(11),12,14,16,18,20(24)-heptaen-23-yl]methanesulfonamide (MsEXT, P6)

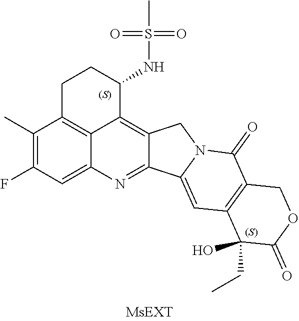

To a solution of Exatecan mesylate (30 mg, 56 μmol) in dry DMA (1 mL) were added successively triethylamine (17 mg, 0.17 mmol) and methanesulfonyl chloride (7.1 mg, 62 μmol) at 0° C., and the reaction mixture was stirred at room temperature for an hour, which was monitored by LCMS. The resulting mixture was directly separated by reversed phase flash chromatography (0-70% acetonitrile in aq. TFA (0.1%)) to give P6 (4.0 mg, 11% yield) as a yellow solid. ESI m/z 514.0 (M+H)⁺. ¹H NMR (400 MHz, DMSO_{d6}) δ 7.81 (d, J=1.6 Hz, 1H), 7.79 (d, J=4.8 Hz, 1H), 7.32 (s, 1H), 6.52 (s, 1H), 5.43-5.42 (m, 3H), 5.13-5.18 (m, 1H), 3.18 (br, 4H), 2.39 (s, 3H), 2.30-2.26 (m, 2H), 1.93-1.83 (m, 3H), 1.16 (t, J=7.2 Hz, 1H), 0.88 (t, J=7.2 Hz, 3H) ppm. ¹⁹F NMR (377 MHz, DMSO_{d6}) δ −73.44, −111.30 ppm.

### Example 1D: Synthesis of (10S,23S)-10-ethyl-18-fluoro-10-hydroxy-23-[(2-methanesulfonylethyl)amino]-19-methyl-8-oxa-4,15-diazahexacyclo[[14.7.1.0²,¹⁴.0⁴,¹³.0⁶,¹¹.0²⁰,²⁴]]tetracosa-1,6(11),12,14,16,18,20(24)-heptaene-5,9-dione (MsEthEXT, P7)

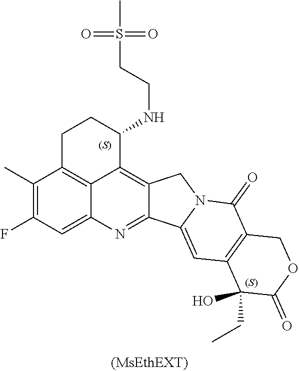

To a solution of Exatecan mesylate (10 mg, 19 μmol) in methanol (2 mL) were added (methylsulfonyl)ethane (5.0 mg, 46 μmol) and triethylamine (10 mg, 92 μmol)), and the reaction mixture was stirred at 65° C. for 4 hours, which was monitored by LCMS. The resulting mixture was directly separated by reversed phase flash chromatography (5-95% acetonitrile in aq. TFA (0.1%)) to give P7 (5.0 mg, 40% yield, TFA salt) as a white solid. ESI m/z 542.5 (M+H)⁺. ¹H NMR (400 MHz, DMSO_{d6}) δ 7.84 (d, J=11.2 Hz, 1H), 7.34 (s, 1H), 6.60 (s, 1H), 5.54-5.36 (m, 5H), 5.00 (s, 1H), 3.55-3.40 (m, 5H), 3.20-3.10 (m, 5H), 2.65-2.60 (m, 1H), 2.40 (s, 3H), 1.89-1.85 (m, 2H), 0.88-0.84 (m, 3H) ppm.

### Example 2: Synthesis of Payloads P9-P11 and P31-P34 (Scheme 2)

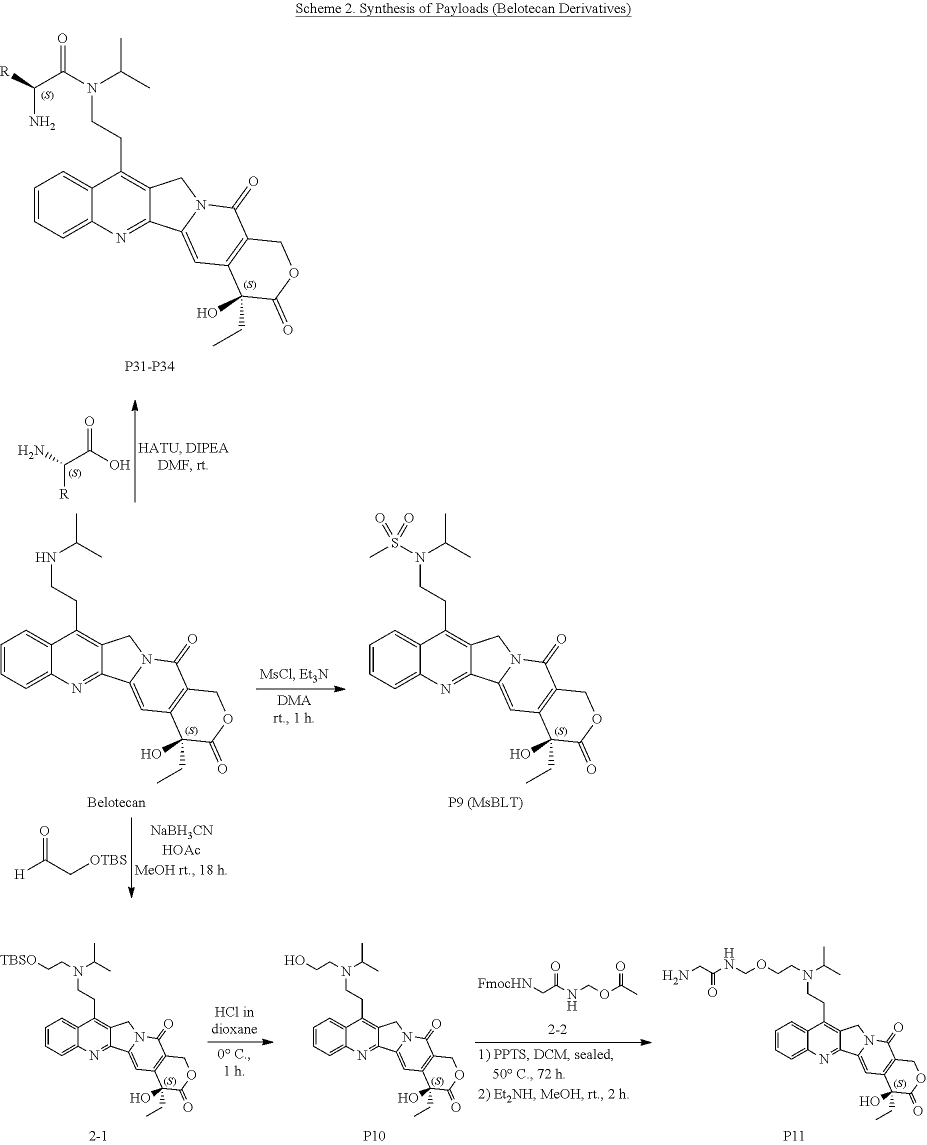

### Example 2A: Synthesis of N-{2-[(19S)-19-ethyl-19-hydroxy-14,18-dioxo-17-oxa-3,13-diazapentacyclo[11.8.0.0²,¹¹.0⁴,⁹.0¹⁵,²⁰]henicosa-1(21),2,4,6,8,10,15(20)-heptaen-10-yl]ethyl}-N-(propan-2-yl)methanesulfonamide (MsBLT, P9)

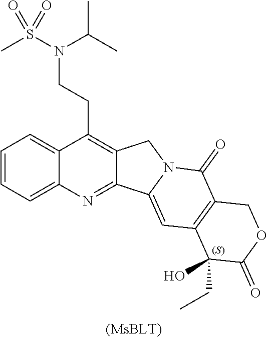

To a solution of Belotecan (30 mg, 64 μmol) in dry DMA (1 mL) were added successively triethylamine (19 mg, 0.19 mmol) and methanesulfonyl chloride (8.0 mg, 70 μmol) at 0° C., and the reaction mixture was stirred at room temperature for an hour, which was monitored by LCMS. The resulting mixture was directly separated by reversed phase flash chromatography (0-70% acetonitrile in aq. TFA (0.1%)) to give P9 (6.0 mg, 18% yield) as a yellow solid. ESI m/z 512.3 (M+H)⁺. ¹H NMR (400 MHz, DMSO_{d6}) δ 8.32 (d, J=8.4 Hz, 1H), 8.20 (d, J=8.4 Hz, 1H), 7.89 (t, J=7.6 Hz, 1H), 7.79 (t, J=7.6 Hz, 1H), 7.35 (s, 1H), 6.54 (s, 1H), 5.48-5.40 (m, 4H), 4.02-3.95 (m, 1H), 3.55-3.46 (m, 2H), 3.40 (s, 2H), 3.00 (s, 3H), 1.93-1.83 (m, 2H), 1.15 (d, J=4.0 Hz, 6H), 0.88 (t, J=7.6 Hz, 3H) ppm.

### Example 2B: Synthesis of (19S)-19-ethyl-19-hydroxy-10-{2-[(2-hydroxyethyl)(propan-2-yl)amino]ethyl}-17-oxa-3,13-diazapentacyclo[11.8.0.0²,¹¹.0⁴,⁹.0¹⁵,²⁰]henicosa-1(21),2,4,6,8,10,15(20)-heptaene-14,18-dione (HOEthBLT, P10)

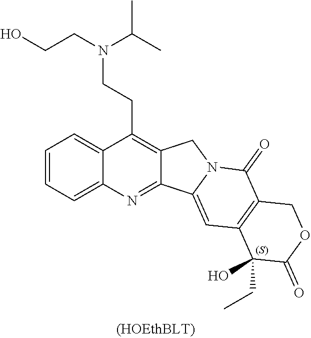

To a solution of Belotecan (60 mg, 0.12 mmol) in methanol were added O-TBS-hydroxyacetaldehyde (24 mg, 0.14 mmol) and acetic acid (0.01 mL, cat.) at room temperature, and the mixture was stirred at room temperature for 30 minutes before the addition of sodium cyanoborohydride (16 mg, 0.24 mmol). The reaction mixture was stirred at room temperature for 18 hours, which was monitored by LCMS. The resulting mixture was directly separated by reversed phase flash chromatography (5-35% acetonitrile in aq. TFA (0.01%)) to give compound 2-1 (43 mg, 51% yield, ESI m/z 592.4 (M+H)⁺) as a yellow solid.

The compound 2-1 (30 g, 42 μmol) obtained above was dissolved in a solution of hydrochloride in dioxane (4 N, 2 mL) at 0° C., and the mixture was stirred at 0° C. for an hour, which was monitored by LCMS. The resulting mixture was neutralized with sat. aq. sodium bicarbonate to pH 6-7 at 0° C. and concentrated in vacuo to remove dioxane. The residual mixture was separated by reversed phase flash chromatography (0-20% acetonitrile in aq. TFA (0.1%)) to give P10 (20 mg, 67% yield) as a yellow solid. ESI m/z 478.3 (M+H)⁺. ¹H NMR (400 MHz, DMSO_{d6}) δ 9.64 (s, 1H), 8.42 (d, J=8.7 Hz, 1H), 8.23 (d, J=8.5 Hz, 1H), 7.91 (t, J=8.3 Hz, 1H), 7.81 (t, J=8.0 Hz, 1H), 7.37 (s, 1H), 6.57 (s, 1H), 6.58 (s, 1H), 5.54-5.39 (m, 4H), 3.90-3.79 (m, 4H), 3.77-3.71 (m, 2H), 2.48-2.41 (m, 2H), 1.93-1.84 (m, 2H), 1.34-1.24 (m, 8H), 0.88 (t, J=7.3 Hz, 3H) ppm.

### Example 2C: Synthesis of 2-amino-N-{[2-({2-[(19S)-19-ethyl-19-hydroxy-14,18-dioxo-17-oxa-3,13-diazapentacyclo[11.8.0.0²,¹¹.0⁴,⁹.0¹⁵,²⁰]henicosa-1(21),2,4,6,8,10,15(20)-heptaen-10-yl]ethyl}(propan-2-yl)amino)ethoxy]methyl}acetamide (ProBLT, P11)

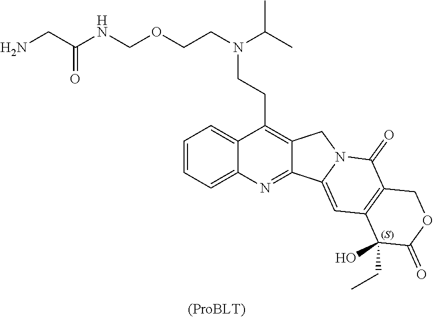

To a solution of P10 (83 mg, 0.14 mmol) in DCM (20 mL) were added compound 2-2 (CAS: 1599440-06-8, 57 mg, 0.14 mmol) and pyridinium p-toluenesulfonate (PPTS) (7.0 mg, 28 μmol) at 50° C. in a sealed tube, and the reaction mixture was stirred at 50° C. for 72 hours. After cooled, the volatiles were removed in vacuo and the residue was separated by reversed phase flash chromatography (0-100% acetonitrile in aq. TFA (0.01%)) to give a light yellow solid (50 mg, ESI m/z 393.6 (M/2+H)⁺), which was dissolved in methanol (2 mL). To the solution was added diethylamine (0.2 mL), and the reaction mixture was stirred at room temperature for 3 hours until Fmoc was totally removed according to LCMS. The resulting mixture was concentrated in vacuo and the residue was purified by prep-HPLC (5-95% acetonitrile in aq. TFA (0.01%)) to give P11 (26 mg, 27% yield from P10) as a light yellow solid. ESI m/z 564.3 (M+H)⁺. ¹H NMR (400 MHz, DMSO_{d6}) δ 9.99 (s, 1H), 9.46 (s, 1H), 8.95-8.81 (m, 1H), 8.37 (d, J=8.4 Hz, 1H), 8.23 (d, J=8.2 Hz, 1H), 8.12 (d, J=7.4 Hz, 1H), 7.90 (dd, J=18.3, 8.1 Hz, 2H), 7.82 (t, J=7.5 Hz, 1H), 7.60 (t, J=9.3 Hz, 3H), 7.51 (t, J=6.0 Hz, 1H), 7.37 (s, 1H), 7.25 (d, J=8.1 Hz, 2H), 6.57 (s, 1H), 5.99 (t, J=5.3 Hz, 1H), 5.55-5.35 (m, 6H), 4.91 (s, 2H), 4.71 (s, 2H), 4.44-4.33 (m, 1H), 4.30-4.19 (m, 2H), 3.90-3.72 (m, 6H), 3.65-3.56 (m, 5H), 3.53-3.30 (m, 16H), 3.26-3.22 (m, 2H), 3.03-2.93 (m, 2H), 2.36 (d, J=7.2 Hz, 1H), 2.29-2.01 (m, 4H), 2.00-1.80 (m, 6H), 1.80-1.53 (m, 7H), 1.46-1.34 (m, 3H), 1.32-1.24 (m, 6H), 0.90-0.82 (m, 9H) ppm.

### Example 3: Synthesis of Payloads P14-P17, P20-P28 and P41-P44 (Scheme 3)

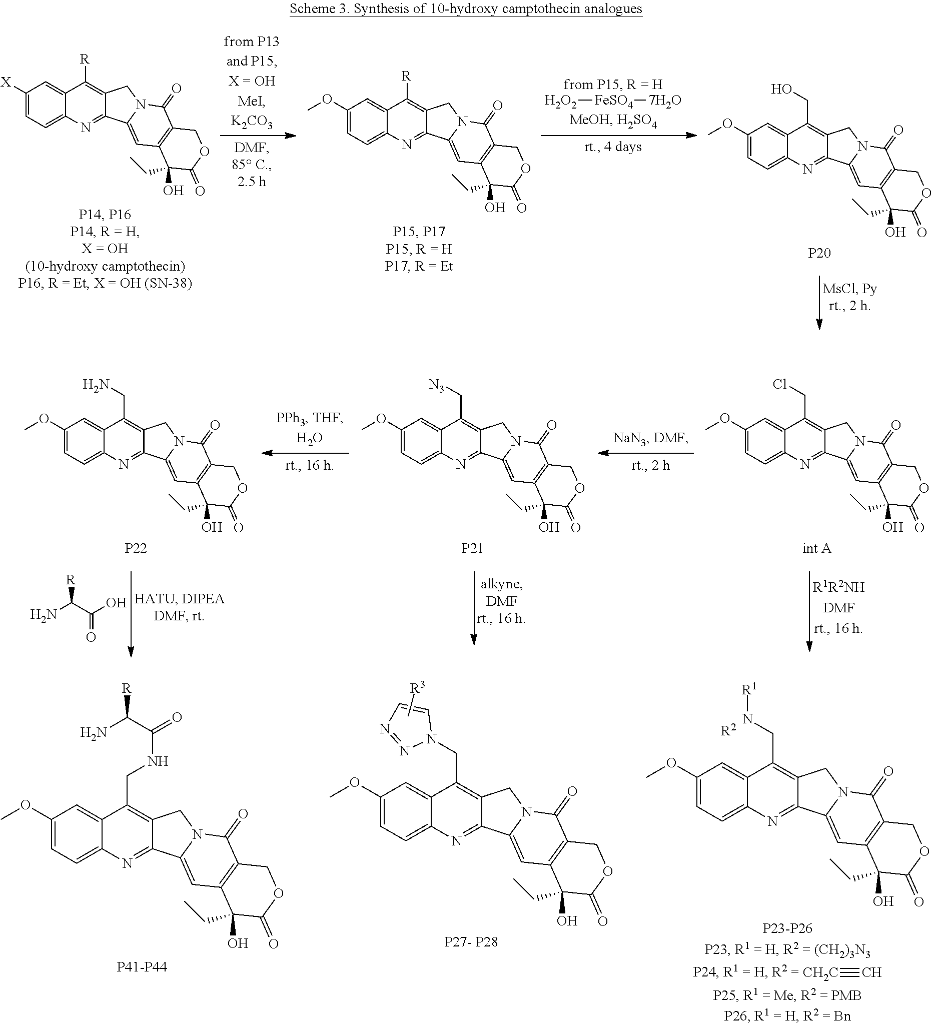

### Example 3A: Synthesis of (19S)-19-Ethyl-19-hydroxy-7-methoxy-17-oxa-3,13-diazapentacyclo[11.8.0.0²,¹¹.0⁴,⁹.0¹⁵,²⁰]henicosa-1(21),2,4,6,8,10,15(20)-heptaene-14,18-dione (P15)

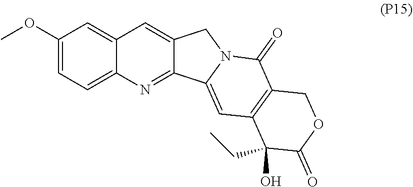 [SMILES: CC[C@@]1(O)C(=O)OCc2c1cc1n(c2=O)Cc2cc3cc(OC)ccc3nc2-1]

A mixture of 10-hydroxycamptothecin P14 (0.73 g, 2.0 mmol) and potassium carbonate (0.55 g, 4.0 mmol) was suspended in DMF (5 mL) and heated to 85° C. until the brown reaction mixture turned clear. Then the solution was cooled to room temperature and methyl iodide (0.73 g, 2.0 mmol) was added into the solution in one portion. The solution was stirred at 85° C. for another 2.5 h under nitrogen balloon until the reaction was completed, which was monitored by LCMS. A light yellow solid was precipitated. After cooled to room temperature, the reaction mixture was diluted with water (100 mL), filtered to collected the light yellow precipitates, which was dried in vacuo to provide compound P15 (0.50 g, 67% yield) as a light yellow solid. ESI m/z: 379.1 (M+H)⁺. ¹H NMR (400 MHz, DMSO_{d6}) δ 8.52 (s, 1H), 8.04 (d, J=10 Hz, 1H), 7.49-7.46 (m, 2H), 7.26 (s, 1H), 6.54 (s, 1H), 5.41 (s, 2H), 5.23 (s, 2H), 3.93 (s, 3H), 1.90-1.82 (m, 2H), 0.88 (t, J=7.6 Hz, 3H) ppm. Reference CN1587265, confirming the identity of P15, is incorporated by reference in its entirety.

### Example 3B: Synthesis of (19S)-10,19-Diethyl-19-hydroxy-7-methoxy-17-oxa-3,13-diazapentacyclo[11.8.0.0²,¹¹.0⁴,⁹.0¹⁵,²⁰]henicosa-1(21),2,4,6,8,10,15(20)-heptaene-14,18-dione (P17)

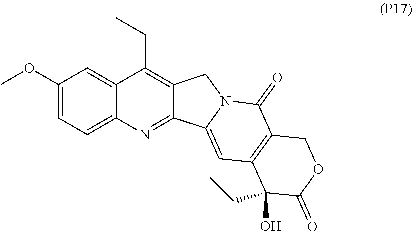 [SMILES: CCc1c2c(nc3ccc(OC)cc13)-c1cc3c(c(=O)n1C2)COC(=O)[C@]3(O)CC]

Following the similar procedure as compound P15, except using 7-ethyl-10-hydroxycamptothecin (SN-38, example 2) instead of 10-hydroxycamptothecin, compound P17 (0.52 g, 84% yield, free base) as a pale yellow solid was obtained and containing lactone-ring-opening product. 20 mg of the free base was further purified by prep-HPLC (5-95% acetonitrile in aq. TFA (0.3%)) to give example 5 (15 mg, TFA salt) as a pale yellow solid. ESI m/z: 407.1 (M+H)⁺. ¹H NMR (400 MHz, DMSO_{d6}) δ 8.08 (d, J=8.8 Hz, 1H), 7.53-7.49 (m, 2H), 7.27 (s, 1H), 6.52 (s, 1H), 5.43 (s, 2H), 5.30 (s, 2H), 3.99 (s, 3H), 3.20 (q, J=7.2 Hz 2H), 1.93-1.81 (m, 2H), 1.33 (t, J=7.6 Hz, 3H), 0.88 (t, J=7.2 Hz, 3H) ppm. (The proton of TFA was not observed). ¹⁹F NMR (376 MHz, DMSO_{d6}) δ −73 ppm. Reference WO2005044821, confirming the identity of P17, is incorporated by reference in its entirety.

### Example 3C: Synthesis of (19S)-19-Ethyl-19-hydroxy-10-(hydroxymethyl)-7-methoxy-17-oxa-3,13-diazapentacyclo[11.8.0.0²,¹¹.0⁴,⁹.0¹⁵,²⁰]henicosa-1(21),2,4,6,8,10,15(20)-heptaene-14,18-dione (P20)

To a cooled (0° C. ice-water bath) suspension of compound P15 (0.30 g, 0.79 mmol) in a co-solvent of methanol (10 mL) and water (10 mL) were added 96% sulfuric acid (5.3 mL, 95 mmol) dropwise and green vitriol (0.27 g, 0.95 mmol). The suspension turned clear and was cooled to −10° C. To the resulting solution was added 30% hydrogen peroxide (1 mL, 9.5 mmol) dropwise. The reaction mixture was stirred at room temperature for 4 days, which was monitored by LCMS. The mixture was diluted with water and a light yellow solid precipitated. After collected by filtration and dried, compound P20 (0.29 g, 90% yield) was obtained as a light yellow solid, which was pure enough without further purification. ESI m/z: 409.1 (M+H)⁺. ¹H NMR (400 MHz, DMSO_{d6}) δ 8.07 (d, J=9.2 Hz, 1H), 7.50 (dd, J=9.2, 2.0 Hz, 1H), 7.38 (s, 1H), 7.27 (s, 1H), 5.43 (s, 2H), 5.38 (s, 2H), 5.37 (s, 2H), 5.23 (br s, 2H), 3.96 (s, 3H), 1.89-1.83 (m, 2H), 0.89 (t, J=7.2 Hz, 3H) ppm.

### Example 3D: Synthesis of (19S)-10-(chloromethyl)-19-ethyl-19-hydroxy-7-methoxy-17-oxa-3,13-diazapentacyclo[11.8.0.0²,¹¹.0⁴,⁹.0¹⁵,²⁰]henicosa-1(21),2,4,6,8,10,15(20)-heptaene-14,18-dione (Int A)

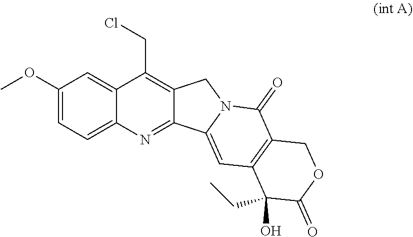 [SMILES: CC[C@@]1(O)C(=O)OCc2c1cc1n(c2=O)Cc2c-1nc1ccc(OC)cc1c2CCl]

To a stirred suspension of compound P20 (0.12 g, 0.29 mmol) in dry pyridine (3 mL) was added methanesulfonyl chloride (67 mg, 0.59 mmol) at room temperature. The suspension was then stirred at room temperature for 2 hours until P20 was totally consumed, which was monitored by LCMS. The reaction mixture was directly purified by reversed phase flash chromatography (0-50% acetonitrile in aq. TFA (0.01%)) to give the chloride (52 mg, crude) as a yellow solid, which was used for the next step. ESI m/z: 427.1 (M+H)⁺.

### Example 3E: Synthesis of (19S)-10-(Azidomethyl)-19-ethyl-19-hydroxy-7-methoxy-17-oxa-3,13-diazapentacyclo[11.8.0.0²,¹¹.0⁴,⁹.0¹⁵,²⁰]henicosa-1(21),2,4,6,8,10,15(20)-heptaene-14,18-dione (P21)

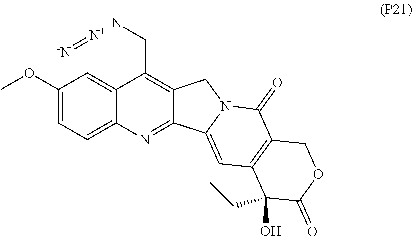

To a stirred yellow solution of int A (52 mg, crude, obtained above) in DMF (1.3 mL) was added sodium azide (30 mg, 0.47 mmol) at room temperature. The reaction mixture was stirred at room temperature for 2 hours until the reaction turned clear. LCMS showed the chloride was consumed then. The reaction mixture was directly purified by reversed phase flash chromatography (0-100% methanol in aq.TFA (0.01%)) to give compound P21 (25 mg, 26% yield from P20) as a pale yellow solid. ESI m/z: 434.1 (M+H)⁺. ¹H NMR (400 MHz, DMSO_{d6}) δ 8.13 (d, J=9.2 Hz, 1H), 7.58 (d, J=2.4 Hz, 1H), 7.57 (dd, J=9.2, 2.8 Hz, 1H), 7.29 (s, 1H), 6.54 (s, 1H), 5.42 (s, 2H), 5.40 (s, 2H), 5.27 (s, 2H), 4.00 (s, 3H), 1.91-1.83 (m, 2H), 0.88 (t, J=7.6 Hz, 3H) ppm.

### Example 3F: Synthesis of (19S)-10-(Aminomethyl)-19-ethyl-19-hydroxy-7-methoxy-17-oxa-3,13-diazapentacyclo[11.8.0.0²,¹¹.0⁴,⁹.0¹⁵,²⁰]henicosa-1(21),2,4,6,8,10,15(20)-heptaene-14,18-dione (P22)

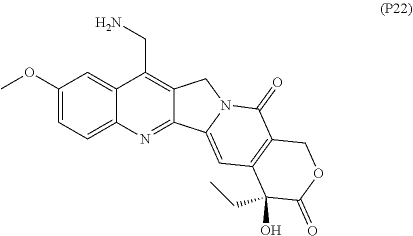 [SMILES: CC[C@@]1(O)C(=O)OCc2c1cc1n(c2=O)Cc2c-1nc1ccc(OC)cc1c2CN]

To a solution of compound P21 (20 mg, 46 μmol) in a co-solvent of THF (4 mL) and water (1 mL) was added triphenyl phosphine (36 mg, 0.14 mmol). The clear solution was stirred at room temperature overnight under nitrogen balloon until the reduction was completed according to LCMS. The reaction solution was concentrated in vacuo and the residue was purified by reversed phase flash chromatography (0-100% methanol in aq. TFA (0.01%)) to give compound P22 (7 mg, 37% yield, TFA salt) as a pale yellow solid. ESI m/z: 408.2 (M+H)⁺. ¹H NMR (400 MHz, DMSO_{d6}) δ 8.44 (s, 3H), 8.16 (d, J=8.8 Hz, 1H), 7.68 (d, J=2.4 Hz, 1H), 7.61 (dd, J=9.2, 2.4 Hz, 1H), 7.31 (s, 1H), 6.56 (s, 1H), 5.53 (s, 2H), 5.45 (s, 2H), 4.73 (s, 2H), 4.05 (s, 3H), 1.92-1.84 (m, 2H), 0.88 (t, J=7.6 Hz, 3H) ppm. ¹⁹F NMR (376 MHz, DMSO_{d6}) δ−73.62 ppm.

### Example 3G: General Procedure B for Synthesis of Payloads P23-26

To a solution of int A (1 equiv.) in dry DMF (10 mM) was added corresponding amine (2.0 equiv.), and the reaction mixture was stirred at room temperature for 2 hours, which was monitored by LCMS. The resulting mixture was directly separated by reversed phase flash chromatography (0-100% acetonitrile in aq. TFA (0.01%)) to give payload P23-P26 as a white solid.

### Example 3G-1: Synthesis of (S)-11-(((3-azidopropyl)amino)methyl)-4-ethyl-4-hydroxy-9-methoxy-1,12-dihydro-14H-pyrano[3′,4′:6,7]indolizino[1,2-b]quinoline-3,14(4H)-dione (P23)

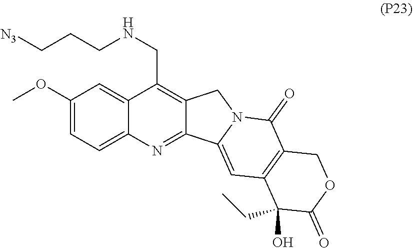 [SMILES: CC[C@@]1(O)C(=O)OCc2c1cc1n(c2=O)Cc2c-1nc1ccc(OC)cc1c2CNCCCN=[N+]=[N-]]

Following the general procedure B, payload P23 (4.0 mg, 17% yield) was obtained as a white solid. ESI m/z: 491.2 (M+H)⁺. ¹H NMR (400 MHz, DMSO_{d6}) δ 8.07 (d, J=8.8 Hz, 1H), 7.54 (s, 1H), 7.52-7.48 (m, 2H), 5.50 (d, J=16.4 Hz, 1H), 5.38 (s, 2H), 5.30 (d, J=16.4 Hz, 1H), 3.98 (s, 3H), 3.43 (t, J=6.0 Hz, 2H), 2.96-2.91 (m, 2H), 1.94-1.84 (m, 5H), 1.21-1.18 (m, 4H), 0.91 (t, J=7.2 Hz, 3H) ppm.

### Example 3G-2: Synthesis of (S)-4-ethyl-4-hydroxy-9-methoxy-11-((prop-2-yn-1-ylamino)methyl)-1,12-dihydro-14H-pyrano[3′,4′:6,7]indolizino[1,2-b]quinoline-3,14(4H)-dione (P24)

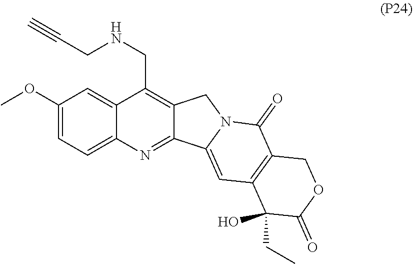 [SMILES: C#CCNCc1c2c(nc3ccc(OC)cc13)-c1cc3c(c(=O)n1C2)COC(=O)[C@]3(O)CC]

Following the general procedure B, payload P24 (1.3 mg, 6% yield) was obtained as a white solid. ESI m/z: 446.3 (M+H)⁺. ¹H NMR (400 MHz, DMSO_{d6}) δ 8.13-8.08 (m, 1H), 7.67 (d, J=2.8 Hz, 1H), 7.54-7.51 (m, 1H), 7.28 (s, 1H), 6.50 (s, 1H), 5.42-5.40 (m, 4H), 4.36 (br s, 1H), 3.97 (s, 3H), 2.00-1.97 (m, 1H), 1.90-1.83 (m, 2H), 1.29-1.18 (m, 4H), 0.88 (t, J=7.2 Hz, 3H) ppm.

### Example 3G-3: Synthesis of (S)-4-ethyl-4-hydroxy-9-methoxy-11-(((4-methoxybenzyl)(methyl)amino)methyl)-1,12-dihydro-14H-pyrano[3′,4′:6,7]indolizino[1,2-b]quinoline-3,14(4H)-dione (P25)

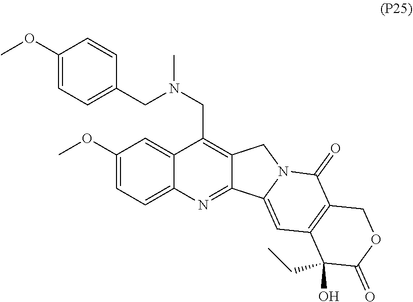 [SMILES: CC[C@@]1(O)C(=O)OCc2c1cc1n(c2=O)Cc2c-1nc1ccc(OC)cc1c2CN(C)Cc1ccc(OC)cc1]

Following the general procedure B, payload P25 (8.0 mg, 31% yield) was obtained as a white solid. ESI m/z: 542.2 (M+H)⁺. ¹H NMR (400 MHz, DMSO_{d6}) δ 7.96 (d, J=9.2 Hz, 1H), 7.48 (s, 1H), 7.41-7.33 (m, 3H), 7.10 (br s, 1H), 6.92-6.89 (m, 2H), 5.48 (d, J=16.0 Hz, 1H), 5.28 (d, J=16.0 Hz, 1H), 5.22 (br, 4H), 4.01-3.97 (m, 1H), 3.77-3.71 (m, 6H), 2.59-2.43 (m, 3H), 1.89-1.83 (m, 2H), 1.21-1.18 (m, 1H), 0.90 (t, J=7.2 Hz, 3H) ppm.

### Example 3G-4: Synthesis of (S)-11-((benzylamino)methyl)-4-ethyl-4-hydroxy-9-methoxy-1,12-dihydro-14H-pyrano[3′,4′:6,7]indolizino[1,2-b]quinoline-3,14(4H)-dione (P26)

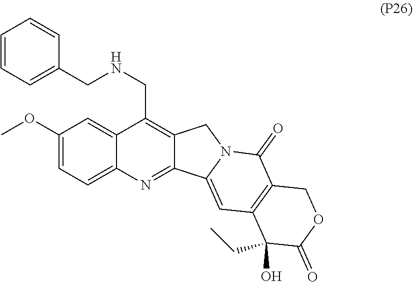 [SMILES: CC[C@@]1(O)C(=O)OCc2c1cc1n(c2=O)Cc2c-1nc1ccc(OC)cc1c2CNCc1ccccc1]

Following the general procedure B, payload P26 (2.0 mg, 8.5% yield) was obtained as a white solid. ESI m/z: 498.2 (M+H)⁺.

### Example 3G-5: Synthesis of 2-((1-(((S)-4-ethyl-4-hydroxy-9-methoxy-3,14-dioxo-3,4,12,14-tetrahydro-1H-pyrano[3′,4′:6,7]indolizino[1,2-b]quinolin-11-yl)methyl)-4,5,6,7,8,9-hexahydro-1H-cycloocta[d][1,2,3]triazol-4-yl)oxy)acetic acid (P27)

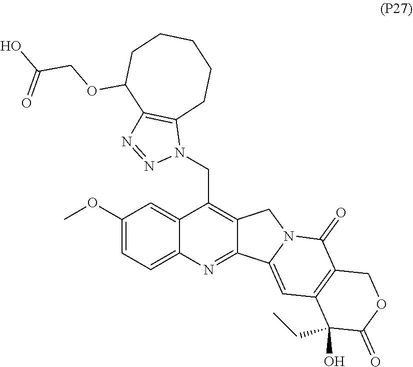 [SMILES: CC[C@@]1(O)C(=O)OCc2c1cc1n(c2=O)Cc2c-1nc1ccc(OC)cc1c2Cn1nnc2c1CCCCCC2OCC(=O)O]

To a solution of P21 (15 mg, 35 μmol) in methanol (3.0 mL) was added COT (13 mg, 0.071 μmol), and the reaction mixture was stirred at room temperature for 20 hours. The resulting mixture was purified by reversed phase flash chromatography (0-100% acetonitrile in aq. TFA (0.01%)) to give P27 (5.5 mg, 26% yield) as a white solid. ESI m/z: 616.3 (M+H)⁺. ¹H NMR (400 MHz, DMSO_{d6}) δ 8.43 (brs, 3H), 8.13 (d, J=9.2 Hz, 1H), 7.56-7.51 (m, 2H), 7.29 (s, 1H), 6.58 (br s, 1H), 6.26 (s, 2H), 5.41 (s, 2H), 5.16-4.98 (m, 3H), 3.89 (s, 3H), 3.73-3.62 (m, 4H), 2.94-2.71 (m, 3H), 1.89-1.80 (m, 4H), 1.45-1.23 (m, 6H), 1.00 (brs, 1H), 0.87 (t, J=7.2 Hz, 3H) ppm.

### Example 3G-6: Synthesis of (S)-11-((4-benzyl-1H-1,2,3-triazol-1-yl)methyl)-4-ethyl-4-hydroxy-9-methoxy-1,12-dihydro-14H-pyrano[3′,4′:6,7]indolizino[1,2-b]quinoline-3,14(4H)-dione (P28)

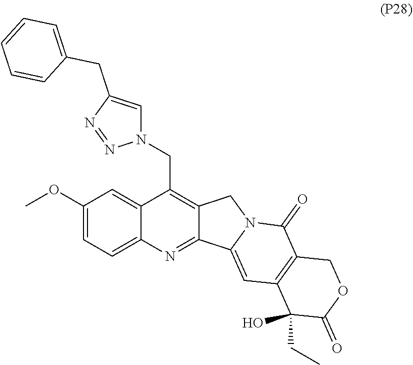 [SMILES: CC[C@@]1(O)C(=O)OCc2c1cc1n(c2=O)Cc2c-1nc1ccc(OC)cc1c2Cn1cc(Cc2ccccc2)nn1]

To a solution of P21 (10 mg, 23 μmol) in THF (3 mL) were added benzylethene (2.7 mg, 23 μmol), sodium ascorbate (9.1 mg, 46 μmol), and aq. copper(II) sulfate (2 mg in 1 mL water), and the reaction mixture was stirred at room temperature in darkness for 20 hours. The resulting mixture was directly separated by reversed phase flash chromatography (0-100% acetonitrile in aq. TFA (0.01%)) to give P28 (4.3 mg, 34% yield) as a white solid. ESI m/z: 550.1 (M+H)⁺. ¹H NMR (400 MHz, DMSO_{d6}) δ 8.51 (br s, 1H), 8.07 (d, J=9.2 Hz, 1H), 7.84 (s, 1H), 7.60 (s, 1H), 7.48-7.42 (m, 2H), 7.23-7.14 (m, 5H), 6.22 (s, 2H), 5.57 (d, J=16.0 Hz, 1H), 5.37 (d, J=16.0 Hz, 1H), 5.36 (s, 2H), 3.85 (s, 3H), 1.97-1.94 (m, 2H), 1.30-1.26 (m, 2H), 0.99 (t, J=7.2 Hz, 3H) ppm.

### Example 4: Synthesis of Payloads P13 and P18 (Scheme 4)

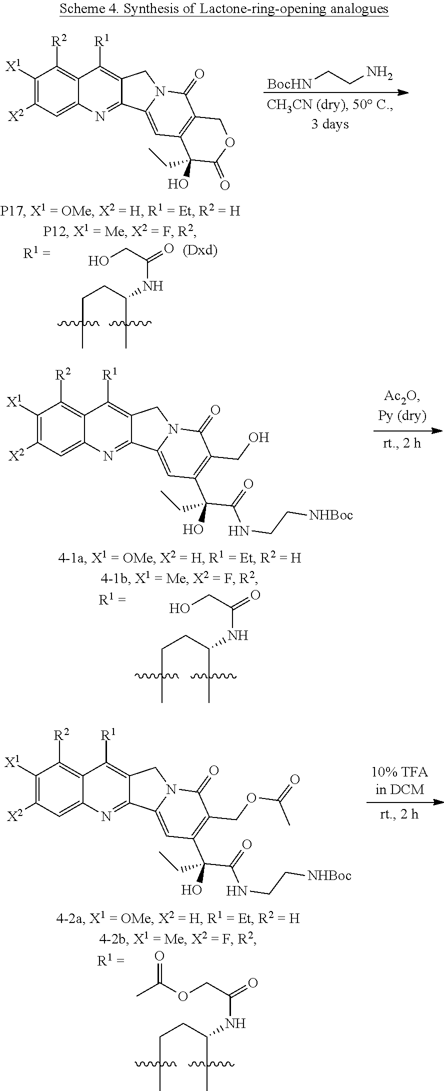

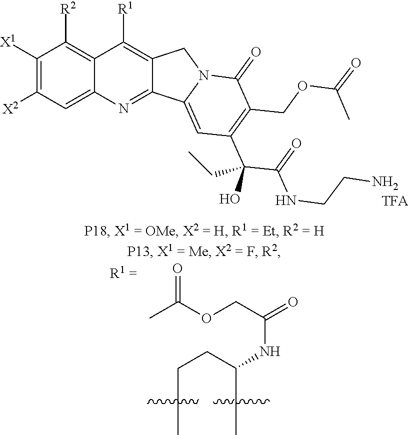

### Example 4A: Synthesis of tert-Butyl N-{2-[(2S)-2-[12-ethyl-8-(hydroxymethyl)-2-methoxy-9-oxo-9H,11H-indolizino[1,2-b]quinolin-7-yl]-2-hydroxybutanamido]ethyl}carbamate (4-1a)

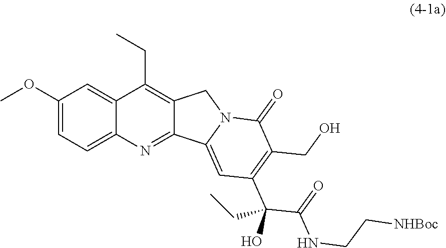 [SMILES: CCc1c2c(nc3ccc(OC)cc13)-c1cc([C@@](O)(CC)C(=O)NCCNC(=O)OC(C)(C)C)c(CO)c(=O)n1C2]

A suspension of compound P17 (0.12 g, 0.30 mmol) and 1-Boc-ethylenediamine (0.20 g, 1.2 mmol) in dry acetonitrile (10 mL) was stirred at 50° C. for 3 days until the suspension turned yellow and clear. The solution was directly purified by reversed phase flash chromatography (0-80% acetonitrile in water) to give compound 4-1a (0.12 g, 71% yield) as a pale yellow solid. ESI m/z: 549.3 (M−H₂O+H)⁺.

### Example 4B: Synthesis of {7-[(1 S)-1-[(2-{[(tert-Butoxy)carbonyl]amino}ethyl)carbamoyl]-1-hydroxypropyl]-12-ethyl-2-methoxy-9-oxo-9H,11H-indolizino[1,2-b]quinolin-8-yl}methyl acetate (4-2a)

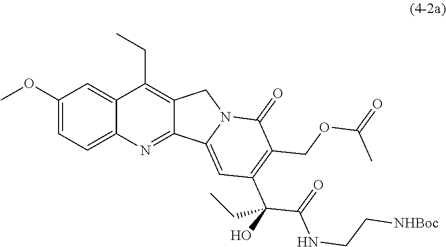 [SMILES: CCc1c2c(nc3ccc(OC)cc13)-c1cc([C@@](O)(CC)C(=O)NCCNC(=O)OC(C)(C)C)c(COC(C)=O)c(=O)n1C2]

To a yellow solution of compound 4-1a (0.12 g, 0.21 mmol) in dry pyridine (2 mL) was added acetic anhydride (0.43 g, 4.2 mmol), and the reaction mixture was stirred at room temperature for 2 hours, which was monitored by LCMS. The reaction mixture was directly purified by reversed phase flash chromatography (0-100% acetonitrile in aq. TFA (0.01%)) to give compound 4-2a (55 mg, 42% yield) as a yellow solid. ESI m/z 549.3 (M-OAc)⁺.

### Example 4C: Synthesis of {7-[(1 S)-1-[(2-Aminoethyl)carbamoyl]-1-hydroxypropyl]-12-ethyl-2-methoxy-9-oxo-9H,11H-indolizino[1,2-b]quinolin-8-yl}methyl acetate (P18)

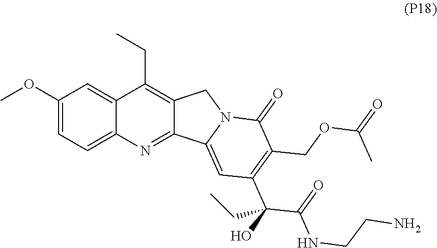 [SMILES: CCc1c2c(nc3ccc(OC)cc13)-c1cc([C@@](O)(CC)C(=O)NCCN)c(COC(C)=O)c(=O)n1C2]

To a stirred yellow solution of compound 4-2a (6.1 mg, 10 μmol) in DCM (1.8 mL) was added TFA (0.2 mL), and the reaction mixture was stirred at room temperature for 2 hours until Boc was totally removed according to LCMS. The resulting solution was then directly purified by reversed phase flash chromatography (0-80% acetonitrile in aq. TFA (0.01%)) to give compound P18 (3.3 mg, 65% yield, TFA salt) as a yellow solid. ESI m/z: 449.2 (M-OAc)⁺. ¹H NMR (400 MHz, DMSO_{d6}) δ 8.31 (t, J=5.6 Hz, 1H), 8.09 (d, J=9.2 Hz, 1H), 7.82 (br s, 3H), 7.53 (d, J=2.8 Hz, 1H), 7.51 (s, 1H), 7.43 (s, 1H), 6.31 (s, 1H), 5.37 (d, J=10.8 Hz, 1H), 5.29 (d, J=10.8 Hz, 1H), 5.29 (s, 2H), 3.99 (s, 3H), 3.43-3.39 (m, 1H), 3.28-3.17 (m, 3H), 2.85 (br s, 2H), 2.22-2.11 (m, 2H), 1.99 (s, 3H), 1.33 (t, J=7.2 Hz, 3H), 0.88 (t, J=7.2 Hz, 3H) ppm.

### Example 4D: Synthesis of tert-Butyl N-{2-[(2S)-2-[(19S)-14-fluoro-19-(2-hydroxyacetamido)-6-(hydroxymethyl)-15-methyl-5-oxo-4,11-diazapentacyclo[10.7.1.0²,¹⁰.0⁴,⁹.0¹⁶,²⁰]icosa-1,6,8,10,12,14,16(20)-heptaen-7-yl]-2-hydroxybutanamido]ethyl}carbamate (4-1b)

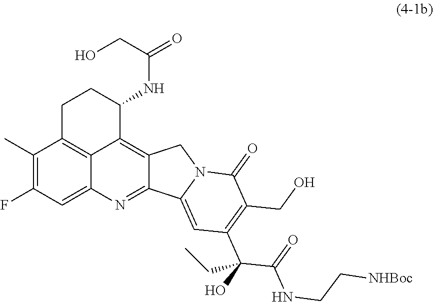 [SMILES: CC[C@@](O)(C(=O)NCCNC(=O)OC(C)(C)C)c1cc2n(c(=O)c1CO)Cc1c-2nc2cc(F)c(C)c3c2c1[C@@H](NC(=O)CO)CC3]

Following the similar procedure as compound 4-1a, except substituting compound DXd (P12) for compound P17, compound 4-1b (0.16 g, 84% yield) was obtained as a pale-yellow solid. ESI m/z: 636.3 (M-OH)⁺. ¹H NMR (400 MHz, DMSO_{d6}) δ8.45 (d, J=9.2 Hz, 1H), 8.14 (t, J=5.6 Hz, 1H), 7.81 (d, J=11.2 Hz, 1H), 7.42 (s, 1H), 6.80 (t, J=5.6 Hz, 1H), 6.48 (s, 1H), 5.64-5.58 (m. 1H), 5.20 (d, J=18.8 Hz, 1H), 5.12 (d, J=18.8 Hz, 1H), 4.82 (d, J=12.0 Hz, 1H), 4.66 (d, J=11.6 Hz, 1H), 3.97 (s, 2H), 3.20-3.10 (m, 4H), 3.03-2.98 (m, 2H), 2.39 (s, 3H), 2.21-2.12 (m, 4H), 1.43-1.38 (m, 9H), 0.87 (t, J=7.2 Hz, 3H) ppm. Reference Chemical & Pharmaceutical Bulletin, 1994, 2518-2525, confirming the identity of 4-1b, is incorporated by reference in its entirety.

### Example 4E: Synthesis of {[(19S)-6-[(Acetyloxy)methyl]-7-[(1 S)-1-[(2-{[(tert-butoxy)carbonyl]amino}ethyl)carbamoyl]-1-hydroxypropyl]-14-fluoro-15-methyl-5-oxo-4,11-diazapentacyclo[10.7.1.0²,¹⁰.0⁴,⁹.0¹⁶,²⁰]icosa-1,6,8,10,12,14,16(20)-heptaen-19-yl]carbamoyl}methyl acetate (4-2b)

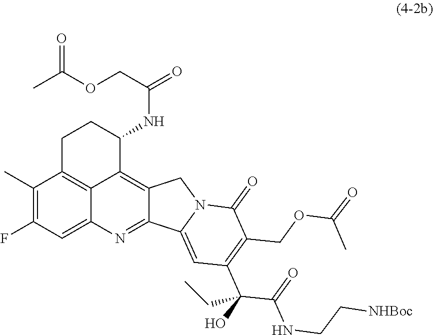 [SMILES: CC[C@@](O)(C(=O)NCCNC(=O)OC(C)(C)C)c1cc2n(c(=O)c1COC(C)=O)Cc1c-2nc2cc(F)c(C)c3c2c1[C@@H](NC(=O)COC(C)=O)CC3]

Following the similar procedure as compound 4-2a, except substituting compound 4-1b for compound 4-1a, compound 4-2b (50 mg, 73% yield, TFA salt) was obtained as a pale-yellow solid. ESI m/z: 678.3 (M-OAc)⁺. ¹H NMR (400 MHz, DMSO_{d6}) δ 8.68 (d, J=8.8 Hz, 1H), 8.05-8.03 (m, 1H), 7.83 (d, J=10.8 Hz, 1H), 7.45 (s, 1H), 6.78 (d, J=4.4 Hz, 1H), 6.21 (br s, 1H), 5.59-5.58 (m, 1H), 5.37 (d, J=11.2 Hz, 1H), 5.30 (d, J=10.8 Hz, 1H), 5.25-5.13 (m, 2H), 4.52 (d, J=2.0 Hz, 2H), 3.20-3.17 (m, 3H), 3.06-2.99 (m, 3H), 2.41 (s, 3H), 2.22-2.10 (m, 4H), 2.09 (s, 3H), 1.98 (s, 3H), 1.38-1.35 (m, 9H), 0.87 (t, J=7.2 Hz, 3H) ppm. ¹⁹F NMR (376 MHz, DMSO_{d6}) δ −74 (TFA), −111 (Ar—F) ppm.

### Example 4F: Synthesis of {[(19S)-6-[(Acetyloxy)methyl]-7-[(1 S)-1-[(2-aminoethyl)carbamoyl]-1-hydroxypropyl]-14-fluoro-15-methyl-5-oxo-4,11-diazapentacyclo[10.7.1.0²,¹⁰.0⁴,⁹.0¹⁶,²⁰]icosa-1,6,8,10,12,14,16(20)-heptaen-19-yl]carbamoyl}methyl acetate (P14)

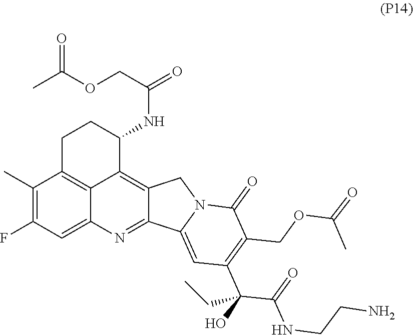 [SMILES: CC[C@@](O)(C(=O)NCCN)c1cc2n(c(=O)c1COC(C)=O)Cc1c-2nc2cc(F)c(C)c3c2c1[C@@H](NC(=O)COC(C)=O)CC3]

Following the similar procedure as compound P18, except substituting compound 4-2b for compound 4-2a, compound P13 (26 mg, 60% yield, TFA salt) was obtained as a white solid. ESI m/z: 638.3 (M+H)⁺. ¹H NMR (400 MHz, DMSO_{d6}) δ8.69 (d, J=8.4 Hz, 1H), 8.32 (t, J=6.0 Hz, 1H), 7.83 (d, J=10.8 Hz, 1H), 7.72 (br s, 3H), 7.48 (s, 1H), 6.34 (s, 1H), 5.61-5.58 (m. 1H), 5.37-5.14 (m, 4H), 4.55 (d, J=14.8 Hz, 1H), 4.50 (d, J=14.8 Hz, 1H), 3.41-3.35 (m, 1H), 3.26-3.23 (m, 1H), 3.22-3.18 (m, 2H), 2.84-2.83 (br s, 2H), 2.42 (s, 3H), 2.21-2.12 (m, 4H), 2.08 (s, 3H), 1.99 (s, 3H), 0.88 (t, J=7.2 Hz, 3H) ppm.

Table 2, below, provides a listing of the exemplary linker-payloads (LPs) of the present disclosure. Table 3, below, provides chemical properties for the exemplified LPs. Table 4, below, provides HCT-15 data of the exemplary payloads and linker-payloads

TABLE 2
List of Linker- Exatecan vs Linker-ProEXT

| # | Linker-name | Structures |
|---|---|---|
| LP1 | COT-PEG4- vcPAB- Exatecan | 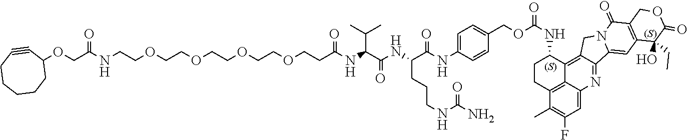 |
| LP2 | DIBAC- PEG4- vcPAB- Exatecan | 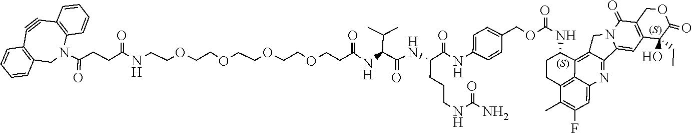 |
| LP3 | COT-EDA- (GLCA)- PAB- Exatecan | 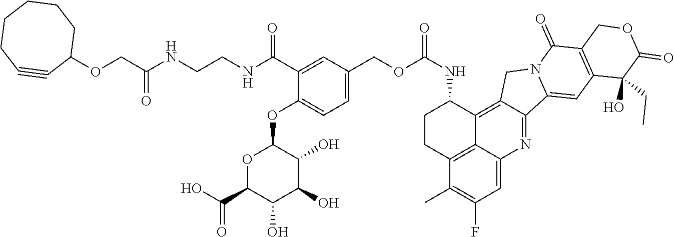 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(C(=O)NCCNC(=O)COC2C#CCCCCC2)c1)CC3] |
| LP4 | COT-EDA- (GLC)PAB- Exatecan | 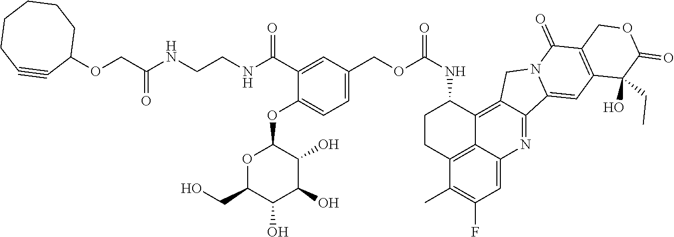 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](CO)[C@@H](O)[C@H](O)[C@H]2O)c(C(=O)NCCNC(=O)COC2C#CCCCCC2)c1)CC3] |
| LP5 | COT-PEG4- vcPAB- ProEXT | 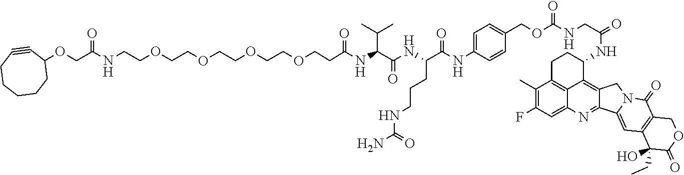 [SMILES: CC[C@@]1(O)C(=O)OCc2c1cc1n(c2=O)Cc2c-1nc1cc(F)c(C)c3c1c2[C@@H](NC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3] |
| LP6 | COT-PEG4- EvcPAB- ProEXT | 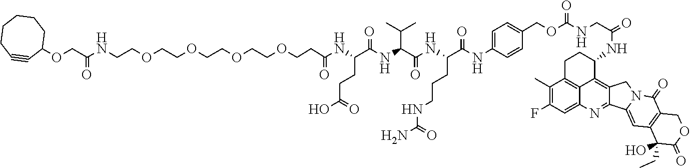 [SMILES: CC[C@@]1(O)C(=O)OCc2c1cc1n(c2=O)Cc2c-1nc1cc(F)c(C)c3c1c2[C@@H](NC(=O)CNC(=O)OCc1ccc(NC(=O)[C@H](CCCNC(N)=O)NC(=O)[C@@H](NC(=O)[C@H](CCC(=O)O)NC(=O)CCOCCOCCOCCOCCNC(=O)COC2C#CCCCCC2)C(C)C)cc1)CC3] |
| LP7 | COT-EDA- (GLC)PAB- ProEXT | 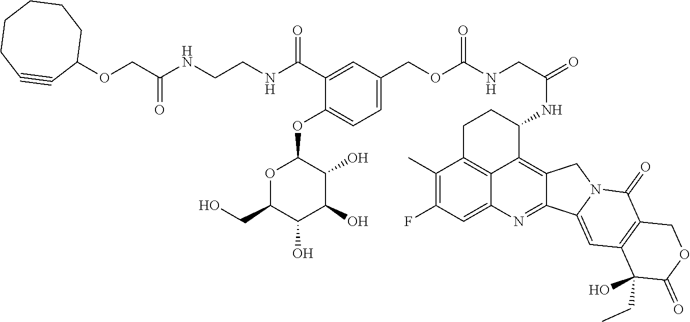 [SMILES: CC[C@@]1(O)C(=O)OCc2c1cc1n(c2=O)Cc2c-1nc1cc(F)c(C)c3c1c2[C@@H](NC(=O)CNC(=O)OCc1ccc(O[C@@H]2O[C@H](CO)[C@@H](O)[C@H](O)[C@H]2O)c(C(=O)NCCNC(=O)COC2C#CCCCCC2)c1)CC3] |
| LP8 | COT- GGGG- ProEXT | 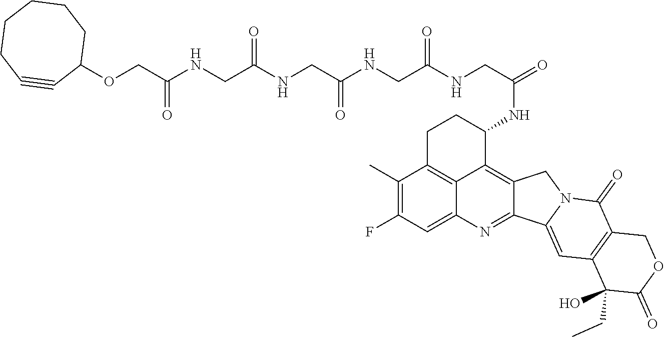 [SMILES: CC[C@@]1(O)C(=O)OCc2c1cc1n(c2=O)Cc2c-1nc1cc(F)c(C)c3c1c2[C@@H](NC(=O)CNC(=O)CNC(=O)CNC(=O)CNC(=O)COC1C#CCCCCC1)CC3] |
| LP9 | COT-PEG4- vcPAB- GluEXT | 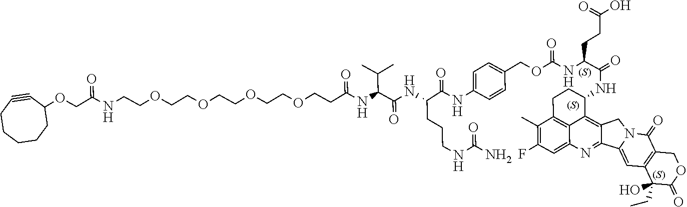 |
| LP10 | COT-PEG4- vcPAB- PheEXT | 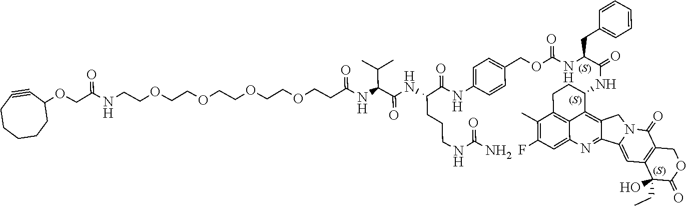 |
| LP11 | COT-PEG4- VCPAB- ^{ε}LysEXT | 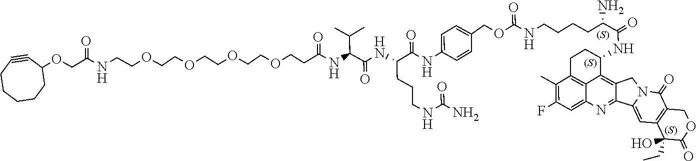 |
| LP12 | COT-PEG4- vcPAB- ProBLT | 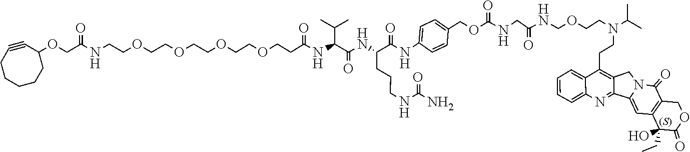 |
| LP13 | COT-PEG4- EvcPAB- NHCH2- HOEthBLT | 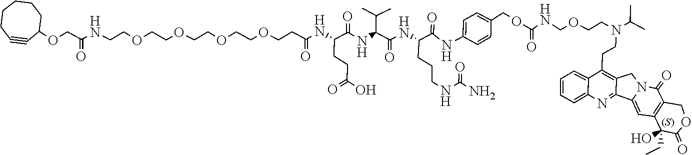 |
| LP14 | COT-PEG4- EvcPAB- NHCH2- DXd | 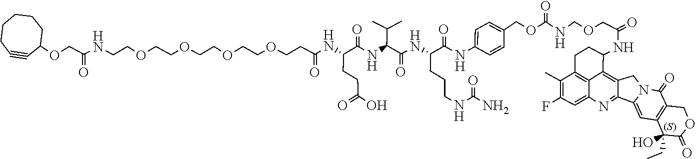 |
| LP15 | COT-PEG4- VAPAB- NHCH2- DXd | 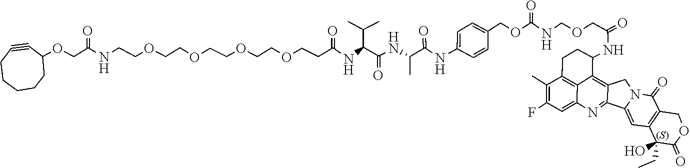 |
| LP16 | COT-BL- [PEG2- (GLC)PAB- EXT]2 | 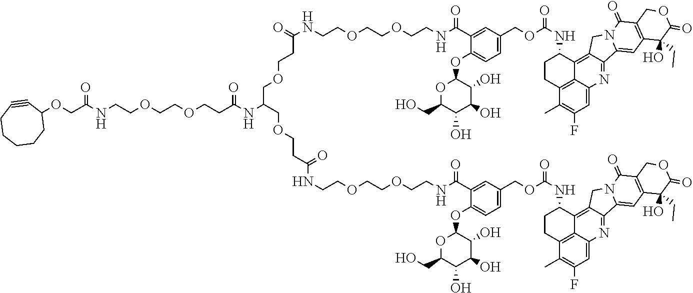 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](CO)[C@@H](O)[C@H](O)[C@H]2O)c(C(=O)NCCOCCOCCNC(=O)CCOCC(COCCC(=O)NCCOCCOCCNC(=O)c2cc(COC(=O)N[C@H]4CCc5c(C)c(F)cc6nc7c(c4c56)Cn4c-7cc5c(c4=O)COC(=O)[C@]5(O)CC)ccc2O[C@@H]2O[C@H](CO)[C@@H](O)[C@H](O)[C@H]2O)NC(=O)CCOCCOCCNC(=O)COC2C#CCCCCC2)c1)CC3] |
| LP17 | COT-BL- [PEG2- EvcPAB- EXT]2 | 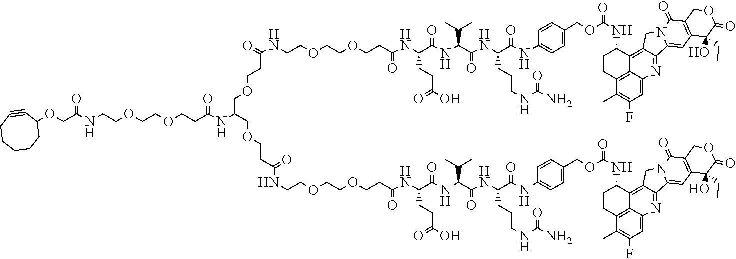 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)[C@H](CCC(=O)O)NC(=O)CCOCCOCCNC(=O)CCOCC(COCCC(=O)NCCOCCOCCC(=O)N[C@@H](CCC(=O)O)C(=O)N[C@H](C(=O)N[C@@H](CCCNC(N)=O)C(=O)Nc2ccc(COC(=O)N[C@H]4CCc5c(C)c(F)cc6nc7c(c4c56)Cn4c-7cc5c(c4=O)COC(=O)[C@]5(O)CC)cc2)C(C)C)NC(=O)CCOCCOCCNC(=O)COC2C#CCCCCC2)C(C)C)cc1)CC3] |
| LP18 | COT-PEG4- vcPAB-P16 | 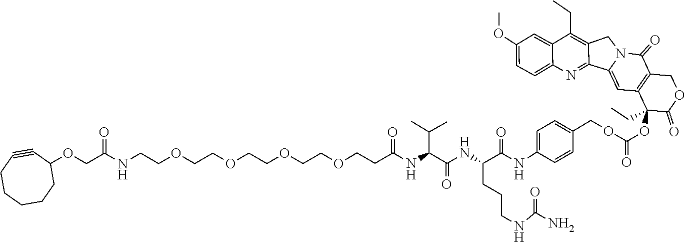 [SMILES: CCc1c2c(nc3ccc(OC)cc13)-c1cc3c(c(=O)n1C2)COC(=O)[C@@]3(CC)OC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1] |
| LP19 | COT-PEG4- vcPAB-P17 | 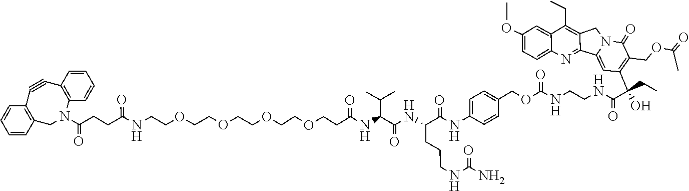 [SMILES: CCc1c2c(nc3ccc(OC)cc13)-c1cc([C@@](O)(CC)C(=O)NCCNC(=O)OCc3ccc(NC(=O)[C@H](CCCNC(N)=O)NC(=O)[C@@H](NC(=O)CCOCCOCCOCCOCCNC(=O)CCC(=O)N4Cc5ccccc5C#Cc5ccccc54)C(C)C)cc3)c(COC(C)=O)c(=O)n1C2] |
| LP20 | COT-PEG4- vcPAB-P21 | 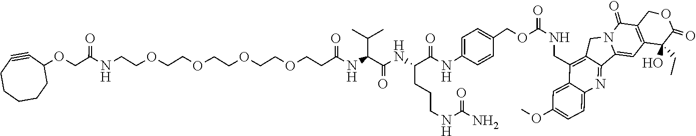 [SMILES: CC[C@@]1(O)C(=O)OCc2c1cc1n(c2=O)Cc2c-1nc1ccc(OC)cc1c2CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1] |
| LP21 | DIBAC- PEG4- vcPAB-P21 | 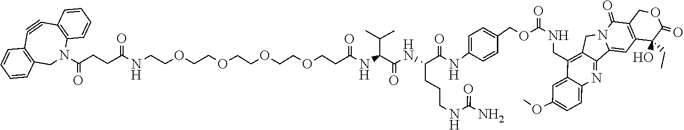 [SMILES: CC[C@@]1(O)C(=O)OCc2c1cc1n(c2=O)Cc2c-1nc1ccc(OC)cc1c2CNC(=O)OCc1ccc(NC(=O)[C@H](CCCNC(N)=O)NC(=O)[C@@H](NC(=O)CCOCCOCCOCCOCCNC(=O)CCC(=O)N2Cc3ccccc3C#Cc3ccccc32)C(C)C)cc1] |
| LP22 | COT- GGFG-P21 | 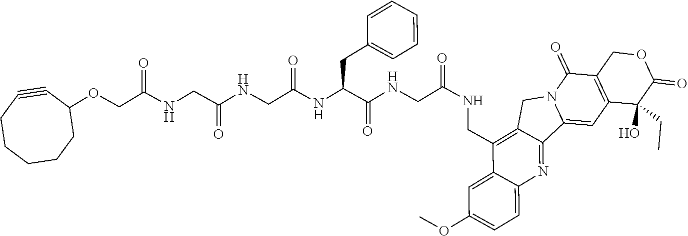 [SMILES: CC[C@@]1(O)C(=O)OCc2c1cc1n(c2=O)Cc2c-1nc1ccc(OC)cc1c2CNC(=O)CNC(=O)[C@H](Cc1ccccc1)NC(=O)CNC(=O)CNC(=O)COC1C#CCCCCC1] |

TABLE 3
Chemical Properties of Linker-Payloads

| | Linker-name | cLogP | MF | MW | Mass ESI m/z |
|---|---|---|---|---|---|
| LP1 | COT-PEG4-vcPAB- | 2.87 | C₆₄H₈₂FN₉O₁₆ | 1252.41 | 626.8 (M/2 + H) |
| | Exatecan | | | | |
| LP2 | DIBAC-PEG4-vcPAB- | 3.61 | C₇₃H₈₃FN₁₀O₁₆ | 1375.52 | 697.3 (M/2 + H) |
| | Exatecan | | (lactone) | | |
| | | | C₇₃H₈₅FN₁₀O₁₇ | 1393.53 | 688.3 (M/2 + H) |
| | | | (acid) | | |
| LP3 | COT-EDA-(GLCA)PAB- | 1.41 | C₅₁H₅₄FN₅O₁₆ | 1012.00 | 1012.5 (M + H) |
| | Exatecan | | | | |
| LP4 | COT-EDA-(GLC)PAB- | 1.49 | C₅₁H₅₆FN₅O₁₅ | 998.01 | 998.5 (M + H) |
| | Exatecan | | | | |
| LP5 | COT-PEG4-vcPAB- | 1.76 | C₆₆H₈₅FN₁₀O₁₇ | 1309.46 | 655.5 (M/2 + H) |
| | ProEXT | | | | |
| LP6 | COT-PEG4-EvcPAB- | 0.76 | C₇₁H₉₂FN₁₁O₂₀ | 1438.57 | 720.0 (M/2 + H) |
| | ProEXT | | | | |
| LP7 | COT-EDA-(GLC)PAB- | 0.38 | C₅₃H₅₉FN₆O₁₆ | 1055.08 | |
| | ProEXT | | | | |
| LP8 | COT-GGGG-ProEXT | −0.80 | C₄₂H₄₆FN₇O₁₀ | 827.85 | 828.5 (M + H) |
| LP9 | COT-PEG4-vcPAB- | 1.85 | C₆₉H₈₉FN₁₀O₁₉ | 1381.52 | 691.3 |
| | GluEXT | | | | |
| LP10 | COT-PEG4-vcPAB- | 3.99 | C₇₃H₉₁FN₁₀O₁₇ | 1399.58 | 700.6 |
| | PheEXT | | | | |
| LP11 | COT-PEG4-vcPAB- | 2.20 | C₇₀H₉₄FN₁₁O₁₇ | 1380.58 | 691.2 |
| | ^{ε}LysEXT | | | | |
| LP12 | COT-PEG4-vcPAB- | 1.71 | C₇₀H₉₇N₁₁O₁₈ | 1380.61 | 690.8 |
| | ProBLT | | | | |
| LP13 | COT-PEG4-EvcPAB- | −0.81 | C₇₃H₁₀₁N₁₁O₂₀ | 1452.67 | 727.3 |
| | NHCH2-HOEthBLT | | | | |
| LP14 | COT-PEG4-EvcPAB- | 0.83 | C₇₂H₉₄FN₁₁O₂₁ | 1468.60 | 734.3 |
| | NHCH2-DXd | | | | |
| LP15 | COT-PEG4-VAPAB- | 2.92 | C₆₄H₈₁FN₈O₁₇ | 1253.39 | 627.5 |
| | NHCH2-DXd | | | | |
| LP16 | COT-BL-[PEG2- | −1.39 | C₁₁₆H₁₄₂F₂N₁₂O₃₉ | 2366.45 | 789.2 (M/3 + H) |
| | (GLC)PAB-EXT]2 | | | | |
| LP17 | COT-BL-[PEG2- | 1.74 | C₁₃₆H₁₇₆F₂N₂₂O₃₉ | 2781.02 | 927.7 (M/3 + H) |
| | EvcPAB-EXT]2 | | | | |
| LP18 | COT-PEG4-vcPAB-P17 | 3.97 | C₆₃H₈₂N₈O₁₇ | 1223.39 | 612.3 |
| LP19 | COT-PEG4-vcPAB-P18 | 2.48 | C₇₆H₉₃N₁₁O₁₈ | 1448.64 | 612.3 ((M- |
| | | | | | OAc)/2 + H) |
| LP20 | COT-PEG4-vcPAB-P22 | 1.51 | C₆₂H₈₁N₉O₁₇ | 1224.38 | 613.0 |
| LP21 | DIBAC-PEG4-vcPAB- | 2.25 | C₇₁H₈₂N₁₀O₁₇ | 1347.49 | 682.7 (acid) |
| | P22 | | | | 674.3 (lactone) |
| LP22 | COT-GGFG-P22 | 0.07 | C₄₇H₅₁N₇O₁ | 889.95 | 890.3 (M + H) |

TABLE 4
HCT-15 data of payloads and linker-payloads

| Payload | | | | | | |
|---|---|---|---|---|---|---|
| | | | HCT-15 + | Linker-payload | | |
| | | HCT-15 | verapamil | | | HCT-15 |
| # | Name | EC₅₀ (nM) | EC₅₀ (nM) | # | Linker-name | EC₅₀ (nM) |
| P1 | EXT | 1.22 | 1.13 | LP1 | COT-PEG4-vcPAB-EXT | 288.92 |
| | | | | LP2 | DIBAC-PEG4-vcPAB-EXT | 144.93 |
| | | | | LP3 | COT-EDA-(GLCA)PAB- | 456.83 |
| | | | | | EXT | |
| | | | | LP4 | COT-EDA-(GLC)PAB-EXT | 362.97 |
| | | | | LP16 | COT-BL-[PEG2- | 140.24 |
| | | | | | (GLC)PAB-EXT]2 | |
| | | | | LP17 | COT-BL-[PEG2-EvcPAB- | (no amt) |
| | | | | | EXT]2 | |
| P2 | GlyEXT | 61.91 | 14.34 | LP5 | COT-PEG4-vcPAB- | >1000 |
| | | | | | GlyEXT | |
| | | | | LP6 | COT-PEG4-EvcPAB- | (no amt) |
| | | | | | GlyEXT | |
| | | | | LP7 | COT-EDA-(GLC)PAB- | (no amt) |
| | | | | | GlyEXT | |
| | | | | LP8 | COT-GGGG-EXT | 836.54 |
| P3 | GluEXT | 610.21 | 596.04 | LP9 | COT-PEG4-vcPAB- | 722.36 |
| | | | | | GluEXT | |
| P5 | PheEXT | 48.25 | 4.20 | LP10 | COT-PEG4-vcPAB- | 851.96 |
| | | | | | PheEXT | |
| P4 | LysEXT | 447.03 | 580.96 | LP11 | COT-PEG4-vcPAB- | 208.38 |
| | | | | | ^{ε}LysEXT | |
| P6 | MsEXT | 7.45 | 3.16 | | | |
| P7 | MsEthEXT | 5.58 | 1.69 | | | |
| P8 | BLT | 20.04 | 3.09 | | | |
| P9 | MsBLT | 8.89 | 3.03 | | | |
| P10 | HOEthBLT | 8.60 | 4.38 | LP13 | COT-PEG4-EvcPAB- | |
| | | | | | NHCH2-HOEtBLT | |
| P11 | ProBLT | | | LP12 | COT-PEG4-vCPAB- | |
| | | | | | ProBLT | |
| P12 | DXd | 7.99 | 4.18 | LP14 | COT-PEG4-EvcPAB- | |
| | | | | | NHCH2-DXd | |
| | | | | LP15 | COT-PEG4-VAPAB- | 96.93 |
| | | | | | NHCH2-DXd | |
| P13 | | 133.91 | | | | |
| P14 | | 11.58 | 6.79 | | | |
| P15 | | 6.99 | 4.47 | | | |
| P16 | SN38 | 6.11 | 4.77 | | | |
| P17 | | 1.59 | 1.44 | LP18 | COT-PEG4-vCPAB-P16 | |
| P18 | | 32.45 | 32.79 | LP19 | COT-PEG4-vCPAB-P17 | |
| P19 | Irinotecan | NA | NA | | | |
| P20 | | 3.69 | 2.40 | | | |
| P21 | | | | | | |
| P22 | | 31.94 | 40.45 | LP20 | COT-PEG4-vCPAB-P21 | |
| | | | | LP21 | DIBAC-PEG4-vcPAB-P21 | |
| | | | | LP22 | COT-GGFG-P21 | |
| P23 | | 36.74 | | | | |
| P24 | | 13.54 | | | | |
| P25 | | 46.79 | | | | |
| P26 | | 51.87 | | | | |
| P27 | | 78.81 | | | | |
| P28 | | 34.19 | | | | |

### Example 5: Synthesis of vcPAB Linker-Payloads (Schemes 5A and 5B)

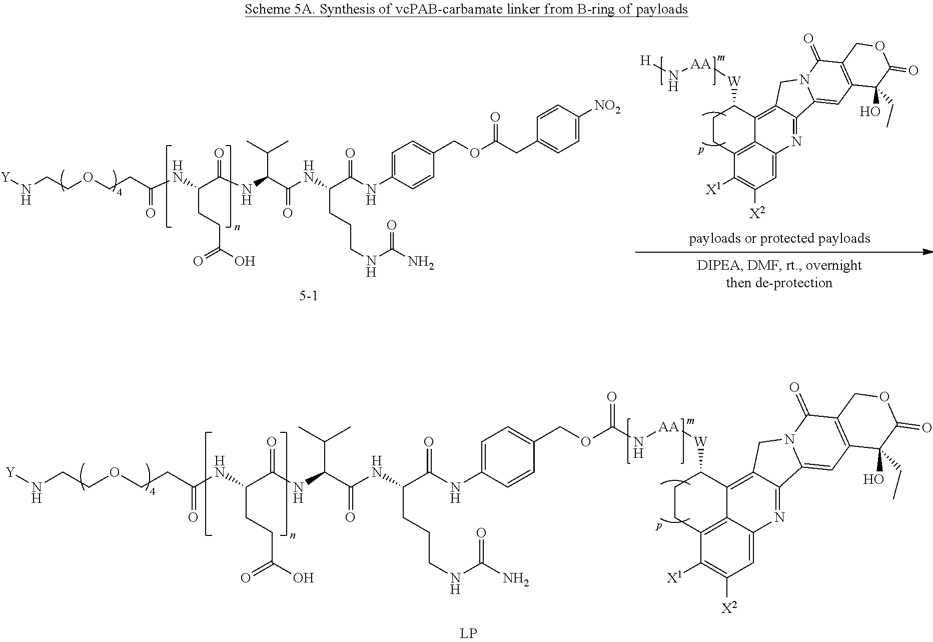

| P# | LP# | Y | n | W | m | AA | p | X1 | X2 |
|---|---|---|---|---|---|---|---|---|---|
| P1 | LP1 | COT | 0 | NH | 0 | / | 1 | Me | F |
| (EXT) | | | | | | | | | |
| P1 | LP2 | DIBAC | 0 | NH | 0 | / | 1 | Me | F |
| P2 | LP5 | COT | 0 | NH | 1 | —CH₂CO— | 1 | Me | F |
| P2 | LP6 | COT | 1 | NH | 1 | —CH₂CO— | 1 | Me | F |
| P3 | LP9 | COT | 0 | NH | 1 | —CH(CH₂CH₂COOH)—CO | 1 | Me | F |
| P5 | LP10 | COT | 0 | NH | 1 | —CH(Bn)CO— | 1 | Me | F |
| P4 | LP11 | COT | 0 | NH | 1 | —(CH₂)₄CH(NH₂)CO— | 1 | Me | F |
| P11 | LP12 | COT | 0 | CH₂N(ⁱPr)(CH₂)₂OCH₂NH | 1 | —CH₂CO— | 1 | H | H |
| P21 | LP20 | COT | 0 | NH | 0 | / | 0 | OMe | H |
| P21 | LP21 | DIBAC | 0 | NH | 0 | / | 0 | OMe | H |

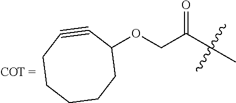 [SMILES: *C(=O)COC1C#CCCCCC1]

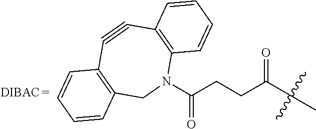 [SMILES: *C(=O)CCC(=O)N1Cc2ccccc2C#Cc2ccccc21]

To a solution of payloads or protected payloads (1.0 equiv.), which containing an amine group, in dry DMF (2 mM) were added compound 5-1 (1 equiv.) and DIPEA (3 equiv.), and the reaction mixture was stirred at room temperature for 16 hours, which was monitored by LCMS. The resulting solution was directly purified by prep-HPLC (0-70% acetonitrile in aq. TFA (0.01%)) to give corresponding linker-payload (23-81% yield, TFA salt) as a solid.

### Example 5A-1: Synthesis of {4-[(2S)-5-(Carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl N-[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamate (LP1)

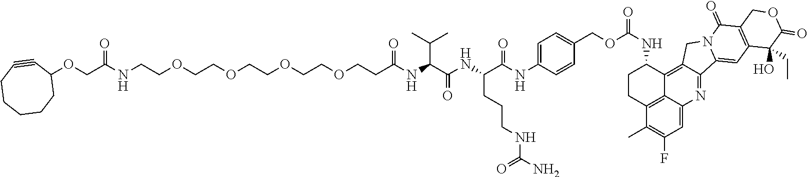 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

Following the general procedure C starting from exatecan with 5-1a, linker-payload LP1 (36 mg, 81% yield, TFA salt) was obtained as a white solid after purification by prep-HPLC (0-100% acetonitrile in aq. ammonium bicarbonate (10 mM)), treated with aq. TFA (0.1 M) to pH 4.0, concentrated in vacuo and lyophilization. ESI m/z: 626.8 (M/2+H)⁺. ¹H NMR (400 MHz, DMSO_{d6}) δ 10.00 (s, 1H), 8.13 (d, J=6.8 Hz, 1H), 8.07 (d, J=9.2 Hz, 1H), 7.88 (d, J=8.4 Hz, 1H), 7.78 (d, J=10.8 Hz, 1H), 7.61 (d, J=8.4 Hz, 3H), 7.36 (d, J=8.4 Hz, 2H), 7.32 (s, 1H), 6.54 (s, 1H), 6.00 (s, 1H), 5.45 (s, 3H), 5.29 (s, 3H), 5.08 (s, 2H), 4.41-4.35 (m, 1H), 4.29-4.21 (m, 2H), 3.87 (d, J=14.4 Hz, 1H), 3.75 (d, J=14.4 Hz, 1H), 3.62-3.57 (m, 2H), 3.50-3.48 (m, 14H), 3.27-3.23 (m, 3H), 3.15-2.90 (m, 4H), 2.38-2.33 (m, 4H), 2.24-2.07 (m, 5H), 1.96-1.67 (m, 9H), 1.59-1.53 (m, 3H), 1.46-1.33 (m, 3H), 0.89-0.82 (m, 9H) ppm. (The proton of TFA was not observed). ¹⁹F NMR (376 MHz, DMSO_{d6}) δ −74 (TFA), −111 (Ar—F) ppm.

### Example 5A-2: Synthesis of {4-[(2S)-2-[(2S)-2-[1-(4-{2-Azatricyclo[10.4.0.0⁴,⁹]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methyl N-[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamate (LP2)

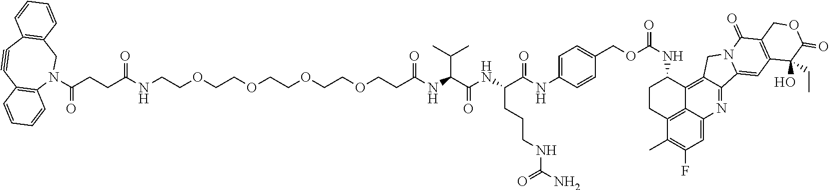 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)CCOCCOCCOCCOCCNC(=O)CCC(=O)N2Cc4ccccc4C#Cc4ccccc42)C(C)C)cc1)CC3]

Following the general procedure C starting from exatecan with 5-1b, linker-payload LP2 (with lactone ring-opening product, 10 mg, 26% yield) was obtained as a white solid after purification by reversed phase flash chromatography (0-100% methanol in aq. ammonium bicarbonate (10 mM)).

Lactone: HPLC purity: 67%, retention time: 8.16 min, ESI m/z: 459.4 (M/3+H)⁺, 688.3 (M/2+H)⁺;

Ring-opening product: HPLC purity: 33%, retention time: 6.98 min, ESI m/z: 465.2 (M/3+H)⁺, 697.3 (M/2+H)⁺.

¹H NMR (400 MHz, DMSO_{d6}) δ9.99 (s, 1H), 8.18-8.11 (m, 1H), 8.06 (d, J=8.8 Hz, 1H), 7.94-7.86 (m. 1H), 7.79-7.75 (m. 2H), 7.68 (d, J=1.6 Hz, 1H), 7.67-7.56 (m. 3H), 7.51-7.42 (m, 3H), 7.38-7.28 (m, 6H), 6.53 (s, 0.6H), 6.05-6.00 (m, 0.4H), 5.99-5.96 (m, 0.6H), 5.45-5.41 (m, 3H), 5.29-5.28 (m, 2H), 5.26-5.21 (m, 1H), 5.08 (s, 2H), 5.02 (d, J=14.0 Hz, 1H), 4.75-4.70 (m, 0.4H), 4.41-4.36 (m, 1H), 4.25-4.21 (m, 1H), 3.61-3.57 (m, 3H), 3.47-3.42 (m, 13H), 3.24-3.22 (m, 2H), 3.21-3.19 (m, 1H), 3.14-3.05 (m, 3H), 3.03-3.00 (m, 1H), 2.97-2.91 (m, 1H), 2.61-2.55 (m, 1H), 2.48-2.44 (m, 1H), 2.41-2.35 (m, 4H), 2.27-2.09 (m, 4H), 2.03-1.95 (m, 2H), 1.90-1.83 (m, 1H), 1.80-1.72 (m, 2H), 1.65-1.54 (m, 1H), 1.47-1.23 (m, 2H), 1.04 (t, J=6.8 Hz, 0.4H), 0.90-0.81 (m, 9H) ppm. ¹⁹F NMR (376 MHz, DMSO_{d6}) δ −111 ppm.

### Example 5A-3: Synthesis of {4-[(2S)-5-(Carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl N-({[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}methyl)carbamate (LP5)

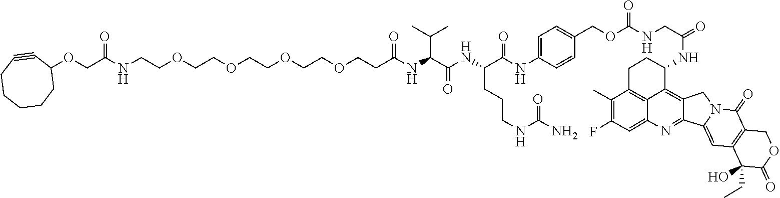 [SMILES: CC[C@@]1(O)C(=O)OCc2c1cc1n(c2=O)Cc2c-1nc1cc(F)c(C)c3c1c2[C@@H](NC(=O)CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1)CC3]

Following the general procedure C starting from P2 with 5-1a, linker-payload LP5 (22 mg, 32% yield) was obtained as a white solid after purification by prep-HPLC (5-95% acetonitrile in aq. TFA (0.1%)). ESI m/z: 655.5 (M/2+H)⁺, 1309.6 (M+H)⁺. ¹H NMR (400 MHz, DMSO_{d6}) δ 9.99 (s, 1H), 8.50 (d, J=8.5 Hz, 1H), 8.13 (d, J=7.5 Hz, 1H), 7.88 (d, J=8.5 Hz, 1H), 7.81 (d, J=10.9 Hz, 1H), 7.59-7.57 (m, 3H), 7.34-7.25 (m, 3H), 6.53 (s, 1H), 5.99-5.95 (m, 1H), 5.60-5.54 (m, 1H), 5.42 (s, 4H), 5.31-5.17 (m, 2H), 4.94 (s, 2H), 4.41-4.34 (m, 1H), 4.27-4.21 (m, 2H), 3.88-3.73 (m, 2H), 3.68-3.58 (m, 4H), 3.56-3.44 (m, 14H), 3.43-3.40 (m, 2H), 3.25-3.23 (m, 2H), 3.19-3.15 (m, 1H), 3.03-2.92 (m, 2H), 2.40-2.38 (m, 4H), 2.23-2.04 (m, 5H), 1.96-1.67 (m, 9H), 1.62-1.52 (m, 3H), 1.45-1.34 (m, 3H), 0.87-0.81 (m, 9H) ppm. ¹⁹F NMR (376 MHz, DMSO_{d6}) δ −111 ppm.

### Example 5A-4: Synthesis of (4S)-4-{[(1 S)-1-{[(1 S)-4-(Carbamoylamino)-1-{[4-({[({[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴] tetracosa-1,6(11),12,14,16,18,20(24)-heptaen-23-yl]carbamoyl}methyl)carbamoyl]oxy}methyl) phenyl]carbamoyl}butyl]carbamoyl}-2-methylpropyl]carbamoyl}-4-{1-[2-(cyclooct-2-yn-1-yloxy)acetamido]-3,6,9,12-tetraoxapentadecan-15-amido}butanoic acid (LP6)

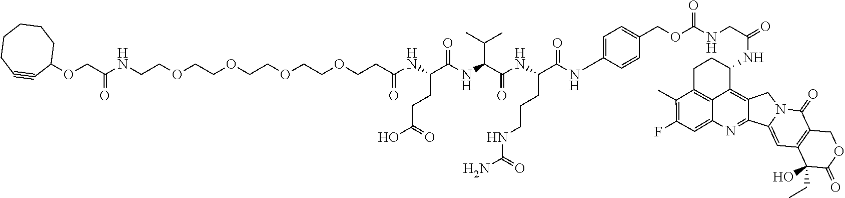 [SMILES: CC[C@@]1(O)C(=O)OCc2c1cc1n(c2=O)Cc2c-1nc1cc(F)c(C)c3c1c2[C@@H](NC(=O)CNC(=O)OCc1ccc(NC(=O)[C@H](CCCNC(N)=O)NC(=O)[C@@H](NC(=O)[C@H](CCC(=O)O)NC(=O)CCOCCOCCOCCOCCNC(=O)COC2C#CCCCCC2)C(C)C)cc1)CC3]

Following the general procedure C starting from P2 with 2-1c, linker-payload methylate-LP6 (15 mg, 45% yield, ESI m/z: 727.0 (M/2+H)⁺) was obtained as a white solid after purification by reversed phase flash chromatography (5-70% acetonitrile in aq. TFA (0.01%)). To a solution of obtained methylate-LP6 (10 mg, 6.9 μmol) in THF (2 mL) was added aq. lithium hydroxide (0.5 mg, 20 mM, 1 mL), and the reaction mixture was stirred at room temperature for an hour, which was monitored by LCMS. The resulting mixture was directly purified by prep-HPLC (10-95% acetonitrile in aq. TFA (0.05%)) to give LP6 (3.0 mg, 30% yield) as a white solid. ESI m/z: 720.0 (M/2+H)⁺. ¹H NMR (400 MHz, DMSO_{d6}) δ 10.03 (s, 1H), 8.51 (d, J=8.8 Hz, 1H), 8.18 (d, J=7.6 Hz, 1H), 8.08 (d, J=8.0 Hz, 1H), 7.81 (d, J=11.2 Hz, 1H), 7.74 (d, J=7.6 Hz, 1H), 7.63-7.56 (m, 3H), 7.44 (t, J=6.0 Hz, 1H), 7.31 (s, 1H), 7.27 (d, J=8.8 Hz, 2H), 6.54 (s, 1H), 6.01-5.96 (m, 1H), 5.60-5.55 (m, 1H), 5.43 (m, 4H), 5.32-5.16 (m, 2H), 4.93 (s, 2H), 4.39-4.31 (m, 2H), 4.29-4.25 (m, 1H), 4.22-4.17 (m, 1H), 3.90-3.84 (m, 1H), 3.78-3.73 (m, 1H), 3.67-3.62 (m, 1H), 3.60-3.55 (m, 2H), 3.51-3.46 (m, 13H), 3.44-3.40 (m, 3H), 3.27-3.23 (m, 3H), 3.06-3.00 (m, 2H), 2.97-2.90 (m, 2H), 2.43-2.39 (m, 4H), 2.36-2.30 (m, 2H), 2.26-2.20 (m, 3H), 2.18-2.15 (m, 1H), 2.08 (s, 1H), 1.95-1.84 (m, 5H), 1.76-1.69 (m, 3H), 1.60-1.55 (m, 2H), 1.43-1.35 (m, 3H), 0.87-0.80 (m, 11H) ppm. (proton of COOH was not revealed)

### Example 5A-5: Synthesis of (4S)-4-{[({4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methoxy)carbonyl]amino}-4-{[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}butanoic acid (LP9)

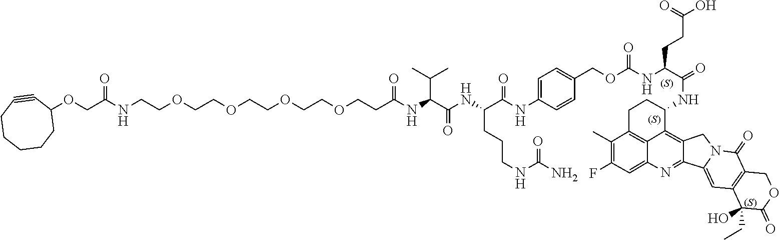

Following the general procedure C starting from P3 with 5-1a, linker-payload LP9 (22 mg, 38% yield) was obtained as a light yellow solid after purification by prep-HPLC (5-95% acetonitrile in aq. TFA (0.01%)). ESI m/z: 691.3 (M/2+H)⁺. ¹H NMR (400 MHz, DMSO_{d6}) δ 10.00 (s, 1H), 8.55 (d, J=8.5 Hz, 1H), 8.13 (d, J=7.1 Hz, 1H), 7.88 (d, J=8.8 Hz, 1H), 7.82 (d, J=10.9 Hz, 1H), 7.69-7.53 (m, 3H), 7.44 (d, J=7.6 Hz, 1H), 7.31 (s, 1H), 7.23 (d, J=8.5 Hz, 2H), 5.99 (s, 1H), 5.52 (s, 1H), 5.42 (s, 2H), 5.33 (d, J=18.7 Hz, 1H), 5.08 (d, J=18.7 Hz, 1H), 4.97-4.87 (m, 2H), 4.43-4.35 (m, 1H), 4.30-4.19 (m, 4H), 4.02-3.95 (m, 1H), 3.87 (d, J=14.7 Hz, 1H), 3.75 (d, J=14.8 Hz, 1H), 3.61-3.57 (m, 2H), 3.54-3.48 (m, 14H), 3.26-3.21 (m, 2H), 3.17 (s, 2H), 3.05-2.94 (m, 2H), 2.46 (d, J=7.0 Hz, 1H), 2.41-2.36 (m, 4H), 2.29-2.02 (m, 7H), 2.01-1.81 (m, 11H), 1.64-1.52 (m, 3H), 1.51-1.30 (m, 3H), 0.97-0.68 (m, 9H) ppm.

### Example 5A-6: Synthesis of {4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl N-[(1 S)-1-{[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}-2-phenylethyl]carbamate (LP10)

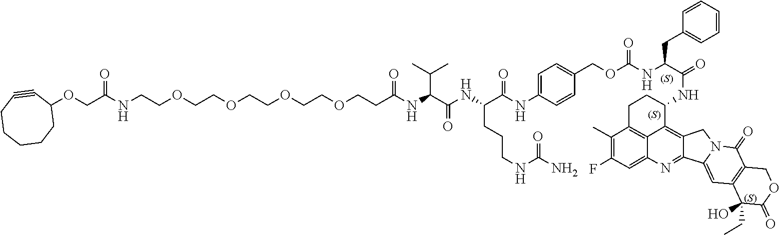

Following the general procedure C starting from P5 with 5-1a, linker-payload LP10 (55 mg, 51% yield) was obtained as a yellow solid after purification by prep-HPLC (5-95% acetonitrile in aq. TFA (0.01%)). ESI m/z: 700.3 (M/2+H)⁺. ¹H NMR (400 MHz, DMSO_{d6}) δ 9.99 (s, 1H), 8.65 (d, J=8.5 Hz, 1H), 8.13 (d, J=7.5 Hz, 1H), 7.88 (d, J=8.6 Hz, 1H), 7.82 (d, J=11.0 Hz, 1H), 7.68-7.49 (m, 4H), 7.31 (s, 1H), 7.26-7.13 (m, 4H), 7.06 (t, J=7.4 Hz, 2H), 7.00-6.94 (m, 1H), 6.00 (s, 1H), 5.58-5.51 (m, 1H), 5.43 (s, 2H), 5.23 (d, J=19.0 Hz, 1H), 4.89 (t, J=9.2 Hz, 2H), 4.42-4.21 (m, 4H), 3.85 (d, J=14.8 Hz, 1H), 3.75 (d, J=14.8 Hz, 1H), 3.67-3.58 (m, 14H), 3.43-3.40 (m, 2H), 3.27-3.17 (m, 4H), 3.09-2.89 (m, 4H), 2.85-2.78 (m, 1H), 2.46 (d, J=7.0 Hz, 1H), 2.43-2.36 (m, 4H), 2.26-2.03 (m, 6H), 1.99-1.81 (m, 6H), 1.80-1.66 (m, 4H), 1.66-1.30 (m, 7H), 0.93-0.78 (m, 9H) ppm.

### Example 5A-7: Synthesis of {4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl N-[(5S)-5-amino-5-{[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}pentyl]carbamate (LP11)

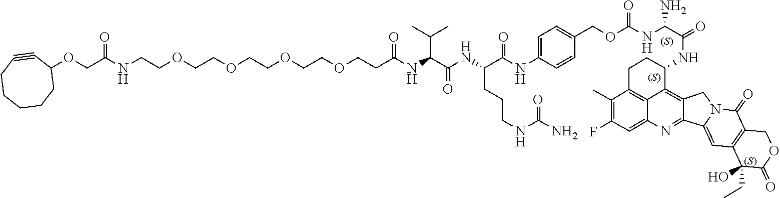

Following the general procedure C starting from 1-3 with 5-1a, linker-payload Fmoc-LP11 (38 mg (>95% purity) and 60 mg (>75% purity), ESI m/z: 802.2 (M+H)⁺) was obtained as a light yellow solid after purification by prep-HPLC (5-95% acetonitrile in aq. TFA (0.01%)). To a solution of Fmoc-LP11 (38 mg, >95% purity, 21 μmol) in dry DMF (1 mL) was added diethylamine (0.1 mL) and the reaction mixture was stirred at room temperature for 2 hours until Fmoc was totally removed, which was monitored by LCMS. The resulting mixture was directly separated by prep-H PLC (5-95% acetonitrile in aq. formic acid (0.1%)) to give LP11 (14 mg, 11% yield from 1-3) as a white solid. ESI m/z: 691.2 (M/2+H)⁺. ¹H NMR (400 MHz, DMSO_{d6}) δ 10.00 (s, 1H), 8.46 (s, 1H), 8.29 (s, 1H), 8.14 (d, J=7.3 Hz, 1H), 7.89 (d, J=8.8 Hz, 1H), 7.81 (d, J=10.9 Hz, 1H), 7.64-7.54 (m, 3H), 7.31 (s, 1H), 7.25 (d, J=8.5 Hz, 2H), 7.17 (s, 1H), 6.54 (s, 1H), 6.01 (s, 1H), 5.53 (s, 1H), 5.42 (m, 4H), 5.28-5.16 (m, 2H), 4.89 (s, 2H), 4.37 (m, 1H), 4.24 (m, 2H), 3.81 (m, 3H), 3.59 (m, 4H), 3.53-3.47 (m, 12H), 3.45-3.40 (m, 2H), 3.24 (m, 2H), 3.17 (m, 2H), 3.02-2.89 (m, 3H), 2.43-2.32 (m, 5H), 2.26-2.15 (m, 3H), 2.13-2.02 (m, 3H), 1.99-1.90 (m, 2H), 1.88-1.79 (m, 3H), 1.79-1.66 (m, 3H), 1.64-1.52 (m, 4H), 1.48-1.33 (m, 7H), 0.91-0.78 (m, 9H) ppm. ¹⁹F NMR (376 MHz, DMSO_{d6}) δ −111.2 ppm.

### Example 5A-8: {4-[(2S)-5-(carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl N-[({[2-({2-[(19S)-19-ethyl-19-hydroxy-14,18-dioxo-17-oxa-3,13-diazapentacyclo[11.8.0.0²,¹¹.0⁴,⁹.0¹⁵,²⁰]henicosa-1(21),2,4,6,8,10,15(20)-heptaen-10-yl]ethyl}(propan-2-yl)amino)ethoxy]methyl}carbamoyl)methyl]carbamate (LP12)

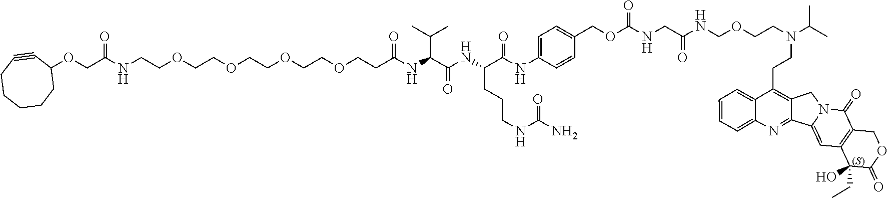

Following the general procedure C starting from P11 with 5-1a, linker-payload LP12 (10 mg, 40% yield) was obtained as a white solid after purification by prep-HPLC (5-95% acetonitrile in aq. TFA (0.01%)). ESI m/z: 690.8 (M/2+H)⁺. ¹H NMR (400 MHz, DMSO_{d6}) δ 9.99 (s, 1H), 9.46 (s, 1H), 8.95-8.81 (m, 1H), 8.37 (d, J=8.4 Hz, 1H), 8.23 (d, J=8.2 Hz, 1H), 8.12 (d, J=7.4 Hz, 1H), 8.00-7.85 (m, 2H), 7.82 (t, J=7.5 Hz, 1H), 7.70-7.48 (m, 4H), 7.37 (s, 1H), 7.25 (d, J=8.1 Hz, 2H), 6.57 (br s, 1H), 5.99 (br t, J=5.3 Hz, 1H), 5.55-5.35 (m, 6H), 4.91 (br s, 2H), 4.71 (br s, 2H), 4.44-4.33 (m, 1H), 4.30-4.19 (m, 2H), 3.90-3.72 (m, 6H), 3.65-3.56 (m, 5H), 3.53-3.45 (m, 16H), 3.26-3.22 (m, 2H), 3.03-2.93 (m, 2H), 2.40-2.32 (m, 1H), 2.29-2.01 (m, 4H), 2.00-1.80 (m, 6H), 1.80-1.53 (m, 7H), 1.46-1.34 (m, 3H), 1.32-1.24 (m, 6H), 0.90-0.82 (m, 9H) ppm.

### Example 5A-9: {4-[(2S)-5-(Carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl N-{[(19S)-19-ethyl-19-hydroxy-7-methoxy-14,18-dioxo-17-oxa-3,13-diazapentacyclo[11.8.0.0²,¹¹.0⁴,⁹.0¹⁵,²⁰]henicosa-1(21),2,4,6,8,10,15(20)-heptaen-10-yl]methyl}carbamate (LP20)

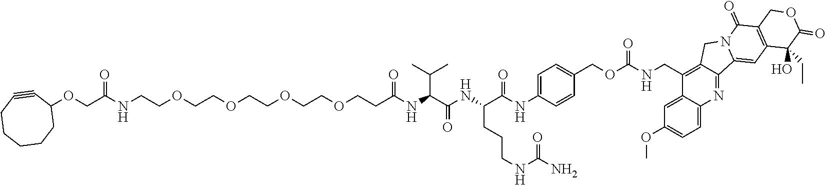 [SMILES: CC[C@@]1(O)C(=O)OCc2c1cc1n(c2=O)Cc2c-1nc1ccc(OC)cc1c2CNC(=O)OCc1ccc(NC(=O)[C@H](CCCNC(N)=O)NC(=O)[C@@H](NC(=O)CCOCCOCCOCCOCCNC(=O)COC2C#CCCCCC2)C(C)C)cc1]

Following the general procedure C starting from P21 with 5-1a, linker-payload LP20 (16 mg, 57% yield, TFA salt) was obtained as a light yellow solid after purification by prep-HPLC (5-95% acetonitrile in aq. TFA (0.01%)). ESI m/z: 613.0 (M/2+H)⁺. ¹H NMR (400 MHz, DMSO_{d6}) δ 9.97 (s, 1H), 8.17 (t, J=6.4 Hz, 1H), 8.10 (d, J=9.2 Hz, 2H), 7.87 (d, J=8.4 Hz, 1H), 7.66 (s, 1H), 7.61-7.56 (m, 3H), 7.53 (dd, J=8.8, 2.4 Hz, 1H), 7.29 (s, 2H), 7.27 (br s, 1H), 6.51 (s, 1H), 5.97 (t, J=6.4 Hz, 1H), 5.48 (s, 2H), 5.44 (s, 2H), 5.41 (s, 2H), 4.97 (s, 2H), 4.79 (d, J=6.0 Hz, 2H), 4.41-4.34 (m, 1H), 4.28-4.20 (m, 2H), 3.96 (s, 3H), 3.87 (d, J=14.8 Hz, 1H), 3.75 (d, J=14.8 Hz, 1H), 3.62-3.58 (m, 2H), 3.50-3.48 (m, 12H), 3.46-3.42 (m, 2H), 3.26-3.20 (m, 2H), 3.03-2.91 (m, 2H), 2.47-2.33 (m, 1H), 2.21-2.03 (m, 4H), 1.98-1.68 (m, 8H), 1.61-1.46 (m, 3H), 1.42-1.31 (m, 3H), 0.91-0.82 (m, 9H) ppm. (The proton of TFA was not observed). ¹⁹F NMR (376 MHz, DMSO_{d6}) δ −74 ppm.

### Example 5A-10: Synthesis of {4-[(2S)-2-[(2S)-2-[1-(4-{2-Azatricyclo[10.4.0.0⁴,9]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methyl N-{[(19S)-19-ethyl-19-hydroxy-7-methoxy-14,18-dioxo-17-oxa-3,13-diazapentacyclo[11.8.0.0²,¹¹.0⁴,⁹.0¹⁵,²⁰]henicosa-1(21),2,4,6,8,10,15(20)-heptaen-10-yl]methyl}carbamate (LP21)

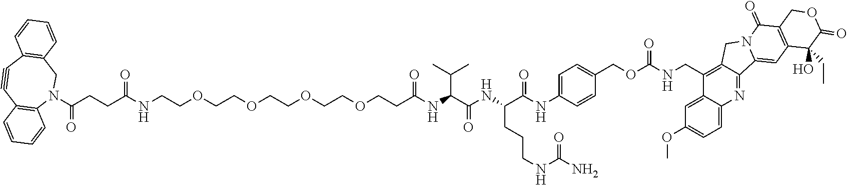 [SMILES: CC[C@@]1(O)C(=O)OCc2c1cc1n(c2=O)Cc2c-1nc1ccc(OC)cc1c2CNC(=O)OCc1ccc(NC(=O)[C@H](CCCNC(N)=O)NC(=O)[C@@H](NC(=O)CCOCCOCCOCCOCCNC(=O)CCC(=O)N2Cc3ccccc3C#Cc3ccccc32)C(C)C)cc1]

Following the general procedure C starting from P21 with 5-1b, linker-payload LP21 (10 mg, 38% yield) was obtained as a light yellow solid after purification by reversed phase flash chromatography (0-100% methanol in aq. ammonium bicarbonate (10 mM)).

Lactone: HPLC purity: 77%, retention time: 7.67 min, ESI m/z: 449.9 (M/3+H)+, 674.3 (M/2+H)+;

Ring-opening product: HPLC purity: 23%, retention time: 6.57 min, ESI m/z: 455.9 (M/3+H)⁺, 683.4 (M/2+H)⁺.

¹H NMR (400 MHz, DMSO_{d6}) δ 9.98 (s, 1H), 8.18 (t, J=6.0 Hz, 1H), 8.13-8.09 (m, 2H), 7.87 (d, J=8.4 Hz, 1H), 7.77 (d, J=5.6 Hz, 1H), 7.69-7.65 (m. 2H), 7.63-7.60 (m, 1H), 7.57 (d, J=8.4 Hz, 2H), 7.54-7.44 (m, 4H), 7.39-7.34 (m, 2H), 7.32-7.27 (m, 4H), 6.51 (s, 1H), 5.99-5.96 (m, 1H), 5.48 (br s, 2H), 5.44 (s, 2H), 5.41 (s, 2H), 5.02 (d, J=13.6 Hz, 1H), 4.97 (s, 2H), 4.79 (d, J=6.0 Hz, 2H), 4.40-4.35 (m, 1H), 4.25-4.21 (m, 1H), 3.96 (s, 3H), 3.62-3.57 (m, 3H), 3.48-3.42 (m, 12H), 3.30-3.28 (m, 2H), 3.09-3.07 (m, 2H), 3.03-2.99 (m, 1H), 2.97-2.91 (m, 1H), 2.60-2.55 (m, 1H), 2.46-2.44 (m, 1H), 2.41-2.37 (m, 1H), 2.28-2.19 (m, 1H), 2.03-1.93 (m, 2H), 1.89-1.83 (m, 2H), 1.80-1.65 (m, 2H), 1.61-1.53 (m, 1H), 1.48-1.31 (m, 2H), 0.90-0.81 (m, 9H) ppm.

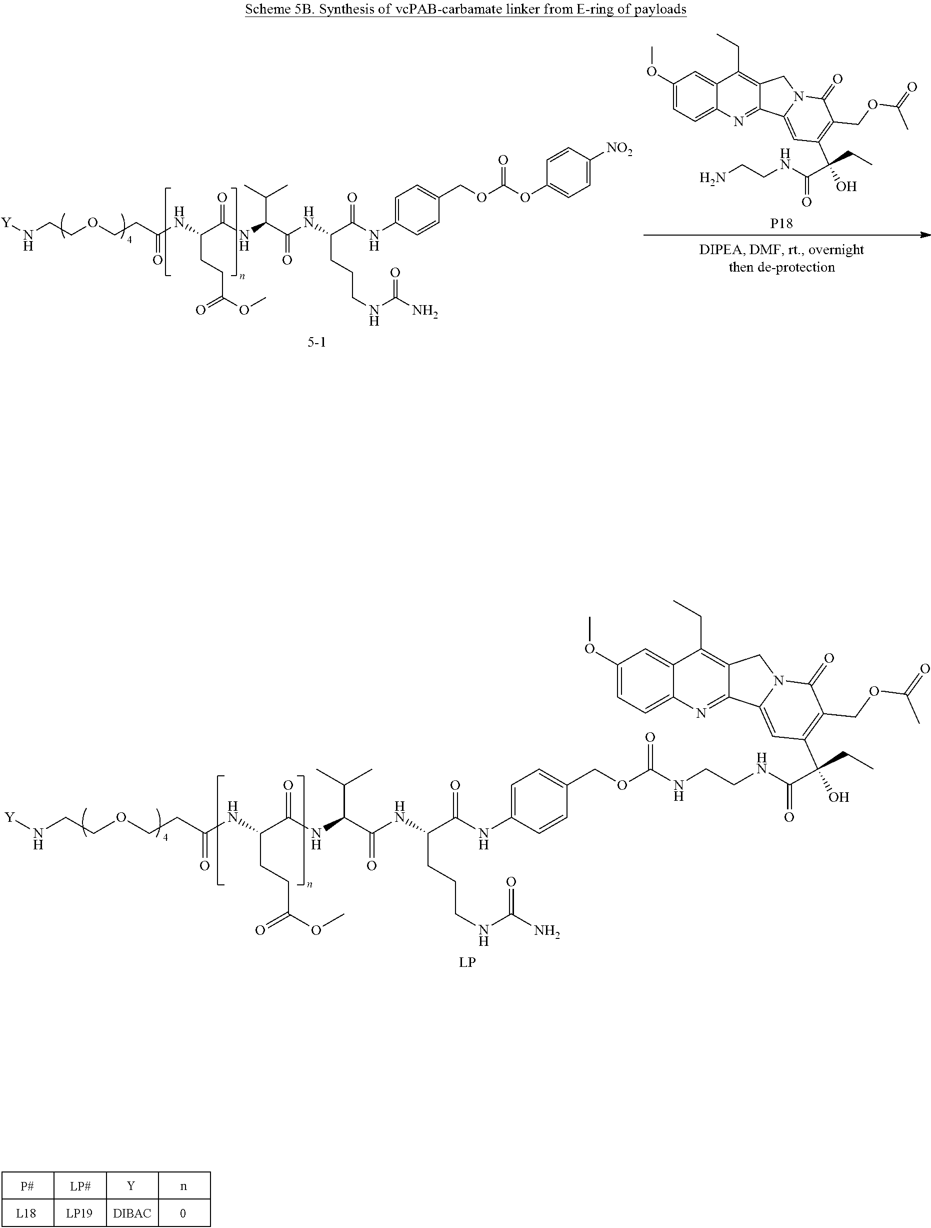

### Example 5B: Synthesis of {7-[(1 S)-1-[(2-{[({4-[(2S)-2-[(2S)-2-[1-(4-{2-Azatricyclo[10.4.0.0⁴,9]hexadeca-1(12),4(9),5,7,13,15-hexaen-10-yn-2-yl}-4-oxobutanamido)-3,6,9,12-tetraoxapentadecan-15-amido]-3-methylbutanamido]-5-(carbamoylamino)pentanamido]phenyl}methoxy)carbonyl]amino}ethyl)carbamoyl]-1-hydroxypropyl]-12-ethyl-2-methoxy-9-oxo-9H,11H-indolizino[1,2-b]quinolin-8-yl}methyl acetate (LP19)

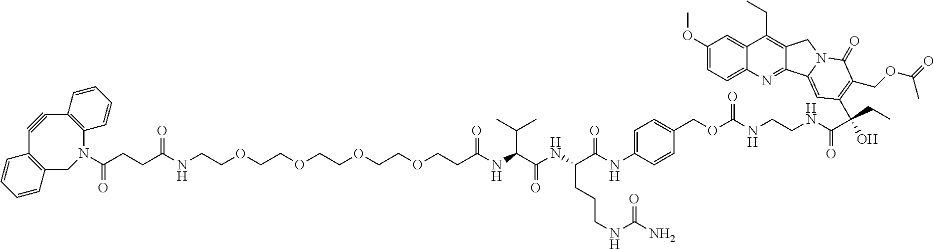 [SMILES: CCc1c2c(nc3ccc(OC)cc13)-c1cc([C@@](O)(CC)C(=O)NCCNC(=O)OCc3ccc(NC(=O)[C@H](CCCNC(N)=O)NC(=O)[C@@H](NC(=O)CCOCCOCCOCCOCCNC(=O)CCC(=O)N4Cc5ccccc5C#Cc5ccccc54)C(C)C)cc3)c(COC(C)=O)c(=O)n1C2]

Following the general procedure C starting from P18 with 5-1b, linker-payload LP19 (19 mg, 82% yield) was obtained as a white solid after purification by reversed phase flash chromatography (0-80% acetonitrile in water). ESI m/z: 695.0 ((M-OAc)/2+H)⁺. ¹H NMR (400 MHz, DMSO_{d6}) δ 9.98 (s, 1H), 8.12-8.09 (m, 2H), 8.06-8.03 (m, 1H), 7.87 (d, J=8.4 Hz, 1H), 7.77 (t, J=6.0 Hz, 1H), 7.69 (dd, J=8.4 and 2.0 Hz, 1H), 7.62 (d, J=7.2 Hz, 1H), 7.57 (d, J=8.4 Hz, 2H), 7.53-7.44 (m, 5H), 7.41-7.39 (m, 1H), 7.38-7.28 (m, 3H), 7.24 (d, J=8.8 Hz, 2H), 7.20-7.18 (m, 1H), 6.18 (s, 1H), 5.98 (t, J=6.0 Hz, 1H), 5.42 (s, 2H), 5.37 (d, J=10.8 Hz, 1H), 5.30 (d, J=10.8 Hz, 1H), 5.29 (s, 2H), 5.03 (d, J=14.0 Hz, 1H), 4.90 (s, 2H), 4.41-4.35 (m, 1H), 4.23 (q, J=6.8 Hz, 1H), 3.99 (s, 3H), 3.62-3.57 (m, 3H), 3.48-3.42 (m, 12H), 3.30-3.28 (m, 1H), 3.22-3.17 (m, 3H), 3.10-3.07 (m, 5H), 3.04-2.93 (m, 2H), 2.68-2.55 (m, 1H), 2.46-2.33 (m, 2H), 2.26-2.11 (m, 4H), 2.10-1.96 (m, 5H), 1.78-1.63 (m, 2H), 1.60-1.53 (m, 1H), 1.47-1.35 (m, 2H), 1.32 (t, J=7.2 Hz, 3H), 0.88-0.81 (m, 9H) ppm.

### Example 6: Synthesis of Sugar-PAB Linker-Payloads LP3, LP4 and LP7 (Scheme 6)

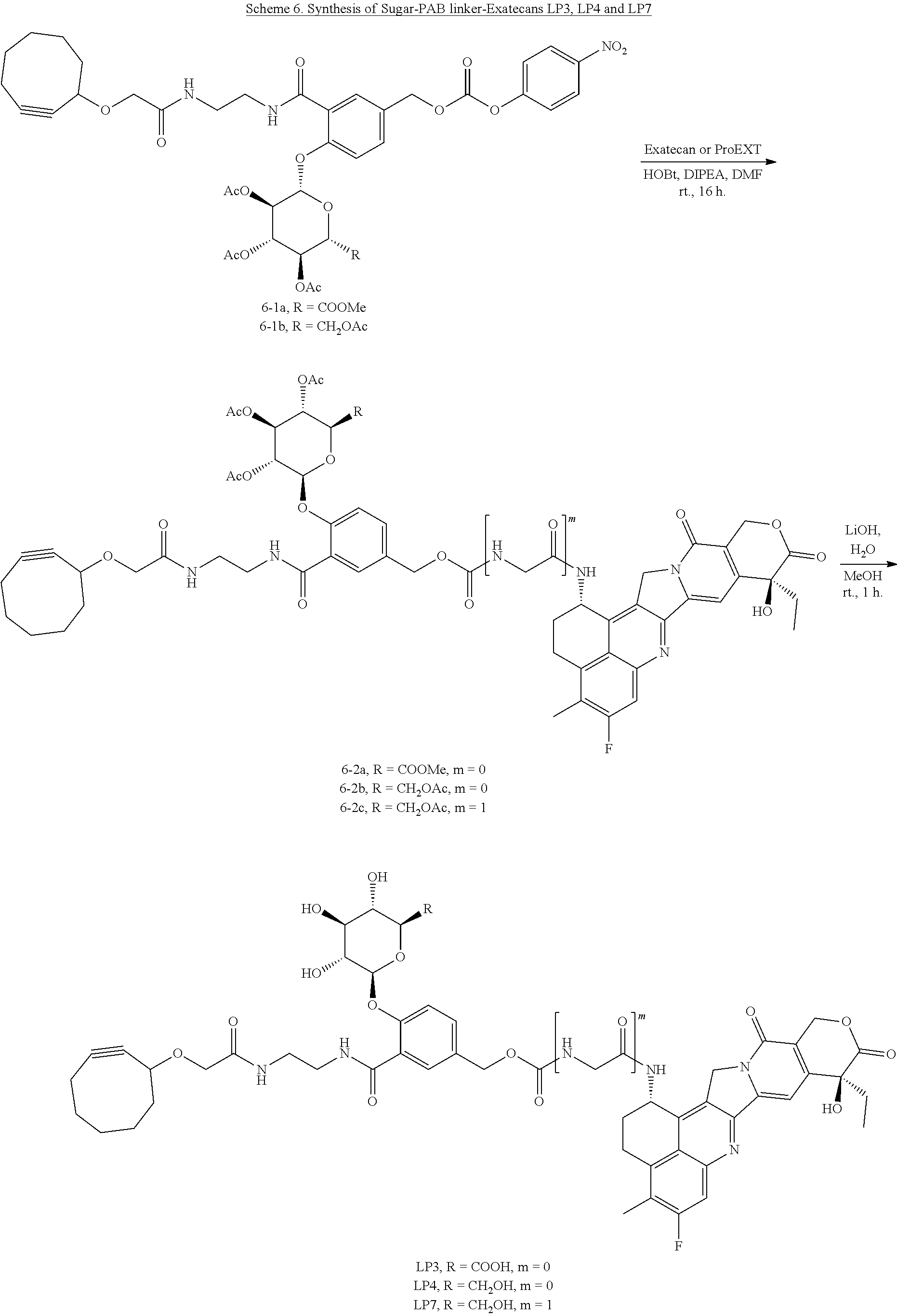

### Example 6A: LP3

### Example 6A-1: Synthesis of methyl (2S,3S,4S,5R,6S)-3,4,5-tris(acetyloxy)-6-[2-({2-[2-(cyclooct-2-yn-1-yloxy)acetamido]ethyl}carbamoyl)-4-[({[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}oxy)methyl]phenoxy]oxane-2-carboxylate (6-2a)

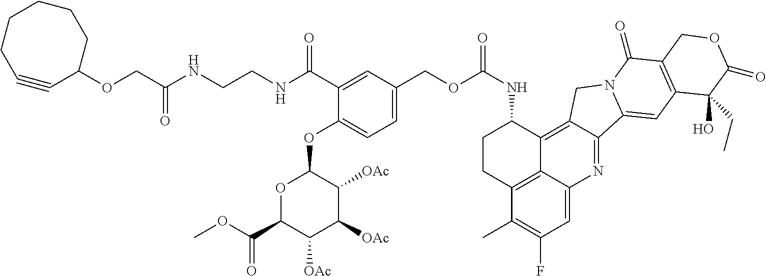 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)OC)[C@@H](OC(C)=O)[C@H](OC(C)=O)[C@H]2OC(C)=O)c(C(=O)NCCNC(=O)COC2C#CCCCCC2)c1)CC3]

To a solution of compound 6-1a (26 mg, 30 μmol) in DMF (1.0 mL) were added Exatecan mesylate (16 mg, 30 μmol), HOBt (4.0 mg, 30 μmol) and DIPEA (7.7 mg, 60 μmol). The reaction mixture was stirred at room temperature for 16 hours, which was monitored by LCMS. The resulting mixture was directly purified by reversed phase flash chromatography (0-100% acetonitrile in aq. TFA(0.01%)) to give compound 6-2a (18 mg, 52% yield) as a yellow solid. ESI m/z: 1152.5 (M+H)⁺.

### Example 6A-2: Synthesis of (2S,3S,4S,5R,6S)-6-[2-({2-[2-(Cyclooct-2-yn-1-yloxy)acetamido]ethyl}carbamoyl)-4-[({[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}oxy)methyl]phenoxy]-3,4,5-trihydroxyoxane-2-carboxylic acid (LP3)

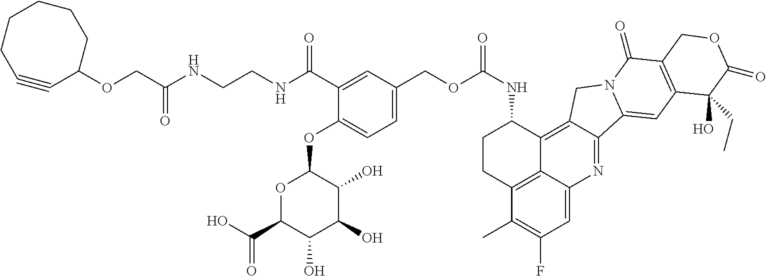 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](C(=O)O)[C@@H](O)[C@H](O)[C@H]2O)c(C(=O)NCCNC(=O)COC2C#CCCCCC2)c1)CC3]

To a mixture of compound 6-2a (22 mg, 19 μmol) in methanol (2 mL) were added lithium hydroxide monohydrate (7.2 mg, 0.17 mmol) and water (2 mL). The reaction mixture was stirred at 25° C. for an hour, which was monitored by LCMS. The resulting mixture was acidified to pH 3-4 with aq. hydrochloride (1 N), and then purified by prep-HPLC (5-95% acetonitrile in aq. TFA (0.1%)) to give LP3 (6.0 mg, 28% yield, TFA salt) as a white solid. ESI m/z: 1012.5 (M+H)⁺. ¹H NMR (400 MHz, DMSO_{d6}) δ 8.56 (s, 1H), 8.09 (d, J=9.2 Hz, 1H), 7.98-7.92 (m, 1H), 7.79-7.76 (m, 2H), 7.54-7.52 (m, 1H), 7.34 (d, J=8.4 Hz, 1H), 7.30 (s, 1H), 6.52 (br s, 1H), 5.66 (br s, 1H), 5.44 (s, 2H), 5.28 (s, 3H), 5.15-5.06 (m, 3H), 4.89 (s, 1H), 4.25 (s, 1H), 3.88-3.71 (m, 2H), 3.25-3.05 (m, 9H), 2.38-2.33 (m, 4H), 2.23-2.01 (m, 6H), 1.98-1.79 (m, 4H), 1.74-1.65 (m, 2H), 1.65-1.46 (m, 2H), 1.28 (m, 1H), 0.88 (t, J=7.2 Hz, 3H) ppm. (protons of acid was not revealed.)

### Example 6B: LP4

### Example 6B-1: Synthesis of [(2R,3R,4S,5R,6S)-3,4,5-Tris(acetyloxy)-6-[2-({2-[2-(cyclooct-2-yn-1-yloxy)acetamido]ethyl}carbamoyl)-4-[({[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}oxy)methyl]phenoxy]oxan-2-yl]methyl acetate (6-2b)

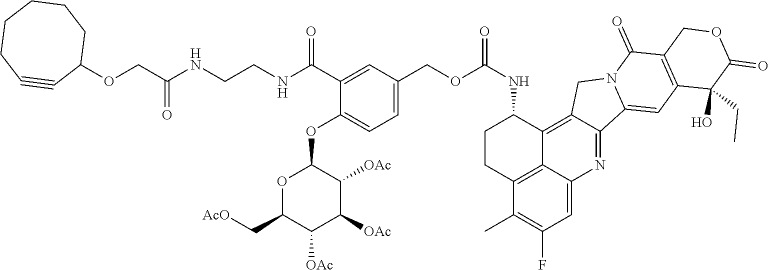 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](COC(C)=O)[C@@H](OC(C)=O)[C@H](OC(C)=O)[C@H]2OC(C)=O)c(C(=O)NCCNC(=O)COC2C#CCCCCC2)c1)CC3]

To a solution of compound 6-1b (55 mg, 63 μmol) in DMF (3.0 mL) were added exatecan mesylate (34 mg, 63 μmol), HOBt (8.5 mg, 63 μmol) and DIPEA (16 mg, 0.13 mmol), and the reaction mixture was stirred at room temperature for 16 hours, which was monitored by LCMS. The resulting mixture was directly purified by reversed phase flash chromatography (0-100% acetonitrile in aq. TFA (0.1%)) to give compound 6-2b (34 mg, 46% yield) as a yellow solid. ESI m/z: 1166.5 (M+H)⁺.

### Example 6B-2: Synthesis of [3-({2-[2-(Cyclooct-2-yn-1-yloxy)acetamido]ethyl}carbamoyl)-4-{[(2S,3R,4S,5S,6R)-3,4,5-trihydroxy-6-(hydroxymethyl)oxan-2-yl]oxy}phenyl]methyl N-[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamate (LP4)

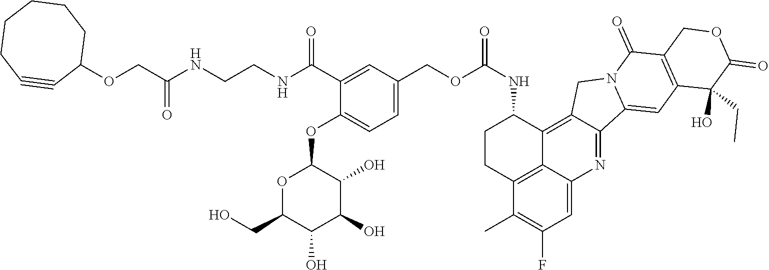 [SMILES: CC[C@@]1(O)C(=O)OCc2c1cc1n(c2=O)Cc2c-1nc1cc(F)c(C)c3c1c2[C@@H](NC(=O)OCc1ccc(O[C@@H]2O[C@H](CO)[C@@H](O)[C@H](O)[C@H]2O)c(C(=O)NCCNC(=O)COC2C#CCCCCC2)c1)CC3]

To a mixture of compound 6-2b (15 mg, 13 μmol) in methanol (2 mL) were added lithium hydroxide monohydrate (4.9 mg, 0.12 mmol) and water (2 mL). The reaction mixture was stirred at 15° C. for an hour, which was monitored by LCMS. The resulting mixture was acidified to pH 3-4 with aq. hydrochloride (1 N), and then purified by prep-HPLC (5-95% acetonitrile in aq. formic acid (0.1%)) to give LP4 (4.0 mg, 31% yield) as a yellow solid. ESI m/z: 998.2 (M+H)⁺. ¹H NMR (400 MHz, DMSO_{d6}) δ 8.49-8.43 (m, 4H), 8.10 (d, J=8.8 Hz, 1H), 7.83-7.72 (m, 3H), 7.54 (d, J=8.4 Hz, 1H), 7.37 (d, J=8.8 Hz, 1H), 7.31 (s, 1H), 6.54 (s, 1H), 5.78 (br s, 1H), 5.44 (s, 2H), 5.33-5.28 (m, 4H), 5.16-5.08 (m, 3H), 4.91 (d, J=8.4 Hz, 1H), 4.68 (br s, 1H), 4.28-4.23 (m, 1H), 3.88-3.72 (m, 4H), 3.20-2.94 (m, 3H), 2.38-2.33 (m, 4H), 2.17-1.97 (m, 8H), 1.96-1.68 (m, 6H), 1.56-1.38 (m, 3H), 0.88 (t, J=7.6 Hz, 3H) ppm.

### Example 3C: LP7

### Example 3C-1: Synthesis of [(2R,3R,4S,5R,6S)-3,4,5-Tris(acetyloxy)-6-[2-({2-[2-(cyclooct-2-yn-1-yloxy)acetamido]ethyl}carbamoyl)-4-({[({[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}methyl)carbamoyl]oxy}methyl)phenoxy]oxan-2-yl]methyl acetate (4-2c)

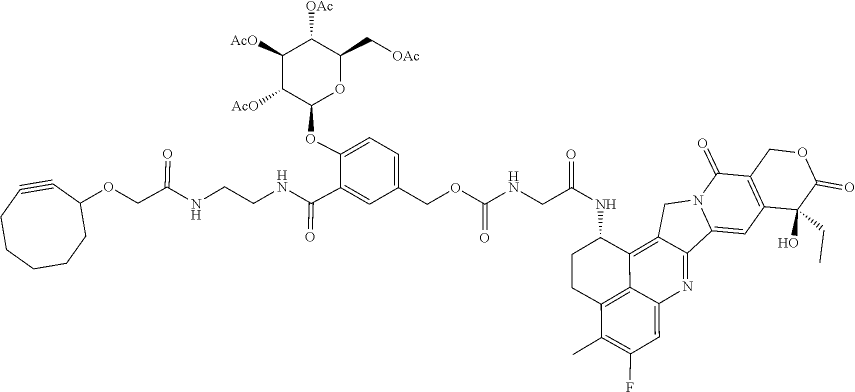 [SMILES: CC[C@@]1(O)C(=O)OCc2c1cc1n(c2=O)Cc2c-1nc1cc(F)c(C)c3c1c2[C@@H](NC(=O)CNC(=O)OCc1ccc(O[C@@H]2O[C@H](COC(C)=O)[C@@H](OC(C)=O)[C@H](OC(C)=O)[C@H]2OC(C)=O)c(C(=O)NCCNC(=O)COC2C#CCCCCC2)c1)CC3]

To a solution of compound 6-1b (31 mg, 36 μmol) in DMF (1.0 mL) were added P2 (17 mg, 36 μmol), HOBt (5.0 mg, 36 μmol) and DIPEA (9.2 mg, 71 μmol), and the reaction mixture was stirred at room temperature for 2 hours, which was monitored by LCMS. The resulting mixture was purified by reversed phase flash chromatography (0-100% acetonitrile in aq. TFA (0.01%)) to give compound 6-2c (43 mg, 88% yield) as a yellow solid. ESI m/z: 1223.5 (M+H)⁺, 1245.5 (M+Na)⁺.

### Example 3C-2: Synthesis of [3-({2-[2-(Cyclooct-2-yn-1-yloxy)acetamido]ethyl}carbamoyl)-4-{[(2S,3R,4S,5S,6R)-3,4,5-trihydroxy-6-(hydroxymethyl)oxan-2-yl]oxy}phenyl]methyl N-({[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}methyl)carbamate (LP7)

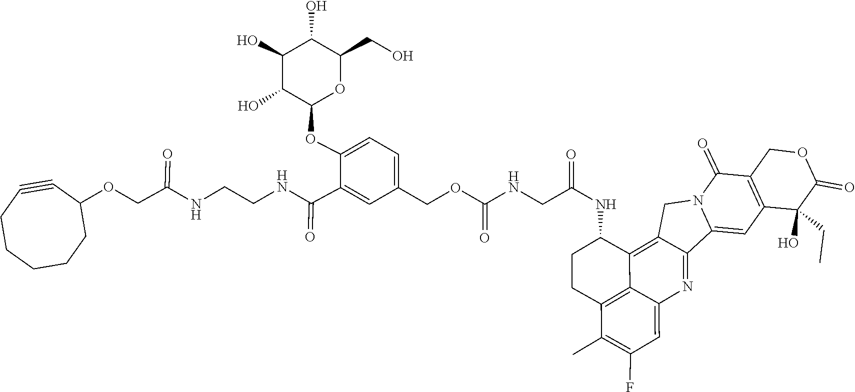 [SMILES: CC[C@@]1(O)C(=O)OCc2c1cc1n(c2=O)Cc2c-1nc1cc(F)c(C)c3c1c2[C@@H](NC(=O)CNC(=O)OCc1ccc(O[C@@H]2O[C@H](CO)[C@@H](O)[C@H](O)[C@H]2O)c(C(=O)NCCNC(=O)COC2C#CCCCCC2)c1)CC3]

To a mixture of compound 6-2c (43 mg, 35 μmol) in methanol (2 mL) was added aq. lithium hydroxide (0.16 mM, 2 mL), and the reaction mixture was stirred at 15° C. for 2 hours, which was monitored by LCMS. The resulting mixture was acidified by aq. hydrochloride (1 N) to pH 3-4 and was then purified by prep-HPLC (5-95% acetonitrile in aq. TFA (0.1%)) to give LP7 (9.8 mg, 57% yield) as a white solid. ESI m/z: 1055.5 (M+H)⁺.

### Example 7: Synthesis of Peptide Linker-Payload LP8 (Schemes 7A and 7B)

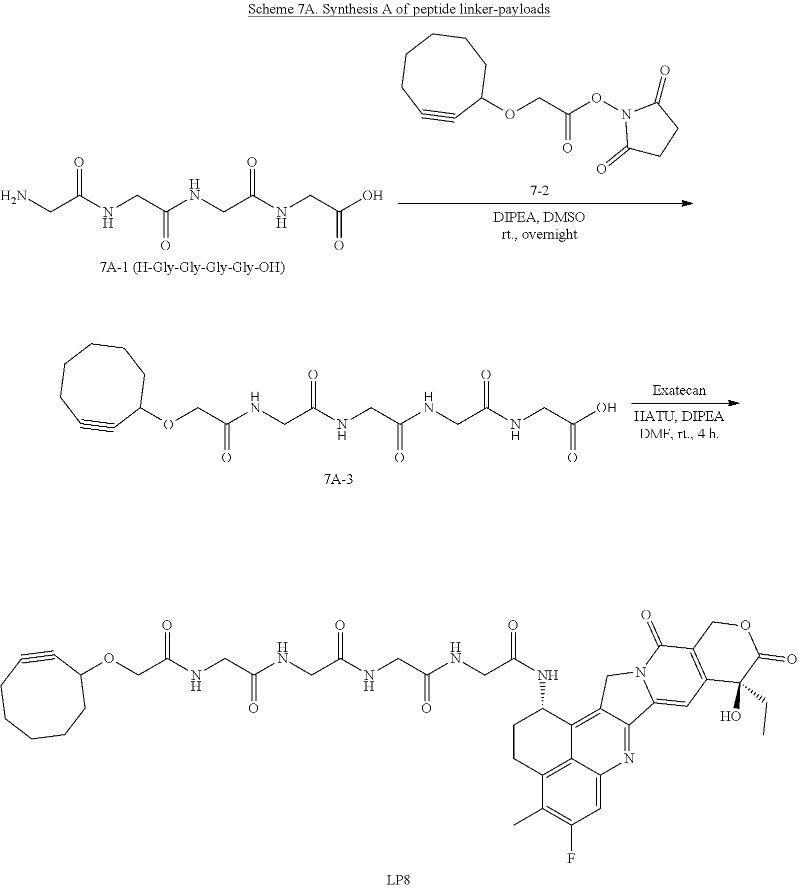 [SMILES: NCC(=O)NCC(=O)NCC(=O)NCC(=O)O ; O=C(COC1C#CCCCCC1)ON1C(=O)CCC1=O ; O=C(O)CNC(=O)CNC(=O)CNC(=O)CNC(=O)COC1C#CCCCCC1 ; CC[C@@]1(O)C(=O)OCc2c1cc1n(c2=O)Cc2c-1nc1cc(F)c(C)c3c1c2[C@@H](NC(=O)CNC(=O)CNC(=O)CNC(=O)CNC(=O)COC1C#CCCCCC1)CC3]

### Example 7A: Synthesis of 2-[2-(2-{2-[2-(Cyclooct-2-yn-1-yloxy)acetamido]acetamido} acetamido)acetamido]acetic acid (7A-3)

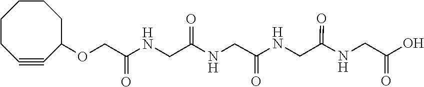 [SMILES: O=C(O)CNC(=O)CNC(=O)CNC(=O)CNC(=O)COC1C#CCCCCC1]

To a solution of compound 7-2 (0.10 g, 0.37 mmol) in DMSO (2 mL) were added peptide 7A-1 (H-Gly-Gly-Gly-Gly-OH, 90 mg, 0.37 mmol) and DIPEA (94 mg, 0.73 mmol), and the reaction mixture was stirred at room temperature overnight. The resulting mixture was directly purified by reversed phase flash chromatography (5-95% acetonitrile in aq. TFA (0.01%)) to give compound 7A-3 (23 mg, 15% yield) as a white solid. ESI m/z: 411.2 (M+H)⁺. ¹H NMR (400 MHz, DMSO_{d6}) δ 8.23-8.07 (m, 3H), 7.83 (t, J=5.7 Hz, 1H), 4.33 (d, J=6.4 Hz, 1H), 3.93 (d, J=14.9 Hz, 1H), 3.77 (m, 9H), 2.25-2.04 (m, 3H), 1.98-1.84 (m, 2H), 1.82-1.71 (m, 2H), 1.59 (s, 2H), 1.40 (m, 1H) ppm. (The proton of TFA was not observed).

### Example 7B: Synthesis of 2-(2-{2-[2-(Cyclooct-2-yn-1-yloxy)acetamido]acetamido}acetamido)-N-({[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}methyl)acetamide (LP8)

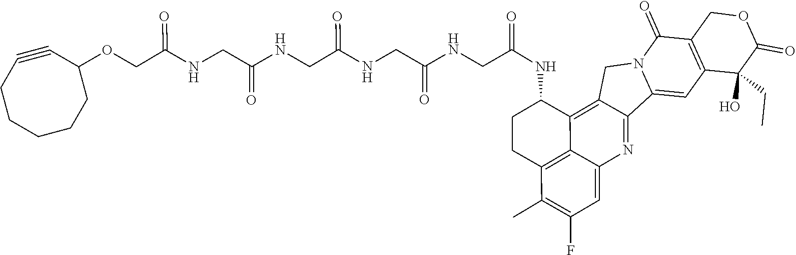 [SMILES: CC[C@@]1(O)C(=O)OCc2c1cc1n(c2=O)Cc2c-1nc1cc(F)c(C)c3c1c2[C@@H](NC(=O)CNC(=O)CNC(=O)CNC(=O)CNC(=O)COC1C#CCCCCC1)CC3]

To a solution of compound 7A-3 (12 mg, 29 μmol) in dry DMF (3 mL) were added HATU (17 mg, 44 μmol) and DIPEA (12 mg, 88 μmol), and the mixture was stirred at room temperature for 10 minutes before the addition of Exatecan (13 mg, 29 μmol). The reaction mixture was stirred at room temperature for 4 hours until Exatecan was totally consumed according to LCMS. The resulting mixture was directly purified by prep-HPLC (0-100% acetonitrile in aq. TFA (0.01%)) to give linker-payload LP8 (11 mg, 39% yield, TFA salt) as a white solid. ESI m/z: 828.5 (M+H)⁺. ¹H NMR (400 MHz, DMSO_{d6}) δ 8.40 (d, J=8.5 Hz, 1H), 8.18-8.09 (m, 3H), 7.84-7.77 (m, 2H), 7.32 (s, 1H), 5.62-5.52 (m, 1H), 5.43 (s, 2H), 5.23 (d, J=3.7 Hz, 2H), 4.31 (t, J=5.6 Hz, 1H), 3.92 (d, J=14.7 Hz, 1H), 3.82-3.63 (m, 9H), 3.18-3.16 (m, 2H), 2.41 (s, 3H), 2.23-2.09 (m, 5H), 1.93-1.75 (m, 6H), 1.60-1.53 (m, 2H), 1.24 (s, 2H), 0.87 (t, J=7.3 Hz, 3H) ppm.

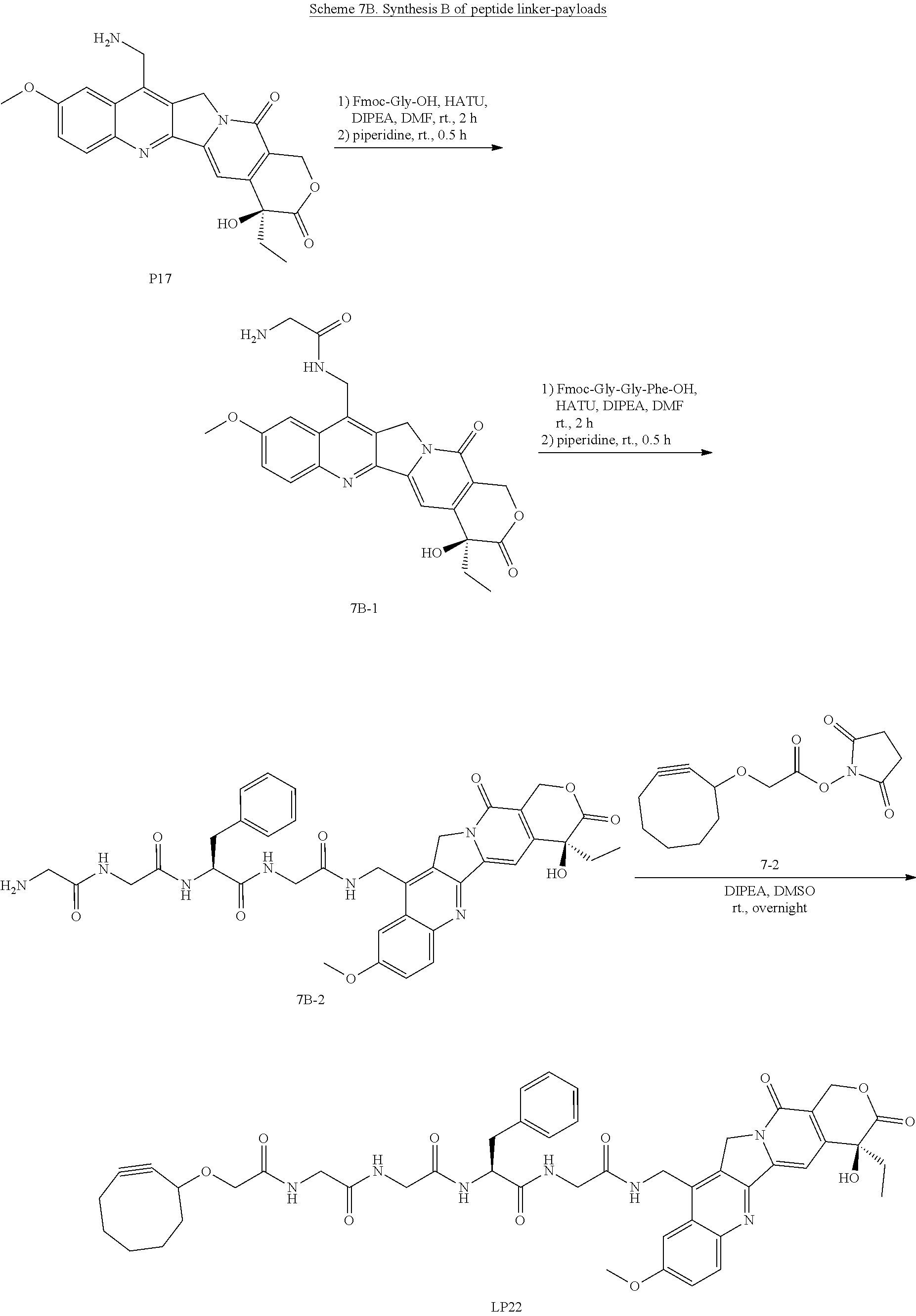

### Example 7C: Synthesis of 2-Amino-N-{[(19S)-19-ethyl-19-hydroxy-7-methoxy-14,18-dioxo-17-oxa-3,13-diazapentacyclo[11.8.0.0²,¹¹.0⁴,⁹.0¹⁵,²⁰]henicosa-1(21),2,4,6,8,10,15(20)-heptaen-10-yl]methyl}acetamide (7B-1)

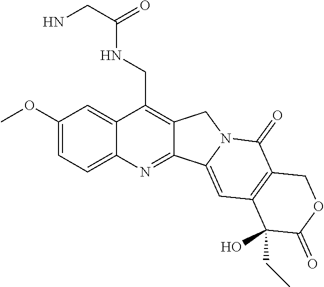 [SMILES: CC[C@@]1(O)C(=O)OCc2c1cc1n(c2=O)Cc2c-1nc1ccc(OC)cc1c2CNC(=O)CN]

To a solution of Fmoc-Gly-OH (37 mg, 0.13 mmol) in DMF (5.0 mL) were added DIPEA (0.04 mL, 0.25 mmol), HATU (56 mg, 0.15 mmol), and the reaction mixture was stirred at room temperature for 15 minutes before the addition of payload P17 (50 mg, 0.12 mmol). The mixture was then stirred at room temperature overnight until P17 was totally consumed, which was monitored by LCMS. To the mixture was then added piperidine (0.3 mL) and the resulting mixture was stirred at room temperature for half an hour until Fmoc was totally removed according to LCMS. The mixture was directly purified by reversed phase flash chromatography (0-100% acetonitrile in aq. TFA (0.01%)) to give 7B-1 (35 mg, 61% yield, TFA salt) as a yellow solid. ESI m/z: 465.2 (M+H)⁺.

### Example 7D: Synthesis of (2S)-2-[2-(2-Aminoacetamido)acetamido]-N-[({[(19S)-19-ethyl-19-hydroxy-7-methoxy-14,18-dioxo-17-oxa-3,13-diazapentacyclo[11.8.0.0²,¹¹.0⁴,⁹.0¹⁵,²⁰]henicosa-1(21),2,4,6,8,10,15(20)-heptaen-10-yl]methyl}carbamoyl)methyl]-3-phenylpropanamide (7B-2)

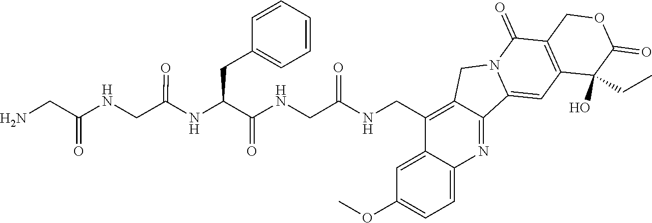 [SMILES: CC[C@@]1(O)C(=O)OCc2c1cc1n(c2=O)Cc2c-1nc1ccc(OC)cc1c2CNC(=O)CNC(=O)[C@H](Cc1ccccc1)NC(=O)CNC(=O)CN]

To a solution of peptide Fmoc-Gly-Gly-Phe-OH (32 mg, 65 μmol) in DMF (6 mL) were added DIPEA (0.02 mL, 0.13 mmol) and HATU (29 mg, 77 μmol), and the reaction mixture was stirred at room temperature for 15 minutes before the addition of compound 7B-1 (30 mg, 65 μmol). The mixture was stirred at room temperature overnight, which was monitored by LCMS. To the reaction mixture was then added piperidine (0.3 mL) and the mixture was stirred at room temperature for half an hour until Fmoc was totally removed according to LCMS. The reaction mixture was directly purified by reversed phase flash chromatography (0-100% acetonitrile in aq. TFA (0.01%)) to give crude 7B-2 (35 mg, TFA salt, crude (68% purity in LCMS)) as a yellow solid, which was used for the next step without further purification. ESI m/z: 363.8 (M/2+H)⁺.

### Example 7E: Synthesis of (2S)-2-(2-{2-[2-(Cyclooct-2-yn-1-yloxy)acetamido]acetamido}acetamido)-N-[({[(19S)-19-ethyl-19-hydroxy-7-methoxy-14,18-dioxo-17-oxa-3,13-diazapentacyclo[11.8.0.0²,¹¹.0⁴,⁹.0¹⁵,²⁰]henicosa-1(21),2,4,6,8,10,15(20)-heptaen-10-yl]methyl}carbamoyl)methyl]-3-phenylpropanamide (LP22)

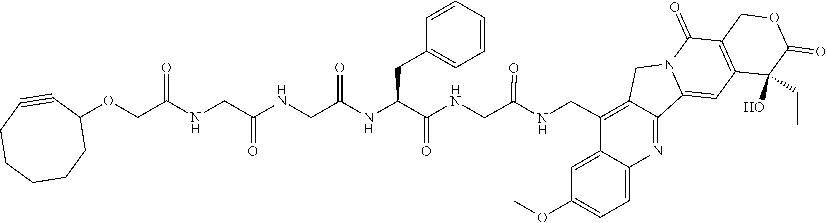 [SMILES: CC[C@@]1(O)C(=O)OCc2c1cc1n(c2=O)Cc2c-1nc1ccc(OC)cc1c2CNC(=O)CNC(=O)[C@H](Cc1ccccc1)NC(=O)CNC(=O)CNC(=O)COC1C#CCCCCC1]

To a solution of compound 7B-2 (crude, 35 mg) obtained above in DMF (3 mL) were added DIPEA (0.03 mL, 0.20 mmol) and compound 7-2 (14 mg, 49 μmol), and the reaction mixture was stirred at room temperature for 1.5 hours, which was monitored by LCMS. The resulting mixture was then directly purified by prep-H PLC (0-100% acetonitrile in aq. TFA (0.01%)) to give linker-payload LP22 (2.0 mg, 5% yield) as an off-white solid. ESI m/z: 890.3 (M+H)⁺.

### Example 8. Synthesis of vcPAB-Carbonate Linker-Payload LP18 (Scheme 8)

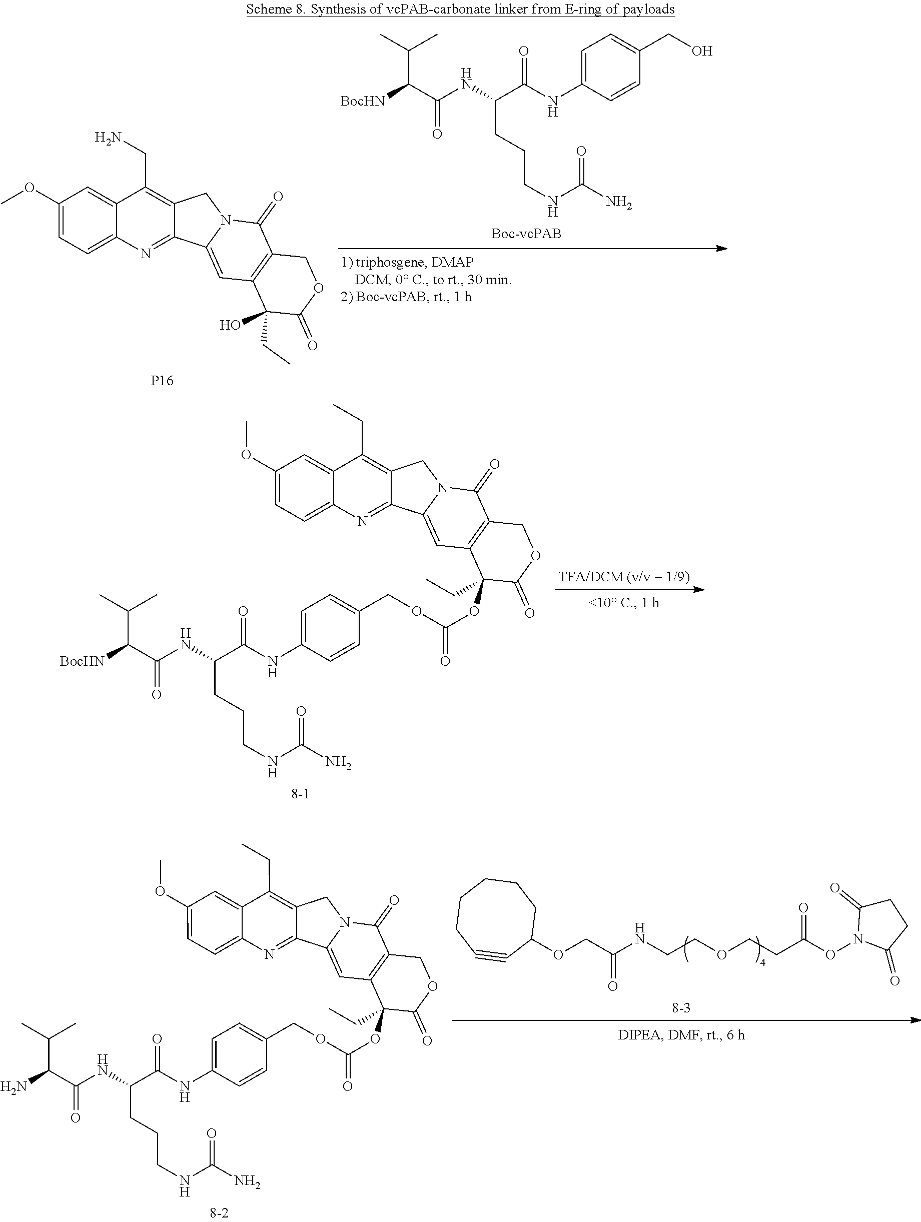

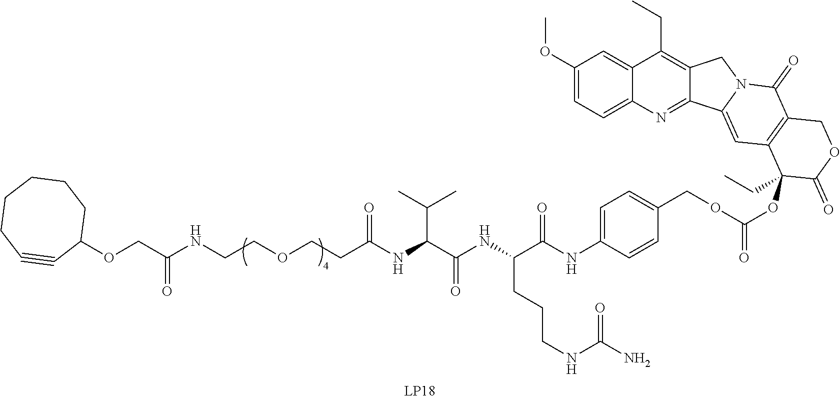

### Example 8A: Synthesis of {4-[(2S)-2-[(2S)-2-{[(tert-Butoxy)carbonyl]amino}-3-methylbutanamido]-5-(carbamoylamino)pentanamido]phenyl}methyl (19S)-10,19-diethyl-7-methoxy-14,18-dioxo-17-oxa-3,13-diazapentacyclo[11.8.0.0²,¹¹.0⁴,⁹.0¹⁵,²⁰]henicosa-1(21),2,4,6,8,10,15(20)-heptaen-19-yl carbonate (8-1)

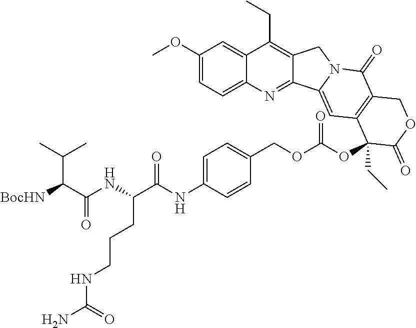 [SMILES: CCc1c2c(nc3ccc(OC)cc13)-c1cc3c(c(=O)n1C2)COC(=O)[C@@]3(CC)OC(=O)OCc1ccc(NC(=O)[C@H](CCCNC(N)=O)NC(=O)[C@@H](NC(=O)OC(C)(C)C)C(C)C)cc1]

A suspension of compound P16 (0.12 g, 0.30 mmol) and DMAP (0.11 g, 0.90 mmol) in dry DCM (18 mL) was cooled in ice-water. To the suspension was added a solution of triphosgene (45 mg, 0.15 mmol) in dry DCM (2 mL) dropwise over 10 minutes. Upon addition, the yellow suspension turned clear. After the reaction solution was stirred at room temperature for 20 minutes until chloroformate intermediate was formed, which was monitored by TLC (Aliquot of reaction solution was quenched with dry methanol and then compared mobility with starting P16 by TLC elenting with MeOH/DCM, v/v=1/15. The methyl carbonate of chloroformate intermediate with R_{f} 0.75, P16 with R_{f} 0.5). To the reaction mixture was then added Boc-vcPAB (0.14 g, 0.30 mmol), and the mixture was stirred at room temperature for 20 minutes, which was monitored by LCMS. The resulting solution was concentrated in vacuo below 30° C. and the residue was purified by reversed phase flash chromatography (0-65% acetonitrile in aq. TFA (0.01%)) to give compound 8-1 (0.15 g, 56% yield) as a white solid. ESI m/z: 812 (M-Boc+H)⁺, 406.7 (fragment, M_{P16}+H)⁺.

### Example 8B: Synthesis of {4-[(2S)-2-[(2S)-2-Amino-3-methylbutanamido]-5-(carbamoylamino)pentanamido]phenyl}methyl (19S)-10,19-diethyl-7-methoxy-14,18-dioxo-17-oxa-3,13-diazapentacyclo[11.8.0.0²,¹¹.0⁴,⁹.0¹⁵,²⁰]henicosa-1(21),2,4,6,8,10,15(20)-heptaen-19-yl carbonate (8-2)

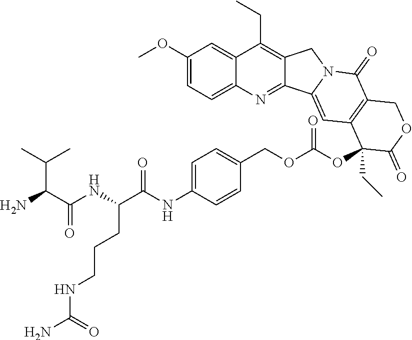 [SMILES: CCc1c2c(nc3ccc(OC)cc13)-c1cc3c(c(=O)n1C2)COC(=O)[C@@]3(CC)OC(=O)OCc1ccc(NC(=O)[C@H](CCCNC(N)=O)NC(=O)[C@@H](N)C(C)C)cc1]

To a cooled (<10° C.) mixture of 8-1 (90 mg, 98 μmol) in DCM (9 mL) was added TFA (1 mL), and the resulting clear yellow solution was stirred for an hour below 10° C. until Boc was removed according to LCMS. The reaction solution was then quenched with sat. aq. sodium bicarbonate to pH 6.0 before the volatiles were removed in vacuo. The residual aq. mixture was purified by reversed phase flash chromatography (0-70% acetonitrile in aq. TFA (0.01%)) to give compound 8-2 (58 mg, 73% yield) as a yellow solid. ESI m/z: 812.3 (M+H)⁺.

### Example 8C: Synthesis of {4-[(2S)-5-(Carbamoylamino)-2-[(2S)-2-{1-[2-(cyclooct-2-yn-1-yloxy)acetamido]-3,6,9,12-tetraoxapentadecan-15-amido}-3-methylbutanamido]pentanamido]phenyl}methyl (19S)-10,19-diethyl-7-methoxy-14,18-dioxo-17-oxa-3,13-diazapentacyclo[11.8.0.0²,¹¹.0⁴,⁹.0¹⁵,²⁰]henicosa-1(21),2,4,6,8,10,15(20)-heptaen-19-yl carbonate (LP18)

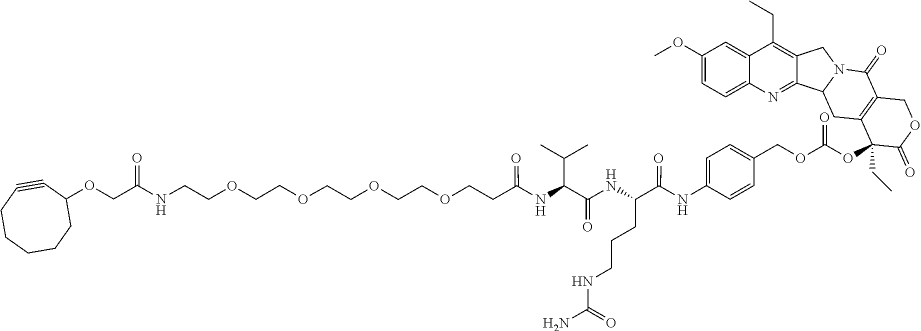 [SMILES: CCc1c2c(nc3ccc(OC)cc13)C1CC3=C(COC(=O)[C@@]3(CC)OC(=O)OCc3ccc(NC(=O)[C@H](CCCNC(N)=O)NC(=O)[C@@H](NC(=O)CCOCCOCCOCCOCCNC(=O)COC4C#CCCCCC4)C(C)C)cc3)C(=O)N1C2]

To a yellow solution of compound 8-2 (20 mg, 25 μmol) in dry DMF (2 mL) were added DIPEA (9.5 mL, 74 μmol) and 8-3 (13 mg, 25 μmol), and the reaction solution was stirred at room temperature for 6 hours until starting material 8-2 was almost consumed, which was monitored by LCMS. The resulting mixture was directly purified by reversed phase flash chromatography (0-70% acetonitrile in aq. TFA (0.01%)) to give linker-payload LP18 (18 mg, 60% yield) as a pale-yellow solid. ESI m/z: 612.5 (M/2+H)⁺. ¹H NMR (400 MHz, DMSO_{d6}) δ 10.03 (s, 1H), 8.14 (d, J=7.2 Hz, 1H), 8.11 (d, J=9.2 Hz, 1H), 7.89 (d, J=8.8 Hz, 1H), 7.60 (d, J=8.4 Hz, 3H), 7.56-7.51 (m, 2H), 7.30 (d, J=8.4 Hz, 2H), 6.97 (s, 1H), 6.00 (br s, 1H), 5.52 (s, 2H), 5.34 (d, J=3.2 Hz, 2H), 5.09 (q, J=12 Hz, 2H), 4.40-4.35 (m, 1H), 4.29-4.22 (m, 2H), 4.00 (s, 3H), 3.87 (d, J=14.8 Hz, 1H), 3.75 (d, J=14.8 Hz, 1H), 3.62-3.57 (m, 2H), 3.50-3.48 (m, 16H), 3.27-3.19 (m, 4H), 3.04-2.91 (m, 2H), 2.46-2.33 (m, 2H), 2.25-2.06 (m, 5H), 2.03-1.66 (m, 6H), 1.61-1.56 (m, 3H), 1.45-1.36 (m, 3H), 1.32 (t, J=7.6 Hz, 3H), 0.92-0.81 (m, 9H) ppm. (The proton of TFA was not observed). ¹⁹F NMR (376 MHz, DMSO_{d6}) δ −73 ppm.

### Example 9. Synthesis of Linker-Payload LP13, LP14, and LP15 (Scheme 9)

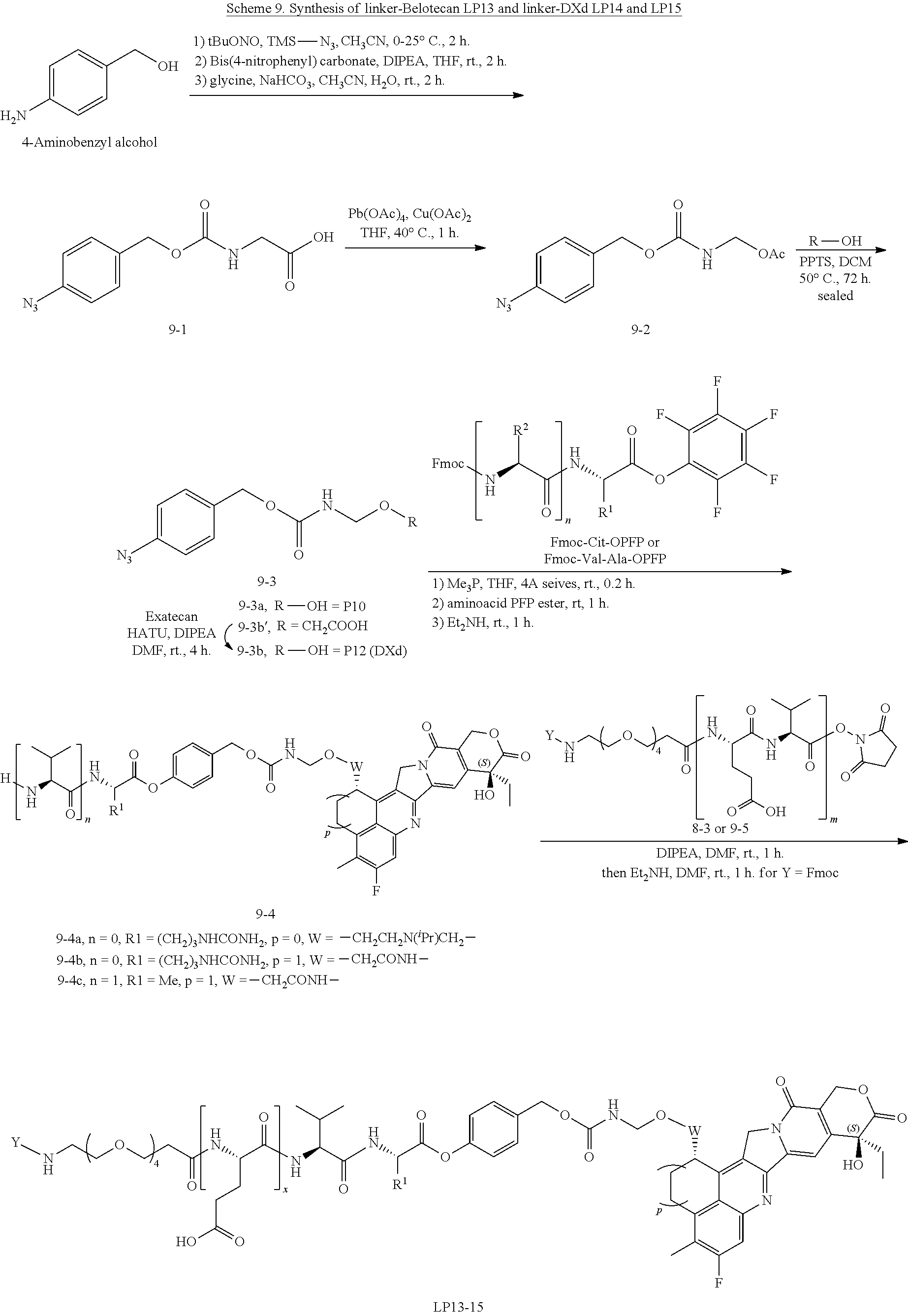

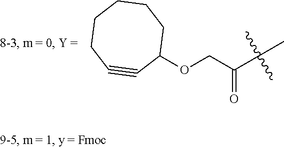 [SMILES: *C(=O)COC1C#CCCCCC1]

### Example 9A-1: Synthesis of 2-({[(4-azidophenyl)methoxy]carbonyl}amino)acetic acid (9-1)

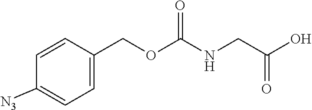 [SMILES: [N-]=[N+]=Nc1ccc(COC(=O)NCC(=O)O)cc1]

To a solution of 4-aminobenzyl alcohol (10 g, 81 mmol) and azido(trimethyl)silane (11 g, 97 mmol, 13 mL) in acetonitrile (600 mL) was added tert-butyl nitrite (13 g, 0.12 mol, 15 mL) dropwise at 0° C. The mixture was stirred at room temperature for 2 hours before the addition of a solution of bis(4-nitrophenyl) carbonate (32 g, 0.11 mol) and DIPEA (21 g, 0.16 mol, 28 mL) in THF (300 mL), and the mixture was stirred at room temperature for 12 hours which was monitored by TLC (25% ethyl acetate in petroleum ether). The volatiles were removed in vacuo and the residue was dissolved in acetonitrile (500 mL). To the solution were added glycine (15 g, 0.20 mol) and aq. sodium bicarbonate (0.8 M, 250 mL, 0.2 mol) dropwise, and the mixture was stirred at room temperature for 16 hours, which was monitored by TLC (25% ethyl acetate in petroleum ether). The resulting mixture was washed with ethyl acetate (150 mL×6). And the aqueous solution was acidified with conc. HCl to pH 2-3, and was then extracted with ethyl acetate (150 mL×3). The combined organic solution was dried over anhydrous sodium sulfate and concentrated in vacuo to give 9-1 (13 g, 61% yield) as a brown solid, which was used in the next step without further purification. ¹H NMR (400 MHz, MeOD_{d4}) δ 7.46-7.34 (m, 2H), 7.12-6.97 (m, 2H), 5.08 (s, 2H), 3.83 (s, 2H) ppm.

### Example 9A-2: ({[(4-azidophenyl)methoxy]carbonyl}amino)methyl acetate (9-2)

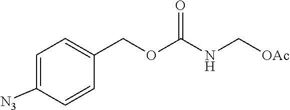 [SMILES: CC(=O)OCNC(=O)OCc1ccc(N=[N+]=[N-])cc1]

To a solution of compound 9-1 (13 g, 51 mmol) in THF (150 mL) were lead acetate (45 g, 0.10 mol) and copper diacetate (0.93 g, 5.1 mmol), and the reaction mixture was stirred at 40° C. for an hour, which was monitored by TLC (25% ethyl acetate in petroleum ether). The reaction was quenched with water (200 mL) and extracted with ethyl acetate (200 mL×2). The combined organic solution was washed with brine (150 mL×2), dried over anhydrous sodium sulfate and concentrated in vacuo. The residue was purified by silica gel flash chromatography (0-25% ethyl acetate in petroleum ether) to give compound 9-2 (7.8 g, 55% yield) as yellow oil. ¹H NMR (400 MHz, CDCl₃) δ 7.36 (br d, J=8.2 Hz, 2H), 7.03 (d, J=8.4 Hz, 2H), 5.93 (br s, 1H), 5.21 (br d, J=7.5 Hz, 2H), 5.11 (s, 2H), 2.07 (s, 3H) ppm.

### Example 9A-3: Synthesis of 2-[({[(4-azidophenyl)methoxy]carbonyl}amino)methoxy]acetic acid (9-3b′)

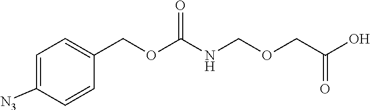 [SMILES: [N-]=[N+]=Nc1ccc(COC(=O)NCOCC(=O)O)cc1]

To a solution of 9-2 (0.20 g, 0.76 mmol) in DCM (3 mL) were added PPTS (38 mg, 0.15 mmol) and hydroxyacetic acid (0.17 g, 2.3 mmol), and the reaction mixture was stirred in a sealed tube at 50° C. for 16 hours, which was monitored by LCMS. The resulting mixture was cooled and the volatiles were removed in vacuo. The residue was purified by prep-HPLC (0-100% acetonitrile in aq. TFA (0.01%)) to give compound 9-3b′ (0.10 g, 47% yield) as a yellow solid. ESI m/z: 303 (M+Na)⁺.

### Example 9A-4: Synthesis of (4-azidophenyl)methyl N-[({[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}methoxy)methyl]carbamate (9-3b)

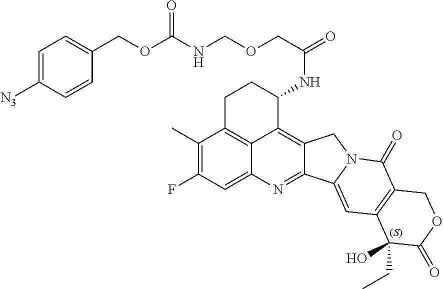

To a yellow solution of compound 9-3b′ (50 mg, 0.13 mmol) and DIPEA (49 mg, 0.38 mmol) in dry DMF (1.5 mL) was added HATU (59 mg, 0.15 mmol), and the mixture was stirred at room temperature for half an hour before the addition of exatecan (60 mg, 0.11 mmol). The reaction mixture was stirred at room temperature for 2 hours, which was monitored by LCMS. The resulting mixture was directly separated by prep-HPLC (5-95% acetonitrile in aq. TFA (0.01%)) to give compound 9-3b (58 mg, 65% yield) as a pale-yellow solid. ESI m/z: 698 (M+H)⁺.

### Example 9A-5: Synthesis of {4-[(2S)-2-[(2S)-2-amino-3-methylbutanamido]propanamido]phenyl}methyl N-[({[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}methoxy)methyl]carbamate (9-4c)

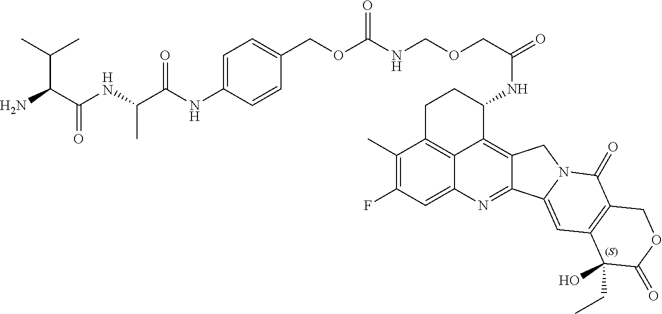

To a 25 mL vial, charged with a stir bar and THF (3 mL), were added 9-3b (42 mg, 52 μmol), 4A molecular sieve (0.50 g) and then trimethylphosphine (0.16 mL, 0.16 mmol). The reaction mixture was stirred for 5 minutes before the addition of Fmoc-Val-Ala-OPFP (33 mg, 57 μmol). The reaction mixture was stirred at room temperature under nitrogen protection for half an hour, which was monitored by LCMS. The resulting mixture was directly separated by prep-HPLC (5-95% acetonitrile in aq. TFA (0.01%)) to give Fmoc-9-4c (53 mg, TFA salt) as a light-yellow solid, which was dissolved in dry DMF (1 mL). To the solution was added diethylamine (0.1 mL). The reaction mixture was stirred at room temperature for an hour until Fmoc was totally removed according to LCMS. The resulting solution was directly separated by prep-HPLC (5-95% acetonitrile in aq. TFA (0.01%)) to give compound 9-4c (28 mg, 57% yield, TFA salt) as a light-yellow solid. ESI m/z: 422 (M/2+H)⁺.

NMR for Fmoc-9-4c: ¹H NMR (400 MHz, DMSO_{d6}) δ 10.03 (s, 1H), 8.48-8.29 (m, 1H), 8.26-8.11 (m, 1H), 7.89 (d, J=7.5 Hz, 2H), 7.79-7.72 (m, 3H), 7.70-7.52 (m, 2H), 7.44-7.41 (m, 3H), 7.34-7.25 (m, 3H), 7.20-7.17 (m, 2H), 6.54 (d, J=9.0 Hz, 1H), 5.59 (s, 2H), 5.42 (s, 2H), 5.21 (s, 1H), 4.87 (s, 1H), 4.54 (d, J=6.5 Hz, 2H), 4.41-4.32 (m, 1H), 4.35-4.17 (m, 3H), 4.00 (s, 1H), 3.98-3.83 (m, 1H), 3.15 (s, 2H), 2.38 (s, 3H), 2.18-2.11 (m, 3H), 2.09-1.95 (m, 2H), 1.93-1.71 (m, 2H), 1.30-1.14 (m, 6H), 0.89-0.77 (m, 9H) ppm. ¹⁹F NMR (376 MHz, DMSO_{d6}) δ −73.45, -111.33 ppm.

NMR for 9-4c: ¹H NMR (400 MHz, DMSO_{d6}) δ 10.20 (br, 1H), 8.84-8.67 (m, 1H), 8.46 (br s, 1H), 8.21 (br s, 2H), 8.06 (s, 2H), 7.80 (d, J=10.5 Hz, 1H), 7.65-7.50 (m, 2H), 7.31 (s, 1H), 7.23 (t, J=8.2 Hz, 1H), 6.53 (s, 1H), 5.60 (s, 1H), 5.42 (s, 2H), 5.22 (s, 1H), 4.88 (d, J=5.8 Hz, 2H), 4.60-4.45 (m, 3H), 4.00 (s, 1H), 3.61 (br s, 1H), 3.17 (br s, 1H), 2.93-2.90 (m, 2H), 2.39 (s, 3H), 2.33-2.06 (m, 2H), 1.93-1.76 (m, 2H), 1.34 (t, J=7.4 Hz, 3H), 1.16 (t, J=7.3 Hz, 3H), 0.97-0.93 (m, 6H), 0.85 (t, J=7.4 Hz, 3H) ppm. ¹⁹F NMR (376 MHz, DMSO_{d6}) δ −73.44, −111.33 ppm.

### Example 9A-6: Synthesis of {4-[(2S)-2-[(2S)-2-{1-[2-(cyclooct-2-yn-1-yloxy)acetamido]-3,6,9,12-tetraoxapentadecan-15-amido}-3-methylbutanamido]propanamido]phenyl}methyl N-[({[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}methoxy)methyl]carbamate (LP15)

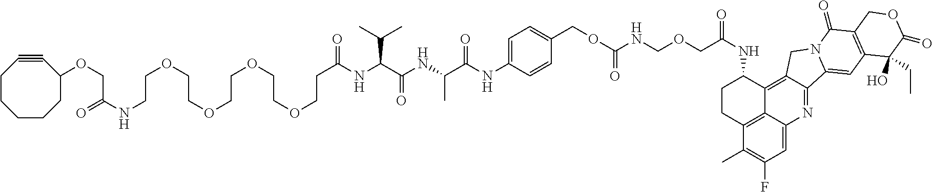 [SMILES: CC[C@@]1(O)C(=O)OCc2c1cc1n(c2=O)Cc2c-1nc1cc(F)c(C)c3c1c2[C@@H](NC(=O)COCNC(=O)OCc1ccc(NC(=O)[C@H](C)NC(=O)[C@@H](NC(=O)CCOCCOCCOCCOCCNC(=O)COC2C#CCCCCC2)C(C)C)cc1)CC3]

To a yellow solution of compound 8-3 (14 mg, 27 μmol) in dry DMF (1.5 mL) were added DIPEA (14 mg, 0.11 mmol) and compound 9-4c (23 mg, 24 μmol), and the reaction mixture was stirred at room temperature for an hour, which was monitored by LCMS. The resulting mixture was directly separated by prep-HPLC (5-95% acetonitrile in aq. formic acid (0.1%)) to give linker-payload LP15 (9.0 mg, 26% yield) as a white solid. ESI m/z: 1253 (M+H)⁺. ¹H NMR (400 MHz, DMSO_{d6}) δ 9.96 (s, 1H), 8.45 (m, 1H), 8.21 (m, 1H), 7.89 (d, J=8.6 Hz, 1H), 7.79 (d, J=10.9 Hz, 1H), 7.66-7.52 (m, 3H), 7.30 (s, 1H), 7.25-7.19 (m, 2H), 6.53 (s, 1H), 5.59 (s, 1H), 5.42 (s, 2H), 5.22 (s, 2H), 4.86 (s, 2H), 4.54 (d, J=6.7 Hz, 2H), 4.45-4.30 (m, 1H), 4.29-4.08 (m, 2H), 4.00 (s, 2H), 3.87 (d, J=14.8 Hz, 1H), 3.75 (d, J=14.7 Hz, 1H), 3.58 (d, J=6.2 Hz, 2H), 3.51-3.45 (m, 12H), 3.44-3.40 (m, 2H), 3.29-3.21 (m, 2H), 3.16 (br s, 2H), 2.45-2.36 (m, 5H), 2.24-2.02 (m, 5H), 2.02-1.70 (m, 7H), 1.61-1.52 (m, 2H), 1.44-1.22 (m, 5H), 0.92-0.77 (m, 9H) ppm. ¹⁹F NMR (376 MHz, DMSO_{d6}) δ −111.33 ppm.

### Example 9B-1: Synthesis of {4-[(2S)-2-amino-5-(carbamoylamino)pentanamido]phenyl}methyl N-[({[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}methoxy)methyl]carbamate (9-4b)

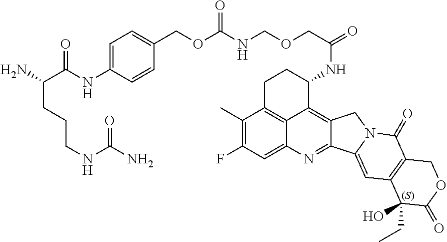

Following the similar procedure as 9-4c except using Fmoc-Cit-OPFP instead of Fmoc-Val-Ala-OPFP, compound 9-4b (41 mg, 50% yield) was obtained as a light-yellow solid. ESI m/z: 829 (M+H).

### Example 9B-2: Synthesis of (4S)-4-{[(2S)-1-[(2,5-dioxopyrrolidin-1-yl)oxy]-3-methyl-1-oxobutan-2-yl]carbamoyl}-4-[1-({[(9H-fluoren-9-yl)methoxy]carbonyl}amino)-3,6,9,12-tetraoxapentadecan-15-amido]butanoic acid (9-5)

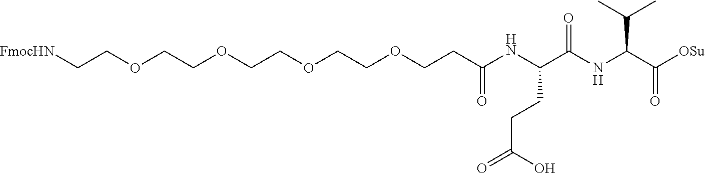

To a solution of H-Glu(O^{t}Bu)-Val-OH (29 g, 56 mmol) in DMF (200 mL) were added a solution of Fmoc-PEG₄-OSu (31 g, 53 mmol) in DCM (200 mL) and DIPEA (7.2 g, 56 mmol, 9.7 mL), and the reaction mixture was stirred at room temperature for 2 hours, which was monitored by LCMS. The volatiles was then removed in vacuo. The residue was diluted with water (100 mL), washed with MTBE (80 mL×3) and acidified with citric acid to pH 5. The mixture was extracted with ethyl acetate (120 mL×2) and the combined organic solution was washed with brine (60 mL×2), dried over anhydrous sodium sulfate and concentrated in vacuo to give Fmoc-PEG₄-Glu(O^{t}Bu)-Val-OH (33 g) as yellow oil. ESI m/z: 772 (M+H)⁺.

To a solution of HOSu (7.9 g, 68 mmol) in DMF (150 mL) and DCM (150 mL) were added DIC (6.5 g, 51 mmol) and Fmoc-PEG₄-Glu(O^{t}Bu)-Val-OH (33 g) obtained above. The reaction mixture was stirred at room temperature for 12 hours, which was monitored by TLC and LCMS. The resulting mixture was filtered and the filtrate was concentrated in vacuo. The residue was diluted with water (200 mL) and extracted with ethyl acetate (150 mL×3). The combined organic solution was washed with brine (150 mL×3), dried over anhydrous sodium sulfate and concentrated in vacuo to give Fmoc-PEG₄-Glu(O^{t}Bu)-Val-OSu (14 g) as yellow oil. ESI m/z 870 (M+H)⁺. ¹H NMR (400 MHz, CDCl₃) δ7.76 (d, J=7.5 Hz, 2H), 7.64-7.58 (m, 2H), 7.42-7.37 (m, 2H), 7.34-7.28 (m, 2H), 4.86-4.76 (m, 1H), 4.56-4.34 (m, 3H), 4.28-4.18 (m, 1H), 3.76-3.73 (m, 3H), 3.66-3.56 (m, 13H), 3.39 (br d, J=4.9 Hz, 1H), 2.81 (br s, 6H), 2.50-2.46 (m, 2H), 2.40-2.32 (m, 2H), 2.14-2.04 (m 1H), 1.97-1.81 (m, 4H), 1.44 (s, 9H), 1.05 (s, 3H), 1.04 (s, 3H) ppm.

To a solution of Fmoc-PEG₄-Glu(O^{t}Bu)-Val-OSu (0.14 g, 0.16 mmol, obtained above) in DCM (0.5 mL) was added TFA (0.72 g, 0.47 mL, 6.3 mmol), and the reaction mixture was stirred at room temperature for an hour, which was monitored by LCMS. The volatiles were removed in vacuo and the residue was triturated in MTBE (10 mL×2). The off-white precipitates were collected by centrifuge to give compound 9-5 (Fmoc-PEG4-Glu-Ala-OSu) (0.13 g, 22% yield from H-Glu(O^{t}Bu)-Val-OH) as an off-white solid. ESI m/z: 813 (M+H)⁺.

### Example 9B-3: Synthesis of (4S)-4-(1-amino-3,6,9,12-tetraoxapentadecan-15-amido)-4-{[(1S)-1-{[(1S)-4-(carbamoylamino)-1-({4-[({[({[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}methoxy)methyl]carbamoyl}oxy)methyl]phenyl}carbamoyl)butyl]carbamoyl}-2-methylpropyl]carbamoyl}butanoic acid (9-6b)

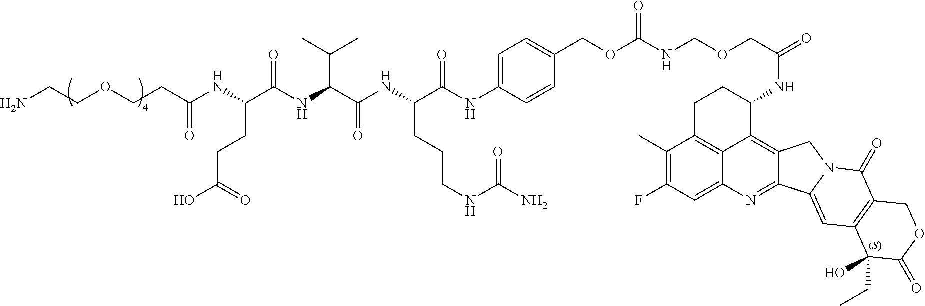

Following the similar procedure as LP15 except using compound 9-5 instead of intermediate 8-3 and 9-4b instead of 9-4c Fmoc-9-6b (26 mg) was obtained as a light-yellow solid, which was dissolved in DMF (2 mL). To the solution was added diethylamine (0.2 mL) and the reaction mixture was stirred at room temperature for an hour until Fmoc was totally removed according to LCMS. The mixture was directly separated by prep-HPLC (5-95% acetonitrile in aq. TFA (0.01%)) to give 9-6b (13 mg, 20% yield) as a white solid. ESI m/z: 653 (M+H)⁺.

### Example 9B-4: Synthesis of (4S)-4-{[(1S)-1-{[(1S)-4-(carbamoylamino)-1-({4-[({[({[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}methoxy)methyl]carbamoyl}oxy)methyl]phenyl}carbamoyl)butyl]carbamoyl}-2-methylpropyl]carbamoyl}-4-{1-[2-(cyclooct-2-yn-1-yloxy)acetamido]-3,6,9,12-tetraoxapentadecan-15-amido}butanoic acid (LP14)

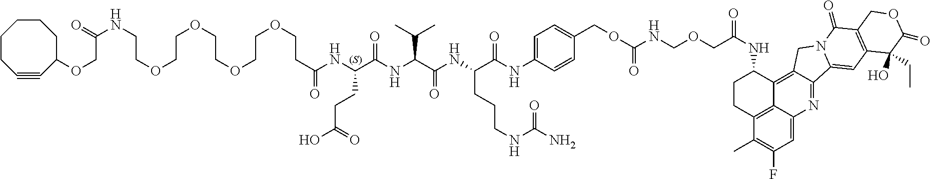

To a yellow solution of compound 7-2 (3.3 mg, 12 μmol) in dry DMF (1 mL) were added DIPEA (2.6 mg, 20 μmol) and compound 9-6b (13 mg, 10 μmol), and the reaction mixture was stirred at room temperature for an hour, which was monitored by LCMS. The resulting mixture was directly separated by prep-HPLC (5-95% acetonitrile in aq. formic acid (0.1%)) to give linker-payload LP14 (4.5 mg, 31% yield) as a white solid. ESI m/z: 735 (M/2+H)⁺.

### Example 9C-1: Synthesis of (4S)-4-{[(1 S)-1-{[(1S)-4-(carbamoylamino)-1-[(4-{[({[2-({2-[(19S)-19-ethyl-19-hydroxy-14,18-dioxo-17-oxa-3,13-diazapentacyclo[11.8.0.0²,¹¹.0⁴,⁹.0¹⁵,²⁰]henicosa-1(21),2,4,6,8,10,15(20)-heptaen-10-yl]ethyl}(propan-2-yl)amino)ethoxy]methyl}carbamoyl)oxy]methyl}phenyl)carbamoyl]butyl]carbamoyl}-2-methylpropyl]carbamoyl}-4-{1-[2-(cyclooct-2-yn-1-yloxy)acetamido]-3,6,9,12-tetraoxapentadecan-15-amido}butanoic acid (LP13)

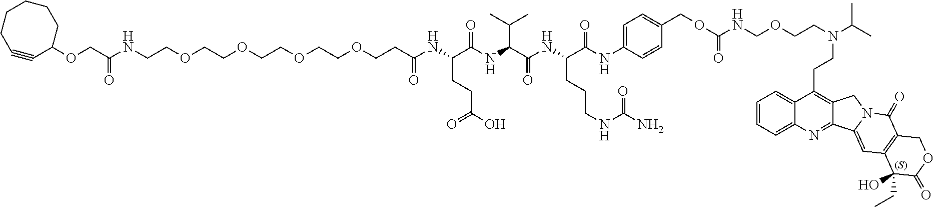

Following the similar procedures as LP14 except using P10 instead of hydroxyacetic acid, linker-payload LP13 (8 mg, 1% yield in 4 steps from P10) was obtained as an off-white solid. ESI m/z: 727.3 (M/2+H)⁺.

### Example 10. Synthesis of Branched Sugar-PAB Linker-Payload (Scheme 10)

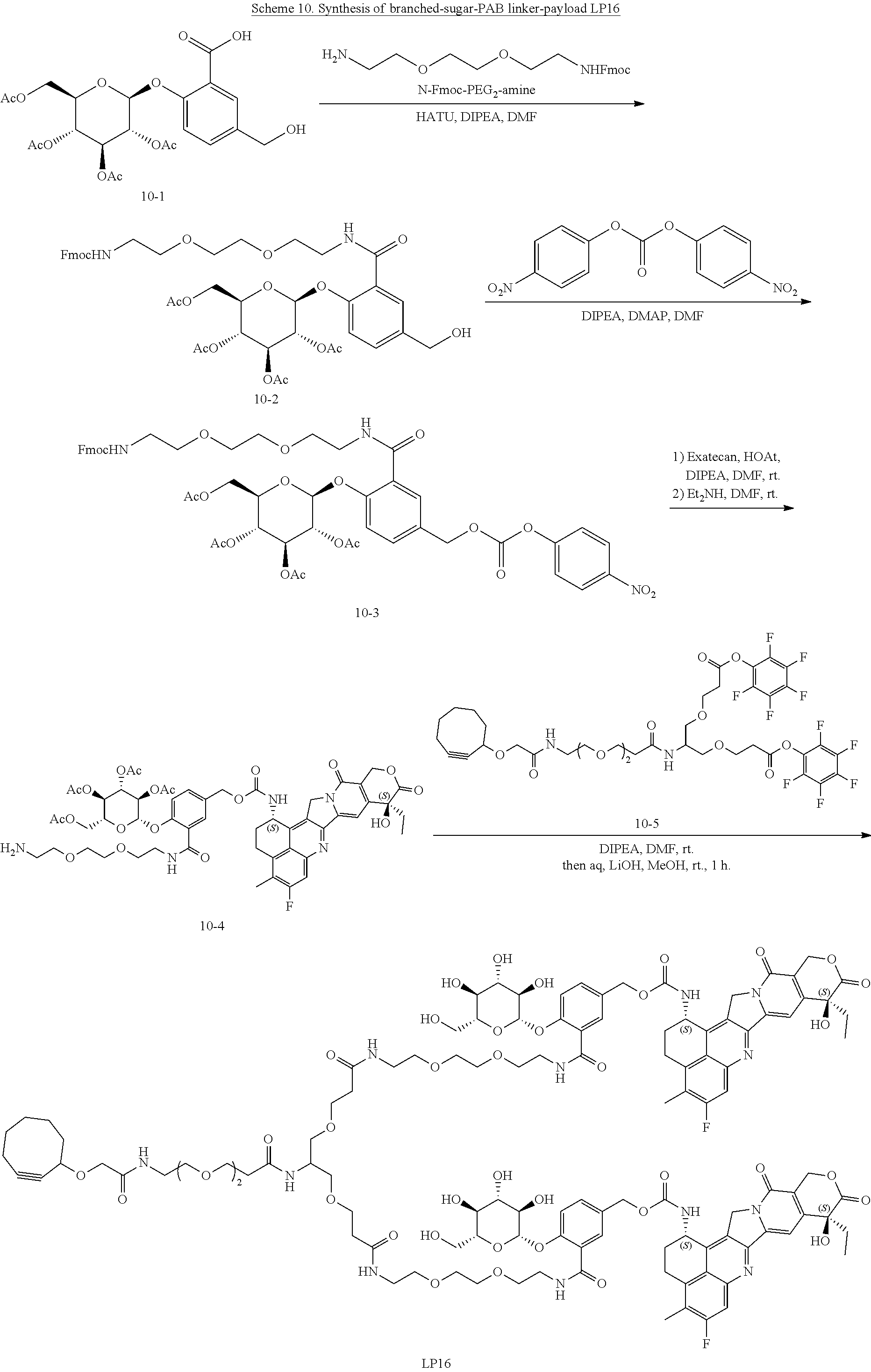

### Example 10A: Synthesis of [(2R,3R,4S,5R,6S)-3,4,5-tris(acetyloxy)-6-{2-[(2-{2-[2-({[(9H-fluoren-9-yl)methoxy]carbonyl}amino)ethoxy]ethoxy}ethyl)carbamoyl]-4-(hydroxymethyl)phenoxy}oxan-2-yl]methyl acetate (10-2)

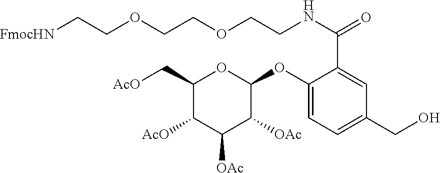

To a solution of compound 10-1 (0.10 g, 0.2 mmol) in DMF (5 mL) were added N-Fmoc-PEG₂-amine (74 mg, 0.2 mmol) and DIPEA (52 mg, 0.4 mmol), and the reaction mixture was stirred at room temperature for 10 minutes before HATU was added into the reaction. The mixture was stirred at room temperature for 4 hours, which was monitored by LCMS. The resulting mixture was purified by prep-HPLC (5-95% acetonitrile in aq. TFA (0.01%)) to give compound 10-2 (0.14 g, 81% yield) as a white solid. ESI m/z: 851.3 (M+H)⁺.

### Example 10B: Synthesis of [(2R,3R,4S,5R,6S)-3,4,5-tris(acetyloxy)-6-{2-[(2-{2-[2-({[(9H-fluoren-9-yl)methoxy]carbonyl}amino)ethoxy]ethoxy}ethyl)carbamoyl]-4-({[(4-nitrophenoxy)carbonyl]oxy}methyl)phenoxy}oxan-2-yl]methyl acetate (10-3)

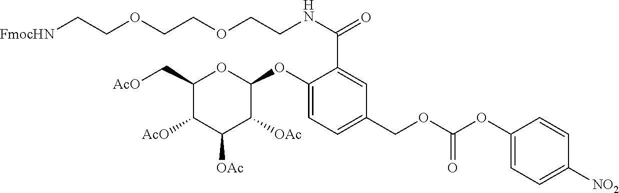

To a solution of 10-2 (0.14 g, 0.16 mmol) and bis(4-nitrophenyl)carbonate (0.25 g, 0.82 mmol) in DMF (5 mL) were added DIPEA (0.11 g, 0.82 mmol) and DMAP (20 mg, 0.16 mmol), and the reaction mixture was stirred at room temperature for 4 hours, which was monitored by LCMS. The resulting mixture was separated by prep-HPLC (5-95% acetonitrile in aq. TFA (0.01%)) to give compound 10-3 (0.13 g, 75% yield) as a white solid. ESI m/z: 1017.2 (M+H)⁺.

### Example 10C: Synthesis of [(2R,3R,4S,5R,6S)-3,4,5-tris(acetyloxy)-6-[2-({2-[2-(2-aminoethoxy)ethoxy]ethyl}carbamoyl)-4-[({[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}oxy)methyl]phenoxy]oxan-2-yl]methyl acetate (10-4)

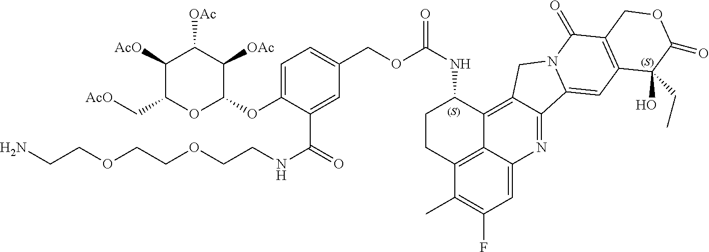

To a solution of 10-3 (0.10 g, 98 μmol) in DMF (5 mL) were added exatecan (43 mg, 98 μmol), HOAt (7 mg, 49 μmol) and DIPEA (26 mg, 0.20 mmol), and the reaction mixture was stirred at room temperature for 4 hours, which was monitored by LCMS. The resulting mixture was purified by prep-HPLC (5-95% acetonitrile in aq. TFA (0.01%)) to give compound Fmoc-10-4 (0.11 g, 86% yield, ESI m/z: 1318.3 (M+H)⁺) as a white solid, which was dissolved in DMF (5 mL). To the solution was added DIPEA (13 mg, 0.17 mmol), and the reaction mixture was stirred at room temperature for an hour. The resulting mixture was separated by reversed phase flash chromatography (85 mg, 78% yield) as a white solid. ESI m/z: 1091.3 (M+H)⁺.

### Example 10D: Synthesis of {3-[(2-{2-[2-(3-{2-[3-(2-{2-[2-(cyclooct-2-yn-1-yloxy)acetamido]ethoxy}ethoxy)propanamido]-3-{2-[(2-{2-[2-({5-[({[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}oxy)methyl]-2-{[(2S,3R,4S,5S,6R)-3,4,5-trihydroxy-6-(hydroxymethyl)oxan-2-yl]oxy}phenyl}formamido)ethoxy]ethoxy}ethyl)carbamoyl]ethoxy}propoxy}propanamido)ethoxy]ethoxy}ethyl)carbamoyl]-4-{[(2S,3R,4S,5S,6R)-3,4,5-trihydroxy-6-(hydroxymethyl)oxan-2-yl]oxy}phenyl}methyl N-[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamate (LP16)

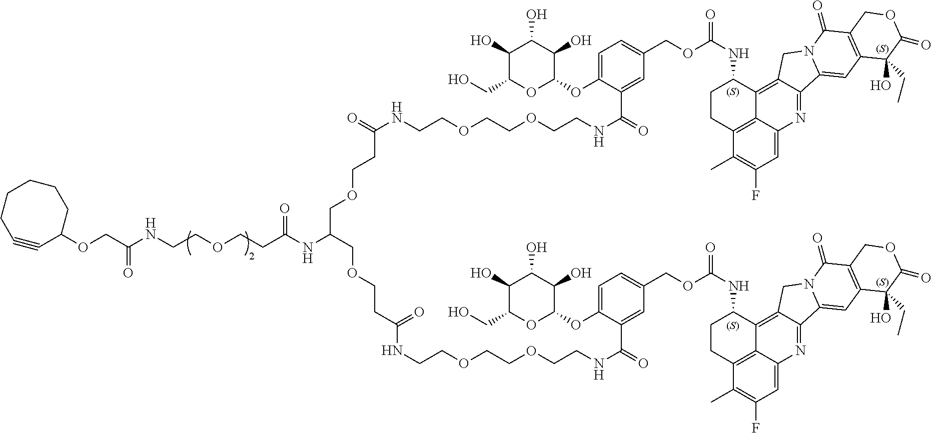

To a stirred mixture of 10-4 (85 mg, 78 μmol) and 10-5 (35 mg, 39 μmol) in DMF (5 mL) was added DIPEA (20 mg, 0.16 mmol), and the mixture was stirred at room temperature for an hour, which was monitored by LCMS. The resulting mixture was purified by reversed phase flash chromatography (0-100% acetonitrile in aq. TFA (0.01%)) to give a white solid, which was dissolved in methanol (5 mL). To the solution was added aq. lithium hydroxide (36 mM, 5 mL). The reaction mixture was stirred at room temperature for 2 hours, which was monitored by LCMS. The resulting mixture was directly separated by reversed phase flash chromatography (5-100% acetonitrile in aq. TFA (0.01%)) to give LP16 (30 mg, 33% yield) as a white solid. ESI m/z: 789.2 (M/3+H)⁺.

### Example 11. Synthesis of Branched vcPAB Linker-Payload (Scheme 11)

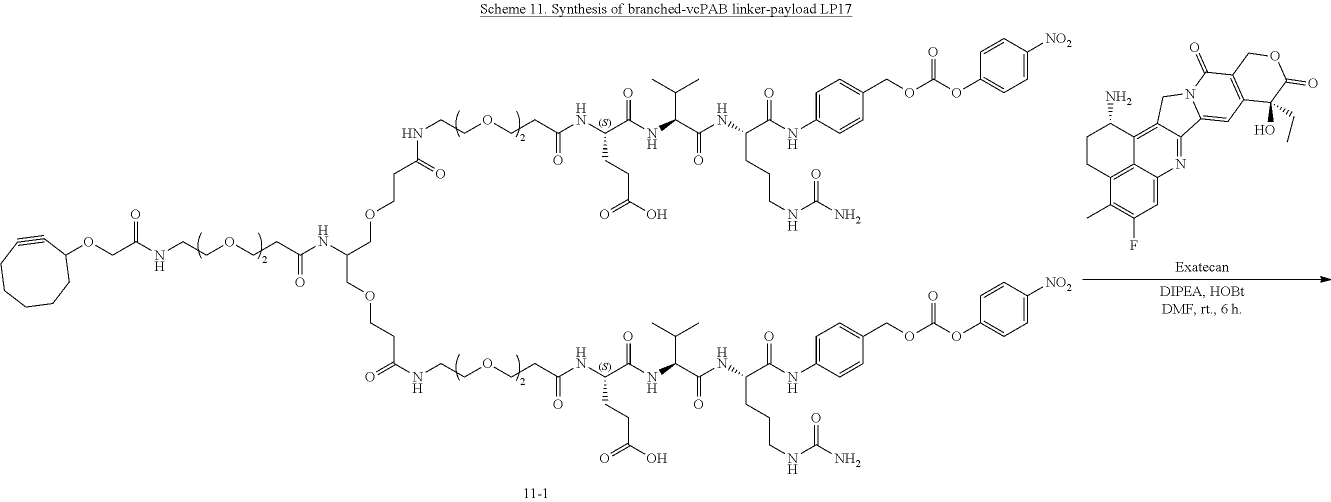

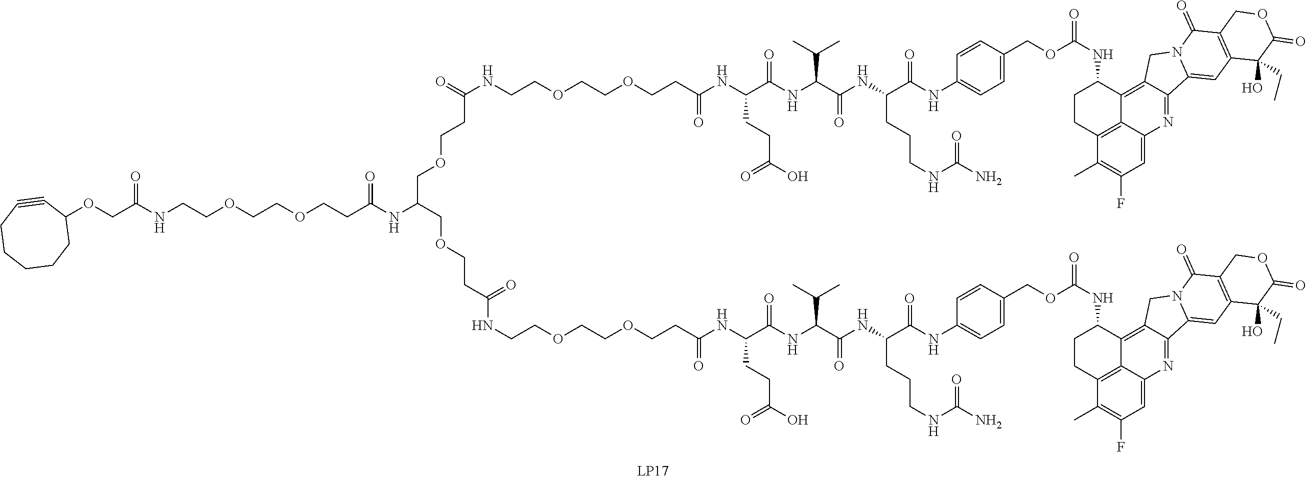 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)[C@H](CCC(=O)O)NC(=O)CCOCCOCCNC(=O)CCOCC(COCCC(=O)NCCOCCOCCC(=O)N[C@@H](CCC(=O)O)C(=O)N[C@H](C(=O)N[C@@H](CCCNC(N)=O)C(=O)Nc2ccc(COC(=O)N[C@H]4CCc5c(C)c(F)cc6nc7c(c4c56)Cn4c-7cc5c(c4=O)COC(=O)[C@]5(O)CC)cc2)C(C)C)NC(=O)CCOCCOCCNC(=O)COC2C#CCCCCC2)C(C)C)cc1)CC3]

### Example 11A: Synthesis of (4S)-4-{[(1S)-1-{[(1S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}oxy)methyl]phenyl}carbamoyl)butyl]carbamoyl}-2-methylpropyl]carbamoyl}-4-(3-{2-[2-(3-{3-[2-({2-[2-(2-{[(1 S)-1-{[(1 S)-1-{[(1 S)-4-(carbamoylamino)-1-({4-[({[(10S,23S)-10-ethyl-18-fluoro-10-hydroxy-19-methyl-5,9-dioxo-8-oxa-4,15-diazahexacyclo[14.7.1.0²,¹⁴.0⁴,¹³.0⁶,¹¹.0²⁰,²⁴]tetracosa-1,6(11),12,14,16,18,20(24)-heptaen-23-yl]carbamoyl}oxy)methyl] phenyl}carbamoyl) butyl] carbamoyl}-2-methyl propyl]carbamoyl}-3-carboxypropyl]carbamoyl}ethoxy)ethoxy]ethyl}carbamoyl)ethoxy]-2-[3-(2-{2-[2-(cyclooct-2-yn-1-yloxy)acetamido]ethoxy}ethoxy)propanamido]propoxy}propanamido)ethoxy]ethoxy}propanamido)butanoic acid (LP17)

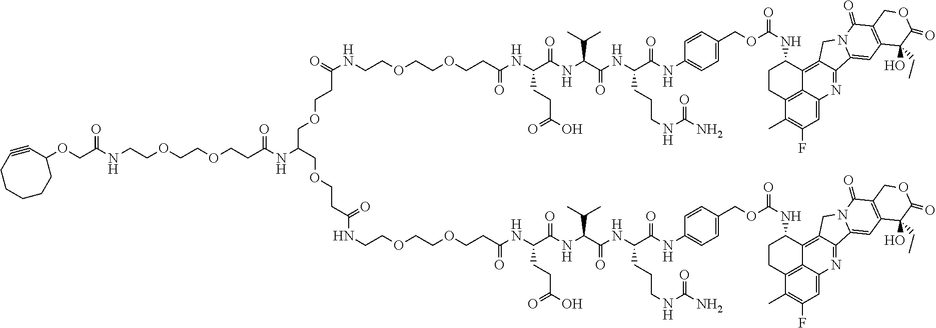 [SMILES: CC[C@@]1(O)C(=O)OCc2c1cc1n(c2=O)Cc2c-1nc1cc(F)c(C)c3c1c2[C@@H](NC(=O)OCc1ccc(NC(=O)[C@H](CCCNC(N)=O)NC(=O)[C@@H](NC(=O)[C@H](CCC(=O)O)NC(=O)CCOCCOCCNC(=O)CCOCC(COCCC(=O)NCCOCCOCCC(=O)N[C@@H](CCC(=O)O)C(=O)N[C@H](C(=O)N[C@@H](CCCNC(N)=O)C(=O)Nc2ccc(COC(=O)N[C@H]4CCc5c(C)c(F)cc6nc7c(c4c56)Cn4c-7cc5c(c4=O)COC(=O)[C@]5(O)CC)cc2)C(C)C)NC(=O)CCOCCOCCNC(=O)COC2C#CCCCCC2)C(C)C)cc1)CC3]

To a yellow solution of 11-1 (50 mg, 23 μmol) in DMF (1.5 mL) were added Exatecan (12 mg, 23 μmol), HOBt (3.1 mg, 23 μmol) and DIPEA (8.9 mg, 69 μmol), and the reaction mixture was stirred at room temperature for 6 hours. The resulting mixture was purified by prep-HPLC (0-100% acetonitrile in aq. TFA (0.01%)) to give LP17 (14 mg, 22% yield) as white solid. ESI m/z: 928 (M/3+H)⁺. ¹H NMR (400 MHz, DMSO_{d6}) δ 10.06 (s, 2H), 8.28 (br s, 2H), 8.14 (d, J=7.2 Hz, 2H), 8.06 (d, J=8.4 Hz, 2H), 7.95 (t, J=5.2 Hz, 2H), 7.84-7.74 (m, 5H), 7.64-7.57 (m, 5H), 7.35 (d, J=8.4 Hz, 2H), 7.30 (s, 2H), 6.52 (s, 2H), 6.22-6.09 (m, 2H), 5.56-5.47 (m, 4H), 5.44 (s, 4H), 5.33-5.21 (m, 6H), 5.07 (s, 4H), 4.42-4.23 (m, 6H, 4.22-4.15 (m, 2H), 3.96-3.90 (m, 1H), 3.88-3.84 (m, 1H), 3.80-3.75 (m, 1H), 3.62-3.53 (m, 11H), 3.47 (br s, 14H), 3.41-3.38 (m, 6H), 3.26-3.15 (m, 10H), 3.13-3.07 (m, 2H), 3.03-2.91 (m, 4H), 2.40 (br s, 6H), 2.35-2.29 (m, 9H), 2.23-2.10 (m, 10H), 2.03-1.95 (m, 2H), 1.93-1.80 (m, 8H), 1.76-1.55 (m, 11H), 1.48-1.33 (m, 6H), 1.07-0.99 (m, 1H), 0.90-0.80 (m, 18H) ppm.

### Example 12. Generic Procedures for Making Site-Specific Conjugates

This example demonstrates a method for site-specific conjugation according to an embodiment of the disclosure, generally, of a payload to an antibody or an antigen-binding fragment thereof. This method includes a two-step process shown in FIG. 1. The first step is microbial transglutaminase (MTG) mediated attachment of an amine linker azide, such as azide-PEG₃-amine (AL, amino-PEG4-azido), to the antibody, wherein an excess of the amine reagent (AL) was used to avoid potential cross-linking of antibody chains. The second step attached the alkyne-linked payload linker payload (LP) to the N₃-tagged conjugate (Ab-AL₄) via a strain-promoted azide-alkyne cycloaddition (SPAAC). The number of LP molecules added to the antibody is dependent on the number of conjugation sites and the number of azide functional groups within AL (n=1). For antibodies with a WT Fc domain that were enzymatically deglycosylated or have an N297D Fc mutation and then azido functionalized with AL linkers, the expected DAR=2 times n times m, where n is the number of azide functional groups on AL, and m is the number of LP payloads, respectively. For antibodies with an N297Q Fc mutation then azido functionalized with AL linkers, the expected OAR=4. All parental antibody (Ab), azido-functionalized antibody containing 2, 4 or 8 azido groups (Ab-(N₃)n), final ADCs generated as specific examples and the corresponding linker-payload (LP), as well as their ES-MS results and OAR values of the ADCs are summarized in Tables 4 and 5.

TABLE 4
List of Site-specific ADCs

| | | Modification | | |
|---|---|---|---|---|
| Antibody | Site of | | MW | DAR by |
| Description | Conjugation | AL, BL, LP # | (g/mol) | ESI-MS |
| Anti-HER2 (degly) | | None | NA | NA |
| Anti-HER2 | Q295, Q297 | [AL]₄ | 218.3 | 4 |
| Anti-HER2 | Q295, Q297 | [AL-LP1]₄ | 1469.7 | 4 |
| Anti-HER2 | Q295, Q297 | [AL-LP2]₄ | 1593.8 | 4 |
| Anti-HER2 | Q295, Q297 | [AL-LP3]₄ | 1230.3 | 4 |
| Anti-HER2 | Q295, Q297 | [AL-LP4]₄ | 1216.3 | 4 |
| Anti-HER2 | Q295, Q297 | [AL-LP8]₄ | 1046.2 | 4 |
| Anti-HER2 | Q295, Q297 | [AL-LP9]₄ | 1614.8 | 4 |
| Isotype Control | | None | NA | NA |
| Isotype Control | Q295, Q297 | [AL]₄ | 218.3 | 4 |
| Isotype Control | Q295, Q297 | [AL-LP3]₄ | 1230.3 | 4 |
| Isotype Control | Q295, Q297 | [AL-LP8]₄ | 1046.2 | 4 |
| Isotype Control | Q295, Q297 | [AL-LP9]₄ | 1614.8 | 4 |

TABLE 5
The structures and DAR vlues of site-specific ADCs

| Anti- | | | |
|---|---|---|---|
| body | ADC | ADC Structure | DAR |
| Anti- HER2 | Ab[AL- LP1]₄ | 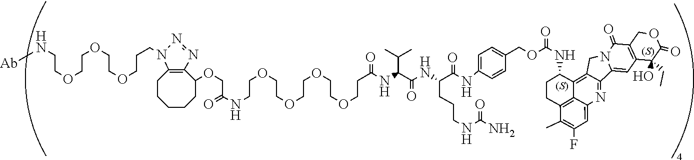 | 3.58 |
| Anti- HER2 | Ab[AL- LP2]₄ | 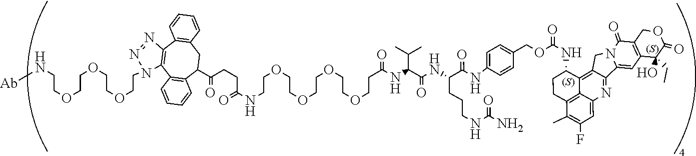 | 4 |
| Anti- HER2 | Ab[AL- LP3]₄ | 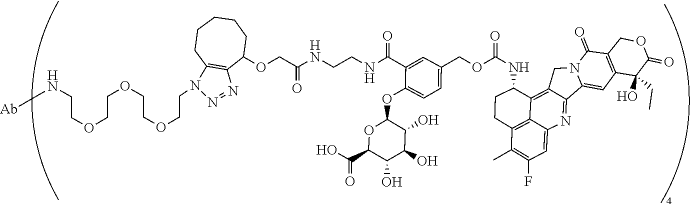 | 4 |
| Anti- HER2 | Ab[AL- LP4]₄ | 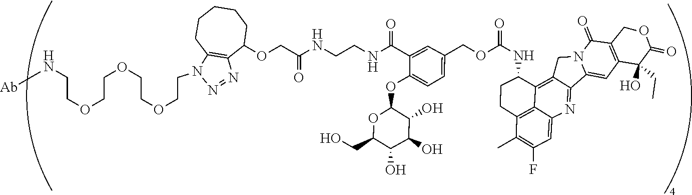 | 4 |
| Anti- HER2 | Ab[AL- LP8]₄ | 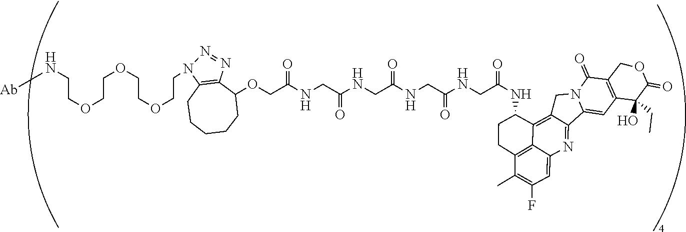 | 4 |
| Anti- HER2 | Ab[AL- LP9]₄ | 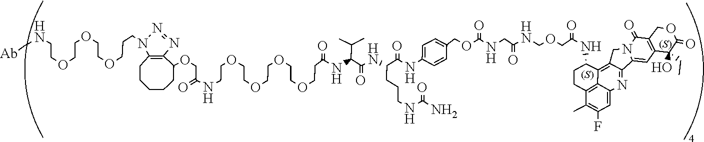 | 4 |
| Isotype Control | Ab[AL- LP3]₄ | 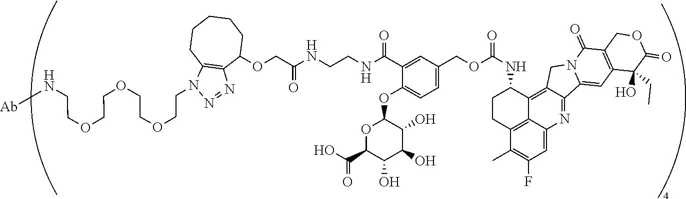 | 4 |
| Isotype Control | Ab[AL- LP8]₄ | 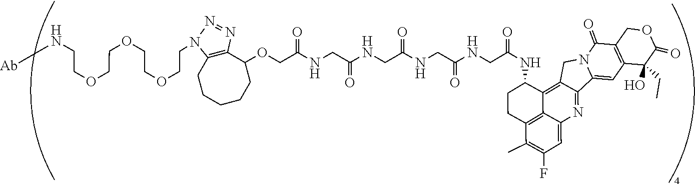 | 4 |
| Isotype Control | Ab[AL- LP9]₄ | 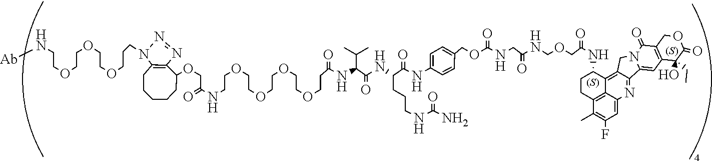 | 4 |
| Anti- HER2 | Ab[AL2- LP1]₄ | 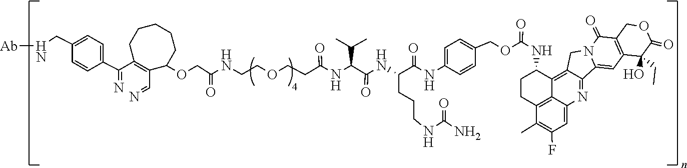 | |
| Anti- HER2 | Ab[AL2- LP17]n | 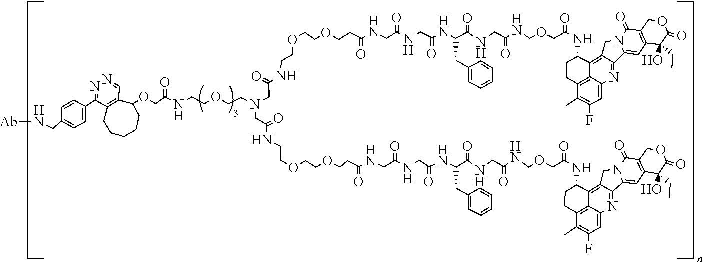 | |
| Isotype Control | Ab[AL2- LP17]n | 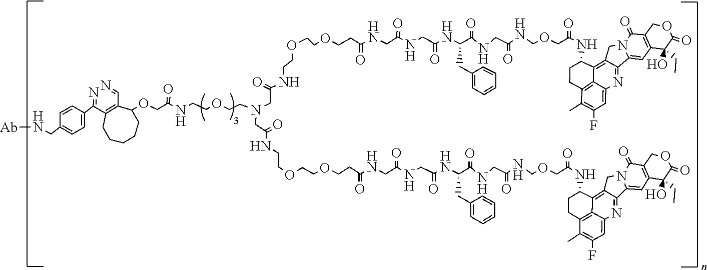 | |

Aglycosylated human antibody IgG (IgG1, IgG4, etc.) containing an N297Q or N297D mutation were used in ADO conjugations. Two Approaches (I & II) were conducted via two-step process (FIG. 1), and the conjugation results with the MS-OAR values were summarized in ADO list (Table 7).

TABLE 7
ADC list

| | DAR | |
|---|---|---|
| ADC# | (n) | Structures |
| aHer2 | | |
| Ab | | |
| Isotype | | |
| Control | | |
| Ab | | |
| aHer2- (Handle) n Isotype Control - | 4 4 | 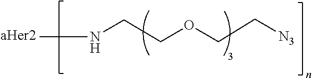 |
| (Handle) | | |
| n | | |
| aHer2- ADC | 6.2 | 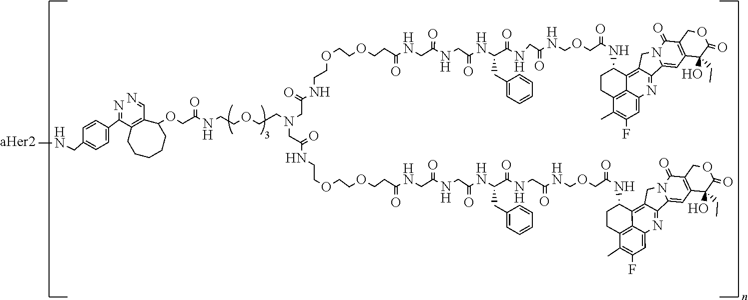 |
| aHer2- ADC | 3.3 | 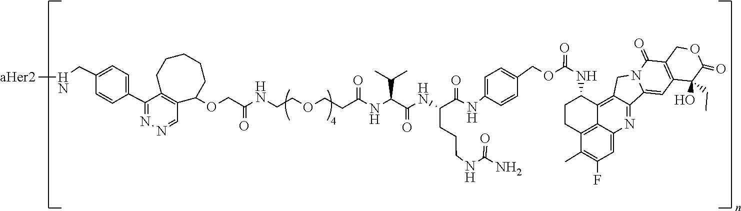 |
| aHer2- ADC Isotype Control - ADC | 7.6 7.0 | 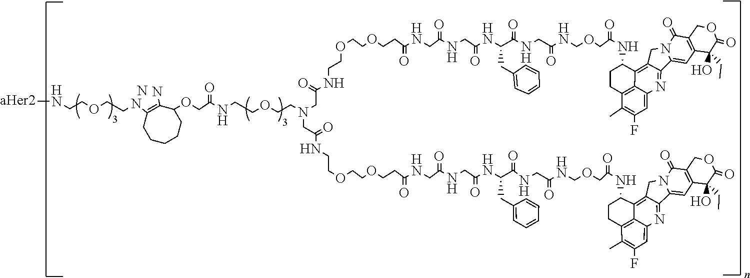 |
| aHer2- ADC | 3.59 | 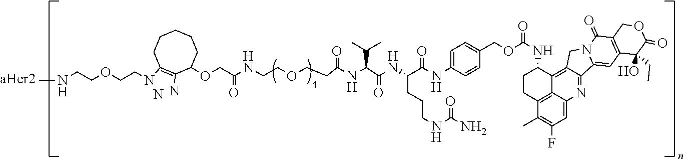 |
| aHer2- ADC | 3.58 | 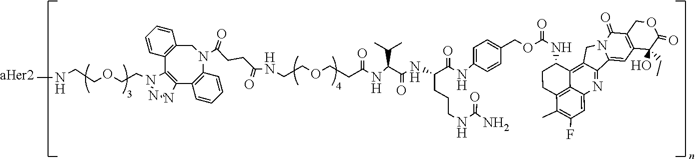 |
| aHer2- ADC | 4.00 | 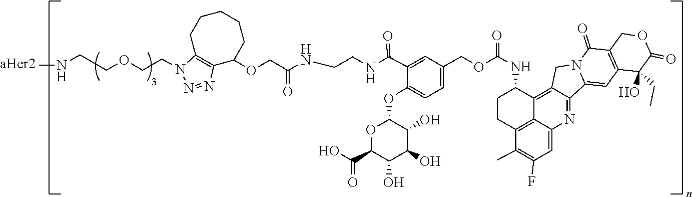 |
| aHer2- ADC | 4.00 | 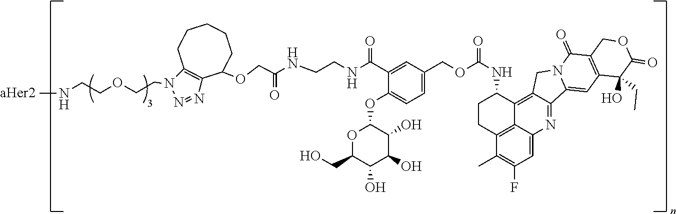 |
| aHer2- ADC | 4.00 | 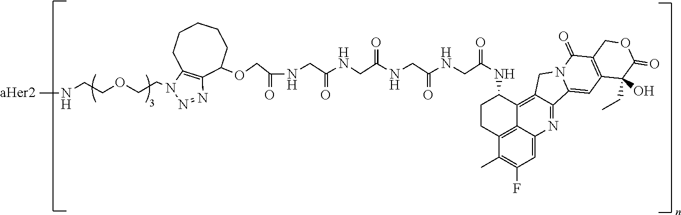 |

Step 1: Site-Specific Conjugation of Handle-Functionalized Amine with an Antibody Generated a Drug Conjugate Containing 2, 4 or 8 Handles Per Antibody.

Aglycosylated human antibody IgG containing an N297Q mutation or N297D mutation in BupH buffer (pH7.4) was mixed with >=100 molar equivalents of non-branched Handle-amine (AL) or branched Handle-amine (BL). The resulting solution was mixed with transglutaminase (25 U/mL; 1 U mTG per mg of antibody, Zedira, Darmstadt, Germany; or 10 U/mL; 5.5 U MTG per mg of antibody, Modernist Pantry-ACTIVA TI contains Maltodextrin from Ajinomoto, Japan) resulting in a final concentration of the antibody at 0.5-20 mg/mL. The reaction mixture was incubated at 25-37° C. for 24 hours while gently shaking while monitored by ESI-MS. Upon the completion, the excess amine and mTG were removed by size exclusion chromatography (SEC) or protein A column chromatography. The conjugate was characterized by UV-Vis, SEC and ESI-MS.

Step 2: Click Reactions Between Handle-Functionalized Antibodies and a Linker-Payload in Table 2 to Generate the Site-Specific ADCs.

The Handle-functionalized antibody (Ab-(AL)ₙ or Ab-(BL)ₙ, 1-20 mg/mL) in PBS (pH7.4) was incubated with >2-10 molar equivalents of a linker-payload (LP) dissolved in an organic solvent such as DMSO or DMA (10 mg/mL) to have the reaction mixture containing 5-15% organic solvent (v/v), at 25-37° C. for 1-48 hours while gently shaking. The reaction was monitored by ESI-MS. Upon completion, the excess amount of LP and organic solvent were removed by desalting column with BupH (pH 7.4) and protein aggregates (if any) were removed by size exclusion chromatography (SEC). The purified conjugate, Ab-(AL-LP)ₙ ADC or Ab-(BL-LP)₄ ADC, was concentrated, sterile filtered and characterized by UV-Vis, SEC and ESI-MS. Conjugates monomer purity was >95% by SEC.

T-DXd was conjugated using our in-house Trastuzumab; both T-DXd and Isotype Ab-DXd ADCs were conjugated with Daiichi's maleimide-tetrapeptide GGFG-linker DXd, Antibody interchain cysteine conjugations with the maleimide linker payload were accomplished using conventional procedures.

All ADCs were purified by SEC using an ÄKTA instrument from Cytiva, using a 16/600 Superdex® 200 column, eluting with DPBS, at a flow rate of 1.5 mL/min at pH 7.4. The DAR values of the ADCs were measured by ESI-MS. A mass increase of 4×LP from Ab-[AL]₄ was observed, correlating to 4DAR ADC. An additional mass increase of 7 to 8×LP from Ab-[BL]₄ was observed, indicating a 7 to 8DAR ADC.

### Example 13. Detailed Conjugation Procedure

A representative 4DAR ADC from Approach I is exemplified following (FIG. 1). The aglycosylated anti-Her2 human IgG antibody containing an N297Q mutation was mixed with >200 molar equivalents of a azido-dPEG3-amine (Handle, MW 708.41 g/moL). The resulting solution was mixed with microbial transglutaminase (10 U/mL; 5,5 U mTG per mg of antibody, Modernist Pantry-ACTIVA TI contains Maltodextrin from Ajinomoto, Japan) resulting in a final concentration of the antibody at 5 mg/mL. The reaction mixture was incubated at 37° C. for 24 hours while gently shaking while monitored by ESI-MS. Upon the completion, the excess amine and mTG were removed by size exclusion chromatography (SEC). The conjugate was characterized by UV-Vis, SEC and ESI-MS. The azido linkers attached antibody resulted in a 808 Da mass increase compared to mAb, indicating 4 Handle was conjugated to the antibody (Ab-(Handle)₄) with 4 azido handles. The site-specific antibody azido conjugate (2.1 mg/mL) in PBS (pH7.4) was mixed with 7 molar equivalents of linker-payload (Linker-Payload) in 2 mM of DMSO to have the reaction mixture containing 5% organic solvent (v/v), and the solution was set at 32° C. for 36 hours while gently shaking. The reaction was monitored by ESI-MS. Upon completion, the excess amount of linker-payload and protein aggregates were removed by size exclusion chromatography (SEC). The purified conjugate was concentrated, sterile filtered and characterized by UV-Vis, SEC and ESI-MS. Conjugates monomer purity was 99.8% by SEC. The drug attached antibody resulting in a mass increase corresponding to the DAR4 conjugate. Conjugates monomer purity was >99% by SEC.

TABLE 8
ADCs in vitro activity

| | | ADC | | |
|---|---|---|---|---|
| | Linker-payload | | | SKBR3 EC₅₀ |
| Payload | Linker Name | ADC# | DAR | (nM) |
| Exatecan | / | / | / | 0.516 |
| ProDXd | COT-PEG₃-BR2-(PEG₂- | | 6.2 | 0.22 |
| | GGFG-NHCH₂-DXd)₂ | | | |
| ProDXd | COT-PEG₃-BR2-(PEG₂- | aHER2- | 7.6 | 0.237 |
| | GGFG-NHCH₂-DXd)₂ | ADC | | |
| | | Isotype | 7.0 | >100 |
| | | Control - | | |
| | | ADC | | |
| Exatecan | COT-PEG4-vcPAB- | aHER2- | 3.58 | 2.365 |
| | Exatecan | ADC | | |
| Exatecan | DIBAC-PEG4-vcPAB- | aHER2- | 4.00 | 1.920 |
| | Exatecan | ADC | | |
| Exatecan | COT-EDA-(GLCA)PAB- | aHER2- | 4.00 | 1.465 |
| | Exatecan | ADC | | |
| Exatecan | COT-EDA-(GLC)PAB- | aHER2- | 4.00 | 4.117 |
| | Exatecan | ADC | | |
| Exatecan | COT-GGGG-Exatecan | aHER2- | 4.00 | 0.170 |
| | | ADC | | |

### Example 6: In Vitro Cell Killing Activity in SKBR3 Cell Lines

To test the ability of anti-Her2 drug conjugates (ADCs) of the present disclosure (see Table 5) to kill human cell lines, an in vitro cytotoxicity assay was performed. In vitro cytotoxicity of the ADCs, isotype control ADCs, and reference free payloads were evaluated using the CellTiter-Glo 2.0 Assay Kit (Promega, Cat #G9243), in which the quantity of ATP present is used to determine the number of viable cells in culture.

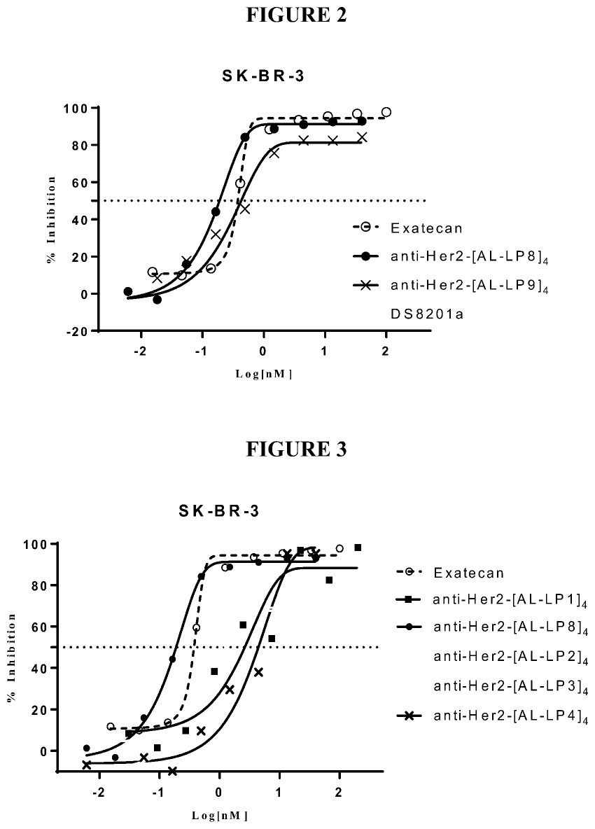

For the assay, SK-BR-3 cells were seeded at 1000 cells/well in poly-D-lysine coated white 96 well BioCoat plates (Corning #356693) in complete growth medium and grown overnight at 37° C. in 5% CO₂. Three-fold serial dilutions of anti-Her2 ADCs or isotype control ADCs were prepared in dilution media (Optimem+0.1% BSA) and added to cells at final concentrations ranging from 100 nM to 0.015 nM (concentrations were corrected for the DAR (drug antibody ratio) and dosed based on the effective payload concentration). Three-fold serial dilutions of free payloads were prepared in 100% DMSO, transferred to fresh dilution media, and then added to the cells at a final constant DMSO concentration of 0.2% and final payload concentrations ranging from 100 nM to 0.015 nM. The last well in each dilution series (untreated wells) served as blank controls containing only the media (ADCs) or media plus 0.2% DMSO (payloads) and was plotted as a continuation of the 3-fold serial dilution. Six days later, 100 μL of CellTiter Glo 2.0 was added to each well, plates were mixed for 2 minutes on an orbital shaker, and plates were incubated at room temperature for 10 minutes. Relative light units (RLUs) were measured on an Envision luminometer (PerkinElmer), and cell viability was expressed as a percentage of the untreated (100% viable) cells. IC₅₀ values were determined using a four-parameter logistic equation over a 10-point dose response curve (GraphPad Prism). The maximum % kill was also determined for each test article as follows: 100−minimum percent viability. One experiment was run and results were shown FIGS. 2 and 3 and the EC₅₀ values and maximum % kill of each test article are reported.

TABLE 9
ADCs in vitro cell-killing results in SKBR3

| | | | ADC | | |
|---|---|---|---|---|---|
| Payload | Linker-payload | | | | SKBR3 EC₅₀ |
| CP# | LP# | Description | Description | DAR | (nM) |
| Exatecan | | / | / | / | 0.516 |
| Exatecan | LP1 | COT-PEG4-vcPAB- | Anti-HER2-AL-LP1 | 3.58 | 2.365 |
| | | Exatecan | | | |
| Exatecan | LP2 | DIBAC-PEG4-vCPAB- | Anti-HER2-AL-LP2 | 4 | 1.920 |
| | | Exatecan | | | |
| Exatecan | LP3 | COT-EDA-(GLCA)PAB- | Anti-HER2-AL-LP3 | 4 | 1.465 |
| | | Exatecan | Control-AL-LP3 | 4 | |
| Exatecan | LP4 | COT-EDA-(GLC)PAB- | Anti-HER2-AL-LP4 | 4 | 4.117 |
| | | Exatecan | | | |
| Exatecan | LP8 | COT-GGGG-ProEXT | Anti-HER2-AL-LP8 | 4 | 0.170 |
| | | | Control-AL-LP8 | 4 | |
| ProDXD | LP9 | COT-PEG4-vCPAB- | Anti-HER2-AL-LP9 | 4 | 0.382 |
| | | ProDXD | Control-AL-LP10 | 4 | 285.967 |
| DXD | LP10 | Mc-GGFG-NHCH2-Dxd | DS8201a | 8 | 0.372 |

As shown in Table 9, above, anti-Her2 ADCs conjugated via glutamines killed high Her2 expressing SK-BR-3 cells with IC₅₀ values ranging from 0.17 nM to 4.1 nM and maximum % kill values ranging from >60% to 95%. Trastuzumab deruxtecan (T-DXD via Cysteine conjugation) killed SK-BR-3 cells was used as a positive control in the assay and with an EC₅₀ value of 372 pM and a maximum % kill value of 90.9%. The unconjugated anti-Her2 antibodies were weakly cytotoxic in all tested lines (result nor shown in the Figures). The free Exatecan payload released from the ADCs killed cells with EC₅₀ values ranging from 516 pM and maximum % kill values near 95%. * * *

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications, and variances which fall within the scope of the appended claims.

All patents, applications, publications, test methods, literature, and other materials cited herein are hereby incorporated by reference in their entirety as if physically present in this specification.

What is claimed is:

1. A compound having a structure according to Formula (A):
BA-(L1-B-L2-P)ₙ (A), wherein:
BA is an antibody or an antigen-binding fragment thereof;
L1 is a first linker;
B is a moiety comprising a triazole;
L2 is a second linker;
P is selected from the group consisting of P-I through P-IV: 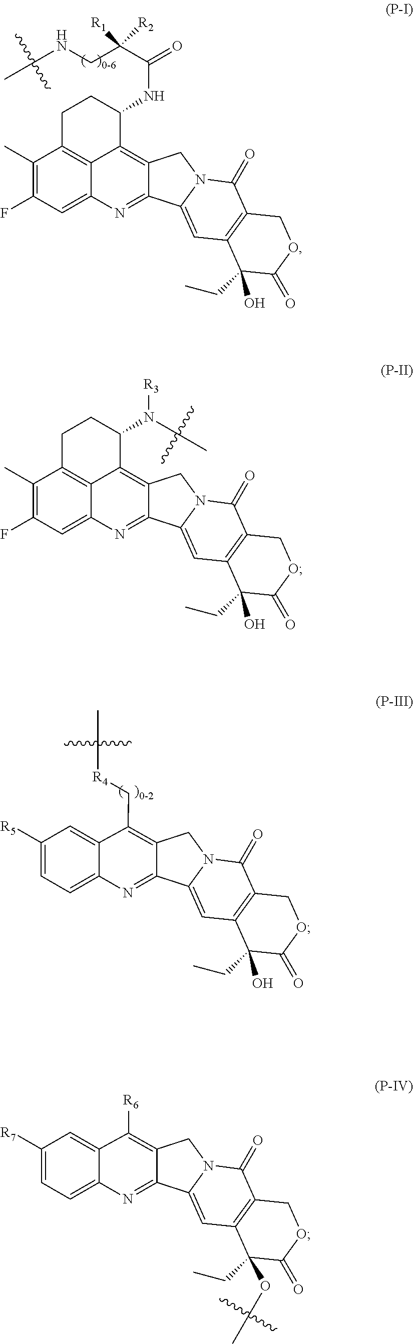
R₁ is hydrogen, C₁₋₆ alkyl, —(CH₂)ᵥ—OH, —(CH₂)ᵥ—NH₂, —(CH₂)ᵥ—C(O)OH, —(CH₂)ᵥ-phenyl, and —(CH₂)ᵥ—SO₂CH₃, wherein v is an integer from 0 to 12;
R₂ is hydrogen, C₁₋₆ alkyl, —(CH₂)ᵥ—OH, —(CH₂)ᵥ—NH₂, —(CH₂)ᵥ—C(O)OH, —(CH₂)ᵥ-phenyl, and —(CH₂)ᵥ—SO₂CH₃, wherein v is an integer from 0 to 12;
R₃ is hydrogen —C₁₋₆ alkyl, —(CH₂)ᵥ—OH, —(CH₂)ᵥ—NH₂, —(CH₂)ᵥ—C(O)OH, —(CH₂)-phenyl, —(CH₂)ᵥ—SO₂CH₃, and —CO—(CH₂)ᵥ—O—COCH₃, wherein v is an integer from 0 to 12;
R₄ is —NH—, —N(—C₁₋₆ alkyl), —N(—C₁₋₆ alkyl)(—SO₂CH₃), —N(—C₁₋₆ alkyl)(—(CH₂)ᵥ—OH), —N(—C₁₋₆ alkyl)(CO—CH₂—NH₂), —N(—C₁₋₆ alkyl)(—(CH₂)ᵥO—CH₂—NH—CO—CH₂—NH₂), —N(—C₁₋₆ alkyl)(—CO—CH(NH₂)—(CH₂)ᵥ—COOH), —N(—C₁₋₆ alkyl)(—CO—CH(NH₂)—(CH₂)ᵥ—NH₂), —N(—C₁₋₆ alkyl)(—CO—CH(NH₂)—(CH₂)ᵥ-phenyl), or 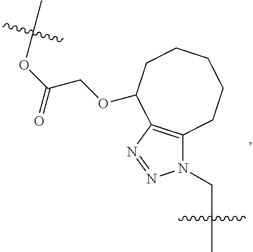
wherein v is an integer from 0 to 12;
R₅ is H, —OH, —OCH₃, or 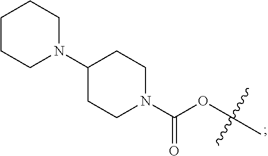 [SMILES: *OC(=O)N1CCC(N2CCCCC2)CC1]
R₆ is hydrogen —C₁₋₆ alkyl, —(CH₂)ᵥ—OH, —(CH₂)ᵥ—NH₂, —(CH₂)-phenyl, —(CH₂)ᵥ—N₃, —(CH₂)ᵥ—NH—CH₂-phenyl, —(CH₂)ᵥ—NMe-CH₂-phenyl-OMe, —(CH₂)ᵥ—NH—(CH₂)ᵥ—N₃, —(CH₂)ᵥ—NH—(CH₂)ᵥ—CCH; —(CH₂)—NH—CO—CH(NH₂)—(CH₂)ᵥ-phenyl, —(CH₂)ᵥ—NH—CO—CH(NH₂)—(CH₂)ᵥ—N₃, —(CH₂)ᵥ—NH—CO—CH(NH₂)—(CH₂)ᵥ—NH₂, —(CH₂)ᵥ—NH—CO—CH(NH₂)—(CH₂)ᵥ—COOH, 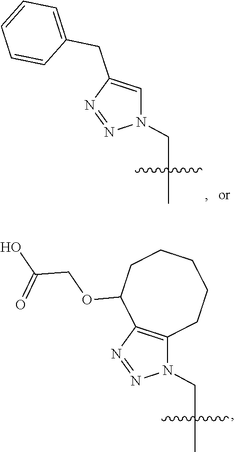
wherein
v is an independently an integer from 0 to 12;
R₇ is H, —OH, —OCH₃, or 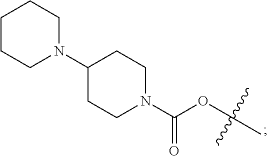 [SMILES: *OC(=O)N1CCC(N2CCCCC2)CC1]
n is an integer from 1 to 12.

2. The compound of claim 1, wherein said first linker L1 is connected to the side chain of a glutamine residue of the BA.

3. (canceled)

4. (canceled)

5. The compound of claim 2, wherein the BA comprises one or more glutamine residues, which glutamine residues are naturally present in said BA, or are introduced to the BA by site-specific modification of one or more amino acids.

6. The compound of claim 1, wherein the BA is an anti-HER2 antibody, an anti-STEAP2 antibody, an anti-MET antibody, an anti-EGFRVIII antibody, an anti-MUC16 antibody, an anti-PRLR antibody, an anti-PSMA antibody, an anti-FGFR2 antibody, an anti-FOLR1 antibody, an anti-HER2/HER2 bispecific antibody, an anti-MET/MET bispecific antibody, or an antigen-binding fragment thereof.

7. (canceled)

8. The compound of claim 1, wherein the BA targets a cancer selected from the group consisting of breast cancer, ovarian cancer, prostate cancer, lung cancer, liver cancer, and brain cancer.

9. (canceled)

10. (canceled)

11. The compound of claim 2, wherein the glutamine residue is Q295 or N297Q.

12. The compound of claim 1, wherein L1 comprises C₁₋₆ alkyl, phenyl, aralkyl-NH—, —C(O)—, —(CH₂)ᵤ—NH—C(O)—, —(CH₂)ᵤ—C(O)—NH—, —(CH₂—CH₂—O)ᵥ—, —(CH₂)ᵤ—(O—CH₂—CH₂)ᵥ—C(O)—NH—, a peptide unit comprising from 2 to 4 amino acids, or combinations thereof, each of which may be optionally substituted with one or more of —S—, —S(O₂)—, —C(O)—, —C(O₂)—; and —CO₂H, wherein subscripts u and v are independently an integer from 1 to 8.

13. The compound of claim 1, wherein L1 is selected from the group consisting of:

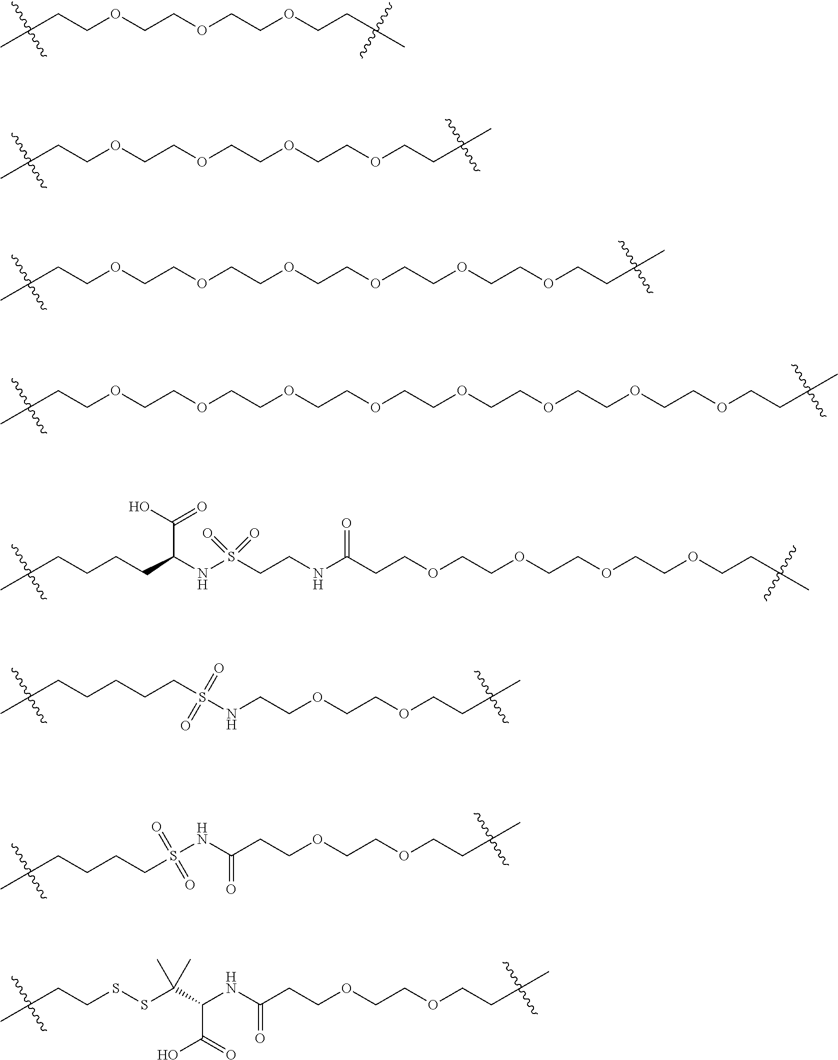

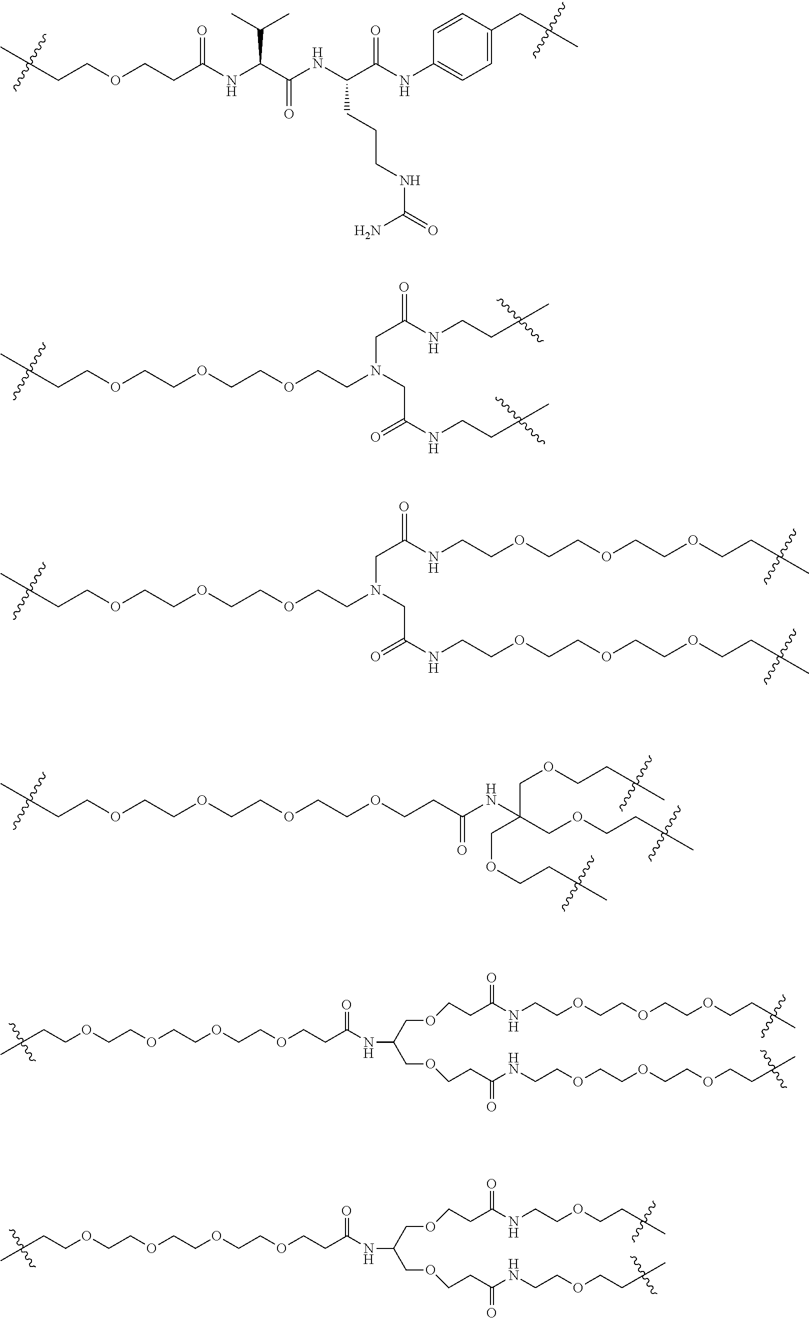 [SMILES: *CCOCCC(=O)N[C@H](C(=O)N[C@@H](CCCNC(N)=O)C(=O)Nc1ccc(C*)cc1)C(C)C ; *CCNC(=O)CN(CCOCCOCCOCC*)CC(=O)NCC* ; *CCOCCOCCOCCNC(=O)CN(CCOCCOCCOCC*)CC(=O)NCCOCCOCCOCC* ; *CCOCCOCCOCCOCCC(=O)NC(COCC*)(COCC*)COCC* ; *CCOCCOCCOCCNC(=O)CCOCC(COCCC(=O)NCCOCCOCCOCC*)NC(=O)CCOCCOCCOCCOCC* ; *CCOCCNC(=O)CCOCC(COCCC(=O)NCCOCC*)NC(=O)CCOCCOCCOCCOCC*]

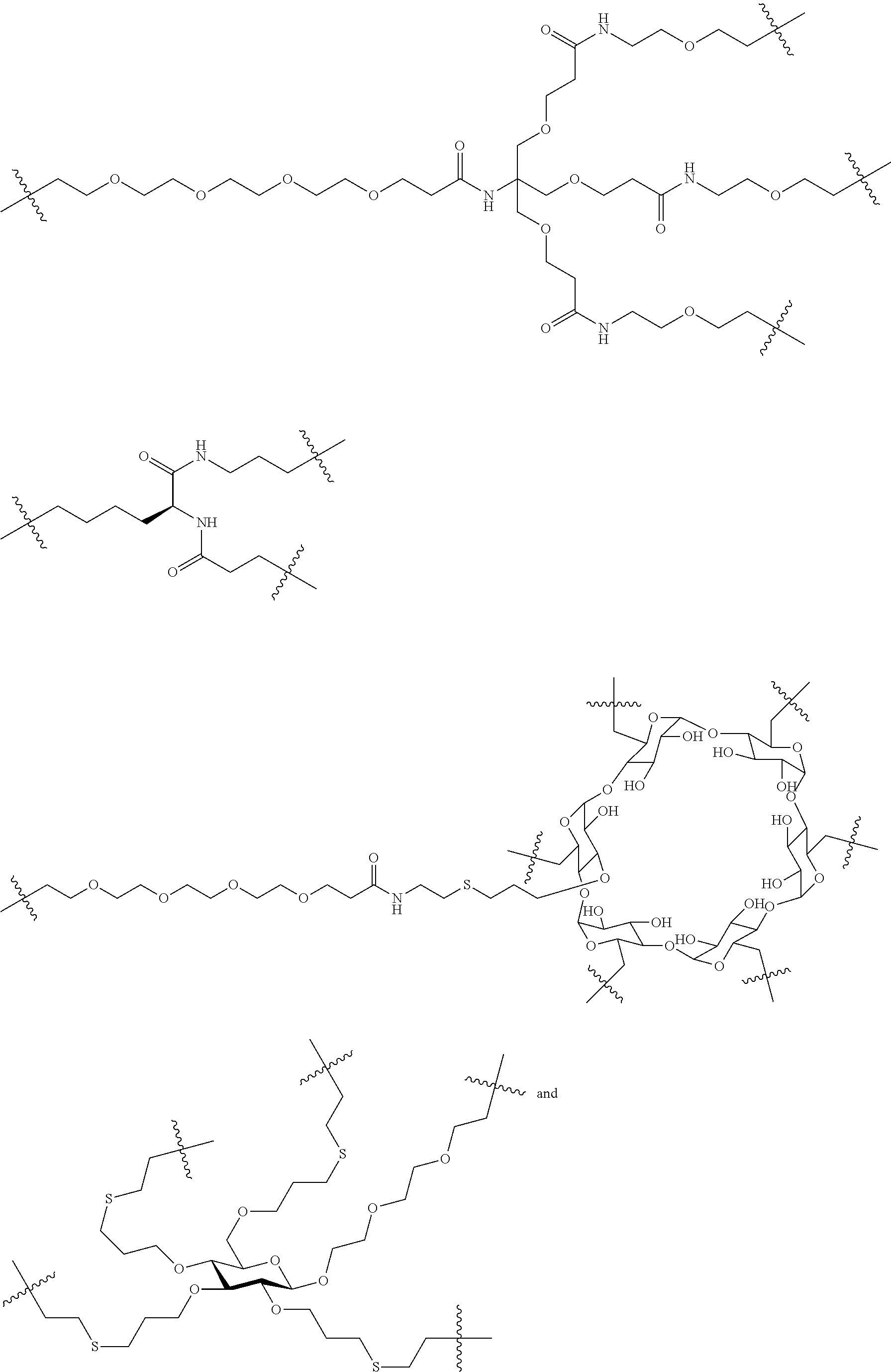

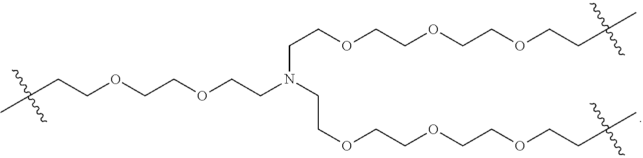 [SMILES: *CCOCCOCCOCCN(CCOCCOCC*)CCOCCOCCOCC*]

14. (canceled)

15. (canceled)

16. The compound of claim 1, wherein B is selected from the group consisting of: 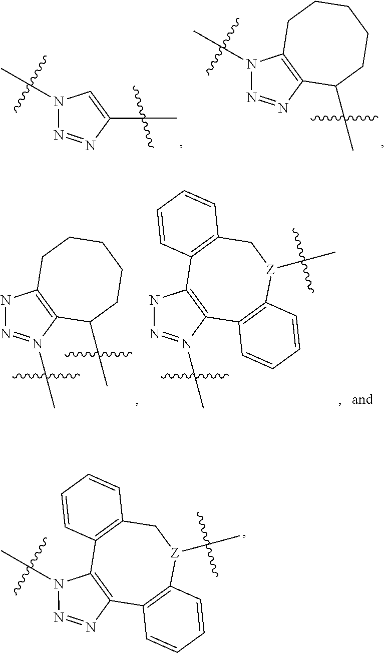
where Z is C or N.

17. (canceled)

18. The compound of claim 1, wherein L2 has a structure according to Formula (L2):
-SP1-AA-SP2- (L2),
wherein:
SP1 is absent or a first spacer unit;
AA is absent or a peptide unit comprising from 2 to 4 amino acids;
SP2 is absent or a second spacer unit covalently attached to the P, provided that at least one of SP1, AA and SP2 is not absent.

19. The compound of any one of claim 18, wherein SP1 is absent or selected from the group consisting of 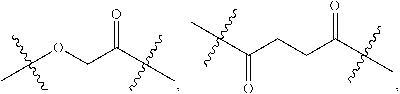 [SMILES: *OCC(*)=O ; *C(=O)CCC(*)=O]
C₁₋₆ alkyl, —(CH₂—CH₂—O)ᵥ—, —NH—, —C(O)—, —NH—C(O)—, —NH—(CH₂)ᵤ—, —NH—(CH₂)ᵤ—C(O)—, —NH—(CH₂—CH₂—O)ᵥ—, —NH—(CH₂—CH₂—O)ᵥ—C(O)—, —NH—(CH₂—CH₂—O)ᵥ—(CH₂)ᵤ—, —NH—(CH₂—CH₂—O)ᵥ—(CH₂)ᵤ—C(O)—, —(CH₂)ᵤ—NH—C(O)—, —NH—(CH₂)ᵤ—NH—C(O)—, —NH—(CH₂)ᵤ—C(O)—NH—, and combinations thereof; wherein subscripts u and v are independently an integer from 1 to 8.

20. The compound of claim 18, wherein AA is a peptide unit comprising from 2 to 4 amino acids selected from alanine, glycine, valine, proline, glutamic acid, lysine, phenylalanine, and citrulline, and combinations thereof.

21. (canceled)

22. The compound of claim 18, wherein SP2 is absent or selected from the group consisting of 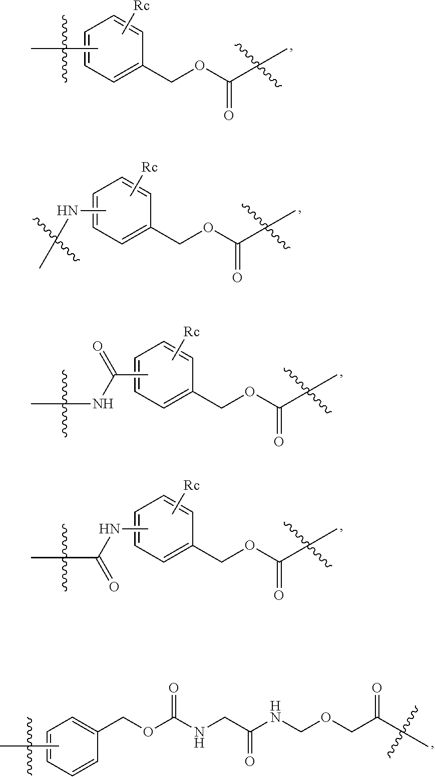
and combinations thereof, wherein R_{c} is independently at each occurrence absent or a group selected from

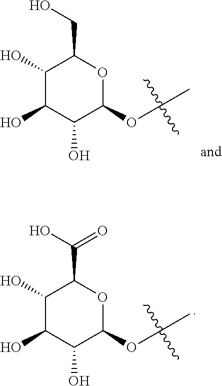 [SMILES: *O[C@@H]1O[C@H](CO)[C@@H](O)[C@H](O)[C@H]1O ; *O[C@@H]1O[C@H](C(=O)O)[C@@H](O)[C@H](O)[C@H]1O]

23. The compound of claim 1, wherein L2 is selected from the group consisting of:

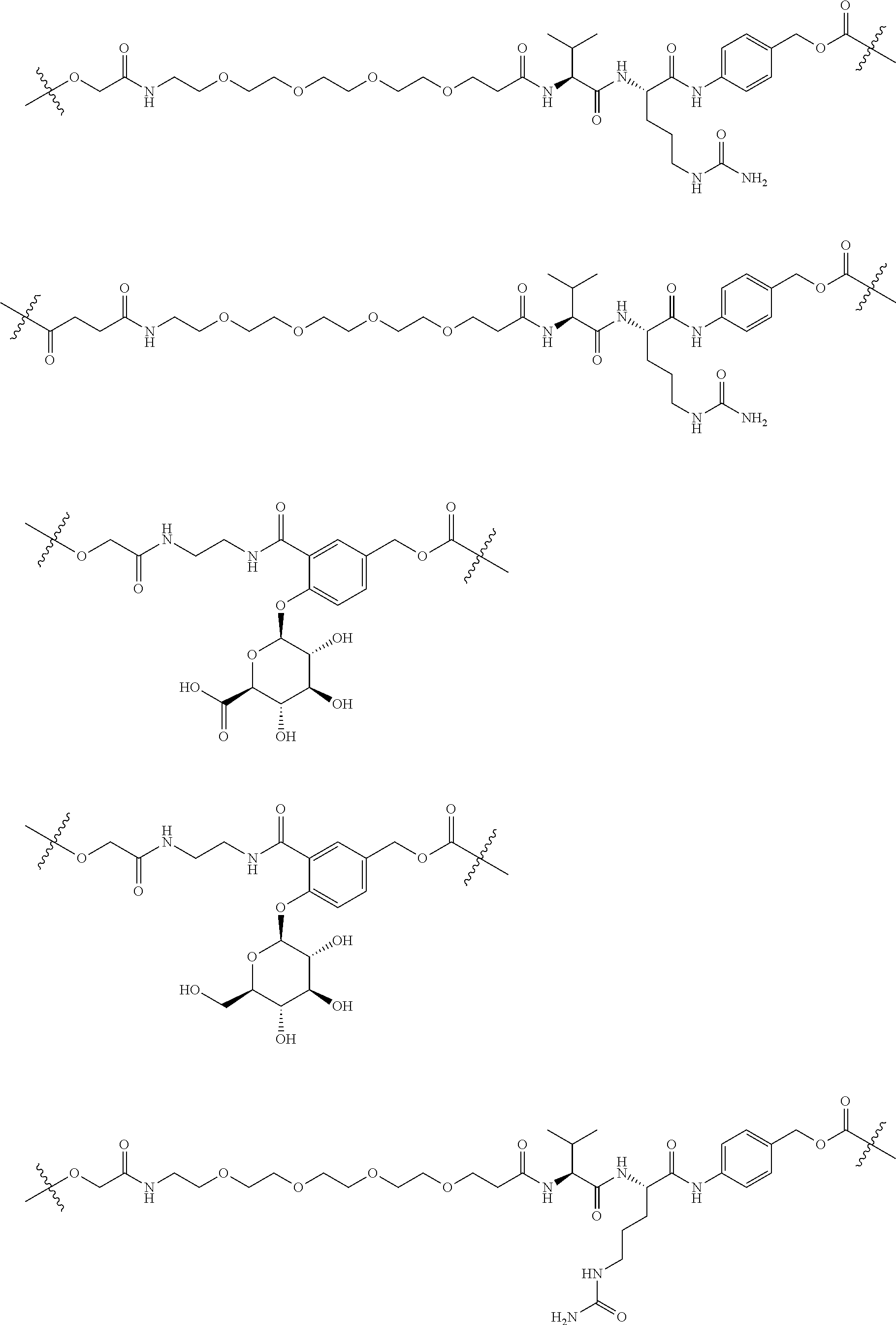 [SMILES: *OCC(=O)NCCOCCOCCOCCOCCC(=O)N[C@H](C(=O)N[C@@H](CCCNC(N)=O)C(=O)Nc1ccc(COC(*)=O)cc1)C(C)C ; *C(=O)CCC(=O)NCCOCCOCCOCCOCCC(=O)N[C@H](C(=O)N[C@@H](CCCNC(N)=O)C(=O)Nc1ccc(COC(*)=O)cc1)C(C)C ; *OCC(=O)NCCNC(=O)c1cc(COC(*)=O)ccc1O[C@@H]1O[C@H](C(=O)O)[C@@H](O)[C@H](O)[C@H]1O ; *OCC(=O)NCCNC(=O)c1cc(COC(*)=O)ccc1O[C@@H]1O[C@H](CO)[C@@H](O)[C@H](O)[C@H]1O ; *OCC(=O)NCCOCCOCCOCCOCCC(=O)N[C@H](C(=O)N[C@@H](CCCNC(N)=O)C(=O)Nc1ccc(COC(*)=O)cc1)C(C)C]

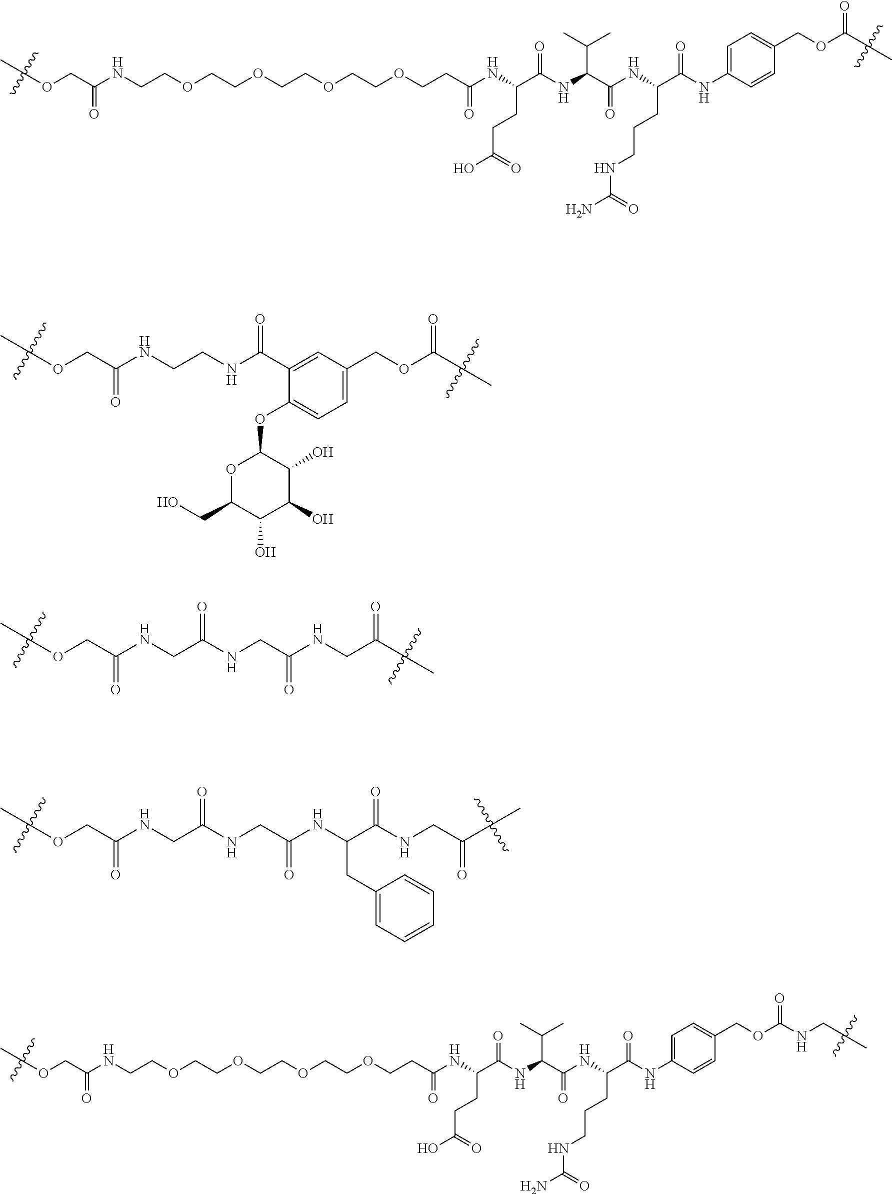 [SMILES: *OCC(=O)NCCOCCOCCOCCOCCC(=O)N[C@@H](CCC(=O)O)C(=O)N[C@H](C(=O)N[C@@H](CCCNC(N)=O)C(=O)Nc1ccc(COC(*)=O)cc1)C(C)C ; *OCC(=O)NCCNC(=O)c1cc(COC(*)=O)ccc1O[C@@H]1O[C@H](CO)[C@@H](O)[C@H](O)[C@H]1O ; *OCC(=O)NCC(=O)NCC(=O)NCC(*)=O ; *OCC(=O)NCC(=O)NCC(=O)NC(Cc1ccccc1)C(=O)NCC(*)=O ; *CNC(=O)OCc1ccc(NC(=O)[C@H](CCCNC(N)=O)NC(=O)[C@@H](NC(=O)[C@H](CCC(=O)O)NC(=O)CCOCCOCCOCCOCCNC(=O)CO*)C(C)C)cc1]

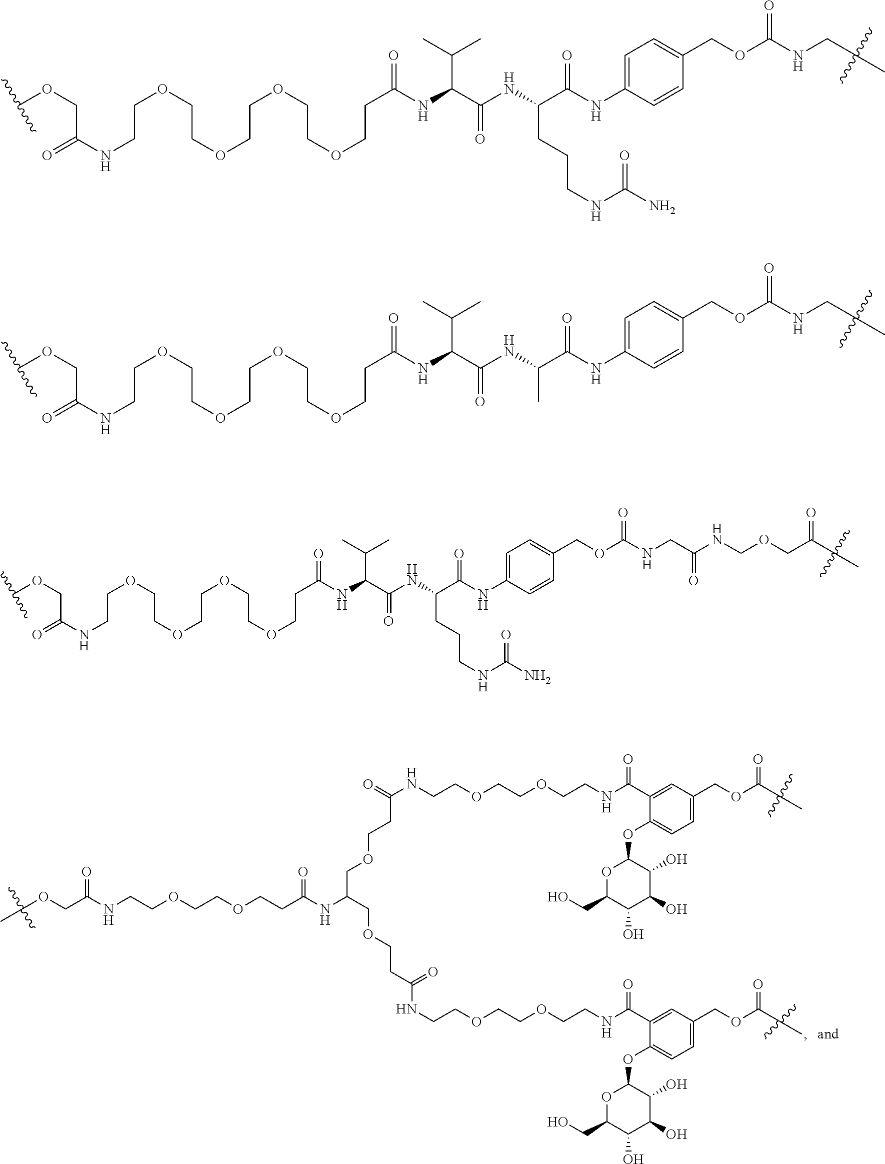 [SMILES: *CNC(=O)OCc1ccc(NC(=O)[C@H](CCCNC(N)=O)NC(=O)[C@@H](NC(=O)CCOCCOCCOCCOCCNC(=O)CO*)C(C)C)cc1 ; *CNC(=O)OCc1ccc(NC(=O)[C@H](C)NC(=O)[C@@H](NC(=O)CCOCCOCCOCCOCCNC(=O)CO*)C(C)C)cc1 ; *OCC(=O)NCCOCCOCCOCCOCCC(=O)N[C@H](C(=O)N[C@@H](CCCNC(N)=O)C(=O)Nc1ccc(COC(=O)NCC(=O)NCOCC(*)=O)cc1)C(C)C ; *OCC(=O)NCCOCCOCCC(=O)NC(COCCC(=O)NCCOCCOCCNC(=O)c1cc(COC(*)=O)ccc1O[C@@H]1O[C@H](CO)[C@@H](O)[C@H](O)[C@H]1O)COCCC(=O)NCCOCCOCCNC(=O)c1cc(COC(*)=O)ccc1O[C@@H]1O[C@H](CO)[C@@H](O)[C@H](O)[C@H]1O]

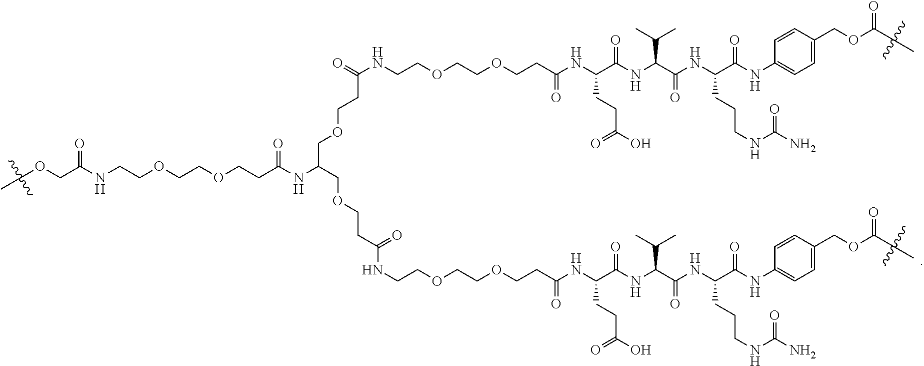 [SMILES: *OCC(=O)NCCOCCOCCC(=O)NC(COCCC(=O)NCCOCCOCCC(=O)N[C@@H](CCC(=O)O)C(=O)N[C@H](C(=O)N[C@@H](CCCNC(N)=O)C(=O)Nc1ccc(COC(*)=O)cc1)C(C)C)COCCC(=O)NCCOCCOCCC(=O)N[C@@H](CCC(=O)O)C(=O)N[C@H](C(=O)N[C@@H](CCCNC(N)=O)C(=O)Nc1ccc(COC(*)=O)cc1)C(C)C]

24. The compound of claim 1, wherein n is selected from 2, 4 and 8.

25. (canceled)

26. (canceled)

27. The compound of claim 1, wherein the compound has a structure selected from the group consisting of: 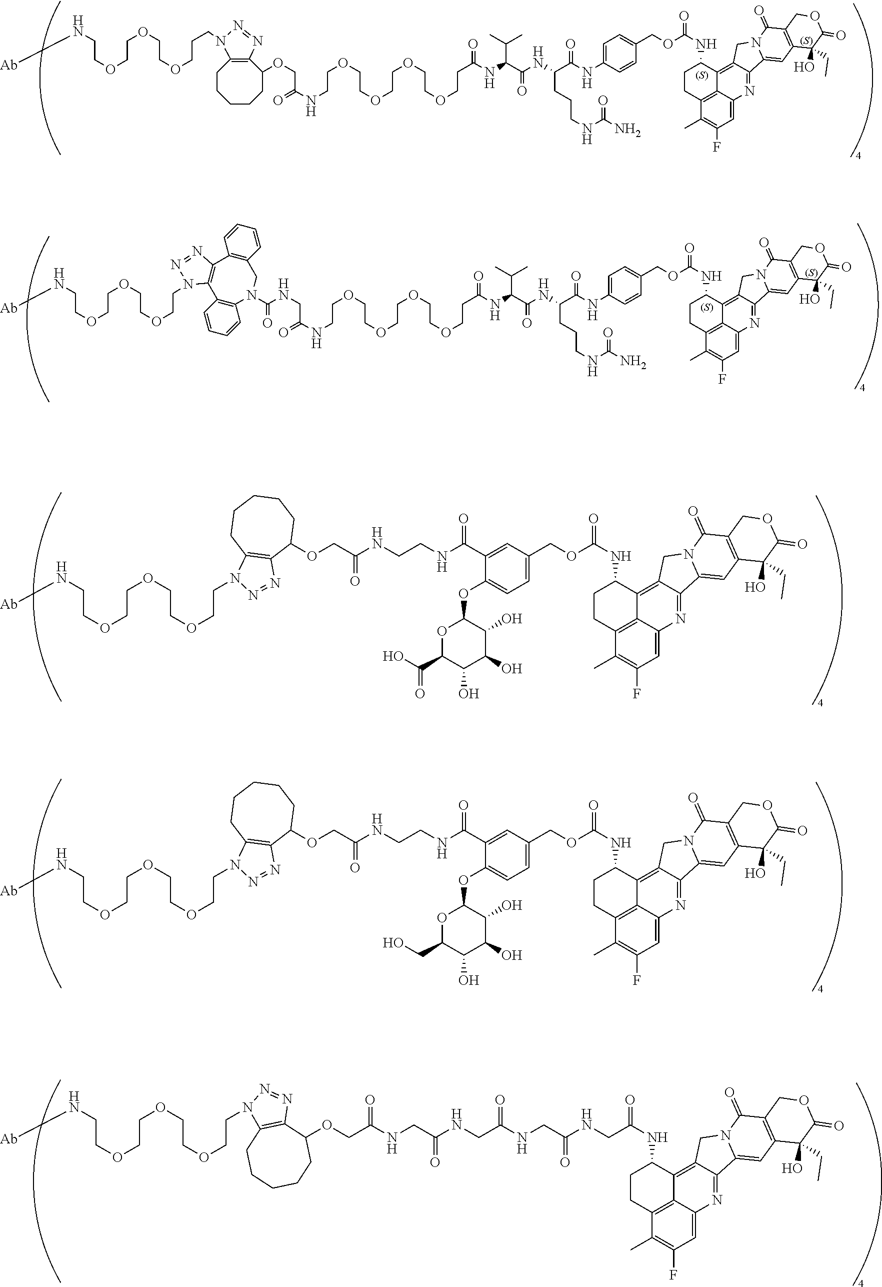 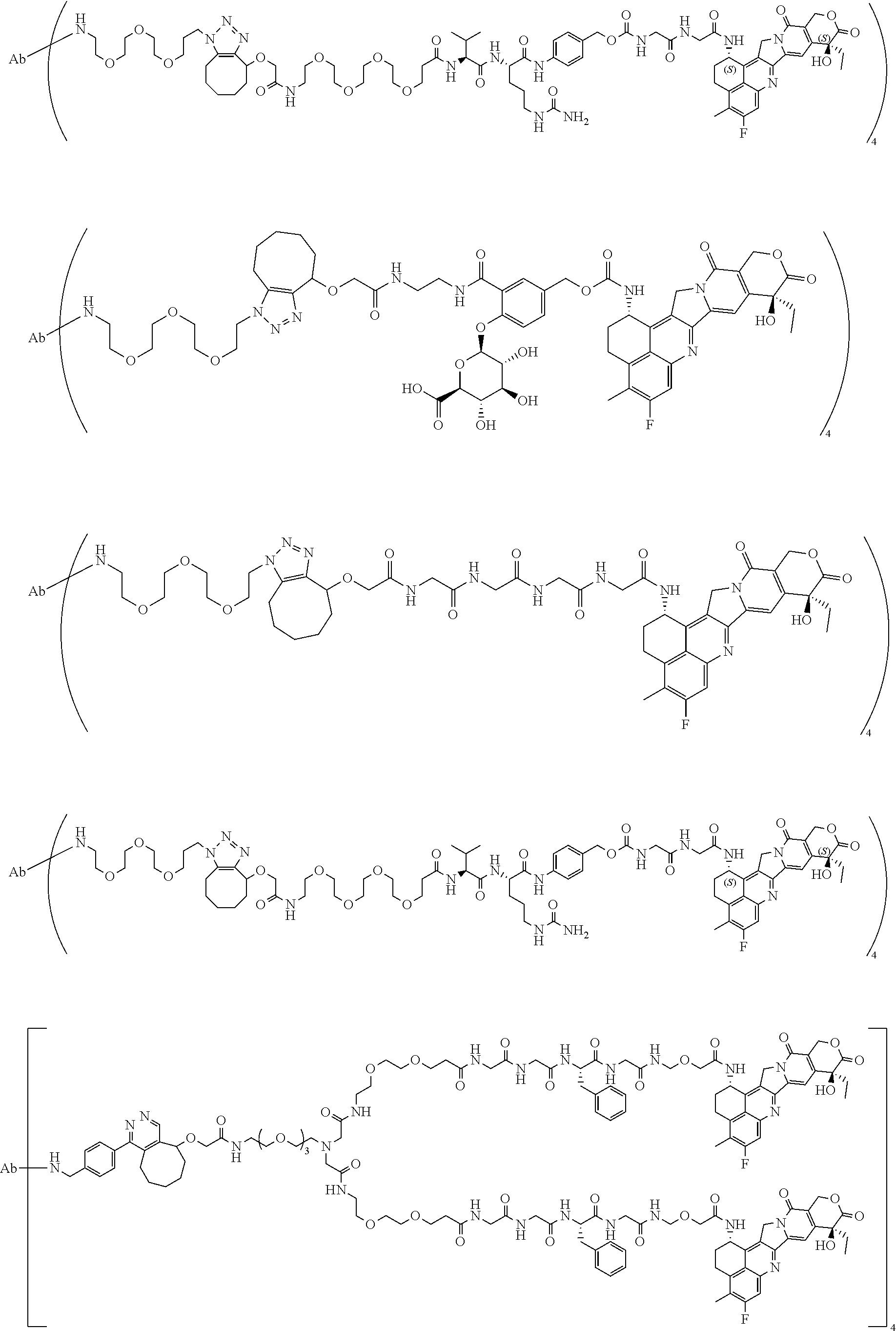 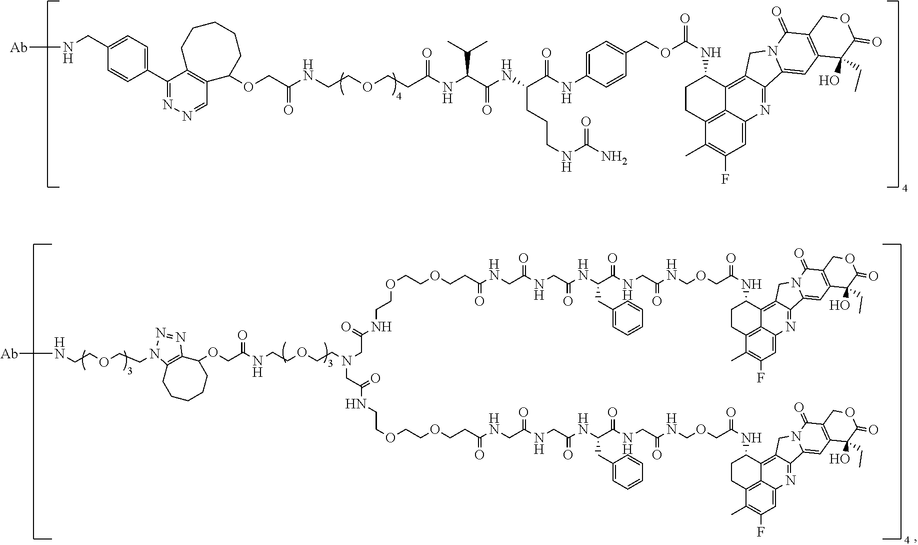
or a pharmaceutically acceptable salt thereof.

28. A compound according to Formula (Alk-1L2-P):
Alk-SP1-AA-SP2-P (Alk-1L2-P), wherein:
Alk is a moiety comprising an alkyne;
SP1 is absent or a first spacer unit;
AA is absent or a peptide unit comprising from 2 to 4 amino acids;
SP2 is absent or a second spacer unit; and
P is selected from the group consisting of P-I through P-IV: 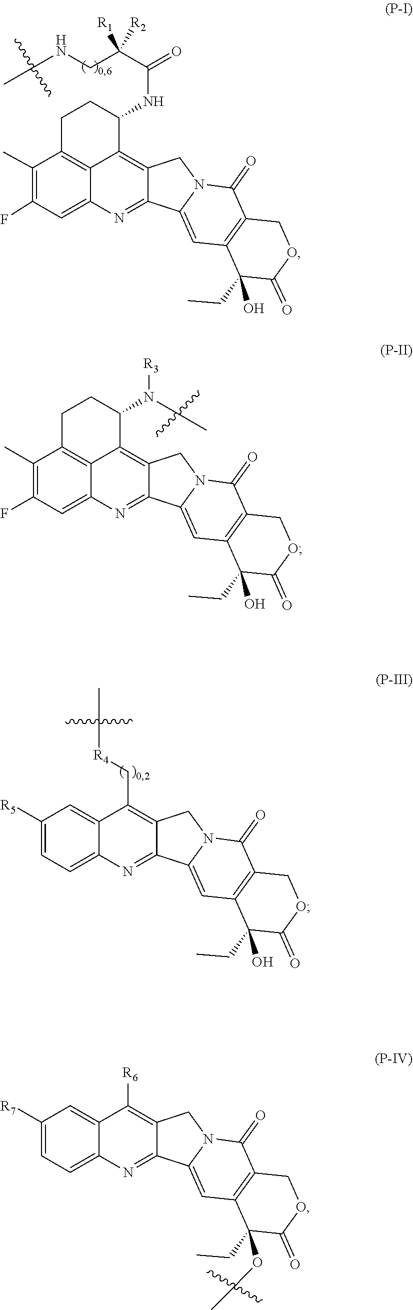
or a pharmaceutically acceptable salt thereof.

29. The compound of claim 28, wherein the compound according to Formula (Alk-L2-P) is selected from the group consisting of: 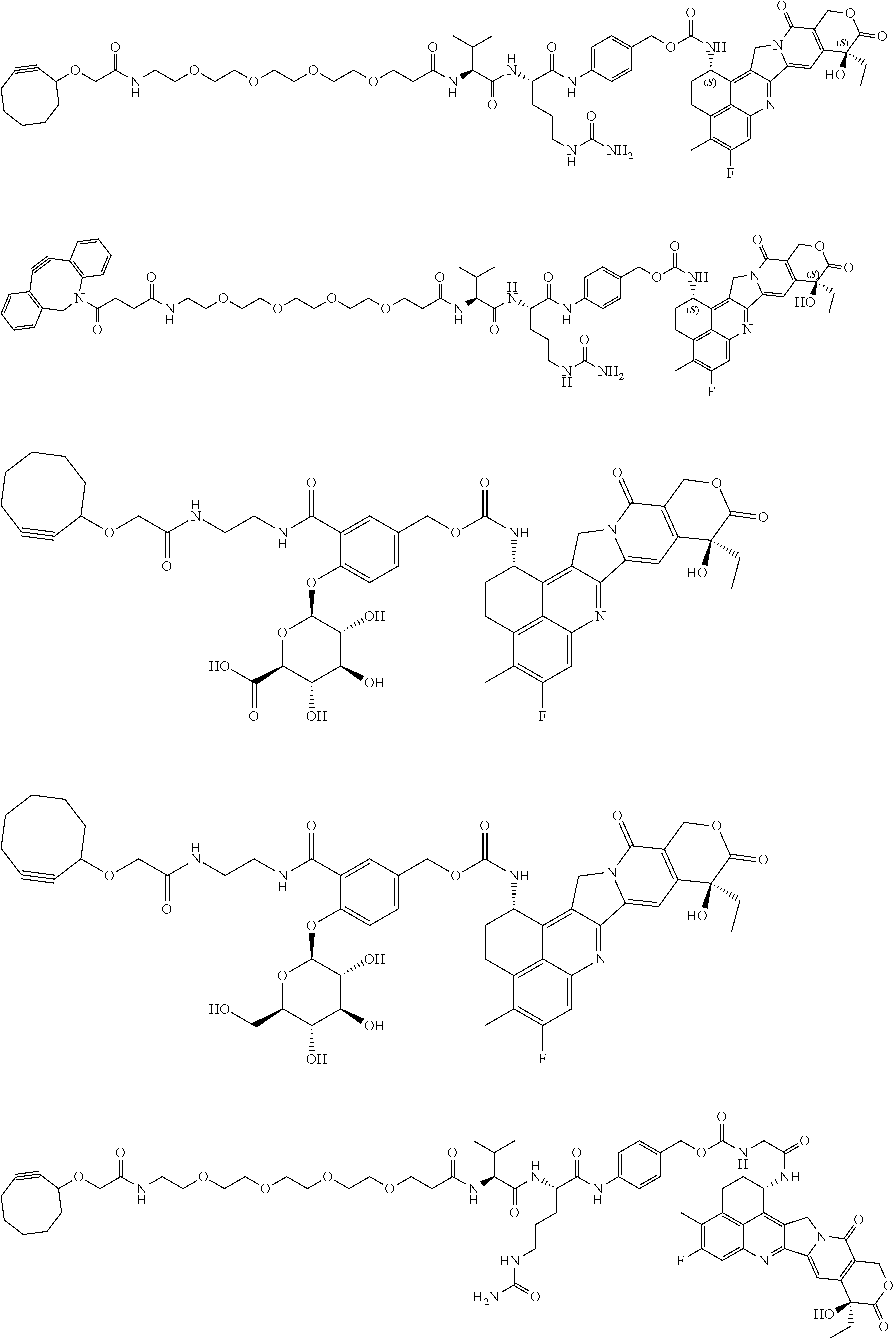 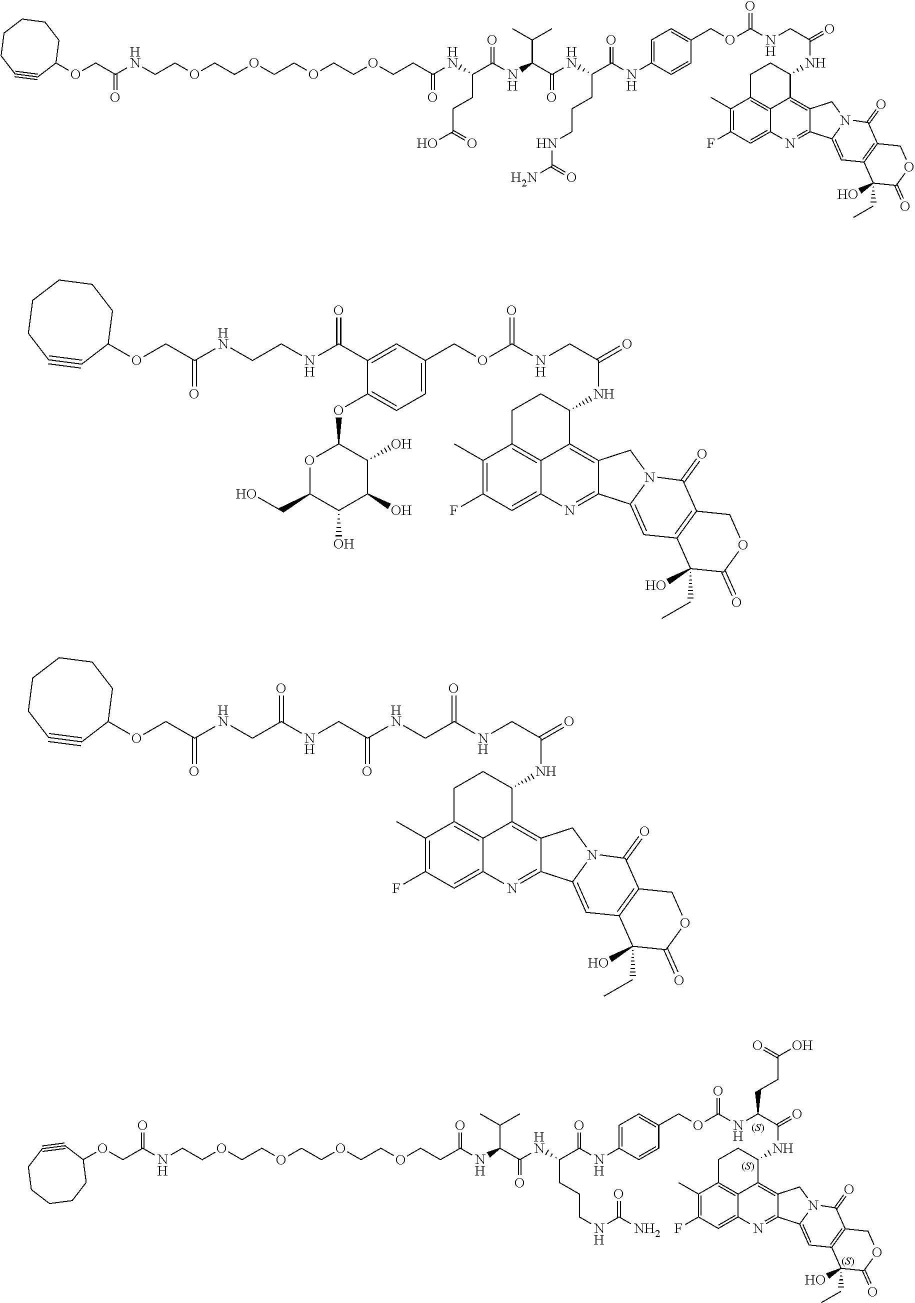 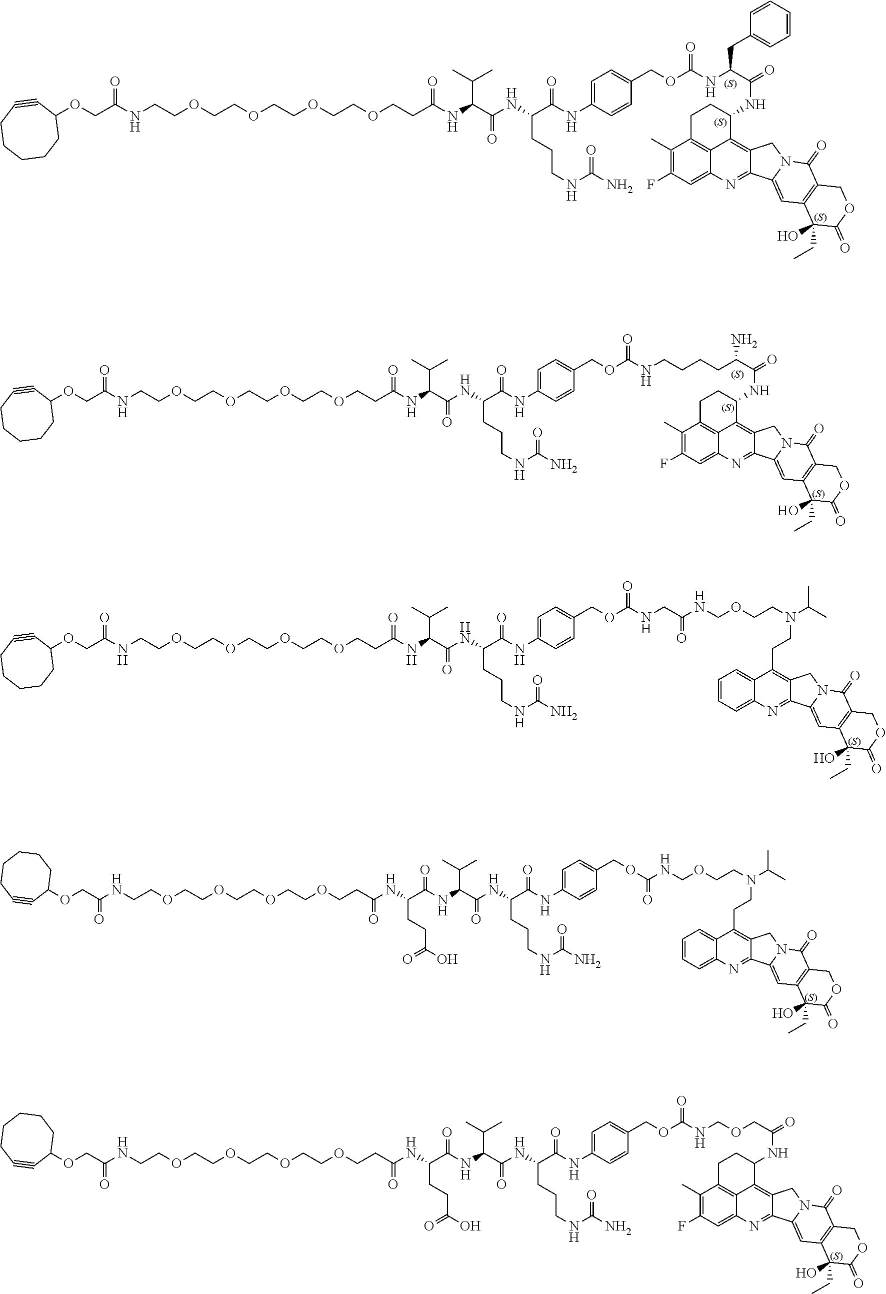 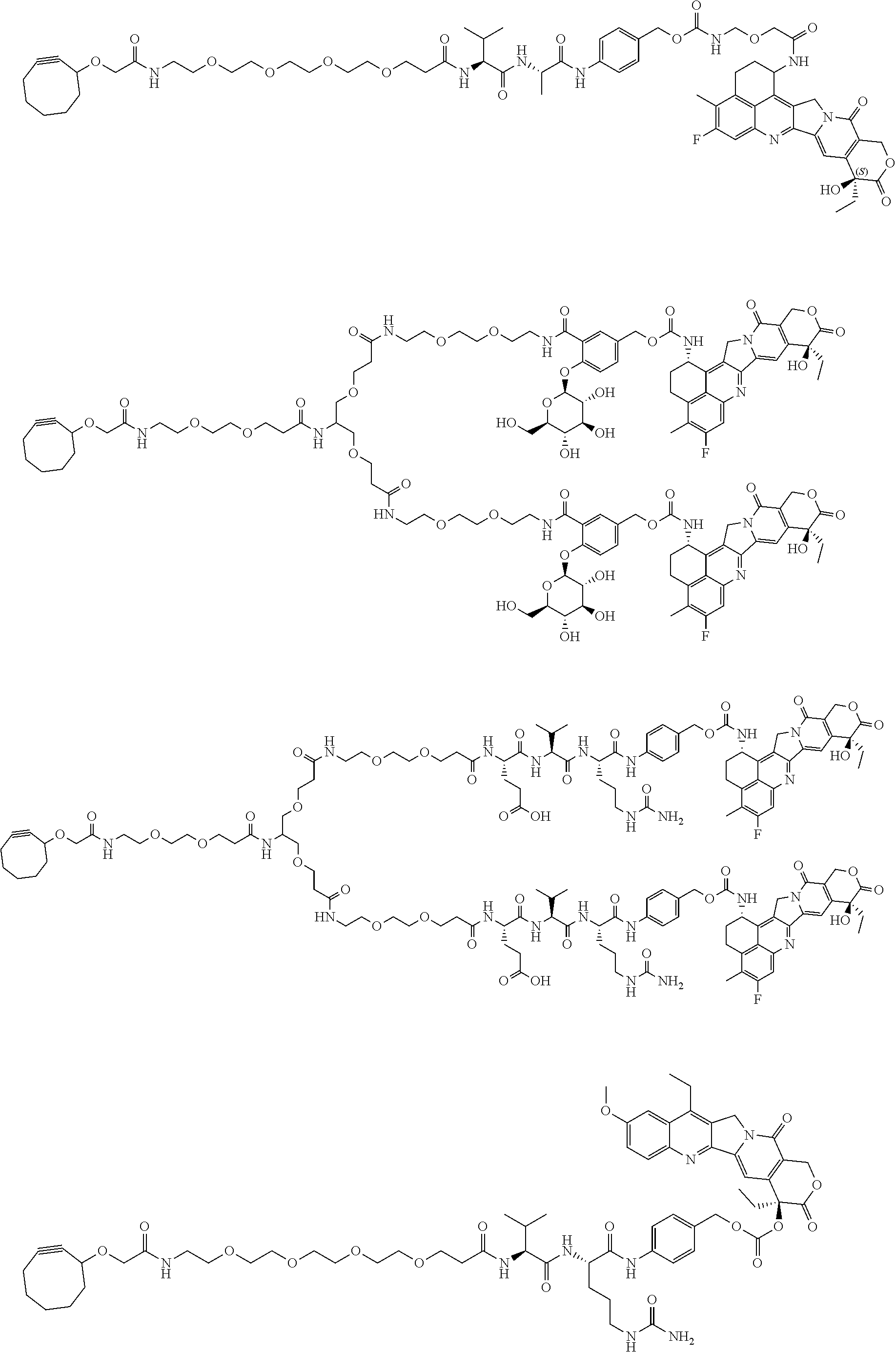 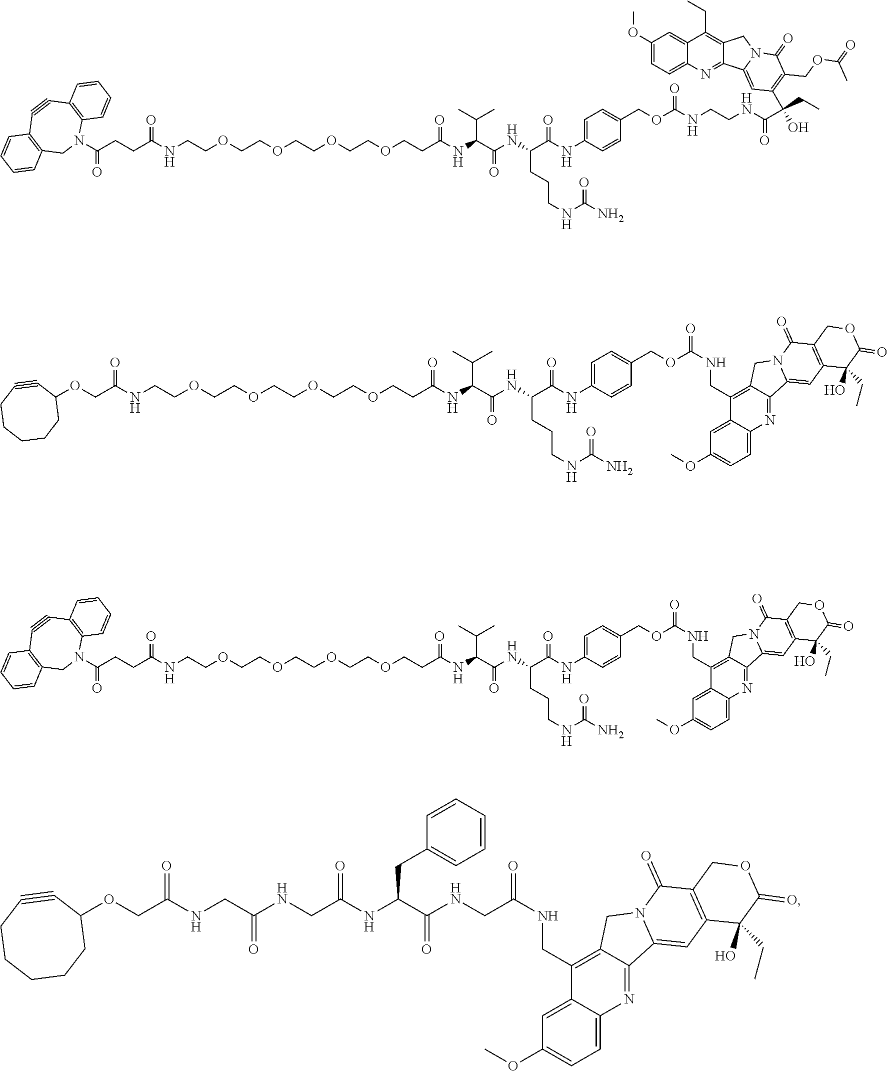
or a pharmaceutically acceptable salt thereof.

30. A composition comprising a population of compounds according to claim 1, having a drug-antibody ratio (DAR) of about 0.5 to about 12.0.

31. The composition of claim 30 having a DAR selected from about 2, about 4 and about 8.

32.-36. (canceled)

37. A compound having a structure selected from the group consisting of: 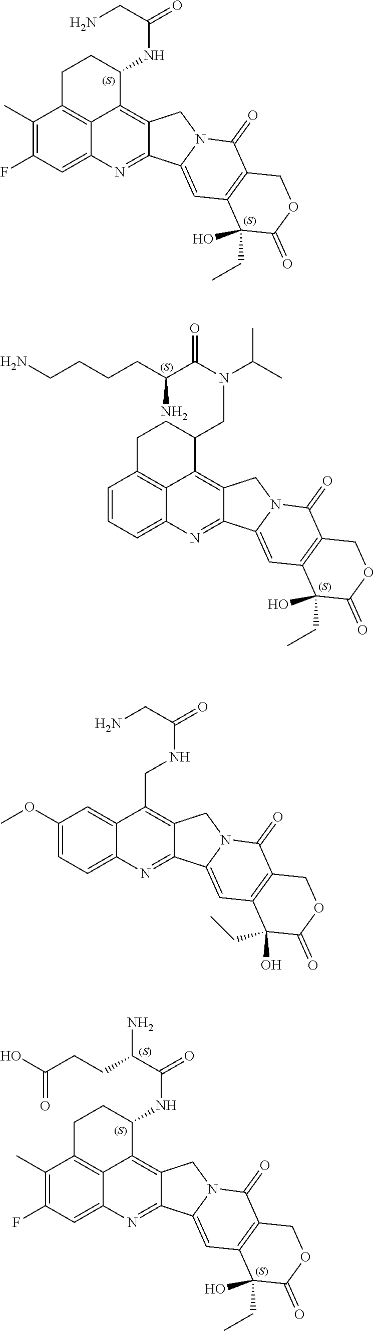 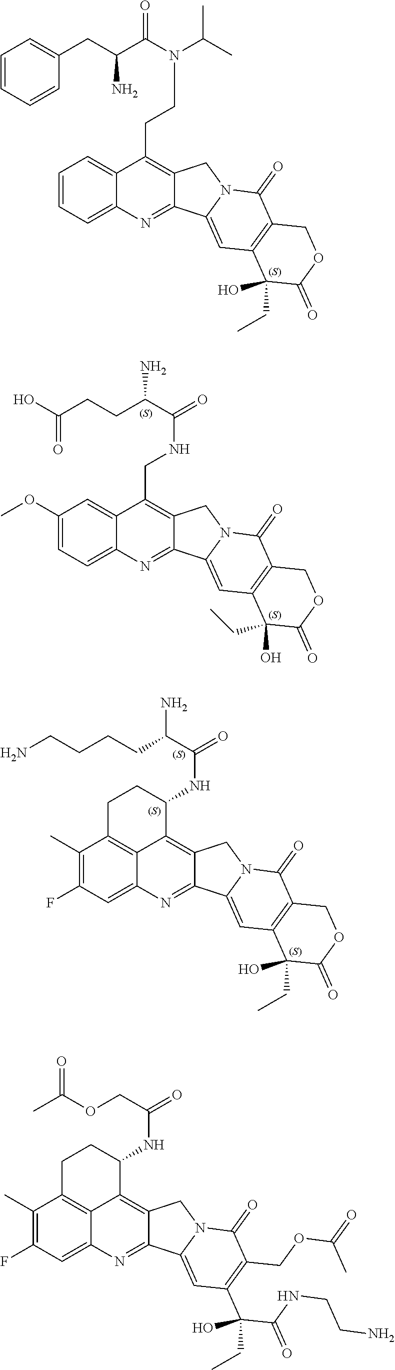 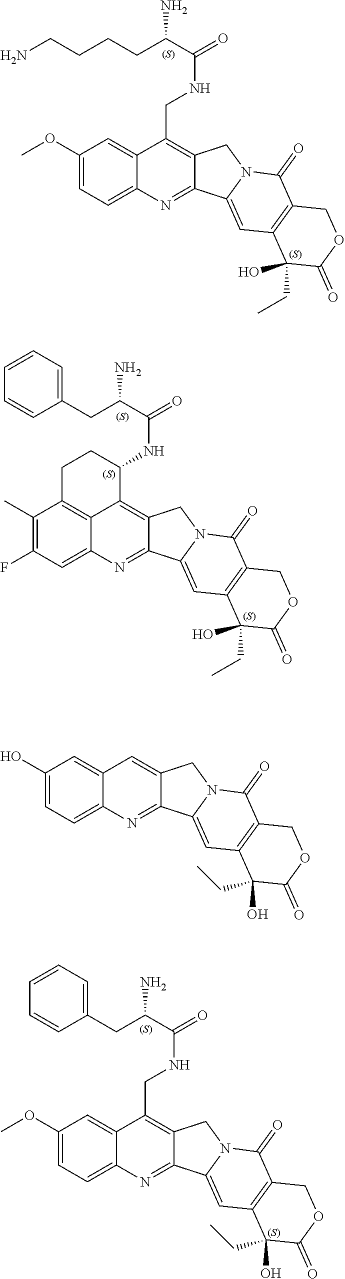 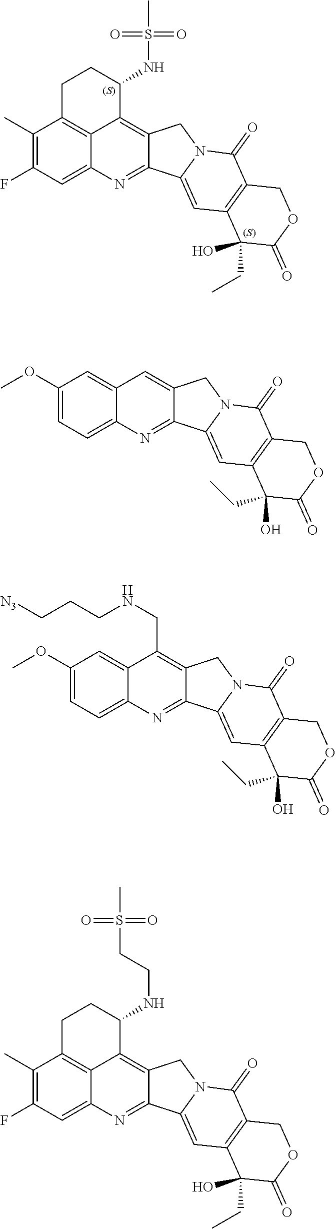 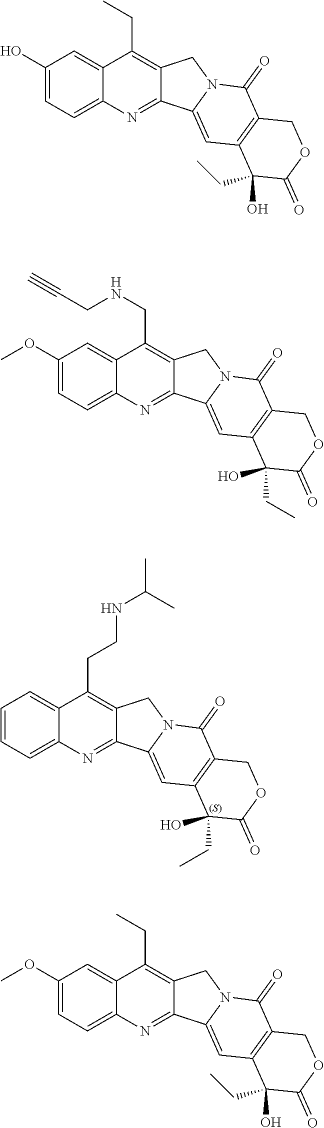 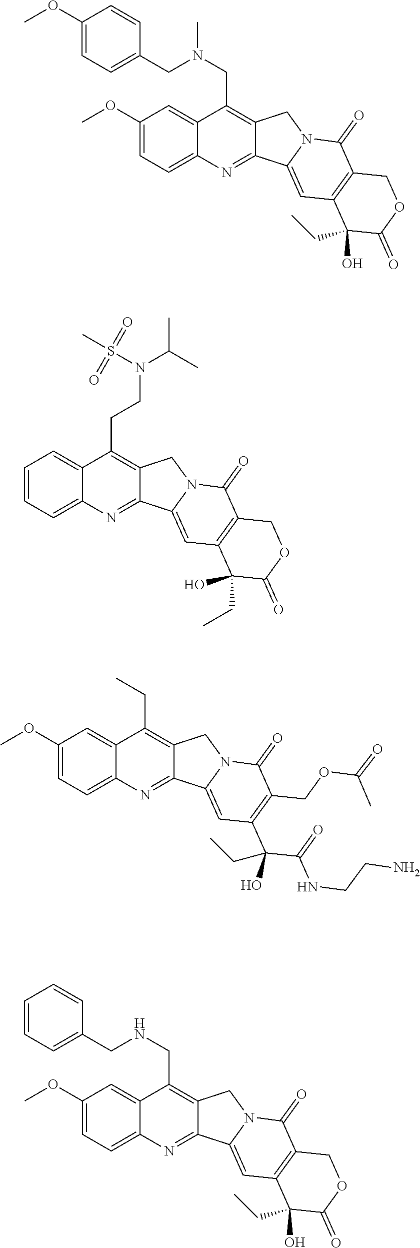 [SMILES: CC[C@@]1(O)C(=O)OCc2c1cc1n(c2=O)Cc2c-1nc1ccc(OC)cc1c2CN(C)Cc1ccc(OC)cc1 ; CC[C@@]1(O)C(=O)OCc2c1cc1n(c2=O)Cc2c-1nc1ccccc1c2CCN(C(C)C)S(C)(=O)=O ; CCc1c2c(nc3ccc(OC)cc13)-c1cc([C@@](O)(CC)C(=O)NCCN)c(COC(C)=O)c(=O)n1C2 ; CC[C@@]1(O)C(=O)OCc2c1cc1n(c2=O)Cc2c-1nc1ccc(OC)cc1c2CNCc1ccccc1] 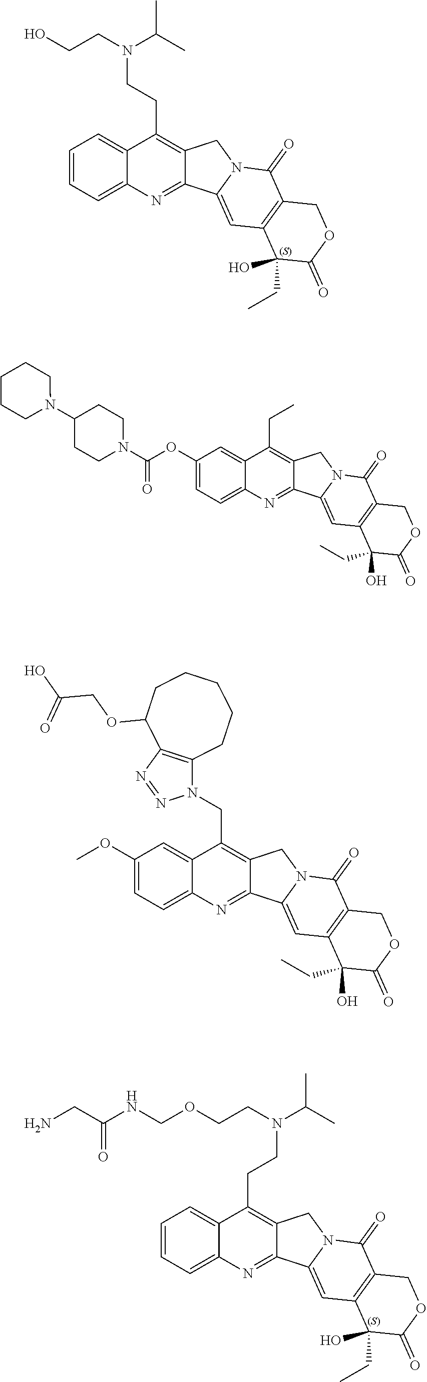 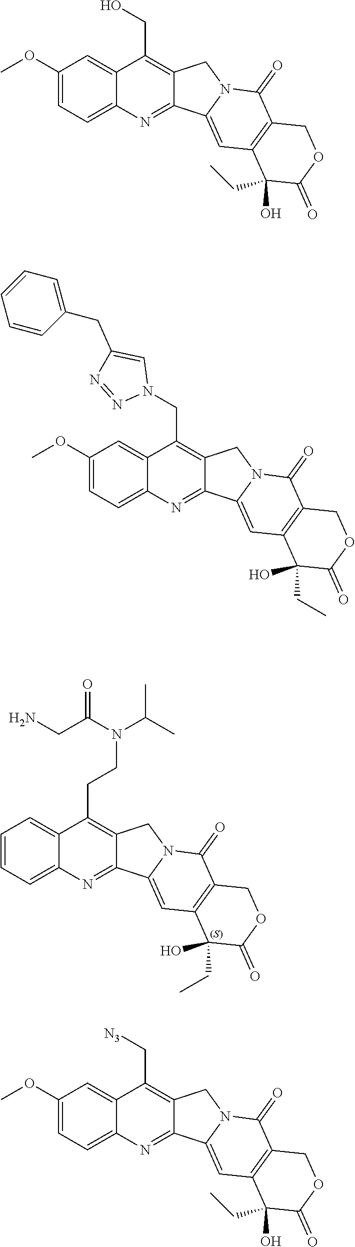 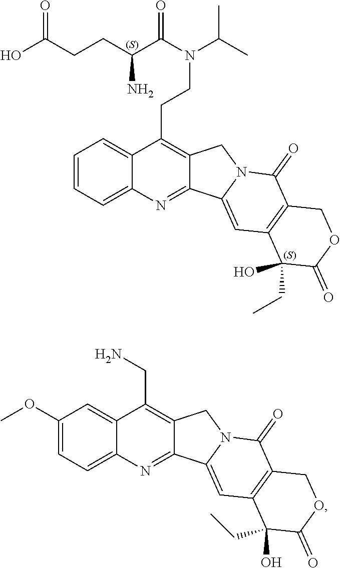
or a pharmaceutically acceptable salt thereof.

38. A pharmaceutical composition comprising the compound according to claim 1, and a diluent, a carrier, and/or an excipient.

39. A method of treating a tumor and/or cancer comprising contacting the tumor and/or cancer with the compound according to claim 37.

40. A method of treating a condition in a subject in need thereof comprising administering to the subject a therapeutically effective amount of the compound according to claim 1, wherein the condition is cancer.

41. (canceled)

42. The method of claim 40, wherein the cancer is selected from the group consisting of breast cancer, ovarian cancer, prostate cancer, lung cancer, liver cancer, or brain cancer.

43. (canceled)

44. A method of selectively delivering a compound into a cell, wherein the compound is according to claim 1.

45. A method of selectively targeting an antigen on a surface of a cell with a compound, wherein the compound is according to claim 1.

46.-49. (canceled)

50. A method of producing a compound having a structure according to Formula (A):
BA-(L1-B-L2-P)ₙ (A),
wherein:
BA is an antibody or an antigen-binding fragment thereof;
L1 is a first linker covalently bound to the side chain of a glutamine residue of the BA;
B is a moiety comprising a triazole;
L2 is a second linker covalently bound to the P;
P is an antitumor agent selected from the group consisting of P-I through P-IV: 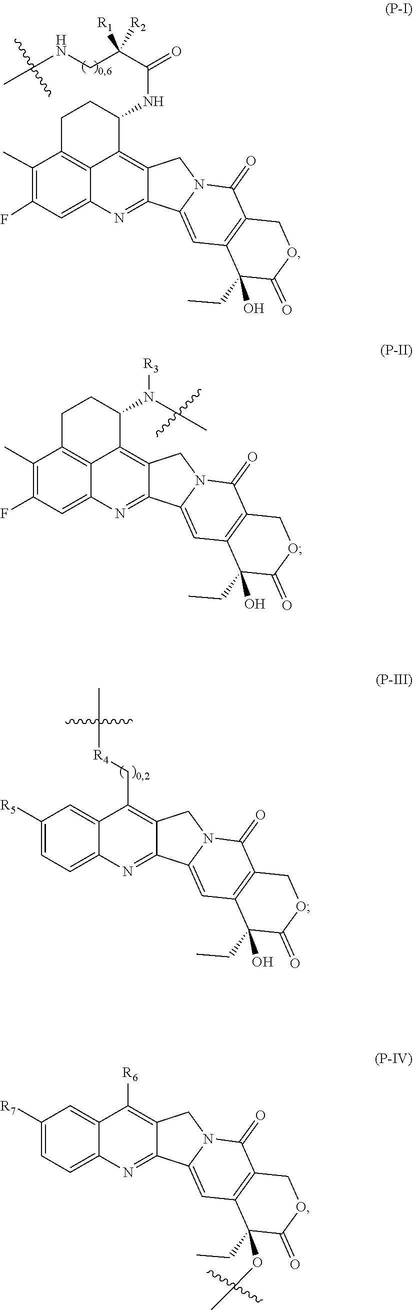
R₁ is hydrogen, C₁₋₆ alkyl, —(CH₂)ᵥ—OH, —(CH₂)ᵥ—NH₂, —(CH₂)ᵥ—C(O)OH, —(CH₂)ᵥ-phenyl, and —(CH₂)ᵥ—SO₂CH₃, wherein v is an integer from 0 to 12;
R₂ is hydrogen, C₁₋₆ alkyl, —(CH₂)ᵥ—OH, —(CH₂)ᵥ—NH₂, —(CH₂)ᵥ—C(O)OH, —(CH₂)ᵥ-phenyl, and —(CH₂)ᵥ—SO₂CH₃, wherein v is an integer from 0 to 12;
R₃ is hydrogen —C₁₋₆ alkyl, —(CH₂)ᵥ—OH, —(CH₂)ᵥ—NH₂, —(CH₂)ᵥ—C(O)OH, —(CH₂)ᵥ-phenyl, —(CH₂)ᵥ—SO₂CH₃, and —CO—(CH₂)ᵥO—COCH₃, wherein v is an integer from 0 to 12;
R₄ is —NH—, —N(—C₁₋₆ alkyl), —N(—C₁₋₆ alkyl)(—SO₂CH₃), —N(—C₁₋₆ alkyl)(—(CH₂)ᵥ—OH), —N(—C₁₋₆ alkyl)(CO—CH₂—NH₂), —N(—C₁₋₆ alkyl)(—(CH₂)ᵥO—CH₂—NH—CO—CH₂—NH₂), —N(—C₁₋₆ alkyl)(—CO—CH(NH₂)—(CH₂)ᵥ—COOH), —N(—C₁₋₆ alkyl)(—CO—CH(NH₂)—(CH₂)ᵥ—NH₂), —N(—C₁₋₆ alkyl)(—CO—CH(NH₂)—(CH₂)ᵥ-phenyl), or 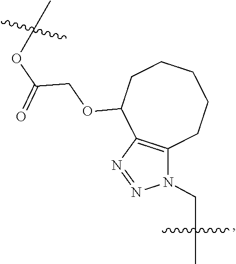
wherein v is an integer from 0 to 12;
R₅ is H, —OH, —OCH₃, or 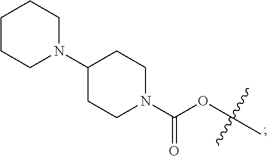 [SMILES: *OC(=O)N1CCC(N2CCCCC2)CC1]
R₆ is hydrogen —C₁₋₆ alkyl, —(CH₂)ᵥ—OH, —(CH₂)ᵥ—NH₂, —(CH₂)-phenyl, —(CH₂)ᵥ—N₃, —(CH₂)ᵥ—NH—CH₂-phenyl, —(CH₂)ᵥ—NMe-CH₂-phenyl-OMe, —(CH₂)ᵥ—NH—(CH₂)ᵥ—N₃, —(CH₂)ᵥ—NH—(CH₂)ᵥ—CCH; —(CH₂)—NH—CO—CH(NH₂)—(CH₂)ᵥ-phenyl, —(CH₂)ᵥ—NH—CO—CH(NH₂)—(CH₂)ᵥ—N₃, —(CH₂)ᵥ—NH—CO—CH(NH₂)—(CH₂)ᵥ—NH₂, —(CH₂)ᵥ—NH—CO—CH(NH₂)—(CH₂)ᵥ—COOH, 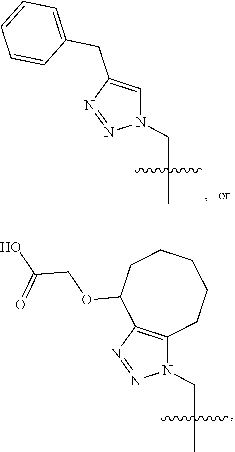
wherein v is an independently an integer from 0 to 12;
R₇ is H, —OH, —OCH₃, or 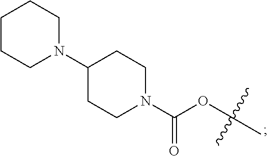 [SMILES: *OC(=O)N1CCC(N2CCCCC2)CC1]
wherein the method comprises the steps of:
wherein the method comprises the steps of:
a) contacting, in the presence of a transglutaminase, the BA comprising at least one glutamine residue with a compound L1-B′,
b) contacting the product of step a) with one or more equivalents of a compound B″-L2-P, wherein the group B″ is capable of covalently attaching to the group B′,
wherein one of the groups B′ and B″ is selected from —N₃ and 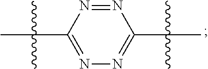 [SMILES: *c1nnc(*)nn1]
and the other of the groups B′ and B″ is selected from 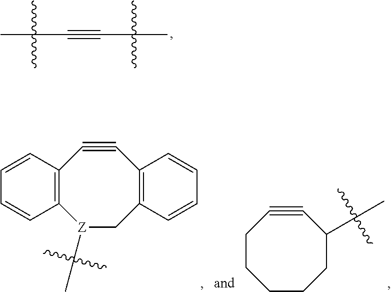
where Z is C or N; and
c) isolating the produced compound of Formula (A).

51. The method according to claim 50, wherein the compound of Formula (A) or has a structure selected from the group consisting of: 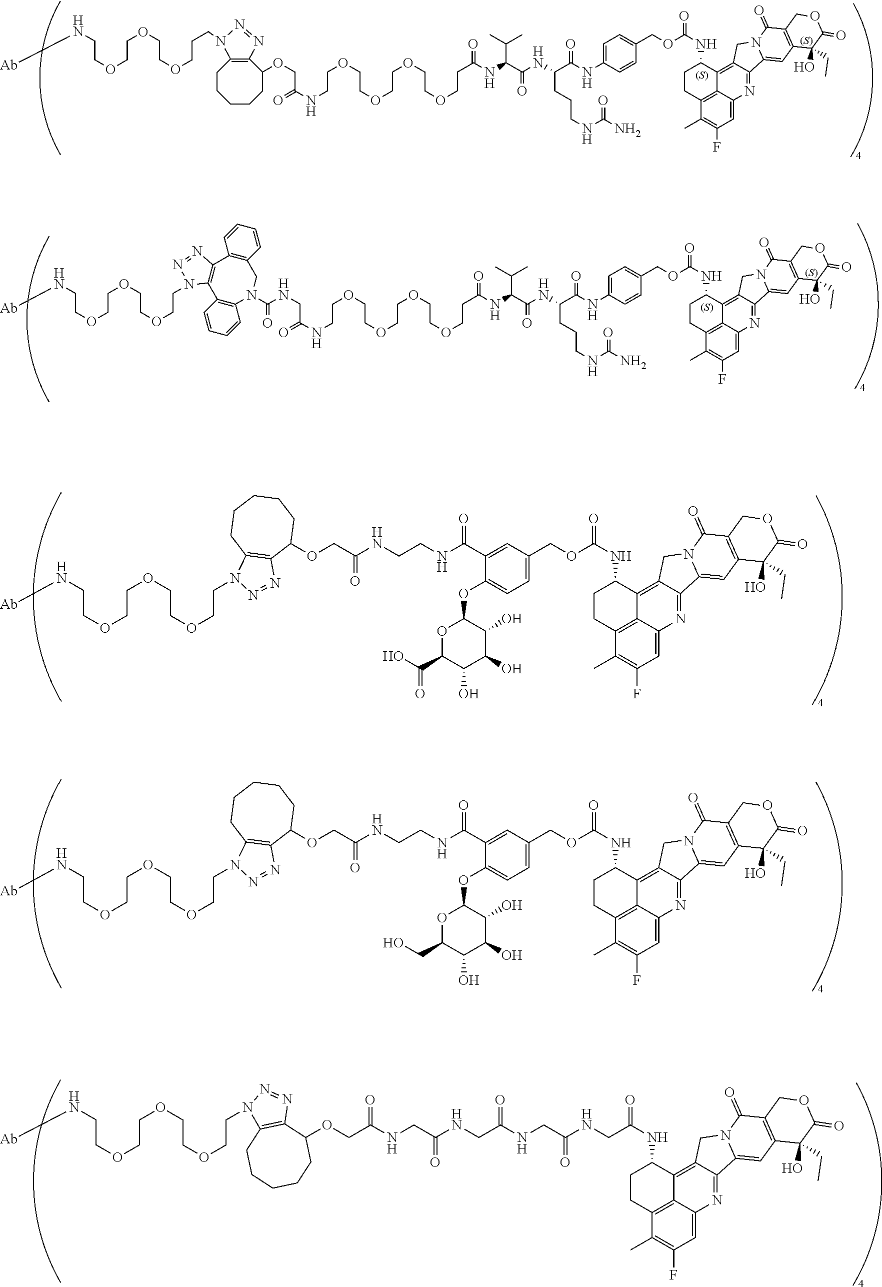 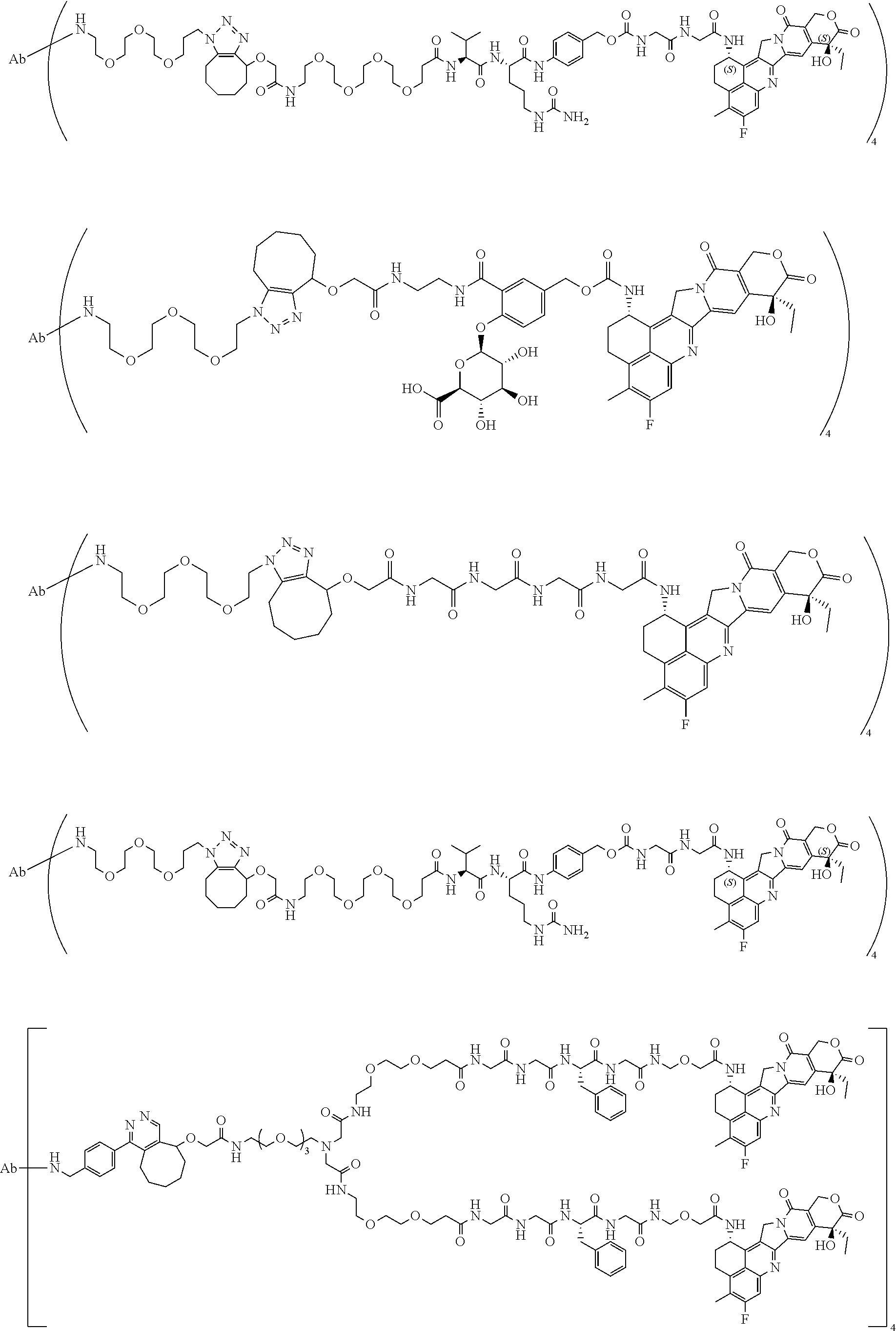 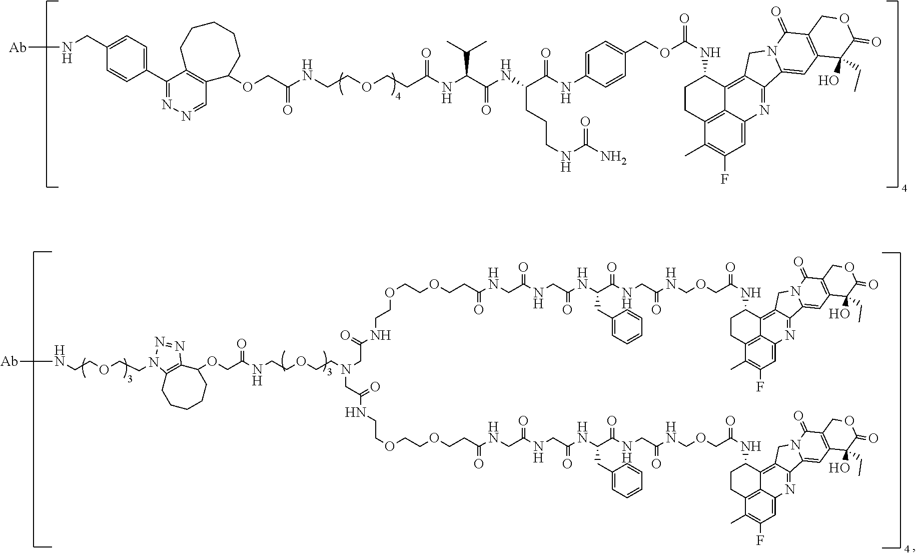
or a pharmaceutically acceptable salt thereof.


SEQUENCE LISTING (23 sequences)

SEQ ID NO: 1 - protein, 5 aa, synthetic construct
LLQGG

SEQ ID NO: 2 - protein, 4 aa, synthetic construct
LLQG

SEQ ID NO: 3 - protein, 6 aa, synthetic construct
LSLSQG

SEQ ID NO: 4 - protein, 8 aa, synthetic construct
GGGLLQGG

SEQ ID NO: 5 - protein, 5 aa, synthetic construct
GLLQG

SEQ ID NO: 6 - protein, 10 aa, synthetic construct
GSPLAQSHGG

SEQ ID NO: 7 - protein, 7 aa, synthetic construct
GLLQGGG

SEQ ID NO: 8 - protein, 6 aa, synthetic construct
GLLQGG

SEQ ID NO: 9 - protein, 4 aa, synthetic construct
GLLQ

SEQ ID NO: 10 - protein, 8 aa, synthetic construct
LLQLLQGA

SEQ ID NO: 11 - protein, 5 aa, synthetic construct
LLQGA

SEQ ID NO: 12 - protein, 7 aa, synthetic construct
LLQYQGA

SEQ ID NO: 13 - protein, 6 aa, synthetic construct
LLQGSG

SEQ ID NO: 14 - protein, 6 aa, synthetic construct
LLQYQG

SEQ ID NO: 15 - protein, 7 aa, synthetic construct
LLQLLQG

SEQ ID NO: 16 - protein, 5 aa, synthetic construct
SLLQG

SEQ ID NO: 17 - protein, 5 aa, synthetic construct
LLQLQ

SEQ ID NO: 18 - protein, 6 aa, synthetic construct
LLQLLQ

SEQ ID NO: 19 - protein, 5 aa, synthetic construct
LLQGR

SEQ ID NO: 20 - protein, 4 aa, synthetic construct
GGGG

SEQ ID NO: 21 - protein, 4 aa, synthetic construct
GGFG

SEQ ID NO: 22 - protein, 6 aa, synthetic construct
HHHHHH

SEQ ID NO: 23 - protein, 14 aa, synthetic construct
  VARIANT 2..13: note = This region may encompass 0-12 residues
GGGGGGGGGGGGGG